(19) United States
(12) Patent Application Publication
(10) Pub. No.: US 2009/0170907 A1
(43) Pub. Date: Jul. 2, 2009

(54) Chemical Compounds

(75) Inventors: Philip Stewart Turnbull, Durham, NC (US); Rodolfo Cadilla, Durham, NC (US); Andrew Lamont Larkin, Durham, NC (US); Eugene Lee Stewart, Durham, NC (US); Katherine Stetson, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 11/916,694

(22) PCT Filed: Jun. 6, 2006
(86) PCT No.: PCT/US2006/021966

§ 371 Date: Jul. 25, 2008

Related U.S. Application Data
(60) Provisional application No. 60/687,895, filed on Jun. 6, 2005.

(51) Int. Cl. A61K 31/44; C07C 255/00; C07D 277/20; C07D 271/10; C07D 271/06; C07D 413/04; A61K 31/445; A61K 31/4245; A61K 31/41

(52) U.S. Cl. 514/340; 558/419; 548/202; 548/143; 548/131; 546/269.4; 546/210; 514/326; 514/364; 514/381

(57) ABSTRACT

This invention relates to non-steroidal compounds that are modulators of androgen, glucocorticoid, mineralocorticoid, and progesterone receptors, and also to the methods for the making and use of such compounds.

### FIELD OF THE INVENTION

This invention relates to non-steroidal compounds that are modulators of androgen, glucocorticoid, mineralocorticoid, and progesterone receptors, and also to the methods for the making and use of such compounds.

### BACKGROUND OF THE INVENTION

Nuclear receptors are a class of structurally related gene expression modulators that act as ligand-dependent transcription factors (R. M. Evans, Science 240, 889 (1988)). The steroid receptors, namely the androgen receptor, the estrogen receptor, the glucocorticoid receptor, the mineralocorticoid receptor, and the progesterone receptor represent a subclass of the nuclear receptor superfamily. Nuclear receptor ligands in this subclass exert their effects by binding to an intracellular steroid hormone receptor. After the receptor-ligand complex is translocated to the nucleus of the cell, the complex binds to recognition sites on DNA, which allows for the modulation of certain genes.

Certain molecules have demonstrated the ability to exhibit their activities in a tissue selective manner. For example, tissue selectivity allows a nuclear receptor ligand to function as an agonist in some tissues, while having no effect or even an antagonist effect in other tissues. The term “selective receptor modulator” (SRM) has been given to these molecules. A synthetic compound that binds to an intracellular receptor and mimics the effects of the native hormone is referred to as an agonist. A compound that inhibits the effect of the native hormone is called an antagonist. The term “modulators” refers to compounds that have a spectrum of activities ranging from full agonism to partial agonism to full antagonism. The molecular basis for this tissue selective activity is not completely understood. Without being limited to any particular explanation, particular ligands put nuclear receptors in different conformational states. These states dictate the ability of coactivators, corepressors, and other proteins to be recruited by the nuclear receptor (NR). The unique cofactor-NR ensembles are the gene transcription factors that are thought to modulate tissue selective effects.

Ligand-mediated effects through the action of nuclear receptors are not limited to the classical genotropic mechanism outlined above. It is thought that some, if not all, of the separation of anabolic and general homeostatic effects from the stimulation of sexual tissues may be explained by a particular ligand's ability to potentiate non-genotropic pathways. One example of liganded nuclear receptor induction of non-genotropic pathways is found in the work of S. C. Manolagas et al., Cell, 104, 719-730. The action of a sex steroid NR on osteoblasts and other cell types is shown to involve the Src/Shc/ERK signaling pathway. This activity is mediated through the ligand binding domain of the sex steroid nuclear receptor alone. The sex steroid NR DNA-binding domain is not required to attenuate etoposide-induced apoptosis in HeLa cells. Typically, an NR lacking its DNA binding domain cannot function as a classic transcription factor.

Nuclear receptor steroid ligands are known to play important roles in the health of both men and women. In regard to men's health, testosterone (T) and dihydrotestosterone (DHT), for example, are endogenous steroidal ligands for the androgen receptor that likely play a role in every tissue type found in the mammalian body. During the development of the fetus, androgens play a role in sexual differentiation and development of male sexual organs. Further sexual development is mediated by androgens during puberty. Androgens play diverse roles in the adult including stimulation and maintenance of male sexual accessory organs and maintenance of the musculoskeletal system. Cognitive function, sexuality, aggression, and mood are some of the behavioral aspects mediated by androgens. Androgens affect the skin, bone, and skeletal muscle, as well as blood lipids and blood cells.

The study of androgen action and male reproductive dysfunction continues to expand significantly. In fact, only recently has the definition of a disease state been associated with hormonal changes that occur in aging men. This syndrome, previously referred to as Andropause, has more recently been described as Androgen Deficiency in the Aging Male, or “ADAM” (A. Morales and J. L. Tenover, Urologic Clinics of North America (2002 Nov.) 29(4) 975.) The onset of ADAM is unpredictable and its manifestations are subtle and variable. Clinical manifestations of ADAM include fatigue, depression, decreased libido, erectile dysfunction as well as changes in cognition and mood.

Published information indicates that androgen replacement therapy (ART) in men may have benefits in terms of improving body composition parameters (e.g. bone mineral density, increasing muscle mass, and strength) as well as improving libido and mood in some men. Therefore, andrologists and other specialists are increasingly using ART for the treatment of the symptoms of ADAM—though there is due caution given androgens', like testosterone, potential side effects. Nonetheless, there is increasing scientific rational of and evidence for androgen deficiency and treatment in the aging male. Current testosterone-based ART therapies include injections, skin patches, gel-based formulations, and oral preparations. All of these therapies are somewhat efficacious in the treatment of ADAM, but, due to the dramatic fluctuations in plasma T-levels following treatment, success with these therapies has been variable.

Testosterone replacement products, such as AndroGel® (1% testosterone gel CIII, marketed by Solvay Pharmaceuticals) are emerging as a treatment of choice among physicians. Such products, however, fail to correctly mimic physiological testosterone levels and have potential side effects including exacerbation of pre-existing sleep apnea, polycythemia, and/or gynecomastia. Furthermore, the longer-term side effects on target organs such as the prostate or the cardiovascular system are yet to be fully elucidated. Importantly, the potential carcinogenic effects of testosterone on the prostate prevent many physicians from prescribing it to older men (i.e. age>60 years) who, ironically, stand to benefit most from treatment. Also, all of the existing treatment options have fundamental problems with their delivery mechanism. The need for a novel selective androgen receptor modulator (SARM) is obviated by the potential side effect profile manifested in conventional treatments. A SARM would ideally have all the beneficial effects of endogenous androgens, while sparing sexual accessory organs, specifically the prostate.

In regard to female health, progesterone, the endogenous ligand for the progesterone receptor (“PR”), plays an important role in female reproduction during the various stages of the ovarian cycle and during pregnancy. Among other things, progesterone prepares the endometrium for implantation, regulates the implantation process, and helps maintain pregnancy. The therapeutic use of synthetic versions of progesterone (progestins) stems from progesterone's ability to regulate endometrial proliferation. In fact, progestins are included as part of hormone replacement therapy (“HRT”) in women to reduce the incidence of endometriosis. Unfortunately, the effectiveness of therapy is tempered by undesired side-effect profiles. Chronic progestin therapy or continuous estrogen replacement regimens are often associated with increased bleeding. Excessive stimulatory effects on the endometrial vasculature may result in proliferation and fragility.

Compounds that modulate the effects of progesterone binding to PR are believed useful in the treatment and/or prophylaxis of endometriosis and uterine fibroid processes. Progesterone receptor antagonists such as mifepristone, also known as RU-486, and other PR modulators may inhibit endometrial proliferation at high estradiol concentrations in primates. Human clinical data with mifepristone supports the efficacy of a PR antagonist in endometriosis (D. R. Grow et. al., J. Clin. Endocrin. Metab. 1996, 81). Despite enthusiasm for its use, RU-486 also acts as a potent ligand for the glucocorticoid receptor (“GR”). This cross-reactivity with the GR is associated with homeostatic imbalances.

Thus, modulators of steroid hormone nuclear receptors that are highly specific for one receptor could offer greater benefit with less side effects in the treatment of both female and male related hormone responsive diseases.

### SUMMARY OF INVENTION

Briefly, in one aspect, the present invention provides compounds of formula (I)

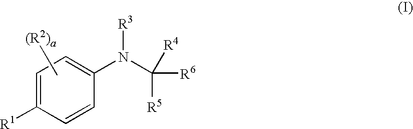

or a salt or solvate thereof, wherein
R¹ is CN, NO₂, or halogen;
a is 0, 1, or 2;
each R² independently is cyano, nitro, halogen, haloalkyl, alkyl, alkenyl, alkynyl, hydroxy, alkoxy, haloalkoxy, or aryl;
R³ is —(R^{x})_{f}R⁷;
f is 0 or 1;
R^{x} is a C₁-C₄ alkylene chain that may be further optionally substituted with one or more alkyl;
R⁷ is H, alkyl, haloalkyl, cycloalkyl, alkenyl, alkynyl, or cyano;
each R⁴ and R⁵ independently is H, alkyl, cycloalkyl, or haloalkyl;
R⁶ is aryl, or heterocyclyl, wherein

(i) when R⁶ is aryl, said aryl is optionally substituted with one or more alkyl, halogen, haloalkyl, alkoxy, haloalkoxy, haloalkylthio, heterocyclyl, cyano, alkylsulfonyl, alkoxycarbonyl, nitro, amide, aryl, aryloxy, substituted aryl or substituted aryloxy, where said substituted aryl or substituted aryloxy is substituted with one or more halogen, and

(ii) when R⁶ is heterocyclyl, said heterocyclyl may be optionally substituted with one or more alkyl, cycloalkylalkyl, halogen, haloalkyl, alkoxy, haloalkoxyalkyl, halogen substituted arylsulfonylalkyl, alkoxycarbonyl, oxo, cyano, cyanoalkyl, hydroxyalkyl, amide, amino, aminoalkyl, alkylsulfonyl, aralkoxyalkyl, acyl, heterocyclylcarbonyl, alkoxyalkyl, alkylthioalkyl, aralkyl, heterocyclylalkyl, heterocyclyl, aryl, R⁸, substituted aryl, or substituted heterocyclyl, where said substituted aryl or substituted heterocyclyl is substituted with one or more alkyl, halogen, haloalkyl, cyano, alkoxy, amino, nitro, alkylthio, alkylsulfonyl, alkylsulfoxide, haloalkoxy, alkoxycarbonyl, amide, acyl, heterocyclylcarbonyl, aminosulfonyl, or —NHC(O)CH₃, or

(iii) R⁴ and R⁶ combine with the carbon from which they are substituted, to form a 6- to 12-membered bicyclic ring, which ring may optionally contain one or more heteroatoms selected from O, S, and N;

R⁸ is —(CH₂)ₙ—(R⁹)—(R¹⁰);
n is 0, 1 or 2;

### R⁹ is —C(O)—, —S(O)₂—, —NHC(O)—, —NHC(O)NHC(O)—, or —NHS(O)₂—; and

R¹⁰ is NH₂, CH₃, alkoxy, or aryl, wherein said aryl may optionally be substituted with alkyl or halogen.

Another aspect of the present invention provides a compound substantially as hereinbefore defined with reference to any one of the Examples.

Another aspect of the present invention provides a pharmaceutical composition comprising a compound of the present invention.

Another aspect of the present invention provides a compound of the present invention for use as an active therapeutic substance.

Another aspect of the present invention provides a compound of the present invention for use in the treatment of conditions or disorders that respond to selective androgen receptor modulation.

Another aspect of the present invention provides a compound of the present invention for use in the treatment of osteoporosis, muscle wasting, frailty, cardiovascular disease, breast cancer, uterine cancer, prostate hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM.

Another aspect of the present invention provides the use of a compound of the present invention in the manufacture of a medicament for use in the treatment of conditions or disorders that respond to selective androgen receptor modulation.

Another aspect of the present invention provides the use of a compound of the present invention in the manufacture of a medicament for use in the treatment of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM.

Another aspect of the present invention provides a method for the treatment of conditions or disorders that respond to selective androgen receptor modulation comprising the administration of a compound of the present invention.

Another aspect of the present invention provides a method for the treatment of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM comprising the administration of a compound of the present invention.

### DETAILED DESCRIPTION OF THE PREFERRED EMBODIMENT

Terms are used within their accepted meanings. The following definitions are meant to clarify, but not limit, the terms defined.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, and n-pentyl.

As used throughout this specification, the preferred number of atoms, such as carbon atoms, will be represented by, for example, the phrase “Cₓ₋C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl and the like.

As used herein the term “alkynyl” refers to a straight or branched chain aliphatic hydrocarbon containing one or more carbon-to-carbon triple bonds. Examples include, but are not limited to, ethynyl and the like.

As used herein, the term “alkylene” refers to a straight or branched chain divalent hydrocarbon radical, preferably having from one to ten carbon atoms. Examples of “alkylene” as used herein include, but are not limited to, methylene (—CH₂—), ethylene (—CH₂—CH₂—), and branched versions thereof such as (—CH(CH₃)—) and the like.

As used herein, the term “acyl” refers to the group —C(O)Rₐ, where Rₐ is alkyl, or aryl, as defined herein.

As used herein, the term “cycloalkyl” refers to a non-aromatic cyclic hydrocarbon ring. Exemplary “cycloalkyl” groups include, but are not limited to, cyclopropyl, cyclobutyl, cyclopentyl, cyclohexyl, cycloheptyl, and the like. As used herein, the term also includes such groups fused with a benzene ring or other aromatic hydrocarbon ring.

As used herein, the term “heterocycle” or “heterocyclyl” refers to a mono- or poly-cyclic ring system containing one or more heteroatoms and optionally containing one or more degrees of unsaturation, including monocyclic five to seven membered aromatic ring, or a fused bicyclic aromatic ring system comprising two of such aromatic rings. Preferred heteroatoms include N, O, and/or S, where N-oxides, sulfur oxides, and dioxides are permissible heteroatom substitutions. Preferably the ring is three to ten-membered. Such rings may be optionally fused to one or more of another “heterocyclic” ring(s), aryl ring(s), or cycloalkyl ring(s). Examples of “heterocyclic” groups include, but are not limited to, tetrahydrofuran, pyran, piperidine, pyrrolidine, pyrrolidinone, morpholine, tetrahydrothiopyran, tetrahydrothiophene, furan, thiophene, pyrrole, imidazole, pyrazole, triazole, tetrazole, thiazole, oxazole, isoxazole, oxadiazole, thiadiazole, isothiazole, pyridine, piperidine, pyridazine, pyrazine, pyrimidine, quinoline, isoquinoline, benzofuran, benzimidazole, benzothiazole, benzothiophene, indole, indazole, and the like. Preferred heterocyclyl groups include pyridyl, piperidyl, furyl, tetrazolyl, benzofuranyl, thiophenyl, imidazolyl, triazolyl, oxazolyl, isoxazolyl, benzothiazolyl, benzothiophenyl, benzimidazolyl, oxadiazolyl, and thiazolyl.

As used herein, the term “aryl” refers to a benzene ring or to a fused benzene ring system, for example anthracene, phenanthrene, or naphthalene ring systems. Examples of “aryl” groups include, but are not limited to, phenyl, 2-naphthyl, 1-naphthyl, biphenyl, and the like. One preferred aryl group is phenyl.

As used herein the term “aralkyl” refers to a group —RₐR_{b}, where Rₐ is an alkylene group and R_{b} is an aryl group as each is herein defined.

As used herein, the term “arylsulfonylalkyl” refers to a group —RₐR_{b}R_{c} where Rₐ is an alkylene group, R_{b} is a sulfonyl group, and R_{c} is an aryl group, as each is defined herein.

As used herein the term “heterocyclylalkyl” refers to a group —RₐR_{b}, where Rₐ is an alkylene group and R_{b} is a heterocyclyl group as each is herein defined.

As used herein the term “halogen” refers to fluorine, chlorine, bromine, or iodine.

As used herein the term “haloalkyl” refers to an alkyl group, as defined herein that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents such as —CF₃, —CH₂—CH₂—F, —CH₂—CF₃, and the like.

As used herein the term “hydroxy” or “hydroxyl” refers to a group —OH.

As used herein the term “hydroxyalkyl” refers to an alkyl group, as defined herein, which is substituted with a hydroxyl group. Examples include —CH₂—OH, —C(CH₃)₂—OH, and the like.

As used herein the term “hydroxyhaloalkyl” refers to a haloalkyl group, as defined herein, which is substituted with a hydroxyl group. Examples include —C(CF₃)₂—OH and the like.

As used herein the term “mercapto” refers to a group —SH.

As used herein, the term “oxo” refers to a group ═O

As used herein the term “alkoxy” refers to a group —ORₐ, where Rₐ is alkyl as herein defined.

As used herein the term “aryloxy” refers to a group —ORₐ, where Rₐ is aryl as herein defined.

As used herein the term “haloalkoxy” refers to a group —ORₐ, where Rₐ is haloalkyl as defined herein.

As used herein the term “alkylthio” refers to a group —SRₐ, where Rₐ is alkyl as herein defined.

As used herein the term “haloalkylthio” refers to a group —SRₐ, where Rₐ is haloalkyl as defined herein.

As used herein the term “alkoxyalkyl” refers to a group —Rₐ—O—R_{b}, where Rₐ is an alkylene group and R_{b} is an alkyl group as each is herein defined.

As used herein the term “aralkoxyalkyl” refers to a group —Rₐ—O—R_{b}, where Rₐ is an alkylene group and R_{b} is an aralkyl group as each is herein defined.

As used herein the term “cycloalkylalkyl” refers to a group —Rₐ—R_{b}, where Rₐ is an alkylene group and R_{b} is a cycloalkyl group as each is herein defined.

As used herein, the term “haloalkoxyalkyl” refers to a group —Rₐ—O—R_{b}, where Rₐ is an alkylene group and R_{b} is a haloalkyl group as each is herein defined.

As used herein the term “alkylthioalkyl” refers to a group —Rₐ—S—R_{b}, where Rₐ is an alkylene group and R_{b} is an alkyl group as each is herein defined.

As used herein, the term “heterocyclylcarbonyl” refers to a group —C(O)—Rₐ, where Rₐ is a heterocyclyl group as herein defined.

As used herein, the term “alkoxycarbonyl” refers to a group —C(O)—O—Rₐ, where Rₐ is an alkyl group as herein defined.

As used herein the term “alkylsulfonyl” refers to a group —SO₂Rₐ, where Rₐ is an alkyl group as herein defined.

As used herein the term “aminosulfonyl” refers to a group —SO₂Rₐ, where Rₐ is an amino group as herein defined.

As used herein the term “nitro” refers to a group —NO₂.

As used herein the term “cyano” refers to a group —CN.

As used herein the term “cyanoalkyl” refers to a group —RₐCN, where Rₐ is an alkylene group as herein defined.

As used herein the term “amino” refers to a group —NH₂, and also refers to a group —N(Rₐ)(R_{b}), where one or both of Rₐ and R_{b} are other than H. For example, the term includes groups such as —N(CH₃)(CH₃), —N(CH₃)(CH₂—CH₃), and the like.

As used herein the term “aminoalkyl” refers to a group —Rₐ—R_{b}, where Rₐ is an alkylene group and R_{b} is an amino group as each is herein defined.

As used herein the term “amide” refers to a group —C(O)NH₂, and also refers to a group —C(O)N(Rₐ)(R_{b}), where one or both of Rₐ and R_{b} are other than H. For example, the term includes groups such as —C(O)N(CH₃)(CH₃), —C(O)N(CH₃)(CH₂—CH₃), and the like.

As used herein throughout the present specification, the phrase “optionally substituted” or variations thereof denote an optional substitution, including multiple degrees of substitution, with one or more substituent group, preferably one or two. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

In one embodiment, the present invention provides compounds of formula (I-A)

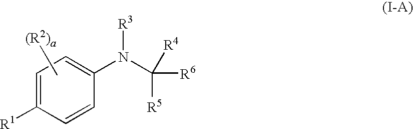

or a salt or solvate thereof, wherein
R¹ is CN, NO₂, or halogen;
a is 0, 1, or 2;
each R² independently is cyano, nitro, halogen, haloalkyl, alkyl, alkenyl, alkynyl, hydroxy, alkoxy, haloalkoxy, or aryl;

### R³ is —(R^{x})f R⁷;

f is 0 or 1;
R^{x} is a C₁-C₄ alkylene chain that may be further optionally substituted with one or more alkyl;
R⁷ is H, alkyl, haloalkyl, cycloalkyl, alkenyl, alkynyl, or cyano;
each R⁴ and R⁵ independently is H, alkyl, cycloalkyl, or haloalkyl;
R⁶ is aryl, or heterocyclyl, wherein

(i) when R⁶ is aryl, said aryl is optionally substituted with one or more alkyl, halogen, haloalkyl, alkoxy, haloalkoxy, haloalkylthio, heterocyclyl, cyano, alkylsulfonyl, alkoxycarbonyl, nitro, amide, aryl, aryloxy, substituted aryl or substituted aryloxy, where said substituted aryl or substituted aryloxy is substituted with one or more halogen, and

(ii) when R⁶ is heterocyclyl, said heterocyclyl may be optionally substituted with one or more alkyl, cycloalkylalkyl, halogen, haloalkyl, alkoxy, haloalkoxyalkyl, halogen substituted arylsulfonylalkyl, alkoxycarbonyl, oxo, cyano, cyanoalkyl, hydroxyalkyl, amide, amino, aminoalkyl, alkylsulfonyl, aralkoxyalkyl, acyl, heterocyclylcarbonyl, alkoxyalkyl, alkylthioalkyl, aralkyl, heteroaralkyl, heterocyclyl, aryl, R⁸, substituted aryl, or substituted heterocyclyl, where said substituted aryl or substituted heterocyclyl is substituted with one or more alkyl, halogen, haloalkyl, cyano, alkoxy, amino, nitro, alkylthio, alkylsulfonyl, alkylsulfoxide, haloalkoxy, alkoxycarbonyl, amide, acyl, heterocyclylcarbonyl, aminosulfonyl, or —NHC(O)CH₃;

R⁸ is —(CH₂)ₙ—(R⁹)—(R¹¹);
n is 0, 1 or 2;

### R⁹ is —C(O)—, —S(O)₂—, —NHC(O)—, —NHC(O)NHC(O)—, or —NHS(O)₂—; and

R¹⁰ is NH₂, CH₃, alkoxy, or aryl, wherein said aryl may optionally be substituted with alkyl or halogen.

In one embodiment, the present invention provides compounds of formula (I) wherein wherein R¹ is cyano.

In another embodiment, a is 1, and R² is C₁-C₆ haloalkyl, cyano, or chloro.

In one embodiment, R² is —CF₃.

In another embodiment, f is 1, R^{x} is a C₁-C₂ alkylene chain, and R⁷ is C₁-C₆ haloalkyl, C₃-C₆ cycloalkyl, or C₁-C₆ alkyl.

In one embodiment, R⁷ is cyclopropyl or —CF₃.

Preferably, R⁷ is —CF₃.

In one embodiment, each of R⁴ and R⁵ are H.

In one embodiment, R⁶ is aryl, or heterocyclyl.

In another embodiment, R⁶ is aryl, optionally substituted with one or more halogen, C₁-C₆ haloalkyl, or cyano.

In one embodiment, R⁶ is furyl, thiazolyl, oxazolyl, oxadiazolyl, tetrazolyl, triazolyl, thiophenyl, benzofuranyl, isoxazolyl, benzothiazolyl, or benzimidazolyl, each independently optionally substituted with one or more cyano, alkyl, haloalkyl, aryl, or heterocyclyl.

In one embodiment, R⁶ is furyl, thiazolyl, oxazolyl, oxadiazolyl, tetrazolyl, triazolyl, thiophenyl, benzofuranyl, isoxazolyl, benzothiazolyl, imidazolyl, benzothiophenyl, or benzimidazolyl, each independently optionally substituted with one or more cyano, alkyl, haloalkyl, aryl, halogen, or heterocyclyl.

In another embodiment, R⁶ is furyl, thiazolyl, oxazolyl, oxadiazolyl, or tetrazolyl, each independently optionally substituted with one or more cyano, alkyl, haloalkyl, aryl, or heterocyclyl.

In another embodiment, R⁶ is furyl, thiazolyl, oxazolyl, oxadiazolyl, or tetrazolyl, each independently optionally substituted with one or more cyano, alkyl, haloalkyl, aryl, halogen, or heterocyclyl.

In another embodiment, R⁶ is furyl, thiazolyl, oxazolyl, oxadiazolyl, or tetrazolyl, optionally substituted with phenyl, which is optionally substituted with one or more halogen or haloalkyl.

In another embodiment, R⁶ is furyl, thiazolyl, oxazolyl, oxadiazolyl, or tetrazolyl, optionally substituted with phenyl, which is optionally substituted with one or more cyano, halogen or haloalkyl.

In another embodiment, R⁶ is furyl, thiazolyl, oxazolyl, oxadiazolyl, or tetrazolyl, each independently optionally substituted with pyridyl, isoxazolyl, furyl, thiophenyl, pyrazolyl, or pyridazyl, of which each is independently optionally substituted with one or more halogen or haloalkyl.

In another embodiment, R⁶ is furyl, thiazolyl, oxazolyl, oxadiazolyl, or tetrazolyl, each independently optionally substituted with piperidyl, which is optionally substituted with aminosulfonyl.

In another embodiment, the present invention provides compounds of formula (I-B)

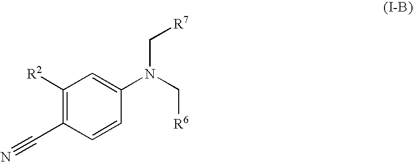

or a salt or solvate thereof, wherein
R² is —CF₃, cyano, or chloro;
R⁷ is —CF₃ or cyclopropyl;
R⁶ is aryl or heterocyclyl, wherein

(i) when R⁶ is aryl, said aryl may optionally be substituted with cyano, and

(ii) when R⁶ is heterocyclyl, said heterocyclyl may optionally be substituted with alkyl, cyano, haloalkyl, heterocyclyl, substituted aryl, or substituted heterocyclyl, wherein said substituted aryl or substituted heterocyclyl is substituted with halogen, haloalkyl, or aminosulfonyl.

In another embodiment, the present invention provides a compound selected from:

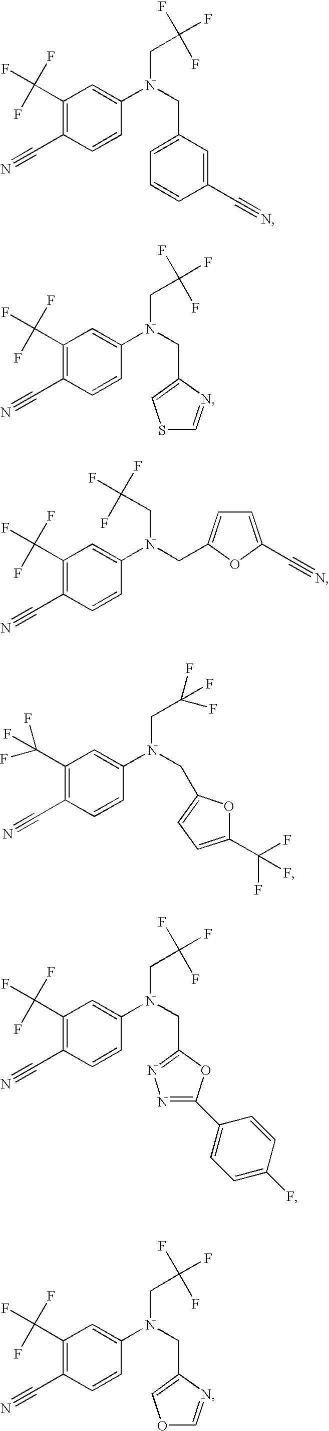

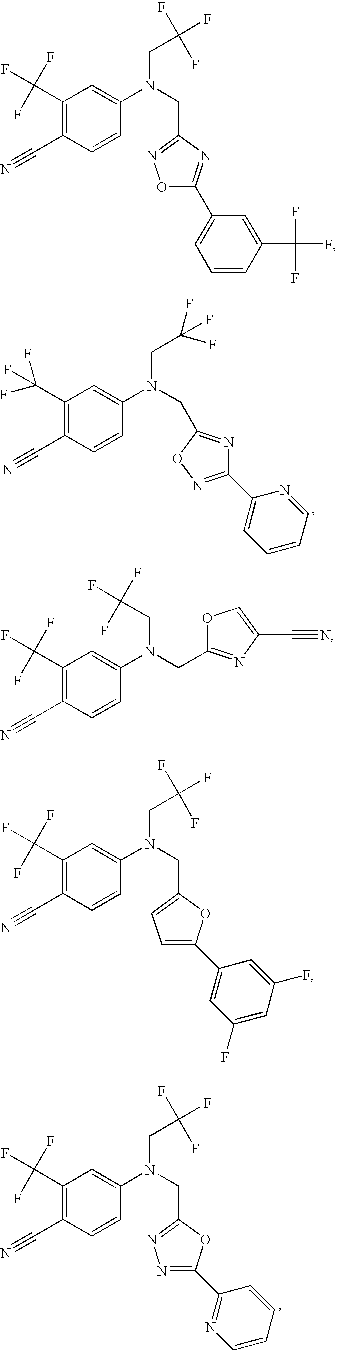

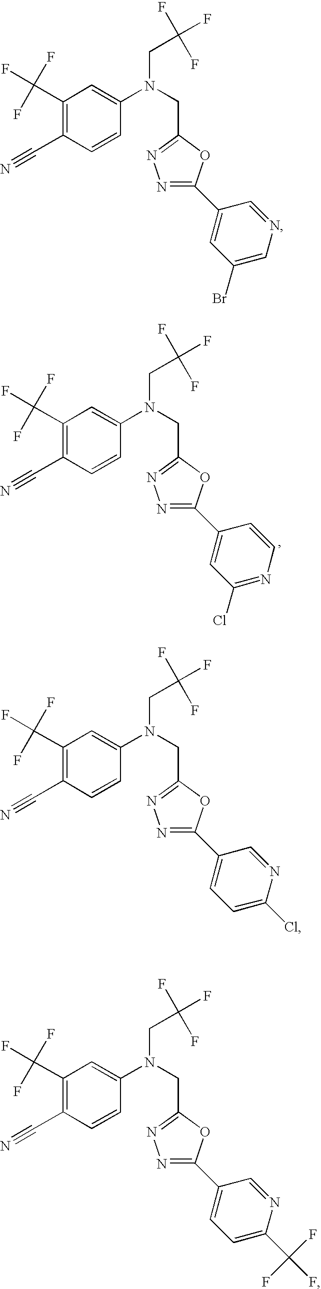

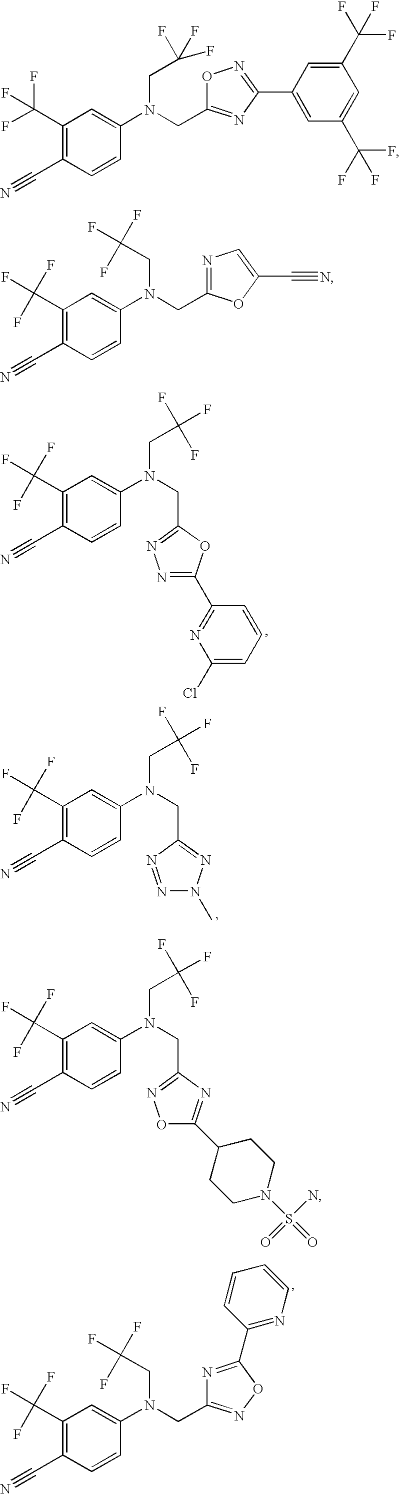

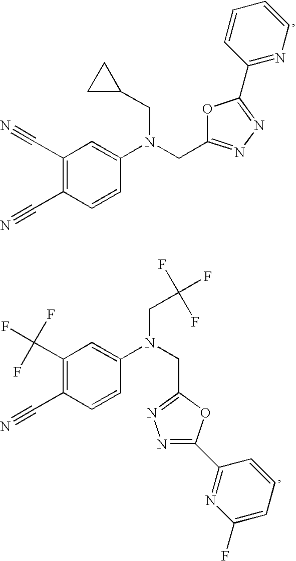

or a salt or solvate thereof.

While the embodiments or preferred groups for each variable have generally been listed above separately for each variable, compounds of this invention include those in which several of each variable in Formula (I) are selected from the embodiments or preferred groups for each variable. Therefore, this invention is intended to include all combinations of embodiments and preferred groups.

The compounds of the present invention are believed to modulate the function of one or more nuclear hormone receptor(s). Particularly, the compounds of the present invention modulate the androgen receptor (“AR”). The present invention includes compounds that are selective agonists, partial agonists, antagonists, or partial antagonists of the AR. Compounds of the present invention are useful in the treatment of AR-associated diseases and conditions, for example, a disease or condition that is prevented, alleviated, or cured through the modulation of the function or activity of AR. Such modulation may be isolated within certain tissues or widespread throughout the body of the subject being treated.

As used herein, the term “treatment” refers to alleviating the specified condition, eliminating or reducing the symptoms of the condition, slowing or eliminating the progression of the condition and preventing or delaying the initial occurrence of the condition in a subject, or reoccurrence of the condition in a previously afflicted subject.

One embodiment of the present invention is the use of the compounds of the present invention for the treatment of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myalgia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstrual syndrome, contraception, uterine fibroid disease, aortic smooth muscle cell proliferation, male hormone replacement, or ADAM.

The compounds of the present invention may crystallize in more than one form, a characteristic known as polymorphism, and such polymorphic forms (“polymorphs”) are within the scope of the present invention. Polymorphism generally may occur as a response to changes in temperature, pressure, or both. Polymorphism may also result from variations in the crystallization process. Polymorphs may be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of the present invention) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The biological or medical response may be considered a prophylactic response or a treatment response. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of the present invention may be administered as the raw chemical. Additionally, the active ingredient may be presented as a pharmaceutical composition.

Accordingly, the invention further provides pharmaceutical compositions that include effective amounts of compounds of the present invention and one or more pharmaceutically acceptable carriers, diluents, or excipients. The compounds of the present invention are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the present invention with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal the actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the present invention, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component may be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents may also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol may be added to the mixture before the encapsulation. A disintegrating or solubilizing agent such as agar-agar, calcium carbonate or sodium carbonate may also be added to improve the availability of the medicament when the capsule is ingested. Moreover, when desired or necessary, suitable binders, lubricants, disintegrating agents, and coloring agents may also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets may be formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture may be wet-granulated with a binder such as syrup, starch paste, acadia mucilage or solutions of cellulosic or polymeric materials, and forcing through a screen. As an alternative to granulating, the powder mixture may be run through the tablet machine and the result is imperfectly formed slugs broken into granules. The granules may be lubricated to prevent sticking to the tablet forming dies by means of the addition of stearic acid, a stearate salt, talc or mineral oil. The lubricated mixture is then compressed into tablets. The compounds of the present invention may also be combined with a free flowing inert carrier and compressed into tablets directly without going through the granulating or slugging steps. A clear or opaque protective coating consisting of a sealing coat of shellac, a coating of sugar or polymeric material, and a polish coating of wax may be provided. Dyestuffs may be added to these coatings to distinguish different unit dosages.

Oral fluids such as solutions, syrups, and elixirs may be prepared in dosage unit form so that a given quantity contains a predetermined amount of the compound. Syrups may be prepared, for example, by dissolving the compound in a suitably flavored aqueous solution, while elixirs may be prepared through the use of a non-toxic alcoholic vehicle. Suspensions may be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like may also be added.

Where appropriate, dosage unit formulations for oral administration may be microencapsulated. The formulation may also be prepared to prolong or sustain the release as for example by coating or embedding particulate material in polymers, wax or the like.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

The compounds of the present invention may also be delivered by the use of monoclonal antibodies as individual carriers to which the compound molecules are coupled.

The compounds may also be coupled with soluble polymers as targetable drug carriers. Such polymers may include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents for the treatment of the above-mentioned conditions. For example, in frailty therapy, combination may be had with other anabolic or osteoporosis therapeutic agents. As one example, osteoporosis combination therapies according to the present invention would thus comprise the administration of at least one compound of the present invention and the use of at least one other osteoporosis therapy. As a further example, combination therapies according to the present invention include the administration of at least one compound of the present invention and at least one other osteoporosis treatment agent, for example, an anti-bone resorption agent. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

Another potential osteoporosis treatment agent is a bone building (anabolic) agent. Bone building agents may lead to increases in parameters such as bone mineral density that are greater than those than may be achieved with anti-resorptive agents. In some cases, such anabolic agents may increase trabecular connectivity leading to greater structural integrity of the bone.

Other potential therapeutic combinations include the compounds of the present invention combined with other compounds of the present invention, growth promoting agents, growth hormone secretagogues, growth hormone releasing factor and its analogs, growth hormone and its analogs, somatomedins, alpha-adrenergic agonists, serotonin 5-HT_{D} agonists, agents that inhibit somatostatin or its release, 5-α-reductase inhibitors, aromatase inhibitors, GnRH agonists or antagonists, parathyroid hormone, bisphosphonates, estrogen, testosterone, SERMs, progesterone receptor agonists or antagonists, and/or with other modulators of nuclear hormone receptors.

One skilled in the art will acknowledge that although the compounds embodied herein will be used as selective agonists, partial agonists, and antagonists, compounds with mixed steroid activities may also be employed.

The compounds of the present invention may be used in the treatment of a variety of disorders and conditions and, as such, the compounds of the present invention may be used in combination with a variety of other suitable therapeutic agents useful in the treatment of those disorders or conditions. Non-limiting examples include combinations of the present invention with anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

An aspect of the present invention is the use of the compounds of the present invention for the treatment of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myalgia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstrual syndrome, contraception, uterine fibroid disease, aortic smooth muscle cell proliferation, male hormone replacement, or ADAM.

In particular, the compounds of the present invention are believed useful, either alone or in combination with other agents, in the treatment of and use as male and female hormone replacement therapy, hypogonadism, osteoporosis, muscle wasting, wasting diseases, cancer cachexia, frailty, prostatic hyperplasia, prostate cancer, breast cancer, menopausal and andropausal vasomotor conditions, urinary incontinence, sexual dysfunction, erectile dysfunction, depression, uterine fibroid disease, and/or endometriosis, treatment of acne, hirsutism, stimulation of hematopoiesis, male contraception, impotence, and as anabolic agents.

Another aspect of the present invention thus also provides compounds of the present invention for use in medical therapy. Particularly, the present invention provides for the treatment of disorders mediated by androgenic activity. More particularly, the present invention provides through the treatment of disorders responsive to tissue-selective anabolic and or androgenic activity. A further aspect of the invention provides a method of treatment of a mammal suffering from a disorder mediated by androgenic activity, which includes administering to said subject an effective amount of a compound of the present invention.

A further aspect of the invention provides a method of treatment of a mammal requiring the treatment of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myalgia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstrual syndrome, contraception, uterine fibroid disease, aortic smooth muscle cell proliferation, male hormone replacement, or ADAM. Preferably the compounds of the present invention are used as male and female hormone replacement therapy or for the treatment or prevention of hypogonadism, osteoporosis, muscle wasting, wasting diseases, cancer cachexia, frailty, prostatic hyperplasia, prostate cancer, breast cancer, menopausal and andropausal vasomotor conditions, urinary incontinence, sexual dysfunction, erectile dysfunction, depression, uterine fibroid disease, and/or endometriosis, treatment of acne, hirsutism, stimulation of hematopoiesis, male contraception, impotence, and as anabolic agents, which use includes administering to a subject an effective amount of a compound of the present invention. The mammal requiring treatment with a compound of the present invention is typically a human being.

The compounds of this invention may be made by a variety of methods, including well-known standard synthetic methods. Illustrative general synthetic methods are set out below and then specific compounds of the invention are prepared in the working Examples.

In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists in compounds of the present invention. Accordingly, the present invention includes all possible stereoisomers and includes not only racemic compounds but the individual enantiomers as well. When a compound is desired as a single enantiomer, such may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

Representative AR modulator compounds, agonists, partial agonists, and antagonists according to the current invention include:
4-{(Cyclopropylmethyl)[(1R)-1-(2-naphthyl)ethyl]amino}-2-(trifluoromethyl)benzonitrile;
4-{[(1R)-1-Phenylpropyl]amino}-2-(trifluoromethyl)benzonitrile;
4-{(Cyclopropylmethyl)[(1S)-2,3-dihydro-1H-inden-1-yl]amino}-2-(trifluoromethyl)benzonitrile;
N-[(1R)-1-(4-Bromophenyl)ethyl]-4-nitro-3-(trifluoromethyl)aniline;
4-{(Cyclopropylmethyl)[(1R)-2,3-dihydro-1H-inden-1-yl]amino}-2-(trifluoromethyl)benzonitrile;
4-[Benzyl(1-cyclopropylethyl)amino]-2-(trifluoromethyl)benzonitrile;
4-{Allyl[(1R)-1-(4-bromophenyl)ethyl]amino}-2-(trifluoromethyl)benzonitrile;
N-Allyl-N-[(1R)-1-(4-bromophenyl)ethyl]-4-nitro-3-(trifluoromethyl)aniline;
4-{Allyl[(1S)-2,3-dihydro-1H-inden-1-yl]amino}-2-(trifluoromethyl)benzonitrile;
4-[Benzyl(2,2,2-trifluoroethyl)amino]-2-(trifluoromethyl)benzonitrile;
4-[(2-Methylbenzyl)(2,2,2-trifluoroethyl)amino]-2-(trifluoromethyl)benzonitrile;
4-{(2,2,2-Trifluoroethyl)[2-(trifluoromethyl)benzyl]amino}-2-(trifluoromethyl)benzonitrile;
4-[(2-Fluorobenzyl)(2,2,2-trifluoroethyl)amino]-2-(trifluoromethyl)benzonitrile;
4-[(3-Methylbenzyl)(2,2,2-trifluoroethyl)amino]-2-(trifluoromethyl)benzonitrile;
4-{(2,2,2-Trifluoroethyl)[3-(trifluoromethyl)benzyl]amino}-2-(trifluoromethyl)benzonitrile;
4-[(3-Fluorobenzyl)(2,2,2-trifluoroethyl)amino]-2-(trifluoromethyl)benzonitrile;
4-[(3-Chlorobenzyl)(2,2,2-trifluoroethyl)amino]-2-(trifluoromethyl)benzonitrile;
4-[(3-Bromobenzyl)(2,2,2-trifluoroethyl)amino]-2-(trifluoromethyl)benzonitrile;
4-[(1,1′-Biphenyl-3-ylmethyl)(2,2,2-trifluoroethyl)amino]-2-(trifluoromethyl)benzonitrile;
4-[(3-Methoxybenzyl)(2,2,2-trifluoroethyl)amino]-2-(trifluoromethyl)benzonitrile;
4-{(2,2,2-Trifluoroethyl)[3-(trifluoromethoxy)benzyl]amino}-2-(trifluoromethyl)benzonitrile;
4-[[3-(4-Fluorophenoxy)benzyl](2,2,2-trifluoroethyl)amino]-2-(trifluoromethyl)benzonitrile;
4-[[3-(2-Fluorophenoxy)benzyl](2,2,2-trifluoroethyl)amino]-2-(trifluoromethyl)benzonitrile;
4-((2,2,2-Trifluoroethyl){3-[(trifluoromethyl)thio]benzyl}amino)-2-(trifluoromethyl)benzonitrile;
4-[(3-Cyanobenzyl)(2,2,2-trifluoroethyl)amino]-2-(trifluoromethyl)benzonitrile;
Methyl 3-{[[4-cyano-3-(trifluoromethyl)phenyl](2,2,2-trifluoroethyl)amino]methyl}benzoate;
3-{[[4-Cyano-3-(trifluoromethyl)phenyl](2,2,2-trifluoroethyl)amino]methyl}benzamide;
4-[(3-Nitrobenzyl)(2,2,2-trifluoroethyl)amino]-2-(trifluoromethyl)benzonitrile;
4-[(4-Methylbenzyl)(2,2,2-trifluoroethyl)amino]-2-(trifluoromethyl)benzonitrile;
4-[(4-tert-Butylbenzyl)(2,2,2-trifluoroethyl)amino]-2-(trifluoromethyl)benzonitrile;
4-{(2,2,2-Trifluoroethyl)[4-(trifluoromethyl)benzyl]amino}-2-(trifluoromethyl)benzonitrile;
4-[(4-Fluorobenzyl)(2,2,2-trifluoroethyl)amino]-2-(trifluoromethyl)benzonitrile;
4-[(4-Methoxybenzyl)(2,2,2-trifluoroethyl)amino]-2-(trifluoromethyl)benzonitrile;
4-{(2,2,2-Trifluoroethyl)[4-(trifluoromethoxy)benzyl]amino}-2-(trifluoromethyl)benzonitrile;
4-[[4-(Methylsulfonyl)benzyl](2,2,2-trifluoroethyl)amino]-2-(trifluoromethyl)benzonitrile;
4-[(4-Cyanobenzyl)(2,2,2-trifluoroethyl)amino]-2-(trifluoromethyl)benzonitrile;
Methyl 4-{[[4-cyano-3-(trifluoromethyl)phenyl](2,2,2-trifluoroethyl)amino]methyl}benzoate;
4-[(4-Nitrobenzyl)(2,2,2-trifluoroethyl)amino]-2-(trifluoromethyl)benzonitrile;
4-[(3,5-Dimethylbenzyl)(2,2,2-trifluoroethyl)amino]-2-(trifluoromethyl)benzonitrile;
4-[[3,5-bis(Trifluoromethyl)benzyl](2,2,2-trifluoroethyl)amino]-2-(trifluoromethyl)benzonitrile;
4-[(3,5-Dimethoxybenzyl)(2,2,2-trifluoroethyl)amino]-2-(trifluoromethyl)benzonitrile;
4-[(3,5-Dichlorobenzyl)(2,2,2-trifluoroethyl)amino]-2-(trifluoromethyl)benzonitrile;
4-[(3,5-Difluorobenzyl)(2,2,2-trifluoroethyl)amino]-2-(trifluoromethyl)benzonitrile;
4-[(2,6-Difluorobenzyl)(2,2,2-trifluoroethyl)amino]-2-(trifluoromethyl)benzonitrile;
4-[(2,4-Difluorobenzyl)(2,2,2-trifluoroethyl)amino]-2-(trifluoromethyl)benzonitrile;
4-[(3,4-Difluorobenzyl)(2,2,2-trifluoroethyl)amino]-2-(trifluoromethyl)benzonitrile;
4-[(2,3-Difluorobenzyl)(2,2,2-trifluoroethyl)amino]-2-(trifluoromethyl)benzonitrile;
4-[[3-Fluoro-4-(trifluoromethyl)benzyl](2,2,2-trifluoroethyl)amino]-2-(trifluoromethyl)benzonitrile;
4-[(2-Naphthylmethyl)(2,2,2-trifluoroethyl)amino]-2-(trifluoromethyl)benzonitrile;
4-[(1-Phenylethyl)(2,2,2-trifluoroethyl)amino]-2-(trifluoromethyl)benzonitrile;
4-[(3-Furylmethyl)(2,2,2-trifluoroethyl)amino]-2-(trifluoromethyl)benzonitrile;
4-[(2-Furylmethyl)(2,2,2-trifluoroethyl)amino]-2-(trifluoromethyl)benzonitrile;
Ethyl 5-{[[4-cyano-3-(trifluoromethyl)phenyl](2,2,2-trifluoroethyl)amino]methyl}-2-furoate;
5-{[[4-Cyano-3-(trifluoromethyl)phenyl](2,2,2-trifluoroethyl)amino]methyl}-2-furamide;
5-{[[4-Cyano-3-(trifluoromethyl)phenyl] (2,2,2-trifluoroethyl)amino]methyl}-2-furonitrile;
4-[[(5-Methyl-2-furyl)methyl](2,2,2-trifluoroethyl)amino]-2-(trifluoromethyl)benzonitrile;
4-((2,2,2-Trifluoroethyl){[5-(trifluoromethyl)-2-furyl]methyl}amino)-2-(trifluoromethyl)benzonitrile;
4-[{[5-(Cyanomethyl)-2-furyl]methyl}(2,2,2-trifluoroethyl)amino]-2-(trifluoromethyl)benzonitrile;
4-[{[5-(Methoxymethyl)-2-furyl]methyl}(2,2,2-trifluoroethyl)amino]-2-(trifluoromethyl)benzonitrile;
4-[({5-[(2,2,2-Trifluoroethoxy)methyl]-2-furyl}methyl)(2,2,2-trifluoroethyl)amino]-2-(trifluoromethyl)benzonitrile;
4-[({5-[(Methylthio)methyl]-2-furyl}methyl)(2,2,2-trifluoroethyl)amino]-2-(trifluoromethyl)benzonitrile;
4-[({5-[(Dimethylamino)methyl]-2-furyl}methyl)(2,2,2-trifluoroethyl)amino]-2-(trifluoromethyl)benzonitrile;
4-[{[5-(1H-Imidazol-1-ylmethyl)-2-furyl]methyl}(2,2,2-trifluoroethyl)amino]-2-(trifluoromethyl)benzonitrile;
4-[{[5-(1H-1,2,4-Triazol-1-ylmethyl)-2-furyl]methyl}(2,2,2-trifluoroethyl)amino]-2-(trifluoromethyl)benzonitrile;
4-[{[5-(2-Cyanoethyl)-2-furyl]methyl}(2,2,2-trifluoroethyl)amino]-2-(trifluoromethyl)benzonitrile;
4-[{[5-(3,5-Difluorophenyl)-2-furanyl]methyl}(2,2,2-trifluoroethyl)amino]-2-(trifluoromethyl)benzonitrile;
Methyl 2-{[[4-cyano-3-(trifluoromethyl)phenyl](2,2,2-trifluoroethyl)amino]methyl}-3-furoate;
2-{[[4-Cyano-3-(trifluoromethyl)phenyl](2,2,2-trifluoroethyl)amino]methyl}-3-furamide;
2-{[[4-Cyano-3-(trifluoromethyl)phenyl](2,2,2-trifluoroethyl)amino]methyl}-3-furonitrile;
4-[{[3-(Hydroxymethyl)-2-furyl]methyl}(2,2,2-trifluoroethyl)amino]-2-(trifluoromethyl)benzonitrile;
4-[[(3-Methyl-2-furyl)methyl](2,2,2-trifluoroethyl)amino]-2-(trifluoromethyl)benzonitrile;
4-[(1-Benzofuran-2-ylmethyl)(2,2,2-trifluoroethyl)amino]-2-(trifluoromethyl)benzonitrile;
4-[[(5-Chlorothien-2-yl)methyl](2,2,2-trifluoroethyl)amino]-2-(trifluoromethyl)benzonitrile;
4-[(Thien-2-ylmethyl)(2,2,2-trifluoroethyl)amino]-2-(trifluoromethyl)benzonitrile;
4-[[(5-Bromothien-2-yl)methyl](2,2,2-trifluoroethyl)amino]-2-(trifluoromethyl)benzonitrile;
5-{[[4-Cyano-3-(trifluoromethyl)phenyl](2,2,2-trifluoroethyl)amino]methyl}thiophene-2-carbonitrile;
4-[{[5-(4-Fluorophenyl)thien-2-yl]methyl}(2,2,2-trifluoroethyl)amino]-2-(trifluoromethyl)benzonitrile;
4-[{[5-(4-Cyanophenyl)thien-2-yl]methyl}(2,2,2-trifluoroethyl)amino]-2-(trifluoromethyl)benzonitrile;
4-[(Thien-3-ylmethyl)(2,2,2-trifluoroethyl)amino]-2-(trifluoromethyl)benzonitrile;
4-[[(5-Chloro-1-benzothien-3-yl)methyl](2,2,2-trifluoroethyl)amino]-2-(trifluoromethyl)benzonitrile;
4-[(1H-Imidazol-2-ylmethyl)(2,2,2-trifluoroethyl)amino]-2-(trifluoromethyl)benzonitrile;
4-[(1,3-Oxazol-2-ylmethyl)(2,2,2-trifluoroethyl)amino]-2-(trifluoromethyl)benzonitrile;
4-[(1,3-Oxazol-4-ylmethyl)(2,2,2-trifluoroethyl)amino]-2-(trifluoromethyl)benzonitrile;
2-{[[4-Cyano-3-(trifluoromethyl)phenyl](2,2,2-trifluoroethyl)amino]methyl}-1,3-oxazole-4-carbonitrile;
2-{[[4-Cyano-3-(trifluoromethyl)phenyl](2,2,2-trifluoroethyl)amino]methyl}-1,3-oxazole-5-carbonitrile;
4-[[(3,5-Dimethylisoxazol-4-yl)methyl](2,2,2-trifluoroethyl)amino]-2-(trifluoromethyl)benzonitrile;
4-[[(5-Methylisoxazol-3-yl)methyl](2,2,2-trifluoroethyl)amino]-2-(trifluoromethyl)benzonitrile;
4-[(Cyclopropylmethyl)(1,3-thiazol-4-ylmethyl)amino]-2-(trifluoromethyl)benzonitrile;
4-[(1,3-Thiazol-4-ylmethyl)(2,2,2-trifluoroethyl)amino]-2-(trifluoromethyl)benzonitrile;
4-[[(2-Methyl-1,3-thiazol-4-yl)methyl](2,2,2-trifluoroethyl)amino]-2-(trifluoromethyl)benzonitrile;
4-[[(2-Phenyl-1,3-thiazol-4-yl)methyl](2,2,2-trifluoroethyl)amino]-2-(trifluoromethyl)benzonitrile;
4-[(2,2,2-Trifluoroethyl)({2-[4-(trifluoromethyl)phenyl]-1,3-thiazol-4-yl}methyl)amino]-2-(trifluoromethyl)benzonitrile;
4-[[(2-Thien-2-yl-1,3-thiazol-4-yl)methyl](2,2,2-trifluoroethyl)amino]-2-(trifluoromethyl)benzonitrile;
4-[(1,3-Thiazol-2-ylmethyl)(2,2,2-trifluoroethyl)amino]-2-(trifluoromethyl)benzonitrile;
4-[[1-(1,3-Thiazol-2-yl)ethyl](2,2,2-trifluoroethyl)amino]-2-(trifluoromethyl)benzonitrile;
4-[[(5-tert-Butyl-1,2,4-oxadiazol-3-yl)methyl](2,2,2-trifluoroethyl)amino]-2-(trifluoromethyl)benzonitrile;
4-[[(5-Phenyl-1,2,4-oxadiazol-3-yl)methyl](2,2,2-trifluoroethyl)amino]-2-(trifluoromethyl)benzonitrile;
4-[{[5-(2-Methoxyphenyl)-1,2,4-oxadiazol-3-yl]methyl}(2,2,2-trifluoroethyl)amino]-2-(trifluoromethyl)benzonitrile;
4-[(2,2,2-Trifluoroethyl)({5-[3-(trifluoromethyl)phenyl]-1,2,4-oxadiazol-3-yl}methyl)amino]-2-(trifluoromethyl)benzonitrile;
4-(3-{[[4-Cyano-3-(trifluoromethyl)phenyl](2,2,2-trifluoroethyl)amino]methyl}-1,2,4-oxadiazol-5-yl)-1-piperidinesulfonamide;
4-[{[5-(2-Pyridinyl)-1,2,4-oxadiazol-3-yl]methyl}(2,2,2-trifluoroethyl)amino]-2-(trifluoromethyl)benzonitrile;
4-[{[3-(4-Fluorophenyl)-1,2,4-oxadiazol-5-yl]methyl}(2,2,2-trifluoroethyl)amino]-2-(trifluoromethyl)benzonitrile;
4-[{[3-(2-Pyridinyl)-1,2,4-oxadiazol-5-yl]methyl}(2,2,2-trifluoroethyl)amino]-2-(trifluoromethyl)benzonitrile;
4-[({3-[3,5-Bis(trifluoromethyl)phenyl]-1,2,4-oxadiazol-5-yl}methyl)(2,2,2-trifluoroethyl)amino]-2-(trifluoromethyl)benzonitrile;
4-[{[5-(4-Methylphenyl)-1,3,4-oxadiazol-2-yl]methyl}(2,2,2-trifluoroethyl)amino]-2-(trifluoromethyl)benzonitrile;
4-[{[5-(2-Pyridinyl)-1,3,4-oxadiazol-2-yl]methyl}(2,2,2-trifluoroethyl)amino]-2-(trifluoromethyl)benzonitrile;
4-[{[5-(5-Bromo-3-pyridinyl)-1,3,4-oxadiazol-2-yl]methyl}(2,2,2-trifluoroethyl)amino]-2-(trifluoromethyl)benzonitrile;
4-[{[5-(2-Chloro-4-pyridinyl)-1,3,4-oxadiazol-2-yl]methyl}(2,2,2-trifluoroethyl)amino]-2-(trifluoromethyl)benzonitrile;
4-[{[5-(6-Chloro-3-pyridinyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[6-(trifluoromethyl)-3-pyridinyl]-1,3,4-oxadiazol-2-yl}methyl)amino]-2-(trifluoromethyl)benzonitrile;
4-[{[5-(6-Chloro-2-pyridinyl)-1,3,4-oxadiazol-2-yl]methyl}(2,2,2-trifluoroethyl)amino]-2-(trifluoromethyl)benzonitrile;
4-[{[5-(6-Fluoro-2-pyridinyl)-1,3,4-oxadiazol-2-yl]methyl}(2,2,2-trifluoroethyl)amino]-2-(trifluoromethyl)benzonitrile;
4-[{[5-(4-Fluorophenyl)-1,3,4-oxadiazol-2-yl]methyl}(2,2,2-trifluoroethyl)amino]-2-(trifluoromethyl)benzonitrile;
4-[[(2-Methyl-2H-tetrazol-5-yl)methyl](2,2,2-trifluoroethyl)amino]-2-(trifluoromethyl)benzonitrile;
   and 4-((Cyclopropylmethyl){[5-(2-pyridinyl)-1,3,4-oxadiazol-2-yl]methyl}amino)-1,2-benzenedicarbonitrile; or a salt or solvate thereof.

Representative AR modulator compounds, agonists, partial agonists, and antagonists according to the current invention also include:
4-{(Cyclopropylmethyl)[(1R)-1-(2-naphthyl)ethyl]amino}-2-(trifluoromethyl)benzonitrile;
4-{[(1R)-1-Phenylpropyl]amino}-2-(trifluoromethyl)benzonitrile;
4-{(Cyclopropylmethyl)[(1S)-2,3-dihydro-1H-inden-1-yl]amino}-2-(trifluoromethyl)benzonitrile;
N-[(1R)-1-(4-Bromophenyl)ethyl]-4-nitro-3-(trifluoromethyl)aniline;
4-{(Cyclopropylmethyl)[(1R)-2,3-dihydro-1H-inden-1-yl]amino}-2-(trifluoromethyl)benzonitrile;
4-[Benzyl(1-cyclopropylethyl)amino]-2-(trifluoromethyl)benzonitrile;
4-{Allyl[(1R)-1-(4-bromophenyl)ethyl]amino}-2-(trifluoromethyl)benzonitrile;
N-Allyl-N-[(1R)-1-(4-bromophenyl)ethyl]-4-nitro-3-(trifluoromethyl)aniline;
4-{Allyl[(1S)-2,3-dihydro-1H-inden-1-yl]amino}-2-(trifluoromethyl)benzonitrile;
4-[Benzyl(2,2,2-trifluoroethyl)amino]-2-(trifluoromethyl)benzonitrile;
4-[(2-Methylbenzyl)(2,2,2-trifluoroethyl)amino]-2-(trifluoromethyl)benzonitrile;
4-{(2,2,2-Trifluoroethyl)[2-(trifluoromethyl)benzyl]amino}-2-(trifluoromethyl)benzonitrile;
4-[(2-Fluorobenzyl)(2,2,2-trifluoroethyl)amino]-2-(trifluoromethyl)benzonitrile;
4-[(3-Methylbenzyl)(2,2,2-trifluoroethyl)amino]-2-(trifluoromethyl)benzonitrile;
4-{(2,2,2-Trifluoroethyl)[3-(trifluoromethyl)benzyl]amino}-2-(trifluoromethyl)benzonitrile;
4-[(3-Fluorobenzyl)(2,2,2-trifluoroethyl)amino]-2-(trifluoromethyl)benzonitrile;
4-[(3-Chlorobenzyl)(2,2,2-trifluoroethyl)amino]-2-(trifluoromethyl)benzonitrile;
4-[(3-Bromobenzyl)(2,2,2-trifluoroethyl)amino]-2-(trifluoromethyl)benzonitrile;
4-[(1,1′-Biphenyl-3-ylmethyl)(2,2,2-trifluoroethyl)amino]-2-(trifluoromethyl)benzonitrile;
4-[(3-Methoxybenzyl)(2,2,2-trifluoroethyl)amino]-2-(trifluoromethyl)benzonitrile;
4-{(2,2,2-Trifluoroethyl)[3-(trifluoromethoxy)benzyl]amino}-2-(trifluoromethyl)benzonitrile;
4-[[3-(4-Fluorophenoxy)benzyl](2,2,2-trifluoroethyl)amino]-2-(trifluoromethyl)benzonitrile;
4-[[3-(2-Fluorophenoxy)benzyl](2,2,2-trifluoroethyl)amino]-2-(trifluoromethyl)benzonitrile;
4-((2,2,2-Trifluoroethyl){3-[(trifluoromethyl)thio]benzyl}amino)-2-(trifluoromethyl)benzonitrile;
4-[(3-Cyanobenzyl)(2,2,2-trifluoroethyl)amino]-2-(trifluoromethyl)benzonitrile;
Methyl 3-{[[4-cyano-3-(trifluoromethyl)phenyl](2,2,2-trifluoroethyl)amino]methyl}benzoate;
3-{[[4-Cyano-3-(trifluoromethyl)phenyl](2,2,2-trifluoroethyl)amino]methyl}benzamide;
4-[(3-Nitrobenzyl)(2,2,2-trifluoroethyl)amino]-2-(trifluoromethyl)benzonitrile;
4-[(4-Methylbenzyl)(2,2,2-trifluoroethyl)amino]-2-(trifluoromethyl)benzonitrile;
4-[(4-tert-Butylbenzyl)(2,2,2-trifluoroethyl)amino]-2-(trifluoromethyl)benzonitrile;
4-{(2,2,2-Trifluoroethyl)[4-(trifluoromethyl)benzyl]amino}-2-(trifluoromethyl)benzonitrile;
4-[(4-Fluorobenzyl)(2,2,2-trifluoroethyl)amino]-2-(trifluoromethyl)benzonitrile;
4-[(4-Methoxybenzyl)(2,2,2-trifluoroethyl)amino]-2-(trifluoromethyl)benzonitrile;
4-{(2,2,2-Trifluoroethyl)[4-(trifluoromethoxy)benzyl]amino}-2-(trifluoromethyl)benzonitrile;
4-[[4-(Methylsulfonyl)benzyl](2,2,2-trifluoroethyl)amino]-2-(trifluoromethyl)benzonitrile;
4-[(4-Cyanobenzyl)(2,2,2-trifluoroethyl)amino]-2-(trifluoromethyl)benzonitrile;
Methyl 4-{[[4-cyano-3-(trifluoromethyl)phenyl](2,2,2-trifluoroethyl)amino]methyl}benzoate;
4-[(4-Nitrobenzyl)(2,2,2-trifluoroethyl)amino]-2-(trifluoromethyl)benzonitrile;
4-[(3,5-Dimethylbenzyl)(2,2,2-trifluoroethyl)amino]-2-(trifluoromethyl)benzonitrile;
4-[[3,5-bis(Trifluoromethyl)benzyl](2,2,2-trifluoroethyl)amino]-2-(trifluoromethyl)benzonitrile;
4-[(3,5-Dimethoxybenzyl)(2,2,2-trifluoroethyl)amino]-2-(trifluoromethyl)benzonitrile;
4-[(3,5-Dichlorobenzyl)(2,2,2-trifluoroethyl)amino]-2-(trifluoromethyl)benzonitrile;
4-[(3,5-Difluorobenzyl)(2,2,2-trifluoroethyl)amino]-2-(trifluoromethyl)benzonitrile;
4-[(2,6-Difluorobenzyl)(2,2,2-trifluoroethyl)amino]-2-(trifluoromethyl)benzonitrile;
4-[(2,4-Difluorobenzyl)(2,2,2-trifluoroethyl)amino]-2-(trifluoromethyl)benzonitrile;
4-[(3,4-Difluorobenzyl)(2,2,2-trifluoroethyl)amino]-2-(trifluoromethyl)benzonitrile;
4-[(2,3-Difluorobenzyl)(2,2,2-trifluoroethyl)amino]-2-(trifluoromethyl)benzonitrile;
4-[[3-Fluoro-4-(trifluoromethyl)benzyl](2,2,2-trifluoroethyl)amino]-2-(trifluoromethyl)benzonitrile;
4-[(2-Naphthylmethyl)(2,2,2-trifluoroethyl)amino]-2-(trifluoromethyl)benzonitrile;
4-[(1-Phenylethyl)(2,2,2-trifluoroethyl)amino]-2-(trifluoromethyl)benzonitrile;
4-[(3-Furylmethyl)(2,2,2-trifluoroethyl)amino]-2-(trifluoromethyl)benzonitrile;
4-[(2-Furylmethyl)(2,2,2-trifluoroethyl)amino]-2-(trifluoromethyl)benzonitrile;
Ethyl 5-{[[4-cyano-3-(trifluoromethyl)phenyl](2,2,2-trifluoroethyl)amino]methyl}-2-furoate;
5-{[[4-Cyano-3-(trifluoromethyl)phenyl](2,2,2-trifluoroethyl)amino]methyl}-2-furamide;
5-{[[4-Cyano-3-(trifluoromethyl)phenyl](2,2,2-trifluoroethyl)amino]methyl}-2-furonitrile;
4-[[(5-Methyl-2-furyl)methyl](2,2,2-trifluoroethyl)amino]-2-(trifluoromethyl)benzonitrile;
4-((2,2,2-Trifluoroethyl){[5-(trifluoromethyl)-2-furyl]methyl}amino)-2-(trifluoromethyl)benzonitrile;
4-[{[5-(Cyanomethyl)-2-furyl]methyl}(2,2,2-trifluoroethyl)amino]-2-(trifluoromethyl)benzonitrile;
4-[{[5-(Methoxymethyl)-2-furyl]methyl}(2,2,2-trifluoroethyl)amino]-2-(trifluoromethyl)benzonitrile;
4-[({5-[(2,2,2-Trifluoroethoxy)methyl]-2-furyl}methyl)(2,2,2-trifluoroethyl)amino]-2-(trifluoromethyl)benzonitrile;
4-[({5-[(Methylthio)methyl]-2-furyl}methyl)(2,2,2-trifluoroethyl)amino]-2-(trifluoromethyl)benzonitrile;
4-[({5-[(Dimethylamino)methyl]-2-furyl}methyl)(2,2,2-trifluoroethyl)amino]-2-(trifluoromethyl)benzonitrile;
4-[{[5-(1H-Imidazol-1-ylmethyl)-2-furyl]methyl}(2,2,2-trifluoroethyl)amino]-2-(trifluoromethyl)benzonitrile;
4-[{[5-(1H-1,2,4-Triazol-1-ylmethyl)-2-furyl]methyl}(2,2,2-trifluoroethyl)amino]-2-(trifluoromethyl)benzonitrile;
4-[{[5-(2-Cyanoethyl)-2-furyl]methyl}(2,2,2-trifluoroethyl)amino]-2-(trifluoromethyl)benzonitrile;
4-[{[5-(3,5-Difluorophenyl)-2-furanyl]methyl}(2,2,2-trifluoroethyl)amino]-2-(trifluoromethyl)benzonitrile;
4-[[(5-Phenyl-2-furanyl)methyl](2,2,2-trifluoroethyl)amino]-2-(trifluoromethyl)benzonitrile;
4-[{[5-(4-Fluorophenyl)-2-furanyl]methyl}(2,2,2-trifluoroethyl)amino]-2-(trifluoromethyl)benzonitrile;
4-[{[5-(3,4-Difluorophenyl)-2-furanyl]methyl}(2,2,2-trifluoroethyl)amino]-2-(trifluoromethyl)benzonitrile;
4-[{[5-(2,4-Difluorophenyl)-2-furanyl]methyl}(2,2,2-trifluoroethyl)amino]-2-(trifluoromethyl)benzonitrile;
4-{(2,2,2-Trifluoroethyl)[(5-{4-[(trifluoromethyl)oxy]phenyl}-2-furanyl)methyl]amino}-2-(trifluoromethyl)benzonitrile;
N-[4-(5-{[[4-Cyano-3-(trifluoromethyl)phenyl](2,2,2-trifluoroethyl)amino]methyl}-2-furanyl)phenyl]acetamide;
4-[{[5-(4-Cyanophenyl)-2-furanyl]methyl}(2,2,2-trifluoroethyl)amino]-2-(trifluoromethyl)benzonitrile;
4-[({5-[4-(Methylsulfonyl)phenyl]-2-furanyl}methyl)(2,2,2-trifluoroethyl)amino]-2-(trifluoromethyl)benzonitrile;
4-[{[5-(3-Cyanophenyl)-2-furanyl]methyl}(2,2,2-trifluoroethyl)amino]-2-(trifluoromethyl)benzonitrile;
5-(5-{[[4-Cyano-3-(trifluoromethyl)phenyl](2,2,2-trifluoroethyl)amino]methyl}-2-furanyl)-2-thiophenecarbonitrile;
4-[{[5-(5-Pyrimidinyl)-2-furanyl]methyl}(2,2,2-trifluoroethyl)amino]-2-(trifluoromethyl)benzonitrile;
4-[{[5-(3,5-Dimethyl-4-isoxazolyl)-2-furanyl]methyl}(2,2,2-trifluoroethyl)amino]-2-(trifluoromethyl)benzonitrile;
4-[{[4-(4-Cyanophenyl)-2-furanyl]methyl}(2,2,2-trifluoroethyl)amino]-2-(trifluoromethyl)benzonitrile;
Methyl 2-{[[4-cyano-3-(trifluoromethyl)phenyl](2,2,2-trifluoroethyl)amino]methyl}-3-furoate;
2-{[[4-Cyano-3-(trifluoromethyl)phenyl](2,2,2-trifluoroethyl)amino]methyl}-3-furamide;
2-{[[4-Cyano-3-(trifluoromethyl)phenyl](2,2,2-trifluoroethyl)amino]methyl}-3-furonitrile;
4-[{[3-(Hydroxymethyl)-2-furyl]methyl}(2,2,2-trifluoroethyl)amino]-2-(trifluoromethyl)benzonitrile;
4-[[(3-Methyl-2-furyl)methyl](2,2,2-trifluoroethyl)amino]-2-(trifluoromethyl)benzonitrile;
4-[(1-Benzofuran-2-ylmethyl)(2,2,2-trifluoroethyl)amino]-2-(trifluoromethyl)benzonitrile;
4-[[(5-Chlorothien-2-yl)methyl](2,2,2-trifluoroethyl)amino]-2-(trifluoromethyl)benzonitrile;
4-[(Thien-2-ylmethyl)(2,2,2-trifluoroethyl)amino]-2-(trifluoromethyl)benzonitrile;
4-[[(5-Bromothien-2-yl)methyl](2,2,2-trifluoroethyl)amino]-2-(trifluoromethyl)benzonitrile;
5-{[[4-Cyano-3-(trifluoromethyl)phenyl](2,2,2-trifluoroethyl)amino]methyl}thiophene-2-carbonitrile;
4-[{[5-(4-Fluorophenyl)thien-2-yl]methyl}(2,2,2-trifluoroethyl)amino]-2-(trifluoromethyl)benzonitrile;
4-[{[5-(4-Cyanophenyl)thien-2-yl]methyl}(2,2,2-trifluoroethyl)amino]-2-(trifluoromethyl)benzonitrile;
4-[(Thien-3-ylmethyl)(2,2,2-trifluoroethyl)amino]-2-(trifluoromethyl)benzonitrile;
4-[[(5-Chloro-1-benzothien-3-yl)methyl](2,2,2-trifluoroethyl)amino]-2-(trifluoromethyl)benzonitrile;
4-[(1H-Imidazol-2-ylmethyl)(2,2,2-trifluoroethyl)amino]-2-(trifluoromethyl)benzonitrile;
1,1-Dimethylethyl 4-{[[4-cyano-3-(trifluoromethyl)phenyl](2,2,2-trifluoroethyl)amino]methyl}-1H-imidazole-1-carboxylate;
4-[(1H-Imidazol-4-ylmethyl)(2,2,2-trifluoroethyl)amino]-2-(trifluoromethyl)benzonitrile;
4-[(1,3-Oxazol-2-ylmethyl)(2,2,2-trifluoroethyl)amino]-2-(trifluoromethyl)benzonitrile;
4-[(1,3-Oxazol-4-ylmethyl)(2,2,2-trifluoroethyl)amino]-2-(trifluoromethyl)benzonitrile;
4-[{[4-(Hydroxymethyl)-1,3-oxazol-2-yl]methyl}(2,2,2-trifluoroethyl)amino]-2-(trifluoromethyl)benzonitrile;
2-{[[4-Cyano-3-(trifluoromethyl)phenyl](2,2,2-trifluoroethyl)amino]methyl}-1,3-oxazole-4-carbonitrile;
2-{[[4-Cyano-3-(trifluoromethyl)phenyl](2,2,2-trifluoroethyl)amino]methyl}-1,3-oxazole-5-carbonitrile;
4-[[(5-{[(Phenylmethyl)oxy]methyl}-1,3-oxazol-2-yl)methyl](2,2,2-trifluoroethyl)amino]-2-(trifluoromethyl)benzonitrile;
4-[{[2-(4-Fluorophenyl)-1,3-oxazol-4-yl]methyl}(2,2,2-trifluoroethyl)amino]-2-(trifluoromethyl)benzonitrile;
4-[[(4-Phenyl-1,3-oxazol-2-yl)methyl](2,2,2-trifluoroethyl)amino]-2-(trifluoromethyl)benzonitrile;
5-{[[4-Cyano-3-(trifluoromethyl)phenyl](2,2,2-trifluoroethyl)amino]methyl}-1,3-oxazole-2-carbonitrile;
4-[{[2-(4-Fluorophenyl)-1,3-oxazol-5-yl]methyl}(2,2,2-trifluoroethyl)amino]-2-(trifluoromethyl)benzonitrile;
4-[{[5-(4-Fluorophenyl)-1,3-oxazol-2-yl]methyl}(2,2,2-trifluoroethyl)amino]-2-(trifluoromethyl)benzonitrile;
4-[(1,3-Oxazol-5-ylmethyl)(2,2,2-trifluoroethyl)amino]-2-(trifluoromethyl)benzonitrile;
4-[[(3,5-Dimethylisoxazol-4-yl)methyl](2,2,2-trifluoroethyl)amino]-2-(trifluoromethyl)benzonitrile;
4-[[(5-Methylisoxazol-3-yl)methyl](2,2,2-trifluoroethyl)amino]-2-(trifluoromethyl)benzonitrile;
4-[(5-Isoxazolylmethyl)(2,2,2-trifluoroethyl)amino]-2-(trifluoromethyl)benzonitrile;
4-[(Cyclopropylmethyl)(1,3-thiazol-4-ylmethyl)amino]-2-(trifluoromethyl)benzonitrile;
4-[(1,3-Thiazol-4-ylmethyl)(2,2,2-trifluoroethyl)amino]-2-(trifluoromethyl)benzonitrile;
4-[[(2-Methyl-1,3-thiazol-4-yl)methyl](2,2,2-trifluoroethyl)amino]-2-(trifluoromethyl)benzonitrile;
4-[[(2-Phenyl-1,3-thiazol-4-yl)methyl](2,2,2-trifluoroethyl)amino]-2-(trifluoromethyl)benzonitrile;
4-[(2,2,2-Trifluoroethyl)({2-[4-(trifluoromethyl)phenyl]-1,3-thiazol-4-yl}methyl)amino]-2-(trifluoromethyl)benzonitrile;
4-[[(2-Thien-2-yl-1,3-thiazol-4-yl)methyl](2,2,2-trifluoroethyl)amino]-2-(trifluoromethyl)benzonitrile;
4-[(1,3-Benzothiazol-2-ylmethyl)(2,2,2-trifluoroethyl)amino]-2-(trifluoromethyl)benzonitrile;
4-[(1,3-Thiazol-5-ylmethyl)(2,2,2-trifluoroethyl)amino]-2-(trifluoromethyl)benzonitrile;
4-[(1,3-Thiazol-2-ylmethyl)(2,2,2-trifluoroethyl)amino]-2-(trifluoromethyl)benzonitrile;
4-[[1,3-Thiazol-2-yl)ethyl](2,2,2-trifluoroethyl)amino]-2-(trifluoromethyl)benzonitrile;
2-Chloro-4-[(1,3-thiazol-4-ylmethyl)(2,2,2-trifluoroethyl)amino]benzonitrile;
4-[(Cyclopropylmethyl)(1,3-thiazol-4-ylmethyl)amino]-1,2-benzenedicarbonitrile;
4-[(1,3-Thiazol-4-ylmethyl)(2,2,2-trifluoroethyl)amino]-1,2-benzenedicarbonitrile;
4-[[(5-tert-Butyl-1,2,4-oxadiazol-3-yl)methyl](2,2,2-trifluoroethyl)amino]-2-(trifluoromethyl)benzonitrile;
4-[[(5-Phenyl-1,2,4-oxadiazol-3-yl)methyl](2,2,2-trifluoroethyl)amino]-2-(trifluoromethyl)benzonitrile;
4-[{[5-(2-Methoxyphenyl)-1,2,4-oxadiazol-3-yl]methyl}(2,2,2-trifluoroethyl)amino]-2-(trifluoromethyl)benzonitrile;
4-[(2,2,2-Trifluoroethyl)({5-[3-(trifluoromethyl)phenyl]-1,2,4-oxadiazol-3-yl}methyl)amino]-2-(trifluoromethyl)benzonitrile;
4-(3-{[[4-Cyano-3-(trifluoromethyl)phenyl](2,2,2-trifluoroethyl)amino]methyl}-1,2,4-oxadiazol-5-yl)-1-piperidinesulfonamide;
4-[(1,2,4-Oxadiazol-3-ylmethyl)(2,2,2-trifluoroethyl)amino]-2-(trifluoromethyl)benzonitrile;
4-[{[5-(1-Methylethyl)-1,2,4-oxadiazol-3-yl]methyl}(2,2,2-trifluoroethyl)amino]-2-(trifluoromethyl)benzonitrile;
4-[[(5-Methyl-1,2,4-oxadiazol-3-yl)methyl](2,2,2-trifluoroethyl)amino]-2-(trifluoromethyl)benzonitrile;
4-[{[5-(1-Methylpropyl)-1,2,4-oxadiazol-3-yl]methyl}(2,2,2-trifluoroethyl)amino]-2-(trifluoromethyl)benzonitrile;
4-((2,2,2-Trifluoroethyl){[5-(trifluoromethyl)-1,2,4-oxadiazol-3-yl]methyl}amino)-2-(trifluoromethyl)benzonitrile;
4-[[(5-Oxo-2,5-dihydro-1,2,4-oxadiazol-3-yl)methyl](2,2,2-trifluoroethyl)amino]-2-(trifluoromethyl)benzonitrile;
4-[{[5-(4-Fluorophenyl)-1,2,4-oxadiazol-3-yl]methyl}(2,2,2-trifluoroethyl)amino]-2-(trifluoromethyl)benzonitrile;
4-{(2,2,2-Trifluoroethyl)[(5-{4-[(trifluoromethyl)oxy]phenyl}-1,2,4-oxadiazol-3-yl)methyl]amino}-2-(trifluoromethyl)benzonitrile;
4-[(2,2,2-Trifluoroethyl)({5-[4-(trifluoromethyl)phenyl]-1,2,4-oxadiazol-3-yl}methyl)amino]-2-(trifluoromethyl)benzonitrile;
4-{(2,2,2-Trifluoroethyl)[(5-{3-[(trifluoromethyl)oxy]phenyl}-1,2,4-oxadiazol-3-yl)methyl]amino}-2-(trifluoromethyl)benzonitrile;
4-[{[5-(5-Isoxazolyl)-1,2,4-oxadiazol-3-yl]methyl}(2,2,2-trifluoroethyl)amino]-2-(trifluoromethyl)benzonitrile;
4-[{[5-(2-Thienyl)-1,2,4-oxadiazol-3-yl]methyl}(2,2,2-trifluoroethyl)amino]-2-(trifluoromethyl)benzonitrile;
4-[{[5-(3,5-Dimethyl-4-isoxazolyl)-1,2,4-oxadiazol-3-yl]methyl}(2,2,2-trifluoroethyl)amino]-2-(trifluoromethyl)benzonitrile;
1,1-Dimethylethyl[(3-{[[4-cyano-3-(trifluoromethyl)phenyl](2,2,2-trifluoroethyl)amino]methyl}-1,2,4-oxadiazol-5-yl)methyl]carbamate;
4-[{[5-(Aminomethyl)-1,2,4-oxadiazol-3-yl]methyl}(2,2,2-trifluoroethyl)amino]-2-(trifluoromethyl)benzonitrile;
N-[(3-{[[4-Cyano-3-(trifluoromethyl)phenyl](2,2,2-trifluoroethyl)amino]methyl}-1,2,4-oxadiazol-5-yl)methyl]acetamide;
N-[(3-{[[4-Cyano-3-(trifluoromethyl)phenyl](2,2,2-trifluoroethyl)amino]methyl}-1,2,4-oxadiazol-5-yl)methyl]methanesulfonamide;
N-[(3-{[[4-Cyano-3-(trifluoromethyl)phenyl](2,2,2-trifluoroethyl)amino]methyl}-1,2,4-oxadiazol-5-yl)methyl]urea;
N-[(3-{[[4-cyano-3-(trifluoromethyl)phenyl](2,2,2-trifluoroethyl)amino]methyl}-1,2,4-oxadiazol-5-yl)methyl]dicarbonimidic diamide;
1,1-Dimethylethyl[2-(3-{[[4-cyano-3-(trifluoromethyl)phenyl](2,2,2-trifluoroethyl)amino]methyl}-1,2,4-oxadiazol-5-yl)ethyl]carbamate;
4-[{[5-(2-Aminoethyl)-1,2,4-oxadiazol-3-yl]methyl}(2,2,2-trifluoroethyl)amino]-2-(trifluoromethyl)benzonitrile;
N-[2-(3-{[[4-Cyano-3-(trifluoromethyl)phenyl](2,2,2-trifluoroethyl)amino]methyl}-1,2,4-oxadiazol-5-yl)ethyl]acetamide;
N-[2-(3-{[[4-Cyano-3-(trifluoromethyl)phenyl](2,2,2-trifluoroethyl)amino]methyl}-1,2,4-oxadiazol-5-yl)ethyl]methanesulfonamide;
N-[2-(3-{[[4-Cyano-3-(trifluoromethyl)phenyl](2,2,2-trifluoroethyl)amino]methyl}-1,2,4-oxadiazol-5-yl)ethyl]urea;
N-[2-(3-{[[4-cyano-3-(trifluoromethyl)phenyl](2,2,2-trifluoroethyl)amino]methyl}-1,2,4-oxadiazol-5-yl)ethyl]dicarbonimidic diamide;
4-[{[5-(1-Acetyl-4-piperidinyl)-1,2,4-oxadiazol-3-yl]methyl}(2,2,2-trifluoroethyl)amino]-2-(trifluoromethyl)benzonitrile;
4-[({5-[1-(5-Isoxazolylcarbonyl)-4-piperidinyl]-1,2,4-oxadiazol-3-yl}methyl)(2,2,2-trifluoroethyl)amino]-2-(trifluoromethyl)benzonitrile;
Methyl 4-(3-{[[4-cyano-3-(trifluoromethyl)phenyl](2,2,2-trifluoroethyl)amino]methyl}-1,2,4-oxadiazol-5-yl)-1-piperidinecarboxylate;
4-(3-{[[4-Cyano-3-(trifluoromethyl)phenyl](2,2,2-trifluoroethyl)amino]methyl}-1,2,4-oxadiazol-5-yl)-1-piperidinecarboxamide;
4-(3-{[[4-Cyano-3-(trifluoromethyl)phenyl](2,2,2-trifluoroethyl)amino]methyl}-1,2,4-oxadiazol-5-yl)-N-methyl-1-piperidinecarboxamide;
4-[({5-[1-(Methylsulfonyl)-4-piperidinyl]-1,2,4-oxadiazol-3-yl}methyl)(2,2,2-trifluoroethyl)amino]-2-(trifluoromethyl)benzonitrile;
4-[{[5-(1-Methyl-4-piperidinyl)-1,2,4-oxadiazol-3-yl]methyl}(2,2,2-trifluoroethyl)amino]-2-(trifluoromethyl)benzonitrile;
1,1-Dimethylethyl 3-(3-{[[4-cyano-3-(trifluoromethyl)phenyl](2,2,2-trifluoroethyl)amino]methyl}-1,2,4-oxadiazol-5-yl)-1-piperidinecarboxylate;
4-[{[5-(3-Piperidinyl)-1,2,4-oxadiazol-3-yl]methyl}(2,2,2-trifluoroethyl)amino]-2-(trifluoromethyl)benzonitrile;
4-[{[5-(1-Acetyl-3-piperidinyl)-1,2,4-oxadiazol-3-yl]methyl}(2,2,2-trifluoroethyl)amino]-2-(trifluoromethyl)benzonitrile;
Methyl 3-(3-{[[4-cyano-3-(trifluoromethyl)phenyl](2,2,2-trifluoroethyl)amino]methyl}-1,2,4-oxadiazol-5-yl)-1-piperidinecarboxylate;
3-(3-{[[4-Cyano-3-(trifluoromethyl)phenyl](2,2,2-trifluoroethyl)amino]methyl}-1,2,4-oxadiazol-5-yl)-1-piperidinecarboxamide;
3-(3-{[[4-Cyano-3-(trifluoromethyl)phenyl](2,2,2-trifluoroethyl)amino]methyl}-1,2,4-oxadiazol-5-yl)-N-methyl-1-piperidinecarboxamide;
4-[({5-[1-(Methylsulfonyl)-3-piperidinyl]-1,2,4-oxadiazol-3-yl}methyl)(2,2,2-trifluoroethyl)amino]-2-(trifluoromethyl)benzonitrile;
4-[{[5-(Phenylcarbonyl)-1,2,4-oxadiazol-3-yl]methyl}(2,2,2-trifluoroethyl)amino]-2-(trifluoromethyl)benzonitrile;
4-[{[5-(2-Furanylcarbonyl)-1,2,4-oxadiazol-3-yl]methyl}(2,2,2-trifluoroethyl)amino]-2-(trifluoromethyl)benzonitrile;
4-[(2,2-Dimethylpropyl)({5-[3-(trifluoromethyl)phenyl]-1,2,4-oxadiazol-3-yl}methyl)amino]-2-(trifluoromethyl)benzonitrile;
4-[{[5-(2-Pyridinyl)-1,2,4-oxadiazol-3-yl]methyl}(2,2,2-trifluoroethyl)amino]-2-(trifluoromethyl)benzonitrile;
4-[{[5-(Cyclopropylmethyl)-1,2,4-oxadiazol-3-yl]methyl}(2,2,2-trifluoroethyl)amino]-2-(trifluoromethyl)benzonitrile;
4-((2,2,2-Trifluoroethyl){[5-(2,2,2-trifluoroethyl)-1,2,4-oxadiazol-3-yl]methyl}amino)-2-(trifluoromethyl)benzonitrile;
4-[({5-[(Dimethylamino)methyl]-1,2,4-oxadiazol-3-yl}methyl)(2,2,2-trifluoroethyl)amino]-2-(trifluoromethyl)benzonitrile;
4-[(2,2,2-Trifluoroethyl)({5-[6-(trifluoromethyl)-3-pyridinyl]-1,2,4-oxadiazol-3-yl}methyl)amino]-2-(trifluoromethyl)benzonitrile;
4-[{[3-(4-Fluorophenyl)-1,2,4-oxadiazol-5-yl]methyl}(2,2,2-trifluoroethyl)amino]-2-(trifluoromethyl)benzonitrile;
4-[{[3-(2-Pyridinyl)-1,2,4-oxadiazol-5-yl]methyl}(2,2,2-trifluoroethyl)amino]-2-(trifluoromethyl)benzonitrile;
4-[[(3-Phenyl-1,2,4-oxadiazol-5-yl)methyl](2,2,2-trifluoroethyl)amino]-2-(trifluoromethyl)benzonitrile;
4-[(2,2,2-Trifluoroethyl)({3-[4-(trifluoromethyl)phenyl]-1,2,4-oxadiazol-5-yl}methyl)amino]-2-(trifluoromethyl)benzonitrile;
4-{(2,2,2-Trifluoroethyl)[(3-{4-[(trifluoromethyl)oxy]phenyl}-1,2,4-oxadiazol-5-yl)methyl]amino}-2-(trifluoromethyl)benzonitrile;
4-[({3-[4-(1,1-Dimethylethyl)phenyl]-1,2,4-oxadiazol-5-yl}methyl)(2,2,2-trifluoroethyl)amino]-2-(trifluoromethyl)benzonitrile;
4-[(2,2,2-Trifluoroethyl)({3-[3-(trifluoromethyl)phenyl]-1,2,4-oxadiazol-5-yl}methyl)amino]-2-(trifluoromethyl)benzonitrile;
4-[{[3-(3-Pyridinyl)-1,2,4-oxadiazol-5-yl]methyl}(2,2,2-trifluoroethyl)amino]-2-(trifluoromethyl)benzonitrile;
4-[({3-[3,5-Bis(trifluoromethyl)phenyl]-1,2,4-oxadiazol-5-yl}methyl)(2,2,2-trifluoroethyl)amino]-2-(trifluoromethyl)benzonitrile;
4-[{[3-(3-Nitrophenyl)-1,2,4-oxadiazol-5-yl]methyl}(2,2,2-trifluoroethyl)amino]-2-(trifluoromethyl)benzonitrile;
4-[[(3-{[(4-Fluorophenyl)sulfonyl]methyl}-1,2,4-oxadiazol-5-yl)methyl](2,2,2-trifluoroethyl)amino]-2-(trifluoromethyl)benzonitrile;
4-[(2,2,2-Trifluoroethyl)({3-[6-(trifluoromethyl)-3-pyridinyl]-1,2,4-oxadiazol-5-yl}methyl)amino]-2-(trifluoromethyl)benzonitrile;
4-[{[3-(2,4-Dichlorophenyl)-1,2,4-oxadiazol-5-yl]methyl}(2,2,2-trifluoroethyl)amino]-2-(trifluoromethyl)benzonitrile;
4-[{[3-(2,3-Dichlorophenyl)-1,2,4-oxadiazol-5-yl]methyl}(2,2,2-trifluoroethyl)amino]-2-(trifluoromethyl)benzonitrile;
4-[{[5-(4-Methylphenyl)-1,3,4-oxadiazol-2-yl]methyl}(2,2,2-trifluoroethyl)amino]-2-(trifluoromethyl)benzonitrile;
4-[{[5-(2-Pyridinyl)-1,3,4-oxadiazol-2-yl]methyl}(2,2,2-trifluoroethyl)amino]-2-(trifluoromethyl)benzonitrile;
4-[{[5-(5-Bromo-3-pyridinyl)-1,3,4-oxadiazol-2-yl]methyl}(2,2,2-trifluoroethyl)amino]-2-(trifluoromethyl)benzonitrile;
4-[{[5-(2-Chloro-4-pyridinyl)-1,3,4-oxadiazol-2-yl]methyl}(2,2,2-trifluoroethyl)amino]-2-(trifluoromethyl)benzonitrile;
4-[{[5-(6-Chloro-3-pyridinyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[6-(trifluoromethyl)-3-pyridinyl]-1,3,4-oxadiazol-2-yl}methyl)amino]-2-(trifluoromethyl)benzonitrile;
4-[{[5-(6-Chloro-2-pyridinyl)-1,3,4-oxadiazol-2-yl]methyl}(2,2,2-trifluoroethyl)amino]-2-(trifluoromethyl)benzonitrile;
4-[{[5-(6-Fluoro-2-pyridinyl)-1,3,4-oxadiazol-2-yl]methyl}(2,2,2-trifluoroethyl)amino]-2-(trifluoromethyl)benzonitrile;
4-[{[5-(4-Fluorophenyl)-1,3,4-oxadiazol-2-yl]methyl}(2,2,2-trifluoroethyl)amino]-2-(trifluoromethyl)benzonitrile;
4-[{[5-(3-Methylphenyl)-1,3,4-oxadiazol-2-yl]methyl}(2,2,2-trifluoroethyl)amino]-2-(trifluoromethyl)benzonitrile;
4-[{[5-(3-Pyridinyl)-1,3,4-oxadiazol-2-yl]methyl}(2,2,2-trifluoroethyl)amino]-2-(trifluoromethyl)benzonitrile;
4-[[(5-Phenyl-1,3,4-oxadiazol-2-yl)methyl](2,2,2-trifluoroethyl)amino]-2-(trifluoromethyl)benzonitrile;
4-[{[5-(2-Furanyl)-1,3,4-oxadiazol-2-yl]methyl}(2,2,2-trifluoroethyl)amino]-2-(trifluoromethyl)benzonitrile;
4-[{[5-(3-Furanyl)-1,3,4-oxadiazol-2-yl]methyl}(2,2,2-trifluoroethyl)amino]-2-(trifluoromethyl)benzonitrile;
4-[{[5-(4-Pyridinyl)-1,3,4-oxadiazol-2-yl]methyl}(2,2,2-trifluoroethyl)amino]-2-(trifluoromethyl)benzonitrile;
4-[({5-[(4-(Dimethylamino)phenyl]-1,3,4-oxadiazol-2-yl}methyl)(2,2,2-trifluoroethyl)amino]-2-(trifluoromethyl)benzonitrile;
4-[{[5-(1,3-Benzodioxol-5-yl)-1,3,4-oxadiazol-2-yl]methyl}(2,2,2-trifluoroethyl)amino]-2-(trifluoromethyl)benzonitrile;
4-[{[5-(3-Thienyl)-1,3,4-oxadiazol-2-yl]methyl}(2,2,2-trifluoroethyl)amino]-2-(trifluoromethyl)benzonitrile;
4-[{[5-(4-Nitrophenyl)-1,3,4-oxadiazol-2-yl]methyl}(2,2,2-trifluoroethyl)amino]-2-(trifluoromethyl)benzonitrile;
4-{2-[(5-(4-Aminophenyl)-1,3,4-oxadiazol-2-yl]-1-[(trifluoromethyl)amino]ethyl}-2-(trifluoromethyl)benzonitrile;
N-[4-(5-{2-[4-Cyano-3-(trifluoromethyl)phenyl]-2-[(trifluoromethyl)amino]ethyl}-1,3,4-oxadiazol-2-yl)phenyl]acetamide;
4-[{[5-(2-Methylphenyl)-1,3,4-oxadiazol-2-yl]methyl}(2,2,2-trifluoroethyl)amino]-2-(trifluoromethyl)benzonitrile;
4-[{[5-(3-Fluorophenyl)-1,3,4-oxadiazol-2-yl]methyl}(2,2,2-trifluoroethyl)amino]-2-(trifluoromethyl)benzonitrile;
4-[({5-[4-(Methyloxy)phenyl]-1,3,4-oxadiazol-2-yl}methyl)(2,2,2-trifluoroethyl)amino]-2-(trifluoromethyl)benzonitrile;
4-[({5-[4-(Methylthio)phenyl]-1,3,4-oxadiazol-2-yl}methyl)(2,2,2-trifluoroethyl)amino]-2-(trifluoromethyl)benzonitrile;
4-{2-{5-[4-(Methylsulfinyl)phenyl]-1,3,4-oxadiazol-2-yl}-1-[(trifluoromethyl)amino]ethyl}-2-(trifluoromethyl)benzonitrile;
4-{2-{5-[4-(Methylsulfonyl)phenyl]-1,3,4-oxadiazol-2-yl}-1-[(trifluoromethyl)amino]ethyl}-2-(trifluoromethyl)benzonitrile;
4-[{[5-(5-Bromo-2-furanyl)-1,3,4-oxadiazol-2-yl]methyl}(2,2,2-trifluoroethyl)amino]-2-(trifluoromethyl)benzonitrile;
4-{(2,2,2-Trifluoroethyl)[(5-{4-[(trifluoromethyl)oxy]phenyl}-1,3,4-oxadiazol-2-yl)methyl]amino}-2-(trifluoromethyl)benzonitrile;
4-[{[5-(3,4-Difluorophenyl)-1,3,4-oxadiazol-2-yl]methyl}(2,2,2-trifluoroethyl)amino]-2-(trifluoromethyl)benzonitrile;
1,1-Dimethylethyl[(5-{[[4-cyano-3-(trifluoromethyl)phenyl](2,2,2-trifluoroethyl)amino]methyl}-1,3,4-oxadiazol-2-yl)methyl]carbamate;
4-[{[5-(3,5-Difluorophenyl)-1,3,4-oxadiazol-2-yl]methyl}(2,2,2-trifluoroethyl)amino]-2-(trifluoromethyl)benzonitrile;
4-[{[5-(6-Bromo-2-pyridinyl)-1,3,4-oxadiazol-2-yl]methyl}(2,2,2-trifluoroethyl)amino]-2-(trifluoromethyl)benzonitrile;
4-[{[5-(2,4-Difluorophenyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[2-(trifluoromethyl)phenyl]-1,3,4-oxadiazol-2-yl}methyl)amino]-2-(trifluoromethyl)benzonitrile;
4-[(2,2,2-Trifluoroethyl)({5-[3-(trifluoromethyl)phenyl]-1,3,4-oxadiazol-2-yl}methyl)amino]-2-(trifluoromethyl)benzonitrile;
4-[{[5-(2-Fluorophenyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[4-(trifluoromethyl)phenyl]-1,3,4-oxadiazol-2-yl}methyl)amino]-2-(trifluoromethyl)benzonitrile;
4-[({5-[2-Chloro-6-(methyloxy)-4-pyridinyl]-1,3,4-oxadiazol-2-yl}methyl)(2,2,2-trifluoroethyl)amino]-2-(trifluoromethyl)benzonitrile;
4-[{[5-(1,3-Dimethyl-1H-pyrazol-5-yl)-1,3,4-oxadiazol-2-yl]methyl}(2,2,2-trifluoroethyl)amino]-2-(trifluoromethyl)benzonitrile;
4-[{[5-(2-Pyrazinyl)-1,3,4-oxadiazol-2-yl]methyl}(2,2,2-trifluoroethyl)amino]-2-(trifluoromethyl)benzonitrile;
4-[{[5-(5-Pyrimidinyl)-1,3,4-oxadiazol-2-yl]methyl}(2,2,2-trifluoroethyl)amino]-2-(trifluoromethyl)benzonitrile;
4-[{[5-(1-Methyl-1H-pyrazol-3-yl)-1,3,4-oxadiazol-2-yl]methyl}(2,2,2-trifluoroethyl)amino]-2-(trifluoromethyl)benzonitrile;
4-{2-(5-Oxo-4,5-dihydro-1,3,4-oxadiazol-2-yl)-1-[(trifluoromethyl)amino]ethyl}-2-(trifluoromethyl)benzonitrile;
4-{2-(4-Methyl-5-oxo-4,5-dihydro-1,3,4-oxadiazol-2-yl)-1-[(trifluoromethyl)amino]ethyl}-2-(trifluoromethyl)benzonitrile;
4-{2-[5-(Dimethylamino)-1,3,4-oxadiazol-2-yl]-1-[(trifluoromethyl)amino]ethyl}-2-(trifluoromethyl)benzonitrile;
4-{2-[5-(4-Morpholinyl)-1,3,4-oxadiazol-2-yl]-1-[(trifluoromethyl)amino]ethyl}-2-(trifluoromethyl)benzonitrile;
4-{2-[5-(1-Piperidinyl)-1,3,4-oxadiazol-2-yl]-1-[(trifluoromethyl)amino]ethyl}-2-(trifluoromethyl)benzonitrile;
4-{2-(5-Amino-1,3,4-oxadiazol-2-yl)-1-[(trifluoromethyl)amino]ethyl}-2-(trifluoromethyl)benzonitrile;
2-Chloro-4-((cyclopropylmethyl){[5-(2-pyridinyl)-1,3,4-oxadiazol-2-yl]methyl}amino)benzonitrile;
2-Chloro-4-[{[5-(2-pyridinyl)-1,3,4-oxadiazol-2-yl]methyl}(2,2,2-trifluoroethyl)amino]benzonitrile;
4-((Cyclopropylmethyl){[5-(2-pyridinyl)-1,3,4-oxadiazol-2-yl]methyl}amino)-1,2-benzenedicarbonitrile,
4-[{[3-(2-Pyridinyl)-1H-1,2,4-triazol-5-yl]methyl}(2,2,2-trifluoroethyl)amino]-2-(trifluoromethyl)benzonitrile;
4-[{[3-(5-Bromo-3-pyridinyl)-1H-1,2,4-triazol-5-yl]methyl}(2,2,2-trifluoroethyl)amino]-2-(trifluoromethyl)benzonitrile;
4-[{[5-(4-Fluorophenyl)-4-(phenylmethyl)-4H-1,2,4-triazol-3-yl]methyl}(2,2,2-trifluoroethyl)amino]-2-(trifluoromethyl)benzonitrile;
4-[{[3-(4-Fluorophenyl)-1H-1,2,4-triazol-5-yl]methyl}(2,2,2-trifluoroethyl)amino]-2-(trifluoromethyl)benzonitrile;
   and 4-[[(2-Methyl-2H-tetrazol-5-yl)methyl](2,2,2-trifluoroethyl)amino]-2-(trifluoromethyl)benzonitrile; or a salt or solvate thereof.

### ABBREVIATIONS

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
rt (room temperature); min (minute);
h (hour); mp (melting point);
TLC (thin layer chromatography); CH₂Cl₂ (methylene chloride);
TEA (triethylamine); TFA (trifluoroacetic acid);
TFAA (trifluoroacetic anhydride); THF (tetrahydrofuran);
CDCl₃ (deuterated chloroform); CD₃OD (deuterated methanol);
SiO₂ (silica); DMSO (dimethylsulfoxide);
EtOAc (ethyl acetate); atm (atmosphere);
HCl (hydrochloric acid); CHCl₃ (chloroform);
DMF (N,N-dimethylformamide); Ac (acetyl);
Cs₂CO₃ (cesium carbonate); Me (methyl);
Et (ethyl); EtOH (ethanol);
MeOH (methanol); t-Bu (tert-butyl);
Et₂O (diethyl ether); N₂ (nitrogen);
MsCl (methanesulphonyl chloride); sat'd (saturated);
K₂CO₃ (potassium carbonate); DMAP (4-(dimethylamino)pyridine);
DCE (1,2-dichloroethane); Ps (polymer supported);
EDCl (1-(3-dimethylaminopropyl)-3-ethylcarbodiimide hydrochloride);
P-BEMP (polymer-supported 2-tert-butylimino-2-diethylamino-1,3-dimethylperhydro-1,3,2-diaza-phosphorine); TsCl (tosyl chloride);
TES (triethylsilane); TBAF (tetrabutylammonium fluoride);
CSA (camphor sulfonic acid); n-BuLi (n-butyllithium);
TBDPSCl (tert-butyldiphenyl silylchloride);
HOAc (acetic acid); AcCl (acetyl chloride);
DIBAL-H (diisobutyl aluminium hydride);
CDI (carbonyl diimidazole); LiHDMS (lithium hexamethyldisilazide);
cat. (catalytic); anh. (anhydrous);
v/v (volume to volume); CCl₄ (carbon tetrachloride);
PPh₃ (triphenylphosphine); ZnBr₂ (zinc bromide);
PdPPh₃ (palladium tetrakis[triphenylphosphine].

Unless otherwise indicated, all temperatures are expressed in ° C. (degrees Centigrade). All reactions conducted under an inert atmosphere at room temperature unless otherwise noted. Reagents employed without synthetic details are commercially available or made according to literature procedures.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or b (broad).

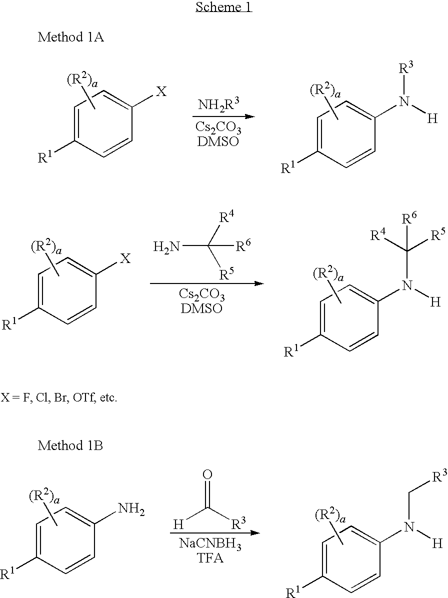

Secondary anilines used for the synthesis of compounds of formula (I) may be prepared by two different methods (Scheme 1). As exemplified by method 1A, electron deficient arenes are treated with primary amines, a non-limiting example is 1-cyclopropylmethanamine, in the presence of a base, a non-limiting example of which is cesium carbonate, to afford the corresponding aniline. A second method of synthesizing secondary anilines is by reductive alkylation of primary anilines using aldehydes or hydrates, a non-limiting example of which is trifluoroacetaldehyde hydrate, and reducing agents, a non-limiting example of which is sodium cyanoborohydride, in the presence of acid such as TFA (method 1B).

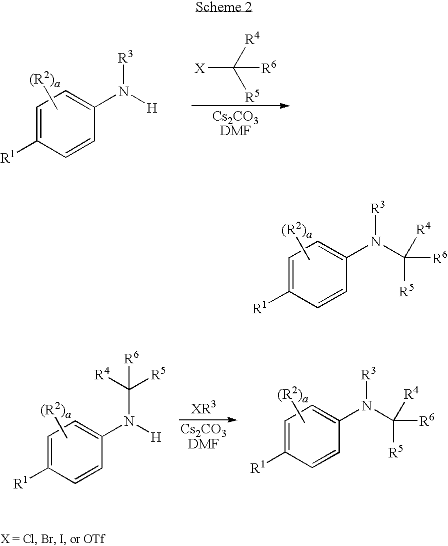

The secondary anilines of Scheme 1 may be further elaborated by alkylation with alkyl halides in the presence of a base. A typical non-limiting example of an alkylating agent would be benzyl bromide, while a typical non-limiting example of a base would be cesium carbonate (Scheme 2).

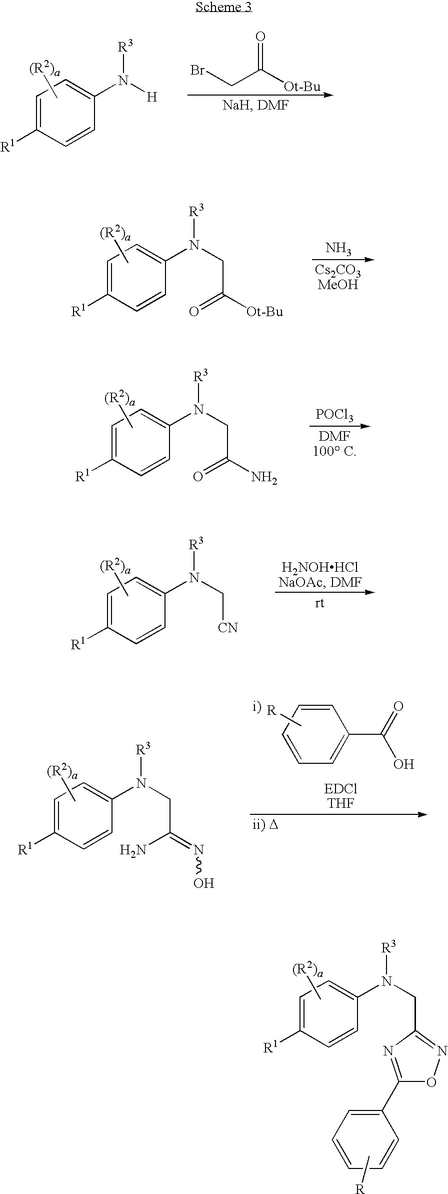

Heterocycle-bearing cyanoarylamines that cannot be made from simple alkylation with, for example, commercially available 3-halomethyl-5-substituted 1,2,4-oxadiazoles (as in Scheme 2) may be synthesized by coupling of an amide oxime with a carboxylic acid followed by thermally-induced ring closure (Scheme 3). The requisite amide oximes are synthesized starting from the alkylation of secondary anilines with α-halo esters such as t-butyl bromoacetate. Conversion of the ester to a primary amide is effected through aminolysis with methanolic ammonia in the presence of a base catalyst such as cesium carbonate. Dehydration affords a nitrile which is then converted to the amide oxime by treatment with hydroxylamine hydrochloride and sodium acetate.

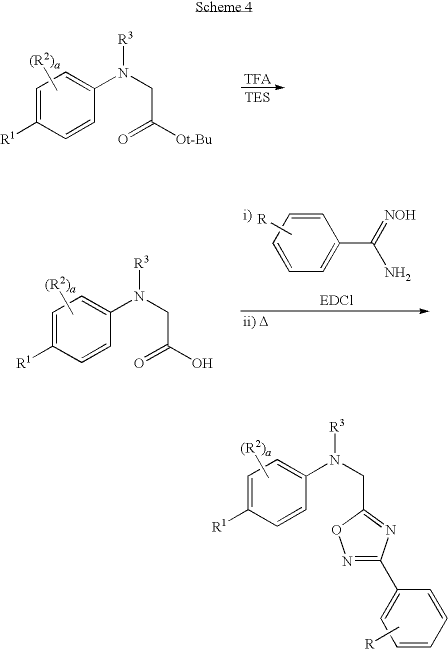

Alternative substitution patterns to those depicted in Scheme 3 are afforded by reversing the carboxylic and amide oxime coupling partners (Scheme 4). De-t-butylation of the glycine ester from the parent benzonitriles affords the corresponding carboxylic acid. The acid is coupled to an amide oxime in the presence of a coupling agent, such as EDCl. Heating affords the cyclized products.

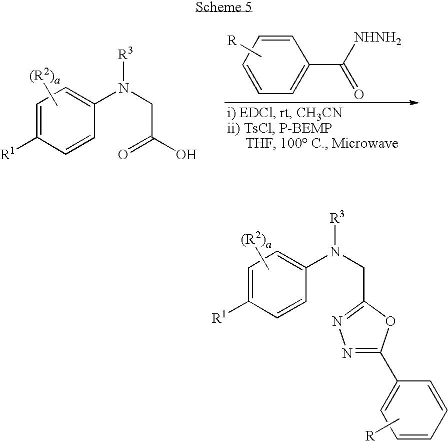

The same carboxylic acid reaction partner depicted in Scheme 4 may be used to afford 1,3,4-oxadiazole bearing compounds (Scheme 5). Reaction of a carboxylic acid with a carbohydrazide in the presence of a coupling agent, such as EDCl, is followed by treatment with tosyl chloride and P-BEMP as a base. The resulting mixture is then heated via microwave to give the desired heterocycles.

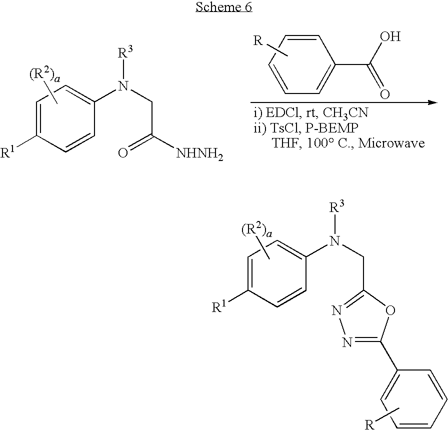

The reaction partners for formation of 1,3,4-oxadiazole bearing compounds can also be reversed, allowing one to utilize a large variety of carboxylic acids to couple to the corresponding phenylamino acetohydrazides.

### EXAMPLES

### Example 1

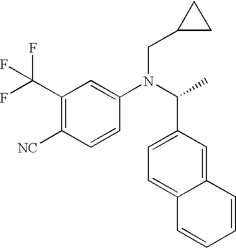

### 4-{(Cyclopropylmethyl)[(1R)-1-(2-naphthyl)ethyl]amino}-2-(trifluoromethyl)benzonitrile

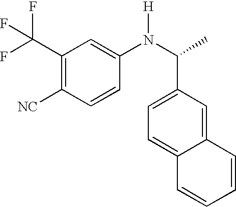

### A. 4-{[(1R)-1-(2-Naphthyl)ethyl]amino}-2-(trifluoromethyl)benzonitrile

A solution of 4-fluoro-2-trifluoromethylbenzonitrile (0.094 g, 0.497 mmol), (R)-(+)-1-(2-naphthyl)ethylamine (0.102 g, 0.596 mmol) and Cs₂CO₃ (0.243 g, 0.750 mmol) in DMSO (0.5 mL) under N₂ was heated to 90° C. for 2 h and then cooled to ambient temperature. H₂O was added and the mixture was extracted with EtOAc. The organic fraction was washed with brine, dried (Na₂SO₄), filtered, and concentrated to an oily residue, which was subjected to chromatography on silica gel using hexanes:EtOAc. The title compound was obtained as a white solid (0.125 g, 74% yield): MS (ES) m/z 339 (M−1).

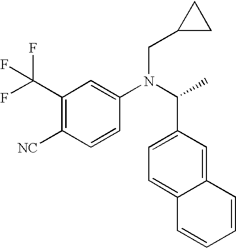

### B. 4-{(Cyclopropylmethyl)[(1R)-1-(2-naphthyl)ethyl]amino}-2-(trifluoromethyl)benzonitrile

A solution of Example 1A (0.125 g, 0.367 mmol) in DMF (2.0 mL) under N₂ was cooled to 0° C. and treated with NaH (0.026 g, 1.10 mmol) and stirred for 30 min at rt. Cyclopropylmethyl bromide (0.053 mL, 0.551 mmol) was added and the reaction mixture was stirred for 4 h. H₂O was added and the mixture was extracted with EtOAc. The organic portion was washed with brine, dried (Na₂SO₄), filtered, and concentrated to an oily residue, which was subjected to chromatography on silica gel using hexanes:EtOAc to provide the title compound as an amber oil (0.095 g, 66% yield): ¹H NMR (400 MHz, CDCl₃) δ 7.85-7.78 (m, 3H), 7.70 (bs, 1H), 7.53 (d, J=9.0 Hz, 1H), 7.50-7.45 (m, 2H), 7.32 (dd, J=8.6, 1.7 Hz, 1H), 7.20 (d, J=2.5 Hz, 1H), 6.96 (dd, J=8.6, 2.6 Hz, 1H), 5.30 (q, J=6.9 Hz, 1H), 3.24 (dd, J=15.4, 5.5 Hz, 1H), 3.11 (dd, J=15.4, 5.9 Hz, 1H), 1.79 (d, J=6.8 Hz, 3H), 0.97-0.90 (m, 1H), 0.61-0.55 (m, 2H), 0.30-0.00 (m, 2H); MS (APCI) m/z 395 (M+1).

### Example 2

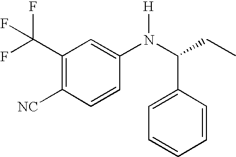

### 4-{[(1R)-1-Phenylpropyl]amino}-2-(trifluoromethyl)benzonitrile

Prepared from (1R)-1-phenylpropan-1-amine in a manner similar to that described in Example 1A: MS (ES) m/z 305 (M+1).

### Example 3

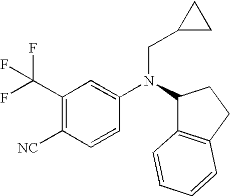

### 4-{(Cyclopropylmethyl)[(1S)-2,3-dihydro-1H-inden-1-yl]amino}-2-(trifluoromethyl)benzonitrile

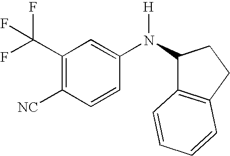

### A. 4-[(1S)-2,3-Dihydro-1H-inden-1-ylamino]-2-(trifluoromethyl)benzonitrile

Prepared from (S)-1-aminoindane in a manner similar to that described in Example 1A: MS (ES) m/z 301 (M−1).

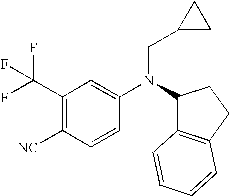

### B. 4-{(Cyclopropylmethyl)[(1S)-2,3-dihydro-1H-inden-1-yl]amino}-2-(trifluoromethyl)benzonitrile

Prepared from 3A and cyclopropylmethyl bromide in a manner similar to that described in Example 1B and obtained as an amber oil: ¹H NMR (400 MHz, CDCl₃) δ 7.54 (d, J=8.7 Hz, 1H), 7.29-7.09 (m, 5H), 6.91 (d, J=8.7 Hz, 1H), 5.44 (t, J=8.0 Hz, 1H), 3.27 (dd, J=15.4, 5.5 Hz, 1H), 3.13-3.06 (m, 2H), 3.00-2.91 (m, 1H), 2.59-2.49 (m, 1H), 2.21-2.11 (m, 1H), 1.00-0.92 (m, 1H), 0.59-0.52 (m, 2H), 0.13-0.05 (m, 2H).

### Example 4

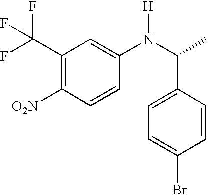

### N-[(1R)-1-(4-Bromophenyl)ethyl]-4-nitro-3-(trifluoromethyl)aniline

Prepared from 5-fluoro-2-nitrobenzonitrile and (R)-1-(4-bromophenethyl)amine in a manner similar to that described in Example 1A and obtained as a yellow oil: ¹H NMR (400 MHz, CDCl₃) δ 7.90 (d, J=9.0 Hz, 1H), 7.48 (d, J=8.5 Hz, 2H), 7.18 (d, J=8.4 Hz, 2H), 6.87 (d, J=2.2 Hz, 1H), 6.48 (dd, J=9.0, 2.2 Hz, 1H), 4.92 (bd, J=4.6 Hz, 1H), 4.60-4.50 (m, 1H), 1.56 (d, J=6.6 Hz, 3H).

### Example 5

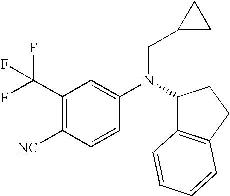

### 4-{(Cyclopropylmethyl)[(1R)-2,3-dihydro-1H-inden-1-yl]amino}-2-(trifluoromethyl)benzonitrile

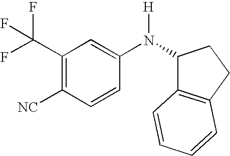

### A. 4-[(1R)-2,3-Dihydro-1H-inden-1-ylamino]-2-(trifluoromethyl)benzonitrile

Prepared from (R)-1-aminoindane in a manner similar to that described in Example 1A: MS (ES) m/z 301 (M−1).

### B. 4-{(Cyclopropylmethyl)[(1R)-2,3-dihydro-1H-inden-1-yl]amino}-2-(trifluoromethyl)benzonitrile

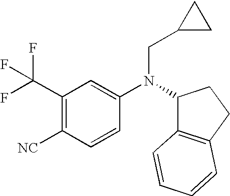

Prepared from Example 5A and cyclopropylmethyl bromide in a manner similar to that described in Example 1B: ¹H NMR (400 MHz, CDCl₃) δ 7.54 (d, J=8.7 Hz, 1H), 7.31-7.10 (m, 5H), 6.91 (d, J=8.7 Hz, 1H), 5.44 (t, J=8.0 Hz, 1H), 3.27 (dd, J=15.4, 5.5 Hz, 1H), 3.13-3.06 (m, 2H), 3.00-2.91 (m, 1H), 2.59-2.49 (m, 1H), 2.21-2.11 (m, 1H), 1.00-0.92 (m, 1H), 0.59-0.52 (m, 2H), 0.13-0.05 (m, 2H).

### Example 6

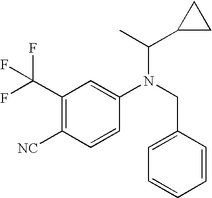

### 4-[Benzyl(1-cyclopropylethyl)amino]-2-(trifluoromethyl)benzonitrile

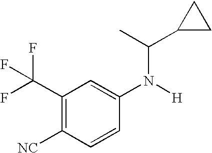

### A. 4-[(1-Cyclopropylethyl)amino]-2-(trifluoromethyl)benzonitrile

Prepared from 1-cyclopropylethyl amine (ref. J. L. Kelly, R. M. Morris, M. P. Krochmal, E. W. McLean, J. A. Linn, M. J. Durcan, D. R. Cooper, J. Med. Chem., 1997, 40(20), 3207-3216) in a manner similar to that described in Example 1A and taken on to the next step as crude material.

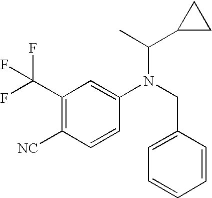

### B. 4-[Benzyl(1-cyclopropylethyl)amino]-2-(trifluoromethyl)benzonitrile

Prepared from Example 6A and benzyl bromide in a manner similar to that described in Example 1B: ¹H NMR (400 MHz, CDCl₃) δ 7.47 (d, J=8.8 Hz, 1H), 7.34-7.29 (m, 2H), 7.27-7.20 (m, 3H), 6.94 (d, J=2.5 Hz, 1H), 6.70 (dd, J=8.8, 2.5 Hz, 1H), 4.75 (d, J=17.7 Hz, 1H), 4.60 (d, J=17.8 Hz, 1H), 3.48-3.38 (m, 1H), 1.31 (d, J=6.6 Hz, 3H), 1.02-0.95 (m, 1H), 0.68-0.59 (m, 1H), 0.45-0.25 (m, 3H).

### Example 7

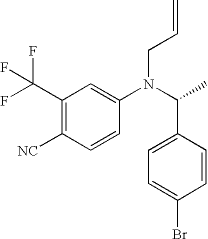

### 4-{Allyl[(1R)-1-(4-bromophenyl)ethyl]amino}-2-(trifluoromethyl)benzonitrile

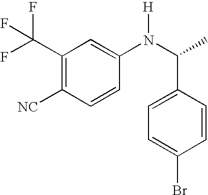

### A. 4-{[(1R)-1-(4-Bromophenyl)ethyl]amino}-2-(trifluoromethyl)benzonitrile

Prepared from (R)-1-(4-bromophenethyl)amine in a manner similar to that described in Example 1A and used for the next step without purification.

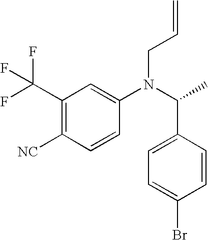

### B. 4-{Allyl[(1R)-1-(4-bromophenyl)ethyl]amino}-2-(trifluoromethyl)benzonitrile

Prepared from Example 7A and allyl bromide in a manner similar to that described in Example 1B: ¹H NMR (400 MHz, CDCl₃) δ 7.48-7.45 (m, 3H), 7.18 (d, J=8.4 Hz, 2H), 6.82 (d, J=2.2 Hz, 1H), 6.51 (dd, J=8.6, 2.2 Hz, 1H), 5.77-5.70 (m, 1H), 5.37 (q, J=6.5 Hz, 1H), 5.22-5.12 (m, 2H), 3.82-3.78 (m, 2H), 1.56 (d, J=6.4 Hz, 3H).

### Example 8

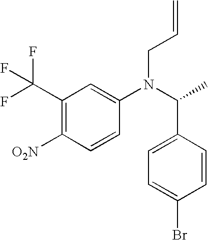

### N-Allyl-N-[(1R)-1-(4-bromophenyl)ethyl]-4-nitro-3-(trifluoromethyl)aniline

Prepared from Example 4 and allyl bromide in a manner similar to that described in Example 1B: ¹H NMR (400 MHz, CDCl₃) δ 7.90 (d, J=9.0 Hz, 1H), 7.48 (d, J=8.5 Hz, 2H), 7.18 (d, J=8.4 Hz, 2H), 6.87 (d, J=2.2 Hz, 1H), 6.48 (dd, J=9.0, 2.2 Hz, 1H), 5.77-5.70 (m, 1H), 5.37 (q, J=6.5 Hz, 1H), 5.22-5.12 (m, 2H), 3.82-3.78 (m, 2H), 1.56 (d, J=6.6 Hz, 3H).

### Example 9

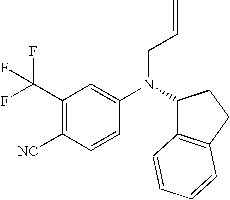

### 4-{Allyl[(1R)-2,3-dihydro-1H-inden-1-yl]amino}-2-(trifluoromethyl)benzonitrile

Prepared from 5A and allyl bromide in a manner similar to that described in Example 1B and obtained as a yellow oil: ¹H NMR (400 MHz, CDCl₃) δ7.57 (d, J=8.8 Hz, 1H), 7.29-7.10 (m, 5H), 6.91 (dd, J=8.8, 2.5 Hz, 1H), 5.80-5.69 (m, 1H), 5.55 (t, J=7.9 Hz, 1H), 5.24-5.10 (m, 2H), 3.81 (bs, 2H), 3.08-3.00 (m, 1H), 3.00-2.89 (m, 1H), 2.54-2.46 (m, 1H), 2.10-2.00 (m, 1H).

### Example 10

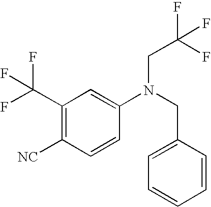

### 4-[Benzyl(2,2,2-trifluoroethyl)amino]-2-(trifluoromethyl)benzonitrile

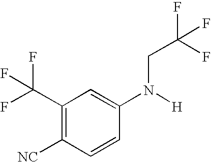

### A. 4-(2,2,2-Trifluoro-ethylamino)-2-trifluoromethyl-benzonitrile

To a slurry of 4-amino-2-(trifluoromethyl)benzonitrile (30.09 g, 162 mmol) and NaBH₃CN (21.35 g, 340 mmol) in CH₂Cl₂ (160 mL) at ice bath temperature was added neat TFA (160 mL, 2.08 mol), dropwise at a rate such that the internal temperature remained below 5° C. (CAUTION: exothermic reaction with hydrogen gas evolution). Trifluoroacetaldehyde hydrate (52.2 g, 405 mmol) was then added over 5 min (CAUTION: slightly exothermic reaction, with gas evolution). After 41 h, the mixture was slowly poured into sat'd NaHCO₃ (1 L) at 0° C. The mixture was then completely neutralized by portionwise addition of solid NaHCO₃. The mixture was stirred 30 min and precipitated solids were collected by filtration. Organic and aqueous phases of the filtrate were separated, and the aqueous layer was extracted with CH₂Cl₂ (3×150 mL). Combined organic extracts were concentrated to dryness, combined with the solids collected previously, dissolved in EtOAc, washed (H₂O, brine), dried over Na₂SO₄, filtered through a short pad of Celite, and concentrated to dryness. Recrystallization from EtOAc/hexanes yielded 32.61 g (95%) of the title compound as slightly tan crystalline plates: ¹H NMR (300 MHz, CD₃OD) δ 7.59 (d, J=8.8 Hz, 1H), 7.05 (d, J=2.2 Hz, 1H), 6.92 (dd, J=8.7, 2.4 Hz, 1H), 3.92 (q, J=9.2 Hz, 2H).

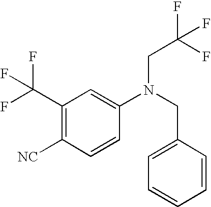

### B. 4-[Benzyl(2,2,2-trifluoroethyl)amino]-2-(trifluoromethyl)benzonitrile

A mixture of Example 10A (0.050 g, 0.186 mmol), Cs₂CO₃ (0.121 g, 0.372 mmol) and benzyl bromide (0.064 g, 0.372 mmol) in CH₃CN (2.5 mL) was heated at 80° C. under nitrogen for 1 h. Upon cooling, the reaction mixture was partitioned between EtOAc (30 mL) and water (20 mL). The organic phase was washed with brine (10 mL), dried (Na₂SO₄) and concentrated in vacuo. The residue was purified by silica gel chromatography (2-30% EtOAc-hexanes gradient) to give the title compound (0.060 g, 90%) as a colorless oil: ¹H NMR (400 MHz, CDCl₃) δ 7.60 (d, J=8.8 Hz, 1H), 7.40-7.28 (m, 3H), 7.15-7.08 (m, 3H), 6.92 (dd, J=8.8, 2.0 Hz, 1H), 4.78 (s, 2H), 4.10 (q, J=8.4 Hz, 2H); MS (ES) m/z 359 (M+1).

### Example 11

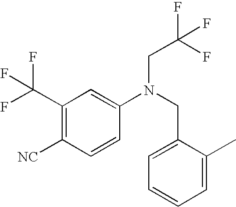

### 4-[(2-Methylbenzyl)(2,2,2-trifluoroethyl)amino]-2-(trifluoromethyl)benzonitrile

Synthesized as described in Example 10B using 1-(bromomethyl)-2-methylbenzene: MS (ES) m/z 373 (M+1).

### Example 12

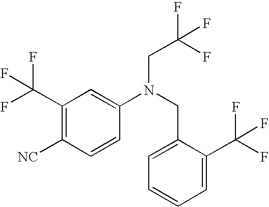

### 4-{(2,2,2-Trifluoroethyl)[2-(trifluoromethyl)benzyl]amino}-2-(trifluoromethyl)benzonitrile

Synthesized as described in Example 10B using 2-(trifluoromethyl)benzyl chloride: MS (ES) m/z 425 (M−1).

### Example 13

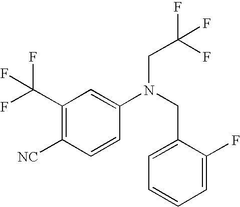

### 4-[(2-Fluorobenzyl)(2,2,2-trifluoroethyl)amino]-2-(trifluoromethyl)benzonitrile

Synthesized as described in Example 10B using 2-fluorobenzyl bromide: MS (ES) m/z 377 (M+1).

### Example 14

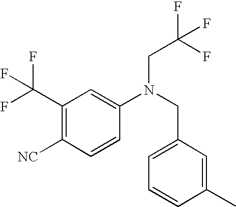

### 4-[(3-Methylbenzyl)(2,2,2-trifluoroethyl)amino]-2-(trifluoromethyl)benzonitrile

Synthesized as described in Example 10B using 1-(bromomethyl)-3-methylbenzene: MS (ES) m/z 373 (M+1).

### Example 15

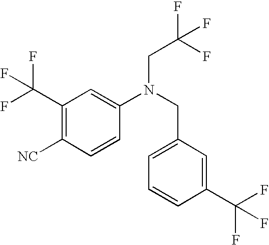

### 4-{(2,2,2-Trifluoroethyl)[3-(trifluoromethyl)benzyl]amino}-2-(trifluoromethyl)benzonitrile

Synthesized as described in Example 10B using 3-(trifluoromethyl)benzyl bromide: MS (ES) m/z 425 (M−1).

### Example 16

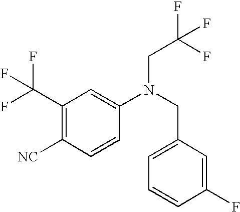

### 4-[(3-Fluorobenzyl)(2,2,2-trifluoroethyl)amino]-2-(trifluoromethyl)benzonitrile

Synthesized as described in Example 10B using 3-fluorobenzyl bromide: MS (ES) m/z 377 (M+1).

### Example 17

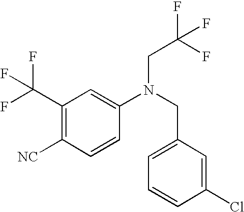

### 4-[(3-Chlorobenzyl)(2,2,2-trifluoroethyl)amino]-2-(trifluoromethyl)benzonitrile

Synthesized as described in Example 10B using 3-chlorobenzyl bromide: MS (ES) m/z 391 (M−1).

### Example 18

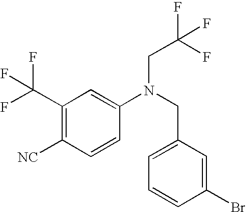

### 4-[(3-Bromobenzyl)(2,2,2-trifluoroethyl)amino]-2-(trifluoromethyl)benzonitrile

Synthesized as described in Example 10B using 3-bromobenzyl bromide: MS (ES) m/z 437, 439 (M+1 isotopes).

### Example 19

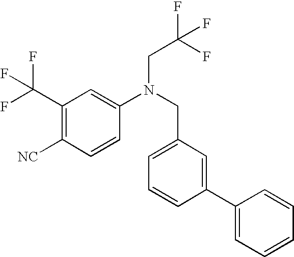

### 4-[(1,1′-Biphenyl-3-ylmethyl)(2,2,2-trifluoroethyl)amino]-2-(trifluoromethyl)benzonitrile

A mixture of Example 18 (0.056 g, 0.128 mmol), phenyl boronic acid (0.047 g, 0.384 mmol), 10% Pd/C (0.100 g), NaHCO₃ (0.032 g, 0.384 mmol) and water (0.8 mL) in 4 mL of DMF was heated under nitrogen at 120° C. for 12 h. Upon cooling, the reaction mixture was filtered and the catalyst was washed with Et₂O. The filtrate was partitioned between diethyl ether and 0.1N NaOH. The organic phase was washed with 0.1N NaOH and brine, dried (Na₂SO₄), and concentrated in vacuo. The residue was purified by silica gel chromatography (5-40% EtOAc-hexanes gradient), followed by a second chromatography (10-60% CH₂Cl₂-hexanes gradient) to give the title compound (0.050 g, 90%): MS (ES) m/z 435 (M+1).

### Example 20

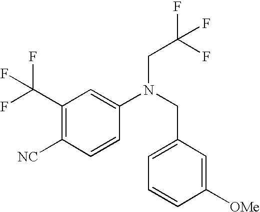

### 4-[(3-Methoxybenzyl)(2,2,2-trifluoroethyl)amino]-2-(trifluoromethyl)benzonitrile

Synthesized as described in Example 10B using 3-methoxybenzyl bromide: MS (ES) m/z 389 (M+1).

### Example 21

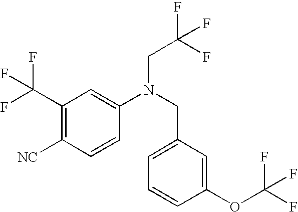

### 4-{(2,2,2-Trifluoroethyl)[3-(trifluoromethoxy)benzyl]amino}-2-(trifluoromethyl)benzonitrile

Synthesized as described in Example 10B using 3-(trifluoromethoxy)benzyl bromide: MS (ES) m/z 443 (M+1).

### Example 22

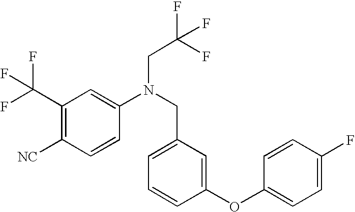

### 4-[[3-(4-Fluorophenoxy)benzyl](2,2,2-trifluoroethyl)amino]-2-(trifluoromethyl)benzonitrile

Synthesized as described in Example 10B using 1-(bromomethyl)-3-(4-fluorophenoxy)benzene: MS (ES) m/z 469 (M+1).

### Example 23

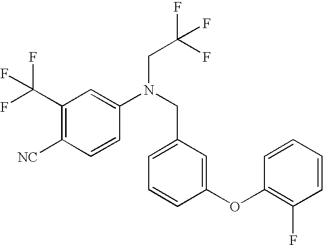

### 4-[[3-(2-Fluorophenoxy)benzyl](2,2,2-trifluoroethyl)amino]-2-(trifluoromethyl)benzonitrile

Synthesized as described in Example 10B using 3-(bromomethyl)phenyl 2-fluorophenyl ether: MS (ES) m/z 469 (M+1).

### Example 24

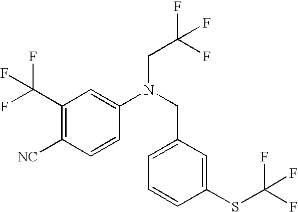

### 4-((2,2,2-Trifluoroethyl){3-[(trifluoromethyl)thio]benzyl}amino)-2-(trifluoromethyl)benzonitrile

Synthesized as described in Example 10B using 1-(bromomethyl)-3-[(trifluoromethyl)thio]benzene: MS (ES) m/z 457 (M−1).

### Example 25

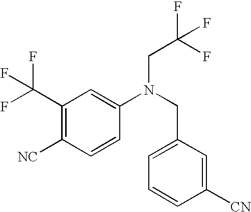

### 4-[(3-Cyanobenzyl)(2,2,2-trifluoroethyl)amino]-2-(trifluoromethyl)benzonitrile

Synthesized as described in Example 10B using 3-cyanobenzyl bromide: MS (ES) m/z 384 (M+1).

### Example 26

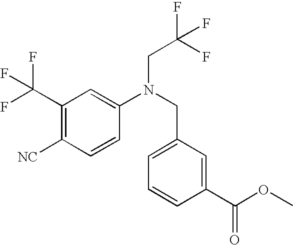

### Methyl 3-{[[4-cyano-3-(trifluoromethyl)phenyl](2,2,2-trifluoroethyl)amino]methyl}benzoate

Synthesized as described in Example 10B using methyl 3-bromomethyl benzoate: MS (ES) m/z 417 (M+1).

### Example 27

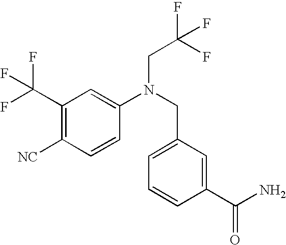

### 3-{[[4-Cyano-3-(trifluoromethyl)phenyl](2,2,2-trifluoroethyl)amino]methyl}benzamide

Synthesized as described in Example 10B using 3-(chloromethyl)benzamide: MS (ES) m/z 402 (M+1).

### Example 28

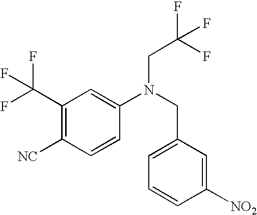

### 4-[(3-Nitrobenzyl)(2,2,2-trifluoroethyl)amino]-2-(trifluoromethyl)benzonitrile

Synthesized as described in Example 10B using 3-nitrobenzyl bromide: MS (ES) m/z 402 (M−1).

### Example 29

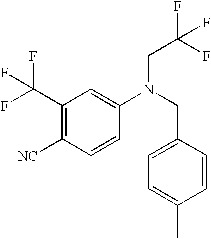

### 4-[(4-Methylbenzyl)(2,2,2-trifluoroethyl)amino]-2-(trifluoromethyl)benzonitrile

Synthesized as described in Example 10B using 1-(bromomethyl)-4-methylbenzene: MS (ES) m/z 373 (M+1).

### Example 30

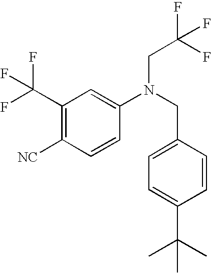

### 4-[(4-tert-Butylbenzyl)(2,2,2-trifluoroethyl)amino]-2-(trifluoromethyl)benzonitrile

Synthesized as described in Example 10B using 4-(tert-butyl)benzyl bromide: MS (ES) m/z 437 (M+Na).

### Example 31

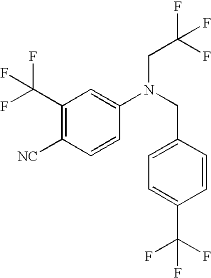

### 4-{(2,2,2-Trifluoroethyl)[4-(trifluoromethyl)benzyl]amino}-2-(trifluoromethyl)benzonitrile

Synthesized as described in Example 10B using 4-(trifluoromethyl)benzyl bromide: MS (ES) m/z 425 (M−1).

### Example 32

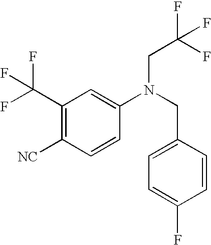

### 4-[(4-Fluorobenzyl)(2,2,2-trifluoroethyl)amino]-2-(trifluoromethyl)benzonitrile

Synthesized as described in Example 10B using 4-fluorobenzyl bromide: MS (ES) m/z 377 (M+1).

### Example 33

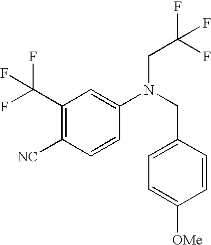

### 4-[(4-Methoxybenzyl)(2,2,2-trifluoroethyl)amino]-2-(trifluoromethyl)benzonitrile

Synthesized as described in Example 10B using 4-methoxybenzyl chloride: MS (ES) m/z 411 (M+Na).

### Example 34

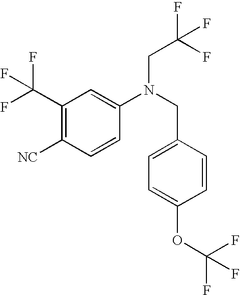

### 4-{(2,2,2-Trifluoroethyl)[(4-(trifluoromethoxy)benzyl]amino}-2-(trifluoromethyl)benzonitrile

Synthesized as described in Example 10B using 4-(trifluoromethoxy)benzyl bromide: MS (ES) m/z 443 (M+1).

### Example 35

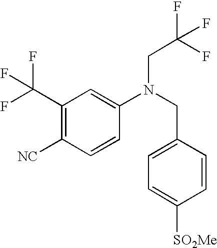

### 4-[[4-(Methylsulfonyl)benzyl](2,2,2-trifluoroethyl)amino]-2-(trifluoromethyl)benzonitrile

Synthesized as described in Example 10B using 4-(methylsulfonyl)benzyl bromide. MS (ES) m/z 435 (M−1).

### Example 36

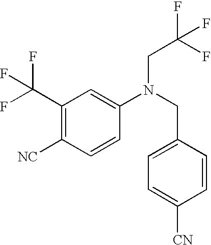

### 4-[(4-Cyanobenzyl)(2,2,2-trifluoroethyl)amino]-2-(trifluoromethyl)benzonitrile

Synthesized as described in Example 10B using 4-cyanobenzyl bromide: MS (ES) m/z 384 (M+1).

### Example 37

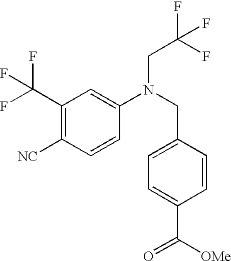

### Methyl 4-{[[4-cyano-3-(trifluoromethyl)phenyl](2,2,2-trifluoroethyl)amino]methyl}benzoate

Synthesized as described in Example 10B using methyl 4-bromomethyl benzoate: MS (ES) m/z 415 (M−1).

### Example 38

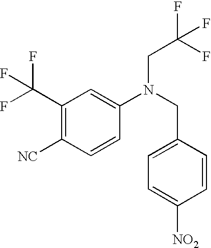

### 4-[(4-Nitrobenzyl)(2,2,2-trifluoroethyl)amino]-2-(trifluoromethyl)benzonitrile

Synthesized as described in Example 10B using 4-nitrobenzyl bromide: MS (ES) m/z 402 (M−1).

### Example 39

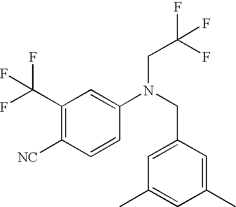

### 4-[(3,5-Dimethylbenzyl)(2,2,2-trifluoroethyl)amino]-2-(trifluoromethyl)benzonitrile

Synthesized as described in Example 10B using 3,5-dimethylbenzyl bromide: MS (ES) m/z 387 (M+1).

### Example 40

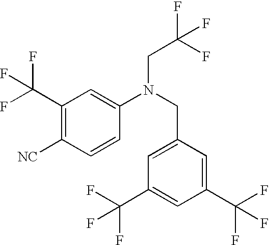

### 4-[[3,5-bis(Trifluoromethyl)benzyl](2,2,2-trifluoroethyl)amino]-2-(trifluoromethyl)benzonitrile

Synthesized as described in Example 10B using 1-(bromomethyl)-3,5-bis(trifluoromethyl)benzene: MS (ES) m/z 493 (M−1).

### Example 41

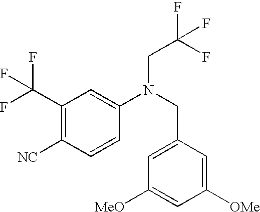

### 4-[(3,5-Dimethoxybenzyl)(2,2,2-trifluoroethyl)amino]-2-(trifluoromethyl)benzonitrile

Synthesized as described in Example 10B using 3,5-dimethoxybenzyl bromide: MS (ES) m/z 419 (M+1).

### Example 42

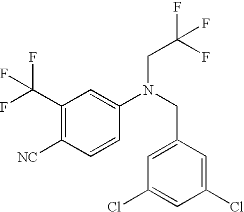

### 4-[(3,5-Dichlorobenzyl)(2,2,2-trifluoroethyl)amino]-2-(trifluoromethyl)benzonitrile

Synthesized as described in Example 10B using 3,5-dichlorobenzyl bromide: MS (ES) m/z 425 (M−1).

### Example 43

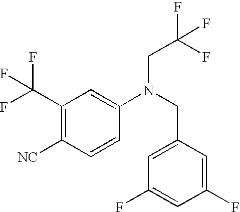

### 4-[(3,5-Difluorobenzyl)(2,2,2-trifluoroethyl)amino]-2-(trifluoromethyl)benzonitrile

Synthesized as described in Example 10B using 3,5-difluorobenzyl bromide: MS (ES) m/z 395 (M+1).

### Example 44

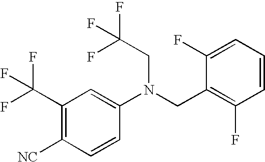

### 4-[(2,6-Difluorobenzyl)(2,2,2-trifluoroethyl)amino]-2-(trifluoromethyl)benzonitrile

Synthesized as described in Example 10B using 2,6-difluorobenzyl bromide: MS (ES) m/z 395 (M+1).

### Example 45

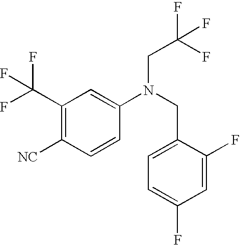

### 4-[(2,4-Difluorobenzyl)(2,2,2-trifluoroethyl)amino]-2-(trifluoromethyl)benzonitrile

Synthesized as described in Example 10B using 2,4-difluorobenzyl bromide: MS (ES) m/z 417 (M+Na).

### Example 46

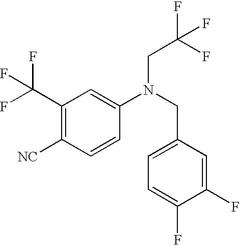

### 4-[(3,4-Difluorobenzyl)(2,2,2-trifluoroethyl)amino]-2-(trifluoromethyl)benzonitrile

Synthesized as described in Example 10B using 3,4-difluorobenzyl bromide: MS (ES) m/z 417 (M+Na).

### Example 47

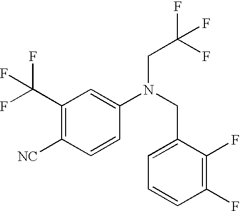

### 4-[(2,3-Difluorobenzyl)(2,2,2-trifluoroethyl)amino]-2-(trifluoromethyl)benzonitrile

Synthesized as described in Example 10B using 2,3-difluoro-benzyl bromide: MS (ES) m/z 395 (M+1).

### Example 48

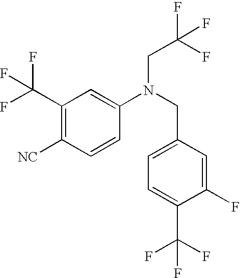

### 4-[[3-Fluoro-4-(trifluoromethyl)benzyl](2,2,2-trifluoroethyl)amino]-2-(trifluoromethyl)benzonitrile

Synthesized as described in Example 10B using 4-(bromomethyl)-2-fluoro-1-(trifluoromethyl)benzene: MS (ES) m/z 443 (M−1).

### Example 49

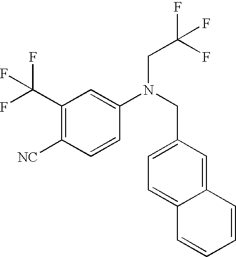

### 4-[(2-Naphthylmethyl)(2,2,2-trifluoroethyl)amino]-2-(trifluoromethyl)benzonitrile

Synthesized as described in Example 10B using 2-(bromomethyl)naphtalene: MS (ES) m/z 431 (M+Na).

### Example 50

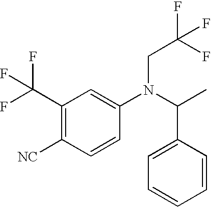

### 4-[(1-Phenylethyl)(2,2,2-trifluoroethyl)amino]-2-(trifluoromethyl)benzonitrile

Synthesized as described in Example 10B using (1-bromoethyl)benzene: MS (ES) m/z 373 (M+1).

### Example 51

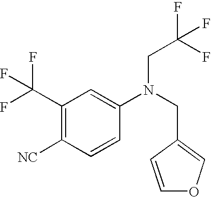

### 4-[(3-Furylmethyl)(2,2,2-trifluoroethyl)amino]-2-(trifluoromethyl)benzonitrile

Synthesized as described in Example 10B using 3-(chloromethyl)furan: MS (ES) m/z 349 (M+1).

### Example 52

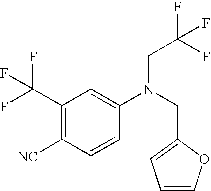

### 4-[(2-Furylmethyl)(2,2,2-trifluoroethyl)amino]-2-(trifluoromethyl)benzonitrile

Synthesized as described in Example 10B using 2-(chloromethyl)furan: MS (ES) m/z 349 (M+1).

### Example 53

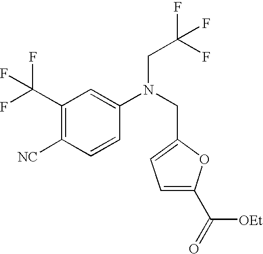

### Ethyl 5-{[[4-cyano-3-(trifluoromethyl)phenyl](2,2,2-trifluoroethyl)amino]methyl}-2-furoate

Synthesized as described in Example 10B using ethyl 5-(chloromethyl)-2-furoate: MS (ES) m/z 421 (M+1).

### Example 54

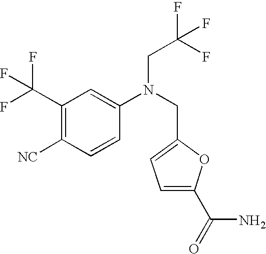

### 5-{[[4-Cyano-3-(trifluoromethyl)phenyl](2,2,2-trifluoroethyl)amino]methyl}-2-furamide

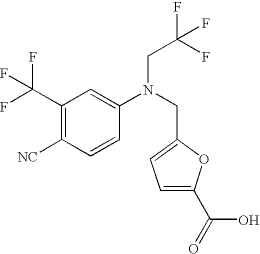

### A. 5-{[[4-Cyano-3-(trifluoromethyl)phenyl](2,2,2-trifluoroethyl)amino]methyl}-2-furoic Acid

To a solution of ethyl 5-{[[4-cyano-3-(trifluoromethyl)phenyl](2,2,2-trifluoroethyl)amino]methyl}-2-furoate, Example 53 (0.038 g, 0.09 mmol) in 1:1 THF/MeOH (4 mL) was added 1N NaOH (0.9 ml, 0.9 mmol) and the mixture was heated at 65° C. for 1 h. Upon cooling, the mixture was acidified with 1N HCl (2 mL) and partitioned between EtOAc and water. The organic phase was washed with brine, dried (Na₂SO₄), and concentrated in vacuo to give the title compound, which was carried on to the next step without further purification.

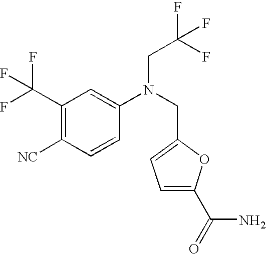

### B. 5-{[[4-Cyano-3-(trifluoromethyl)phenyl](2,2,2-trifluoroethyl)amino]methyl}-2-furamide

A solution of Example 54A (0.035 g, 0.09 mmol) in CH₂Cl₂ (5 mL), under N₂, in an ice bath was treated with oxalyl chloride (0.015 g, 0.12 mmol) and DMF (cat.). The mixture was heated at 40° C. for 35 min. Upon cooling, additional oxalyl chloride (0.015 g, 0.12 mmol) and DMF (cat.) were added and the mixture was heated at 40° C. for another 30 min. Upon cooling, NH₄OH (28% in water, 6 mL) was added and the mixture was stirred vigorously for 15 min. The mixture was partitioned between CH₂Cl₂ and water. The organic phase was washed with water and brine, dried (Na₂SO₄), and concentrated in vacuo. The residue was purified by silica gel chromatography (1-6% MeOH—CH₂Cl₂ gradient) and the product was crystallized from CH₂Cl₂-hexanes to give the title compound as a white solid (0.025 g, 71% overall yield): MS (ES) m/z 392 (M+1).

### Example 55

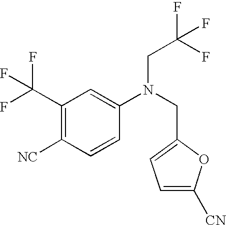

### 5-{[[4-Cyano-3-(trifluoromethyl)phenyl](2,2,2-trifluoroethyl)amino]methyl}-2-furonitrile

Polymer-supported triphenyl phosphine (0.051 g, 0.153 mmol, 3 mmol/g) was added to a solution of 5-{[[4-cyano-3-(trifluoromethyl)phenyl](2,2,2-trifluoroethyl)amino]methyl}-2-furamide, Example 54B (0.030 g, 0.076 mmol) in 1:1 CCl₄/DCE (3 mL) and the mixture was heated at 90° C. for 1.5 h. Upon cooling, the resin was filtered off and washed with CHCl₃ (2×10 mL). The filtrate was concentrated and the residue was purified by silica gel chromatography (5-40% EtOAc-hexanes gradient). The product was crystallized from CH₂Cl₂-hexanes to give the title compound as a white solid (0.021 g, 75% yield): MS (ES) m/z 374 (M+1).

### Example 56

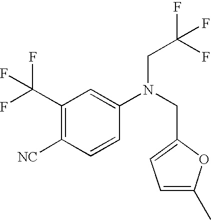

### 4-[[(5-Methyl-2-furyl)methyl](2,2,2-trifluoroethyl)amino]-2-(trifluoromethyl)benzonitrile

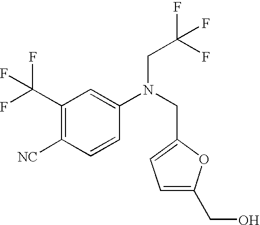

### A. 4-[{[5-(Hydroxymethyl)-2-furyl]methyl}(2,2,2-trifluoroethyl)amino]-2-(trifluoromethyl)benzonitrile

An ice-cold solution of ethyl 5-{[[4-cyano-3-(trifluoromethyl)phenyl](2,2,2-trifluoroethyl)amino]methyl}-2-furoate, Example 53 (0.140 g, 0.33 mmol) in THF (5 mL) was treated under nitrogen with LiBH₄ (0.5 mL of 2M THF solution, 1.0 mmol) and stirred at rt for 12 h. The mixture was cooled in an ice bath and treated dropwise, slowly with water (2 mL) and 1N HCl (1 mL) (exothermic). The mixture was partitioned between EtOAc and water. The organic phase was washed with brine, dried (Na₂SO₄), and concentrated in vacuo. The residue was purified by silica gel chromatography (5-40% EtOAc-hexanes gradient) to give the title compound (0.115 g, 90% yield): MS (ES) m/z 401 (M+Na).

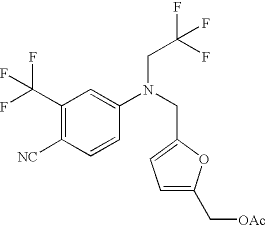

### B. (5-{[[4-Cyano-3-(trifluoromethyl)phenyl](2,2,2-trifluoroethyl)amino]methyl}-2-furyl)methyl acetate

A solution of Example 56A (0.037 g, 0.10 mmol) in CH₂Cl₂ (3 mL) was treated with TEA (0.011 g, 0.11 mmol) and acetic anhydride (0.011 g, 0.11 mmol) and stirred at rt. After 1 h, DMAP (0.012 mg, 0.1 mmol) was added and the reaction was stirred for 45 min. The mixture was concentrated in vacuo and the residue was partitioned between EtOAc and 0.5N HCl. The organic phase was washed with 0.5N HCl, water and brine, dried (Na₂SO₄) and concentrated in vacuo. The residue was purified by silica gel chromatography (5-50% EtOAc-hexanes gradient) to give the title compound (0.040 g, 95% yield): ¹H NMR (400 MHz, CDCl₃) δ 7.65 (d, J=8.8 Hz, 1H), 7.2 (d, J=2.6 Hz, 1H), 7.03 (dd, J=8.8, 2.7 Hz, 1H), 6.34 (d, J=3.3 Hz, 1H), 6.21 (d, J=3.3 Hz, 1H), 4.98 (s, 2H), 4.65 (s, 2H), 4.05 (q, J=8.4 Hz, 2H), 2.06 (s, 3H).

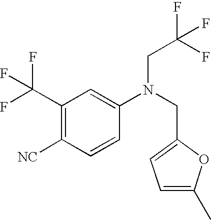

### C. 4-[[(5-Methyl-2-furyl)methyl](2,2,2-trifluoroethyl)amino]-2-(trifluoromethyl)benzonitrile

5% Pd/C (0.020 mg) in MeOH (3 mL) was stirred under H₂ (balloon pressure) for 10 min. Next, TEA (0.016 mg, 0.16 mmol) was added followed by a solution of Example 56B (0.022 g, 0.052 mmol) in MeOH (3 mL). After 30 min, the catalyst was filtered off and washed with MeOH and EtOAc. The filtrate was concentrated in vacuo and the residue was purified by silica gel chromatography (5-40% EtOAc-hexanes gradient) to give the title compound (0.015 g, 78% yield): MS (ES) m/z 363 (M+1).

### Example 57

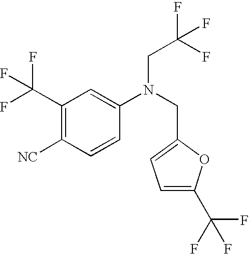

### 4-((2,2,2-Trifluoroethyl){[5-(trifluoromethyl)-2-furyl]methyl}amino)-2-(trifluoromethyl)benzonitrile

Synthesized as described in Example 10B using 2-(bromomethyl)-5-(trifluoromethyl)furan: MS (ES) m/z 417 (M+1).

### Example 58

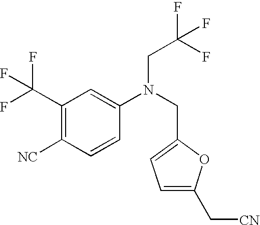

### 4-[{[5-(Cyanomethyl)-2-furyl]methyl}(2,2,2-trifluoroethyl)amino]-2-(trifluoromethyl)benzonitrile

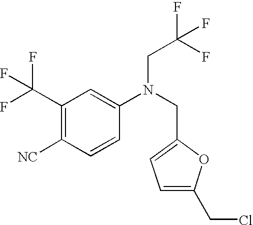

### A. 4-[{[5-(Chloromethyl)-2-furyl]methyl}(2,2,2-trifluoroethyl)amino]-2-(trifluoromethyl)benzonitrile

A solution of 4-[{[5-(hydroxymethyl)-2-furyl]methyl}(2,2,2-trifluoroethyl)amino]-2-(trifluoromethyl)benzonitrile, Example 56A (0.043 g, 0.113 mmol) in CCl₄ (3 mL) was treated with polymer-supported triphenyl phosphine (3 mmol/g, 0.076 g, 0.227 mmol) and heated at 80° C. for 1 h. Upon cooling, the resin was filtered off and washed with CH₂Cl₂. The filtrate was concentrated in vacuo to give the title compound which was used in the next reaction without purification.

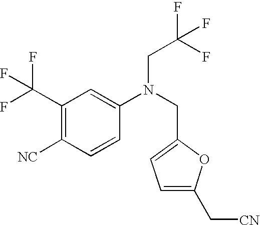

### B. 4-[{[5-(Cyanomethyl)-2-furyl]methyl}(2,2,2-trifluoroethyl)amino]-2-(trifluoromethyl)benzonitrile

A mixture of Example 58A (0.045 g, 0.11 mmol), NaCN (0.028 g, 0.57 mmol), and DMSO (3 mL) was stirred at rt for 20 min. The mixture was partitioned between EtOAc and water. The organic phase was washed with brine, dried (Na₂SO₄) and concentrated in vacuo. The residue was purified by silica gel chromatography (5-40% EtOAc-hexanes gradient) and the product was crystallized from CH₂Cl₂-hexanes to give the title compound as a light yellow solid (0.019 g, 43% overall yield): MS (ES) m/z 388 (M+1).

### Example 59

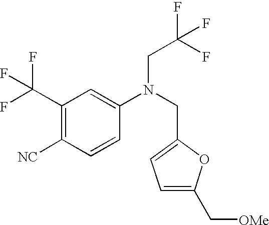

### 4-[{[5-(Methoxymethyl)-2-furyl]methyl}(2,2,2-trifluoroethyl)amino]-2-(trifluoromethyl)benzonitrile

A solution of 4-[{[5-(hydroxymethyl)-2-furyl]methyl}(2,2,2-trifluoroethyl)amino]-2-(trifluoromethyl)benzonitrile, Example 56A (0.035 g, 0.093 mmol) in THF (2 mL) was treated with sodium bis(trimethylsilyl)amide (1M in THF, 0.1 mL, 0.1 mmol) and methyl iodide (0.020 g, 0.14 mmol). After stirring at rt for 20 min, the mixture was heated at 60° C. for 20 min. Upon cooling, additional sodium bis(trimethylsilyl)amide (1M in THF, 0.1 mL, 0.1 mmol) and methyl iodide (0.020 g, 0.14 mmol) were added and the mixture was heated again 60° C. for 30 min. This treatment was repeated once more. Upon cooling, the mixture was partitioned between EtOAc and 0.05N HCl. The organic phase was washed with water and brine, dried (Na₂SO₄), and concentrated in vacuo. The residue was purified by silica gel chromatography (5-40% EtOAc-hexanes gradient) to give the title compound (0.007 g, 19% yield): MS (ES) m/z 415 (M+Na).

### Example 60

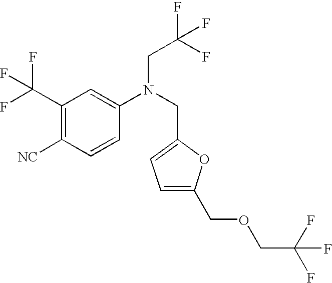

### 4-[({5-[(2,2,2-Trifluoroethoxy)methyl]-2-furyl}methyl)(2,2,2-trifluoroethyl)amino]-2-(trifluoromethyl)benzonitrile

A solution of 4-[{[5-(hydroxymethyl)-2-furyl]methyl}(2,2,2-trifluoroethyl)amino]-2-(trifluoromethyl)benzonitrile, Example 56A (0.050 g, 0.132 mmol) in toluene (3 mL) was treated with 1,1′-(azodicarbonyl)dipiperidine (0.067 g, 0.264 mmol), 2,2,2-trifluoroethanol (0.0132 g, 1.32 mmol) and tributylphosphine (0.053 g, 0.264 mmol) and stirred at rt for 15 min. The mixture was diluted with Et₂O and the solids filtered off and washed with Et₂O. The filtrate was partitioned between Et₂O and water. The organic phase was washed with water (twice) and brine, dried (Na₂SO₄), and concentrated in vacuo.
The residue was purified by silica gel chromatography (5-80% CH₂Cl₂-hexanes gradient) to give the title compound (0.023 g, 38% yield): MS (ES) m/z 483 (M+Na).

### Example 61

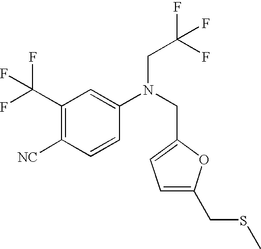

### 4-[({5-[(Methylthio)methyl]-2-furyl}methyl)(2,2,2-trifluoroethyl)amino]-2-(trifluoromethyl)benzonitrile

A suspension of sodium thiomethoxide (0.020 g, 0.28 mmol) in DMF (1 mL) was treated with a solution of 4-[{[5-(chloromethyl)-2-furyl]methyl}(2,2,2-trifluoroethyl)amino]-2-(trifluoromethyl)benzonitrile, Example 58A (0.074 g, 0.187 mmol, crude) in DMF (2 mL) and heated at 60° C. for 45 min. Upon cooling, the mixture was partitioned between Et₂O and 0.05N HCl. The organic phase was washed with 0.05N HCl, saturated NaHCO₃, and brine, dried (Na₂SO₄), and concentrated in vacuo. The residue was purified by silica gel chromatography (5-50% EtOAc-hexanes gradient) to give the title compound (0.017 g, 22% yield): MS (ES) m/z 431 (M+Na).

### Example 62

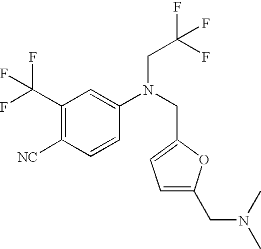

### 4-[({5-[(Dimethylamino)methyl]-2-furyl}methyl)(2,2,2-trifluoroethyl)amino]-2-(trifluoromethyl)benzonitrile

A solution of 4-[{[5-(chloromethyl)-2-furyl]methyl}(2,2,2-trifluoroethyl)amino]-2-(trifluoromethyl)benzonitrile, Example 58A (0.044 g, 0.11 mmol, crude) in THF (2 mL) was treated with dimethylamine (0.55 mL of 2M THF solution, 1.1 mmol). After stirring at rt for 15 min, additional dimethylamine (0.7 mL of 2M THF solution) was added and the mixture was heated in a sealed tube at 80° C. for 1 h. Upon cooling, the mixture was partitioned between EtOAc and 0.01N NaOH. The organic phase was washed with brine, dried (Na₂SO₄), and concentrated in vacuo. The residue was purified by silica gel chromatography (1-10% MeOH—CH₂Cl₂ gradient) to give the title compound (0.014 g, 31% yield): MS (ES) m/z 406 (M+1).

### Example 63

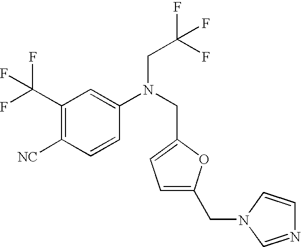

### 4-[{[5-(1H-Imidazol-1-ylmethyl)-2-furyl]methyl}(2,2,2-trifluoroethyl)amino]-2-(trifluoromethyl)benzonitrile

A solution of imidazole sodium salt (0.013 g, 0.186 mmol) in DMF (2 mL) was treated with a solution of 4-[{[5-(chloromethyl)-2-furyl]methyl}(2,2,2-trifluoroethyl)amino]-2-(trifluoromethyl)benzonitrile, Example 58A (0.037 g, 0.093 mmol, crude) in DMF (1 mL) and heated at 60° C. for 45 min. Upon cooling, additional imidazole sodium salt (0.013 g, 0.186 mmol) was added and the mixture heated at 60° C. for 20 min. The mixture was partitioned between Et₂O and 0.1N NaOH. The organic phase was washed with 0.1N NaOH and brine, dried (Na₂SO₄) and concentrated in vacuo. The residue was purified by silica gel chromatography (1-6% MeOH—CH₂Cl₂ gradient) followed by a second chromatography (sequentially 20-100% EtOAc-hexanes gradient and 2-10% MeOH-EtOAc gradient) to give the title compound (0.010 g, 25% yield): MS (ES) m/z 429 (M+1).

### Example 64

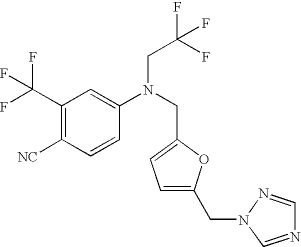

### 4-[{[5-(1H-1,2,4-Triazol-1-ylmethyl)-2-furyl]methyl}(2,2,2-trifluoroethyl)amino]-2-(trifluoromethyl)benzonitrile

Prepared in a manner similar to Example 63 using 1,2,4-triazole sodium salt: MS (ES) m/z 430 (M+1).

### Example 65

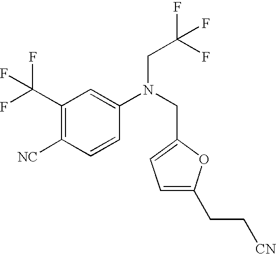

### 4-[{[5-(2-Cyanoethyl)-2-furyl]methyl}(2,2,2-trifluoroethyl)amino]-2-(trifluoromethyl)benzonitrile

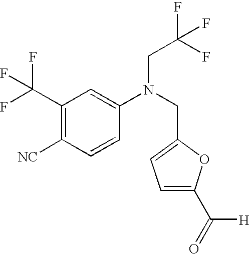

### A. 4-[[(5-Formyl-2-furyl)methyl](2,2,2-trifluoroethyl)amino]-2-(trifluoromethyl)benzonitrile

A mixture of 4-[{[5-(hydroxymethyl)-2-furyl]methyl}(2,2,2-trifluoroethyl)amino]-2-(trifluoromethyl)benzonitrile, Example 56A (0.030 g, 0.079 mmol) and activated manganese dioxide (0.138 g, 1.58 mmol) in acetonitrile (3 mL) was stirred at rt for 2 h. The solids were filtered off and washed with acetonitrile. The filtrate was concentrated to give the title compound (0.030 g, 100% yield), which was used in the next reaction without purification.

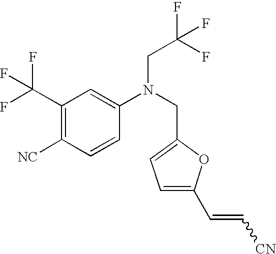

### B. 4-[({5-[2-Cyanoethenyl]-2-furyl}methyl)(2,2,2-trifluoroethyl)amino]-2-(trifluoromethyl)benzonitrile

A solution of diethyl (cyanomethyl)phosphonate (0.029 g, 0.16 mmol) in THF (1 mL) was treated under nitrogen, with sodium bis(trimethylsilyl)amide (0.16 mL of 1M THF solution, 0.16 mmol). After 30 min, a solution of 4-[[(5-formyl-2-furyl)methyl](2,2,2-trifluoroethyl)amino]-2-(trifluoromethyl)benzonitrile, Example 65A (0.030 g, 0.079 mmol) in THF (1 mL) was added and stirred at rt for 1 h. The mixture was partitioned between EtOAc and water. The organic phase was washed with water and brine, dried (Na₂SO₄) and concentrated in vacuo to give the title compound as a crude mixture of cis and trans isomers (0.031 g). This material was used in the next reaction without purification.

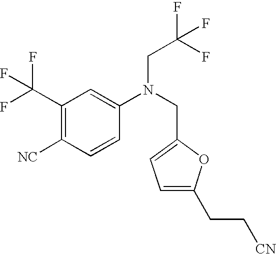

### C. 4-[{[5-(2-Cyanoethyl)-2-furyl]methyl}(2,2,2-trifluoroethyl)amino]-2-(trifluoromethyl)benzonitrile

A suspension of 5% Pd/C (0.020 g) in MeOH (2 mL) was stirred under H₂ (balloon pressure) for 10 min. Next, a solution of TEA (0.024 g, 0.237 mmol) and Example 65B (0.031 g, 0.078 mmol) in MeOH (2 mL) was added. After 45 min, the catalyst was filtered off and washed with MeOH and EtOAc. The filtrate was concentrated in vacuo and the residue purified by silica gel chromatography (5-40% EtOAc-hexanes gradient) to give the title compound (0.016 g, 50% overall yield): MS (ES) m/z 424 (M+Na).

### Example 66

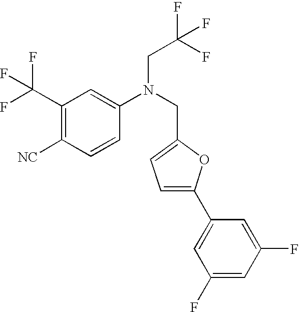

### 4-[{[5-(3,5-Difluorophenyl)-2-furanyl]-methyl}(2,2,2-trifluoroethyl)amino]-2-(trifluoromethyl)benzonitrile

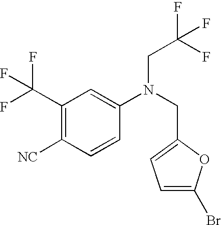

### A. 4-[[(5-Bromo-2-furanyl)methyl](2,2,2-trifluoroethyl)amino]-2-(trifluoromethyl)benzonitrile

Synthesized in a manner similar to example 10B using 2-bromo-5-(chloromethyl)furan: MS (ES) m/z 427 and 429 (M+1 isotopes).

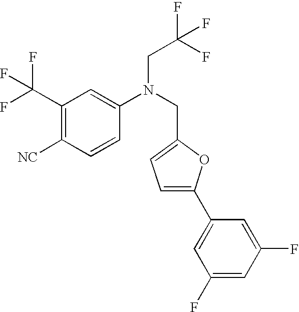

### B. 4-[{[5-(3,5-Difluorophenyl)-2-furanyl]methyl}(2,2,2-trifluoroethyl)amino]-2-(trifluoromethyl)benzonitrile

A mixture of 4-[[(5-bromo-2-furanyl)methyl](2,2,2-trifluoroethyl)amino]-2-(trifluoromethyl)benzonitrile, Example 66A (0.053 g, 0.120 mmol), 3,5-difluoro-phenylboronic acid (0.059 g, 0.370 mmol), 10% Pd/C (0.053 g), NaHCO₃ (0.031 g, 0.370 mmol) and water (0.5 mL) in DMF (2 mL) was heated at 100° C. under N₂. After 4 h, another portion of 10% Pd/C (0.053 g) was added and heating was continued for another 10 h. Upon cooling, the reaction mixture was filtered and the catalyst was washed with Et₂O. The filtrate was partitioned between Et₂O and 0.1N NaOH. The organic phase was washed with 0.1N NaOH and brine, dried (Na₂SO₄) and concentrated in vacuo. The residue was purified by silica gel chromatography (5-60% EtOAc-hexanes gradient) to give the title compound (0.044 g, 77% yield): ¹H NMR (400 MHz, CDCl₃) δ 7.68 (d, J=8.8 Hz, 1H), 7.30 (d, J=2.6 Hz, 1H), 7.07-7.04 (m, 3H), 6.71 (tt, J=8.8, 2.4 Hz, 1H), 6.63 (d, J=3.3 Hz, 1H), 6.38 (d, J=3.5 Hz, 1H), 4.72 (s, 2H), 4.08 (q, J=8.5 Hz, 2H).

### Example 67

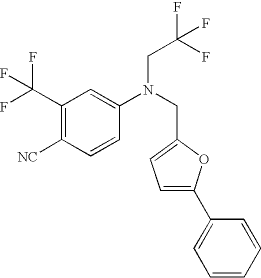

### 4-[[(5-Phenyl-2-furanyl)methyl](2,2,2-trifluoroethyl)amino]-2-(trifluoromethyl)benzonitrile

Synthesized in a manner similar to Example 66B using phenylboronic acid: ¹H NMR (400 MHz, CDCl₃) δ 7.68 (d, J=8.8 Hz, 1H), 7.56 (d, J=7.3 Hz, 2H), 7.39-7.32 (m, 3H), 7.29-7.25 (m, 1H, overlaps with CHCl₃), 7.09 (dd, J=8.8, 2.6 Hz, 1H), 6.59 (d, J=3.5 Hz, 1H), 6.37 (d, J=3.3 Hz, 1H), 4.71 (s, 2H), 4.08 (q, J=8.5 Hz, 2H).

### Example 68

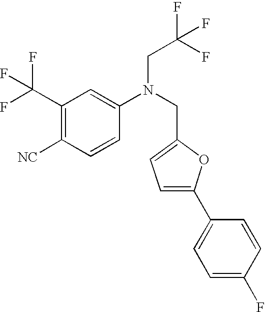

### 4-[{[5-(4-Fluorophenyl)-2-furanyl]methyl}(2,2,2-trifluoroethyl)amino]-2-(trifluoromethyl)benzonitrile

Synthesized in a manner similar to Example 66B using (4-fluorophenyl)boronic acid: ¹H NMR (400 MHz, CDCl₃) δ 7.68 (d, J=9.0 Hz, 1H), 7.53 (dd, J=8.8, 5.3 Hz, 2H), 7.34 (d, J=2.6 Hz, 1H), 7.10-7.04 (m, 3H), 6.52 (d, J=3.5 Hz, 1H), 6.35 (d, J=3.3 Hz, 1H), 4.70 (s, 2H), 4.07 (q, J=8.5 Hz, 2H).

### Example 69

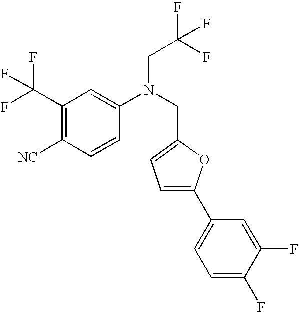

### 4-[{[5-(3,4-Difluorophenyl)-2-furanyl]methyl}(2,2,2-trifluoroethyl)amino]-2-(trifluoromethyl)benzonitrile

Synthesized in a manner similar to Example 66B using (3,4-difluorophenyl)boronic acid: MS (ES) m/z 459 (M−1).

### Example 70

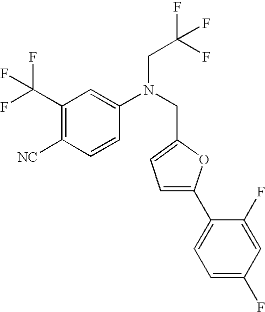

### 4-[{[5-(2,4-Difluorophenyl)-2-furanyl]methyl}(2,2,2-trifluoroethyl)amino]-2-(trifluoromethyl)benzonitrile

Synthesized in a manner similar to Example 66B using (2,4-difluorophenyl)boronic acid: MS (ES) m/z 461 (M+1).

### Example 71

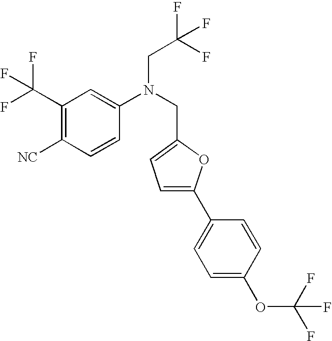

### 4-{(2,2,2-Trifluoroethyl)[(5-{4-[(trifluoromethyl)oxy]phenyl}-2-furanyl)methyl]amino}-2-(trifluoromethyl)benzonitrile

Synthesized in a manner similar to Example 66B using ({4-[(trifluoromethyl)oxy]phenyl}boronic acid: ¹H NMR (400 MHz, CDCl₃) δ 7.67 (d, J=8.8 Hz, 1H), 7.58 (d, J=8.8 Hz, 2H), 7.33 (d, J=2.6 Hz, 1H), 7.21 (d, J=8.4 Hz, 2H), 7.08 (dd, J=8.8, 2.6 Hz, 1H), 6.6 (d, J=3.5 Hz, 1H), 6.38 (d, J=3.3 Hz, 1H), 4.72 (s, 2H), 4.07 (q, J=8.6 Hz, 2H).

### Example 72

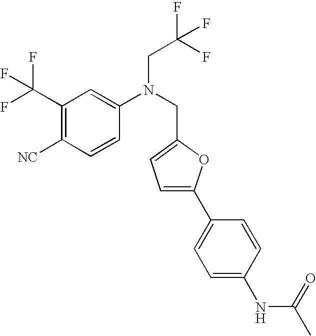

### N-[4-(5-{[[4-Cyano-3-(trifluoromethyl)phenyl](2,2,2-trifluoroethyl)amino]methyl}-2-furanyl)phenyl]acetamide

Synthesized in a manner similar to Example 66B using [4-(acetylamino)phenyl]boronic acid: MS (ES) m/z 482 (M+1).

### Example 73

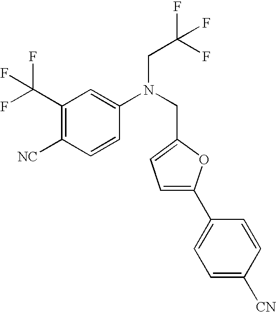

### 4-[{[5-(4-Cyanophenyl)-2-furanyl]methyl}(2,2,2-trifluoroethyl)amino]-2-(trifluoromethyl)benzonitrile

Synthesized in a manner similar to Example 66B using (4-cyanophenyl)boronic acid: MS (ES) m/z 448 (M−1).

### Example 74

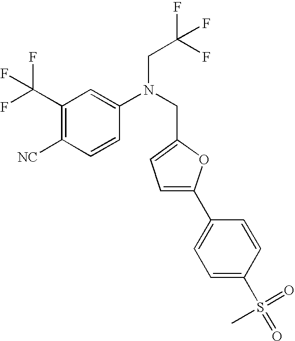

### 4-[({5-[4-(Methylsulfonyl)phenyl]-2-furanyl}methyl)(2,2,2-trifluoroethyl)amino]-2-(trifluoromethyl)benzonitrile

Synthesized in a manner similar to Example 66B using [4-(methylsulfonyl)phenyl]boronic acid: MS (ES) m/z 501 (M−1).

### Example 75

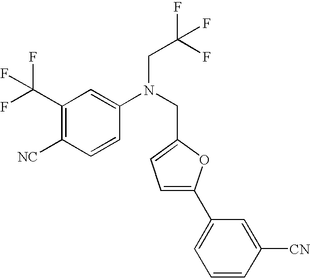

### 4-[{[5-(3-Cyanophenyl)-2-furanyl]methyl}(2,2,2-trifluoroethyl)amino]-2-(trifluoromethyl)benzonitrile

Synthesized in a manner similar to Example 66B using (3-cyanophenyl)boronic acid: MS (ES) m/z 448 (M−1).

### Example 76

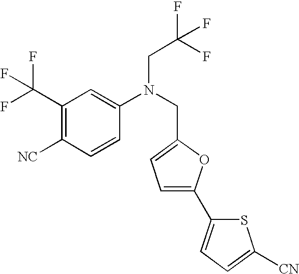

### 5-(5-{[[4-Cyano-3-(trifluoromethyl)phenyl](2,2,2-trifluoroethyl)amino]methyl}-2-furanyl)-2-thiophenecarbonitrile

Synthesized in a manner similar to Example 66B using (5-cyano-2-thienyl)boronic acid and tetrakis(triphenylphosphine)palladium(0) as catalyst: MS (ES) m/z 454 (M−1).

### Example 77

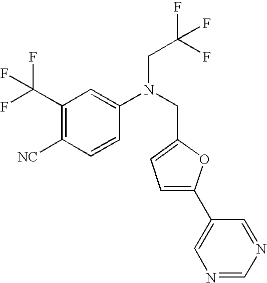

### 4-[{[5-(5-Pyrimidinyl)-2-furanyl]methyl}(2,2,2-trifluoroethyl)amino]-2-(trifluoromethyl)benzonitrile

Synthesized in a manner similar to Example 66B using 5-pyrimidinylboronic acid and tetrakis(triphenylphosphine)palladium(0) as catalyst: MS (ES) m/z 427 (M+1).

### Example 78

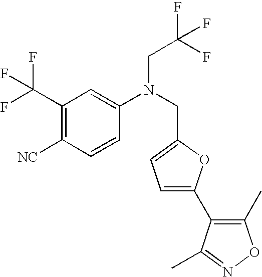

### 4-[{[5-(3,5-Dimethyl-4-isoxazolyl)-2-furanyl]methyl}(2,2,2-trifluoroethyl)amino]-2-(trifluoromethyl)benzonitrile

Synthesized in a manner similar to Example 66B using (3,5-dimethyl-4-isoxazolyl)boronic acid and tetrakis(triphenylphosphine)palladium(0) as catalyst: MS (ES) m/z 444 (M+1).

### Example 79

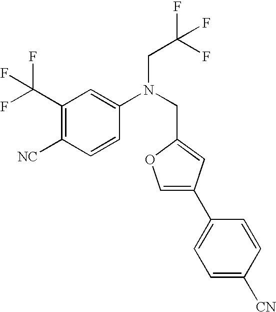

### 4-[{[4-(4-Cyanophenyl)-2-furanyl]methyl}(2,2,2-trifluoroethyl)amino]-2-(trifluoromethyl)benzonitrile

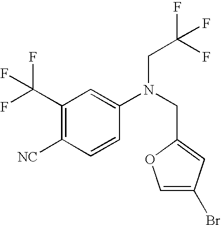

### A. 4-[[(4-Bromo-2-furanyl)methyl](2,2,2-trifluoroethyl)amino]-2-(trifluoromethyl)benzonitrile

Synthesized in a manner similar to Example 10B using 4-bromo-2-(chloromethyl)furan: ¹H NMR (400 MHz, CDCl₃) δ 7.67 (d, J=8.8 Hz, 1H), 7.39 (s, 1H), 7.18 (d, J=2.6 Hz, 1H), 7.02 (dd, J=8.8, 2.6 Hz, 1H), 6.33 (s, 1H), 4.64 (s, 2H), 4.03 (q, J=8.4 Hz, 2H).

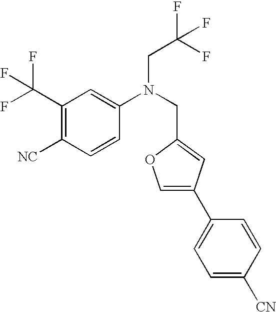

### B. 4-[{[4-(4-Cyanophenyl)-2-furanyl]methyl}(2,2,2-trifluoroethyl)amino]-2-(trifluoromethyl)benzonitrile

Synthesized in a manner similar to Example 66B from 4-[[(4-bromo-2-furanyl)methyl](2,2,2-trifluoroethyl)amino]-2-(trifluoromethyl)benzonitrile and (4-cyanophenyl)boronic acid: MS (ES) m/z 472 (M+Na).

### Example 80

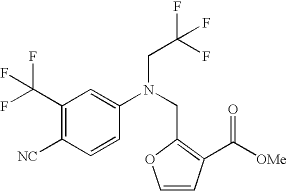

### Methyl 2-{[[4-cyano-3-(trifluoromethyl)phenyl](2,2,2-trifluoroethyl)amino]methyl}-3-furoate

Synthesized as described in Example 10B using methyl-2-(bromomethyl)-3-furoate: MS (ES) m/z 405 (M−1).

### Example 81

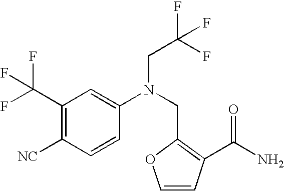

### 2-{[[4-Cyano-3-(trifluoromethyl)phenyl](2,2,2-trifluoroethyl)amino]methyl}-3-furamide

Prepared from methyl 2-{[[4-cyano-3-(trifluoromethyl)phenyl](2,2,2-trifluoroethyl)amino]methyl}-3-furoate, Example 80 as described for Example 54: MS (ES) m/z 392 (M+1).

### Example 82

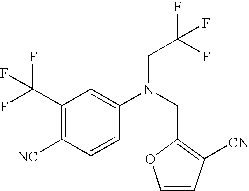

### 2-{[[4-Cyano-3-(trifluoromethyl)phenyl](2,2,2-trifluoroethyl)amino]methyl}-3-furonitrile

Prepared from 2-{[[4-cyano-3-(trifluoromethyl)phenyl](2,2,2-trifluoroethyl)amino]methyl}-3-furamide, Example 81 as described for Example 55: MS (ES) m/z 374 (M+1).

### Example 83

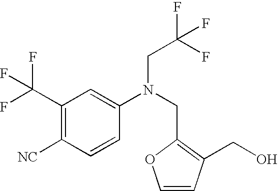

### 4-[{[3-(Hydroxymethyl)-2-furyl]methyl}(2,2,2-trifluoroethyl)amino]-2-(trifluoromethyl)benzonitrile

Prepared from methyl 2-{[[4-cyano-3-(trifluoromethyl)phenyl](2,2,2-trifluoroethyl)amino]methyl}-3-furoate, Example 80 as described for Example 56A: MS (ES) m/z 379 (M+1).

### Example 84

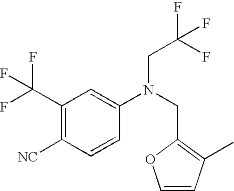

### 4-[[(3-Methyl-2-furyl)methyl](2,2,2-trifluoroethyl)amino]-2-(trifluoromethyl)benzonitrile

Prepared from 4-[{[3-(hydroxymethyl)-2-furyl]methyl}(2,2,2-trifluoroethyl)amino]-2-(trifluoromethyl)benzonitrile, Example 83 as described in steps B and C of Example 56: MS (ES) m/z 363 (M+1).

### Example 85

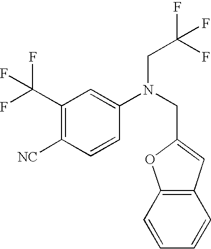

### 4-[(1-Benzofuran-2-ylmethyl)(2,2,2-trifluoroethyl)amino]-2-(trifluoromethyl)benzonitrile

Synthesized as described in Example 10B using 2-(chloromethyl)-1-benzofuran: MS (ES) m/z 399 (M+1).

### Example 86

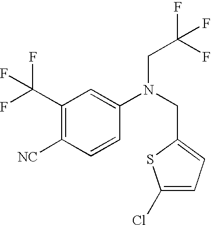

### 4-[[(5-Chlorothien-2-yl) methyl](2,2,2-trifluoroethyl)amino]-2-(trifluoromethyl)benzonitrile

Synthesized as described in Example 10B using 2-chloro-5-(chloromethyl)thiophene: MS (ES) m/z 399 (M+1).

### Example 87

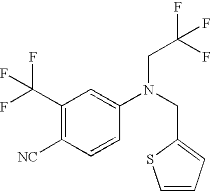

### 4-[(Thien-2-ylmethyl)(2,2,2-trifluoroethyl)amino]-2-(trifluoromethyl)benzonitrile

A mixture of 4-[[(5-chlorothien-2-yl)methyl](2,2,2-trifluoroethyl)amino]-2-(trifluoromethyl)benzonitrile, Example 86 (0.042 g, 0.105 mmol), TEA (0.021 g, 0.211 mmol) and 5% Pd/C (0.030 g) in MeOH (4 mL) was hydrogenated under balloon pressure for 1 h. The catalyst was filtered off and washed with EtOAc and MeOH. The filtrate was concentrated in vacuo and the residue was purified by silica gel chromatography (5-40% EtOAc-hexanes gradient) to give the title compound (0.032 g, 84% yield): MS (ES) m/z 365 (M+1).

### Example 88

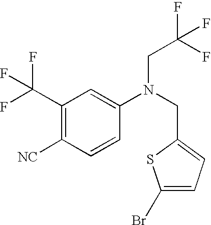

### 4-[[(5-Bromothien-2-yl)methyl](2,2,2-trifluoroethyl)amino]-2-(trifluoromethyl)benzonitrile

Synthesized as described in Example 10B using 2-bromo-5-(chloromethyl)thiophene: MS (ES) m/z 443 and 445 (M+1 isotopes).

### Example 89

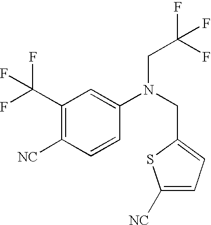

### 5-{[[4-Cyano-3-(trifluoromethyl)phenyl](2,2,2-trifluoroethyl)amino]methyl}thiophene-2-carbonitrile

A mixture of 4-[[(5-bromothien-2-yl)methyl](2,2,2-trifluoroethyl)amino]-2-(trifluoromethyl)benzonitrile, Example 88 (0.050 g, 0.112 mmol) and CuCN (0.048 g, 0.536 mmol) in DMF (3 mL) was heated in a microwave at 180° C. for 30 min and subsequently at 200° C. for 30 min. Upon cooling, the mixture was partitioned between Et₂O and 0.05N HCl. The organic phase was washed with 0.05N HCl, water and brine, dried (Na₂SO₄) and concentrated in vacuo. The residue was purified by silica gel chromatography (0-50% EtOAc-hexanes gradient) to give the title compound (0.029 g, 66% yield): MS (ES) m/z 390 (M+1).

### Example 90

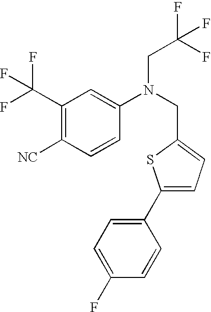

### 4-[{[5-(4-Fluorophenyl)thien-2-yl]methyl}(2,2,2-trifluoroethyl)amino]-2-(trifluoromethyl)benzonitrile

A mixture of 4-[[(5-bromothien-2-yl)methyl](2,2,2-trifluoroethyl)amino]-2-(trifluoromethyl)benzonitrile, Example 88 (0.047 g, 0.106 mmol), 4-fluoro-phenylboronic acid (0.045 g, 0.318 mmol), 10% Pd/C (0.047 g), NaHCO₃ (0.027 g, 0.318 mmol) and water (0.5 mL) in DMF (2 mL) was heated at 10° C. under nitrogen for 12 h. Upon cooling, the reaction mixture was filtered and the catalyst was washed with Et₂O. The filtrate was partitioned between Et₂O and 0.1N NaOH. The organic phase was washed with 0.1N NaOH and brine, dried (Na₂SO₄) and concentrated in vacuo. The residue was purified by silica gel chromatography (5-60% EtOAc-hexanes gradient) to give the title compound (0.039 g, 80% yield): MS (ES) m/z 457 (M−1).

### Example 91

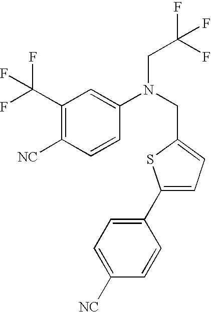

### 4-[{[5-(4-Cyanophenyl)thien-2-yl]methyl}(2,2,2-trifluoroethyl)amino]-2-(trifluoromethyl)benzonitrile

Synthesized as described in Example 90 using 4-cyano-phenylboronic acid: MS (ES) m/z 464 (M−1).

### Example 92

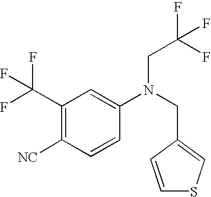

### 4-[(Thien-3-ylmethyl)(2,2,2-trifluoroethyl)amino]-2-(trifluoromethyl)benzonitrile

Synthesized as described in Example 10B using 3-(chloromethyl)thiophene: MS (ES) m/z 365 (M+1).

### Example 93

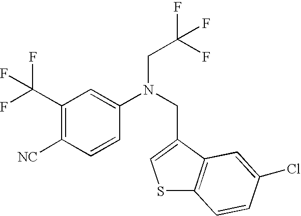

### 4-[[(5-Chloro-1-benzothien-3-yl)methyl](2,2,2-trifluoroethyl)amino]-2-(trifluoromethyl)benzonitrile

Synthesized as described in Example 10B using 3-(bromomethyl)-5-chloro-1-benzothiophene: MS (ES) m/z 449 (M+1).

### Example 94

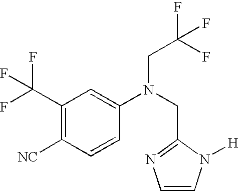

### 4-[(1H-Imidazol-2-ylmethyl)(2,2,2-trifluoroethyl)amino]-2-(trifluoromethyl)benzonitrile

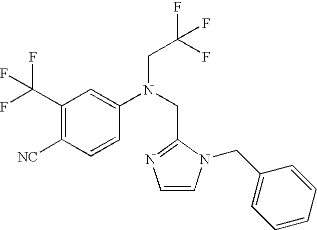

### A. 4-[{[1-(Phenylmethyl)-1H-imidazol-2-yl]methyl}(2,2,2-trifluoroethyl)amino]-2-(trifluoromethyl)benzonitrile

Synthesized as described in Example 10B from 2-(chloromethyl)-1-(phenylmethyl)-1H-Imidazole hydrochloride: MS (APCI) m/z 439 (M+1).

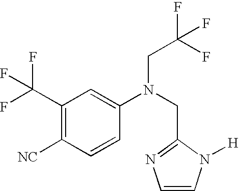

### B. 4-[(1H-Imidazol-2-ylmethyl)(2,2,2-trifluoroethyl)amino]-2-(trifluoromethyl)benzonitrile

A mixture of 4-[{[1-(phenylmethyl)-1H-imidazol-2-yl]methyl}(2,2,2-trifluoroethyl)amino]-2-(trifluoromethyl)benzonitrile, Example 94A (0.139 g, 0.317 mmol) and 10% w/w Pd/C (0.050 g) was stirred under an atmosphere of H₂ at rt. After 16 h, the mixture was filtered through Celite and concentrated in vacuo. The residue was purified by radial chromatography (CH₂Cl₂/MeOH), affording 0.080 g of the title compound as a white solid: MS (APCI) m/z 348 (M⁺).

### Example 95

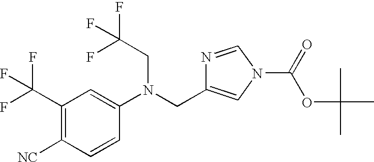

### 1,1-Dimethylethyl 4-{[[4-cyano-3-(trifluoromethyl)phenyl](2,2,2-trifluoroethyl)amino]methyl}-1H-imidazole-1-carboxylate

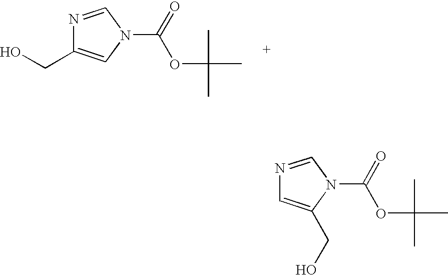

### A. 1,1-Dimethylethyl 4-(hydroxymethyl)-1H-imidazole-1-carboxylate and 1,1-dimethylethyl 5-(hydroxymethyl)-1H-imidazole-1-carboxylate

To a slurry of 4-hydroxymethylimidazole hydrochloride (1.35 g, 10.0 mmol) and TEA (2.09 mL, 15 mmol) in DMF (5 mL) at 0° C. was added a solution of di-tert-butyl dicarbonate (2.18 g, 10.0 mmol) in THF (15 mL), dropwise over 5 min. The cooling bath was removed, the mixture was stirred at rt overnight and concentrated in vacuo. The residue was partitioned between EtOAc/H₂O and the layers were separated. The organic layer was washed (2×H₂O, brine), dried over Na₂SO₄, filtered and concentrated in vacuo affording 1.14 g colorless oil which was determined (LC/MS) to be a 3:1 mixture of the title compounds, which was used without further purification: Major regioisomer: MS (ES) m/z 199 (M+H, 9%), 221 (M+Na, 13%), 143 (100%). Minor regioisomer: MS (ES) m/z 199 (M+H, 15%), 113 (100%).

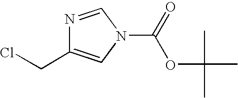

### B. 1,1-Dimethylethyl 4-(chloromethyl)-1H-imidazole-1-carboxylate

To a solution of 1,1-dimethylethyl 4-(hydroxymethyl)-1H-imidazole-1-carboxylate and 1,1-dimethylethyl 5-(hydroxymethyl)-1H-imidazole-1-carboxylate (1.13 g, 5.71 mmol) and DMF (1 drop) in anhyd. CH₂Cl₂ (25 mL) at 0° C. was added SOCl₂ (0.76 mL, 10.4 mmol), dropwise over 3 min. The mixture was stirred 45 min and concentrated in vacuo. The residue was partitioned between EtOAc/sat'd NaHCO₃, and the layers were separated. The organic layer was washed (H₂O, brine), dried over Na₂SO₄, filtered and concentrated in vacuo. The residue was purified by flash chromatography (EtOAc/hexanes), affording 0.720 g of the title compound as a colorless oil: ¹H NMR (400 MHz, CDCl₃) δ 8.05 (d, J=1.3 Hz, 1H), 7.39 (m, 1H), 4.55 (partially resolved d, J=0.8 Hz, 2H), 1.62 (s, 9H). The minor, regioisomeric product was not collected from the flash chromatography column.

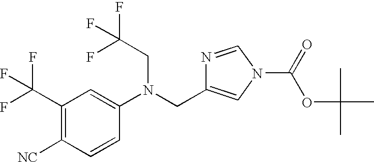

### C. 1,1-Dimethylethyl 4-{[[4-cyano-3-(trifluoromethyl)phenyl](2,2,2-trifluoroethyl)amino]methyl}-1H-imidazole-1-carboxylate

Synthesized as described in Example 10B from 1,1-dimethylethyl 4-(chloromethyl)-1H-imidazole-1-carboxylate and 4-[(2,2,2-trifluoroethyl)amino]-2-(trifluoromethyl)benzonitrile (Example 10A): MS (ES) 449 (M+H, 4%), 349 ([M+H]-CO₂t-Bu, 100%).

### Example 96

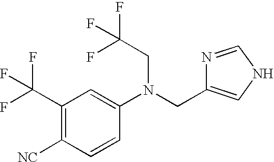

### 4-[(1H-Imidazol-4-ylmethyl)(2,2,2-trifluoroethyl)amino]-2-(trifluoromethyl)benzonitrile

To a solution of 1,1-dimethylethyl 4-{[[4-cyano-3-(trifluoromethyl)phenyl](2,2,2-trifluoroethyl)amino]methyl}-1H-imidazole-1-carboxylate (0.0926 g, 0.207 mmol; Example 95C) and Et₃SiH (0.08 mL, 0.52 mmol) in CH₂Cl₂ (3 mL) at rt was added TFA (1 mL). The mixture was stirred 30 min and concentrated in vacuo. The residue was partitioned between EtOAc/sat'd Na₂CO₃, the layers were separated, the organic layer was dried over Na₂SO₄, filtered and concentrated in vacuo. The residue was purified by flash chromatography (MeOH/CH₂Cl₂), affording 0.054 g of the title compound as a colorless gum: MS (ES) m/z 349 (M+H).

### Example 97

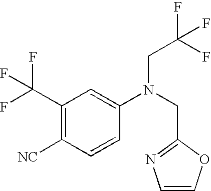

### 4-[(1,3-Oxazol-2-ylmethyl)(2,2,2-trifluoroethyl)amino]-2-(trifluoromethyl)benzonitrile

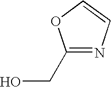

### A. 1,3-Oxazol-2-ylmethanol

To a solution of oxazole (0.66 mL, 10.0 mmol) in anh. THF (35 mL) at rt was added BH₃.THF (10.0 mL of a 1.0M solution in THF, 10 mmol). The solution was stirred 1 h at rt, cooled to −78° C., and n-BuLi (4.40 mL of a 2.5M solution in hexanes, 11.0 mmol) was added dropwise over 5 min. The mixture was stirred 1 h 15 min at −78° C. and anh. DMF (0.85 mL, 11.0 mmol) was added dropwise. The mixture was gradually warmed to approx. −10° C. over 5 h, cooled to −78° C. and quenched by addition of 5% v/v HOAc/EtOH (35 mL). The cooling bath was removed, the mixture was stirred 18 h at rt and concentrated in vacuo. The residue was slurried in EtOAc and filtered through a coarse sinter. The filtrate was concentrated in vacuo and purified by flash chromatography (EtOAc/hexanes), affording 0.450 g of the title compound: ¹H NMR (400 MHz, CDCl₃) δ 7.64 (s, 1H), 7.09 (s, 1H), 4.75 (s, 2H), 4.55 (bs, 1H).

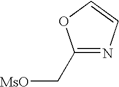

### B. 1,3-Oxazol-2-ylmethyl methanesulfonate

To a solution of 1,3-oxazol-2-ylmethanol (0.106 g, 1.07 mmol) in dry CH₂Cl₂ (5 mL) at 0° C., was added TEA (0.16 mL, 1.2 mmol), followed by MsCl (0.090 mL, 1.2 mmol). The mixture was stirred 45 min, then diluted with CH₂Cl₂, washed (water, satd NaHCO₃, brine), dried over Na₂SO₄, filtered and concentrated in vacuo, affording 0.151 g of the title compound as a colorless oil: ¹H NMR (400 MHz, CDCl₃) δ 7.75 (s, 1H), 7.20 (s, 1H), 5.32 (s, 2H), 3.11 (s, 3H).

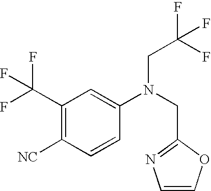

### C. 4-[(1,3-Oxazol-2-ylmethyl)(2,2,2-trifluoroethyl)amino]-2-(trifluoromethyl)benzonitrile

Synthesized as described in Example 10B using 1,3-oxazol-2-ylmethyl methanesulfonate: ¹H NMR (400 MHz, CDCl₃) δ 7.70-7.66 (overlapping s and d, 2H), 7.22 (d, J=2.8 Hz, 1H), 7.11 (s, 1H), 7.08 (dd, J=8.7, 2.8 Hz, 1H), 4.79 (s, 2H), 4.17 (q, J=8.5 Hz, 2H).

### Example 98

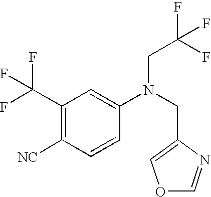

### 4-[(1,3-Oxazol-4-ylmethyl)(2,2,2-trifluoroethyl)amino]-2-(trifluoromethyl)benzonitrile

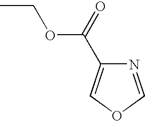

### A. Ethyl 1,3-oxazole-4-carboxylate

To a solution of formic acid (1.73 mL, 46.0 mmol) in anhydrous THF (50 mL) at rt was added 1,1′-carbonyldiimidazole (7.45 g, 46.0 mmol), portionwise over 3 min. The resulting solution was stirred 30 min under N₂ and ethyl isocyanoacetate (5.00 mL, 46.0 mmol) was added, followed by TEA (12.8 mL, 92 mmol). The resulting solution was stirred for 1 h at rt and brought to reflux. After 20 h, the mixture was cooled and concentrated in vacuo. The residue was dissolved in Et₂O and washed with water. The aqueous layer was extracted with EtOAc (×4), combined organics were washed with brine, dried over Na₂SO₄, filtered and concentrated in vacuo. The residue was purified by flash chromatography (EtOAc/hexanes), affording 5.01 g of the title compound as a pale yellow oil: ¹H NMR (400 MHz, CDCl₃) δ 8.28 (d, J=1.1 Hz, 1H), 7.95 (d, J=1.0 Hz, 1H), 4.41 (q, J=7.1 Hz, 2H), 1.40 (t, J=7.1 Hz, 3H).

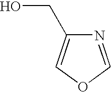

### B. 1,3-Oxazol-4-ylmethanol

To a solution of ethyl 1,3-oxazole-4-carboxylate (0.178 g, 1.26 mmol) in anhydrous THF (5 mL) at −78° C. was added DIBAL-H (2.52 mL of a 1.0 M solution in hexanes, 2.52 mmol), dropwise over 3 min. After 1 h, the cooling bath was removed, and the solution warmed to 0° C. After stirring 2.5 h at 0° C., the reaction was quenched with MeOH, MgSO₄ (ca. 0.3 g) was added and the mixture stirred 30 min. The whole mixture was filtered through Celite (EtOAc wash), concentrated in vacuo and allowed to stand at rt over night. The residue was slurried in EtOAc and filtered again through Celite. The filtrate was concentrated in vacuo, affording 0.103 g of the title compound as a pale yellow oil which was used without further purification: ¹H NMR (400 MHz, CDCl₃) δ 7.90 (s, 1H), 7.64 (app. q, J=0.9 Hz, 1H), 4.64 (d, J=6.0 Hz, 2H), 3.40 (t, J=6.1 Hz, 1H).

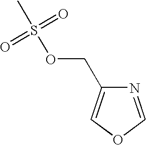

### C. 1,3-Oxazol-4-ylmethyl methanesulfonate

To a solution of 1,3-oxazol-4-ylmethanol (0.0973 g, 0.98 mmol) in anhydrous CH₂Cl₂ (5 mL) at 0° C. was added TEA (0.15 mL, 1.1 mmol), followed by MsCl (0.083 mL, 1.1 mmol). The mixture was stirred 15 min and poured into water. The layers were separated, and the aqueous layer was extracted with CH₂Cl₂ (×2). Combined organics portions were washed (sat'd NaHCO₃, brine), dried over Na₂SO₄, filtered, and concentrated in vacuo, affording 0.141 g of the title compound as a colorless syrup which was used without further purification: ¹H NMR (300 MHz, CDCl₃) δ 7.93 (bs, 1H), 7.83 (q, J=0.8 Hz, 1H), 5.22 (d, J=0.5 Hz, 2H), 3.07 (s, 3H).

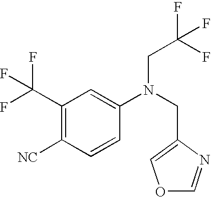

### D. 4-[(1,3-Oxazol-4-ylmethyl)(2,2,2-trifluoroethyl)amino]-2-(trifluoromethyl)benzonitrile

An oven-dried 10 mL conical vial was charged with 4-[(2,2,2-trifluoroethyl)amino]-2-(trifluoromethyl)benzonitrile (0.055 g, 0.205 mmol), 1,3-oxazol-4-ylmethyl methanesulfonate (0.044 g, 0.25 mmol), anhydrous Cs₂CO₃ (0.100 g, 0.31 mmol), and anhydrous acetonitrile (3 mL). The vial was sealed with a septum cap and the mixture was heated to 65° C. with vigorous stirring. After 4 h, the mixture was cooled, and the mixture was partitioned between EtOAc/water. The organic layer was washed with brine, dried over Na₂SO₄, filtered and concentrated in vacuo. The residue was purified by flash chromatography (EtOAc/hexanes), affording 0.058 g of the title compound as a colorless solid: ¹H NMR (400 MHz, CDCl₃) δ 7.87 (s, 1H), 7.65 (d, J=8.8 Hz, 1H), 7.58 (app. q, J=1.1 Hz, 1H), 7.18 (d, J=2.6, Hz, 1H), 7.03 (dd, J=6.0, 2.8 Hz, 1H), 4.65 (s, 2H), 4.12 (q, J=8.6 Hz, 2H).

### Example 99

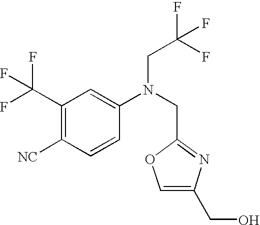

### 4-[{[4-(Hydroxymethyl)-1,3-oxazol-2-yl]methyl}(2,2,2-trifluoroethyl)amino]-2-(trifluoromethyl)benzonitrile

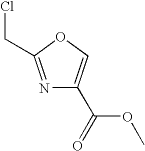

### A. Methyl 2-(chloromethyl)-1,3-oxazole-4-carboxylate

According to a literature procedure (Cardwell. K.; Hermitage, S.; Sjolin, A. Tetrahedron Lett. 2000, 41, 4239), a solution of NaOMe (1.15 mL of a 25 wt % solution in MeOH, 4.95 mmol) was diluted with MeOH (10 mL), cooled to −10° C. and dichloroacetonitrile (4.0 mL, 49.7 mmol) was added dropwise over 15 min. The resulting mixture was stirred 20 min under N₂ and DL-serine methyl ester hydrochloride (7.74 g, 49.7 mmol) and MeOH (8 mL) were added. The resulting mixture was stirred in the cooling bath over night, gradually warming to rt. CH₂Cl₂ (30 mL) was added, the mixture was poured into water (20 mL), the layers were separated and the aqueous layer was extracted with CH₂Cl₂ (20 mL). Combined organics portions were washed with brine, dried over Na₂SO₄, filtered and concentrated in vacuo, affording 9.37 g of the crude dichloromethyl oxazoline intermediate which was used without further purification: ¹H NMR (400 MHz, CDCl₃) δ 6.29 (s, 1H), 4.91 (dd, J=10.8, 8.3 Hz, 1H), 4.76 (overlapping dd, J=8.8, 8.3 Hz, 1H), 4.67 (dd, J=10.8, 8.8 Hz, 1H), 3.83 (s, 3H). The crude oxazoline (9.37 g) was dissolved in MeOH (9 mL) and added to a pre-cooled solution (0° C.) of NaOMe (10.4 mL of a 25 wt % solution in MeOH, 44.6 mmol) in MeOH (8 mL), dropwise at such a rate that the internal temperature was maintained below 10° C. The resulting mixture was stirred for 14 h, gradually warming to rt. CH₂Cl₂ (50 mL) was added, the mixture was poured into water (50 mL), the layers were separated and the aqueous layer was extracted with CH₂Cl₂ (×1). Combined organics portions were washed with brine, dried over Na₂SO₄, filtered and concentrated in vacuo. The residue was dissolved in toluene (20 mL), (±)-CSA (1.39 g, 6 mmol) was added and the mixture heated at 70° C. for 1 h. The solution was cooled, poured into satd Na₂CO₃ and extracted with EtOAc (×3). Combined organics portions were washed (water, brine), dried over Na₂SO₄, filtered and concentrated in vacuo. The residue was purified by flash chromatography (EtOAc/hexanes), affording 2.55 g of the title compound as a pale, waxy yellow solid: ¹H NMR (400 MHz, CDCl₃) δ 8.27 (s, 1H), 4.65 (s, 2H), 3.94 (s, 3H).

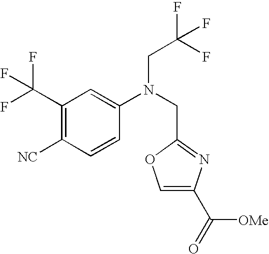

### B. Methyl 2-{[[4-cyano-3-(trifluoromethyl)phenyl](2,2,2-trifluoroethyl)amino]methyl}-1,3-oxazole-4-carboxylate

To a slurry of hexanes-washed NaH (0.172 g, 4.31 mmol) in DMF (4 mL) at 0° C. was added a solution of 4-[(2,2,2-trifluoroethyl)amino]-2-(trifluoromethyl)benzonitrile, Example 10A (0.770 g, 2.87 mmol) in DMF (2 mL), dropwise over 5 min. The resulting solution was stirred 15 min and a solution of methyl 2-(chloromethyl)-1,3-oxazole-4-carboxylate (0.503 g, 2.87 mmol) in DMF (2 mL) was added dropwise over 3 min. The resulting mixture was stirred in the cooling bath 2 h, poured into water and extracted with Et₂O (×3). Combined organics were washed (water, brine), dried over Na₂SO₄, filtered and concentrated in vacuo. The title compound (0.258 g colorless gum) was the minor reaction product and was isolated by flash chromatography (EtOAc/hexanes): ¹H NMR (400 MHz, CDCl₃) δ 8.23 (s, 1H), 7.20 (d, J=8.7 Hz, 1H), 7.20 (d, J=2.7 Hz, 1H), 7.09 (dd, J=8.8, 2.7 Hz, 1H), 4.84 (s, 2H), 4.20 (q, J=8.3 Hz, 2H), 3.92 (s, 3H).

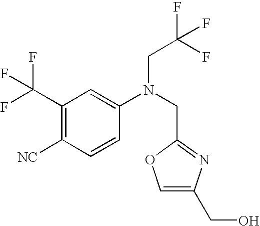

### C. 4-[{[4-(Hydroxymethyl)-1,3-oxazol-2-yl]methyl}(2,2,2-trifluoroethyl)amino]-2-(trifluoromethyl)benzonitrile

To a solution of methyl 2-{[[4-cyano-3-(trifluoromethyl)phenyl](2,2,2-trifluoroethyl)amino]methyl}-1,3-oxazole-4-carboxylate (0.125 g, 0.307 mmol) in anhydrous THF (3 mL) at −78° C. was added DIBAL-H (0.34 mL of a 1.0M solution in hexanes, 0.34 mmol), dropwise over 3 min. An additional portion of DIBAL-H (0.31 mmol) was added after 50 min and the resulting solution was stirred, gradually warming to rt. After 19 h, the solution was cooled again to −78° C. and an additional portion of DIBAL-H (0.15 mmol) was added. The resulting solution was stirred 1 h at −78° C. and warmed to approximately 0° C. by removal of the cooling bath. The reaction was quenched by dropwise addition of sat'd Rochelle's salt solution (5 mL), layered with EtOAc (5 mL) and stirred until clean phase separation occurred (ca. 30 min). The layers were separated and the aqueous layer was extracted with EtOAc (×2). Combined organics were washed (water, brine), dried over Na₂SO₄, filtered and concentrated in vacuo. The residue was purified by flash chromatography (EtOAc/hexanes), affording 0.0854 g of the title compound as a colorless gum: ¹H NMR (400 MHz, CDCl₃) δ 7.67 (d, J=8.8 Hz, 1H), 7.60 (app. t, J=1.1 Hz, 1H), 7.21 (d, J=2.7 Hz, 1H), 7.06 (dd, J=8.9, 2.8 Hz, 1H), 4.77 (s, 2H), 4.56 (d, J=0.9 Hz, 2H), 4.18 (q, J=8.5 Hz, 2H).

### Example 100

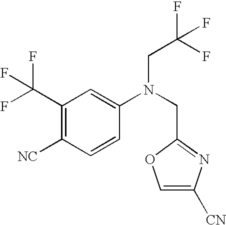

### 2-{[[4-Cyano-3-(trifluoromethyl)phenyl](2,2,2-trifluoroethyl)amino]methyl}-1,3-oxazole-4-carbonitrile

To a solution of 4-[{[4-(hydroxymethyl)-1,3-oxazol-2-yl]methyl}(2,2,2-trifluoroethyl)amino]-2-(trifluoromethyl)benzonitriles (Example 99) (0.076 g, 0.20 mmol) in CH₂Cl₂ (5 mL) at rt was added Dess-Martin periodinane (0.093 g, 0.22 mmol) in one portion and the mixture was stirred at rt. An additional portion of periodinane (0.042 g, 0.10 mmol) was added to the mixture after 45 min. After 90 min, the mixture was diluted with CH₂Cl₂ and washed with 1 N NaOH. The aqueous wash was extracted with CH₂Cl₂ (×1), combined organics were washed (water, brine), dried over Na₂SO₄, filtered and concentrated in vacuo. The residue was dissolved in MeOH (5 mL), and to the resulting solution was added NH₂OH.HCl (0.021 g, 0.30 mmol) and a solution of K₂CO₃ (0.5 mL of a 10 wt % aqueous solution). The mixture was stirred over night at rt and concentrated in vacuo. The residue was partitioned between CH₂Cl₂/water, the layers were separated and the aqueous layer was extracted with CH₂Cl₂ (×1). Combined organics were washed with brine, dried over Na₂SO₄ and filtered. To the filtrate was added 1,1′-carbonyldiimidazole (0.049 g, 0.30 mmol) and the solution was stirred at rt. An additional portion of 1,1′-carbonyldiimidazole (0.025 g, 0.15 mmol) was added after 4 h. After 5 h, the whole was adsorbed onto a small amount of silica gel and subjected to flash chromatography (EtOAc/hexanes), affording 0.030 g of the title compound as a colorless solid: ¹H NMR (400 MHz, CDCl₃) δ 8.19 (s, 1H), 7.71 (d, J=8.8 Hz, 1H), 7.18 (d, J=2.8 Hz, 1H), 7.05 (dd, J=8.7, 2.9 Hz, 1H), 4.84 (s, 2H). 4.17 (q, J=8.4 Hz, 2H).

### Example 101

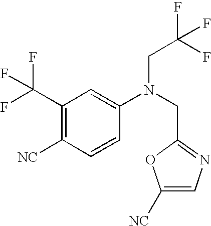

### 2-{[[4-Cyano-3-(trifluoromethyl)phenyl](2,2,2-trifluoroethyl)amino]methyl}-1,3-oxazole-5-carbonitrile

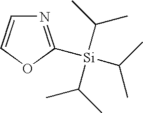

### A. 2-Triisopropylsilyl-1,3-oxazole

According to a literature procedure (Miller, R.; Smith, R.; Karady, S.; Reamer, R. Tetrahedron Lett. 2002, 43, 935), to a solution of oxazole (0.79 mL; 12 mmol) in anhydrous THF (35 mL) at −40° C. was added n-BuLi (5.04 mL of a 2.5M solution in hexanes; 12.6 mmol) dropwise over 5 min. The solution was stirred 5 min at −40° C., warmed to −10° C., and triisopropylsilyl trifluoromethanesulfonate (3.38 mL, 12.6 mmol) was added dropwise over 3 min. The cooling bath was removed and the solution was stirred 20 min at rt, poured into water and extracted with EtOAc (×3). Combined organic portions were washed (water, brine), dried over Na₂SO₄, filtered and concentrated in vacuo. The residue was purified by flash chromatography (EtOAc/hexanes), affording 2.29 g of the title compound as a pale yellow oil: ¹H NMR (400 MHz, CDCl₃) δ 7.82 (d, J=0.7 Hz, 1H), 7.22 (d, J=0.7 Hz, 1H), 1.42 (sept, J=7.4 Hz, 3H), 1.14 (d, J=7.4 Hz, 18H).

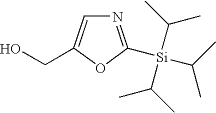

### B. {2-[Triisopropylsilyl]-1,3-oxazol-5-yl}methanol

To a solution of 2-triisopropylsilyl-1,3-oxazole (0.796 g, 3.54 mmol) in anhydrous THF (15 mL) at −78° C. was added n-BuLi (1.49 mL of a 2.5M solution in hexanes, 3.72 mmol), dropwise over 3 min. The resulting mixture was stirred 30 min and DMF (0.30 mL, 3.89 mmol) was added via syringe. The mixture was warmed to rt over 30 min by gradually removing the cooling bath and quenched by addition of satd NH₄Cl solution. The mixture was poured into water and extracted with Et₂O (×3). Combined organic portions were washed (water, brine), dried over Na₂SO₄, filtered and concentrated in vacuo. The residue was dissolved in MeOH (10 mL), cooled to 0° C. and NaBH₄ (0.134 g; 3.54 mmol) was added in one portion. The mixture was stirred 15 min and quenched by dropwise addition of sat'd NH₄Cl solution. The mixture was poured into water and extracted with CH₂Cl₂ (×3). Combined organic portions were washed (water, brine), dried over Na₂SO₄, filtered and concentrated in vacuo. The residue was purified by flash chromatography (EtOAc/hexanes), affording 0.638 g of the title compound as a colorless solid: ¹H NMR (400 MHz, CDCl₃) δ 7.07 (t, J=0.8 Hz, 1H), 4.72 (d, J=5.2 Hz, 2H), 2.05 (bt, J=5.7 Hz, 1H), 1.41 (sept, J=7.5 Hz, 3H), 1.13 (d, J=7.4 Hz, 18H).

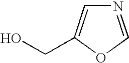

### C. 1,3-Oxazol-5-ylmethanol

To a solution of {2-[triisopropylsilyl]-1,3-oxazol-5-yl}methanol (0.985 g, 3.86 mmol) in THF (10 mL) at rt was added TBAF (3.9 mL of a 1.0M solution in THF, 3.9 mmol). The solution was stirred 30 min and adsorbed onto a small amount of silica gel and subjected to flash chromatography (EtOAc/hexanes), affording 0.321 g of the title compound as a colorless, opaque liquid: ¹H NMR (400 MHz, CDCl₃) δ 7.87 (s, 1H), 7.00 (s, 1H), 4.69 (s, 2H), 3.26 (bs, 1H).

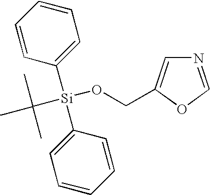

### D. 5-({[(Tert-butyl)(diphenyl)silyl]oxy}methyl)-1,3-oxazole

To a solution of 1,3-oxazol-5-ylmethanol (0.203 g; 2.05 mmol) and imidazole (0.279 g, 4.1 mmol) in anhydrous DMF (5 mL) at rt was added TBDPS-Cl (0.53 mL, 2.05 mmol). The resulting solution was stirred 30 min under N₂, poured into water and extracted with Et₂O (×3). Combined organic portions were washed (water, brine), dried over Na₂SO₄, filtered and concentrated in vacuo. The residue was purified by flash chromatography (EtOAc/hexanes), affording 0.559 g of the title compound as a colorless syrup: ¹H NMR (400 MHz, CDCl₃) δ 7.81 (s, 1H), 7.71-7.66 (m, 4H), 7.48-7.37 (m, 6H), 6.86 (bs, 1H), 4.69 (d, J=0.9 Hz, 2H), 1.05 (s, 9H).

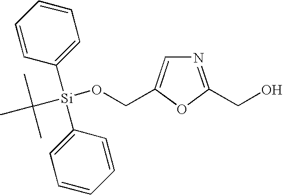

### E. [5-({[(Tert-butyl)(diphenyl)silyl]oxy}methyl)-1,3-oxazol-2-yl]methanol

To a solution of 5-({[(tert-butyl)(diphenyl)silyl]oxy}methyl)-1,3-oxazole (0.556 g, 1.65 mmol) in anhydrous THF (6 mL) at rt was added BH₃.THF (1.65 mL of a 1.0M solution in THF, 1.65 mmol). After 45 min a small amount of unchanged starting material remained (TLC), thus an additional portion of BH₃.THF (0.25 mL, 0.25 mmol) was added and stirring continued at rt. After 2 h, the solution was cooled to −78° C. and n-BuLi (0.80 mL of a 2.5M solution in hexanes, 1.98 mmol) was added, dropwise over 3 min. The solution was stirred 30 min and 4-formylmorpholine (0.20 mL, 2.0 mmol) was added via syringe. The reaction was slowly warmed to ca. −25° C. over 2.5 h and quenched with 5% v/v HOAc in EtOH (5 mL). The mixture was stirred 60 h at rt and concentrated in vacuo. The residue was partitioned between EtOAc/satd NaHCO₃ solution and the layers were separated. The organic layer was washed with brine, dried over Na₂SO₄, filtered and concentrated in vacuo. The residue was purified by flash chromatography, affording 0.233 g of the title compound as a colorless gum: ¹H NMR (400 MHz, CDCl₃) δ 7.69-7.65 (m, 4H), 7.47-7.37 (m, 6H), 6.78 (bs, 1H), 4.68-4.65 (overlapping s, 4H total, 2×2H), 1.05 (s, 9H).

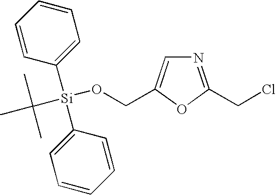

### F. 2-(Chloromethyl)-5-({[(tert-butyl)(diphenyl)silyl]oxy}methyl)-1,3-oxazole

To a solution of [5-({[(tert-butyl)(diphenyl)silyl]oxy}methyl)-1,3-oxazol-2-yl]methanol (0.217 g, 0.59 mmol) in benzene (2 mL) and CCl₄ (2 mL) was added PPh₃ (0.232 g, 0.89 mmol) in one portion and the mixture was heated to 80° C. After 12 h, the mixture was cooled, and solids were removed by filtration. The filtrate was adsorbed onto a small amount of SiO₂ and subjected to flash chromatography (EtOAc/hexanes), affording 0.161 g of the title compound as a colorless gum: ¹H NMR (400 MHz, CDCl₃) δ 7.70-7.65 (m, 4H), 7.48-7.36 (m, 6H), 6.81 (t, J=0.8 Hz, 1H), 4.68 (d, J=0.8 Hz, 2H), 4.56 (s, 2H), 1.06 (s, 9H).

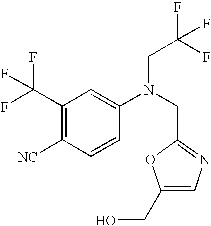

### G. 4-[{[5-(Hydroxymethyl)-1,3-oxazol-2-yl]methyl}(2,2,2-trifluoroethyl)amino]-2-(trifluoromethyl)benzonitrile

To a slurry of hexanes-washed NaH (0.030 g, 0.76 mmol) in anhydrous DMF (1 mL) at 0° C. was added a solution of 4-[(2,2,2-trifluoroethyl)amino]-2-(trifluoromethyl)benzonitrile (0.102 g, 0.38 mmol) in DMF (1 mL), dropwise over 3 min. The mixture was stirred 20 min and a solution of 2-(chloromethyl)-5-({[(tert-butyl)(diphenyl)silyl]oxy}methyl)-1,3-oxazole (0.161 g, 0.42 mmol) was added. The mixture was stirred 2.5 h at rt, poured into water and extracted with Et₂O (×3). Combined organic portions were washed (water, brine), dried over Na₂SO₄, filtered and concentrated in vacuo. Flash chromatography (EtOAc/hexanes) afforded 0.121 g of a yellow oil, which was a mixture of the expected 3° aniline product and unchanged 4-[(2,2,2-trifluoroethyl)amino]-2-(trifluoromethyl)benzonitrile (ca. 4:1 ratio). The mixture was dissolved in MeOH (1 mL) and added to a solution of HCl in MeOH (prepared by addition of 0.29 mL AcCl to 10 mL MeOH at 0° C.). The resulting solution was stirred 4.5 h at rt and concentrated in vacuo. The residue was partitioned between EtOAc/satd NaHCO₃ solution and the layers were separated. The organic layer was washed with brine, dried over Na₂SO₄, filtered and concentrated in vacuo. The residue was purified by flash chromatography (EtOAc/hexanes), affording 0.0455 g of the title compound as a colorless gum: ¹H NMR (400 MHz, CDCl₃) δ 7.67 (d, J=8.8 Hz, 1H), 7.22 (d, J=2.7 Hz, 1H), 7.08 (dd, J=8.8, 2.8 Hz, 1H), 6.98 (bs, 1H), 4.77 (s, 2H), 4.66 (d, J=5.6 Hz, 2H), 4.18 (q, J=8.5 Hz, 2H), 1.98 (t, J=5.9 Hz, 1H).

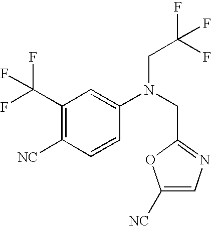

### H. 2-{[[4-Cyano-3-(trifluoromethyl)phenyl](2,2,2-trifluoroethyl)amino]methyl}-1,3-oxazole-5-carbonitrile

Synthesized from 4-[{[5-(hydroxymethyl)-1,3-oxazol-2-yl]methyl}(2,2,2-trifluoroethyl)amino]-2-(trifluoromethyl)benzonitrile according to the procedure described Example 100: ¹H NMR (400 MHz, CDCl₃) δ 7.71 (d, J=8.8 Hz, 1H, partially overlapping 7.70), 7.70 (s, 1H), 7.19 (d, J=2.8 Hz, 1H), 7.04 (dd, J=8.8, 2.9 Hz, 1H), 4.87 (s, 2H), 4.18 (q, J=8.3 Hz, 2H).

### Example 102

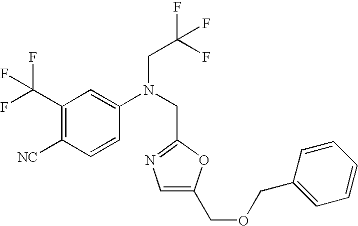

### 4-[[(5-{[(Phenylmethyl)oxy]methyl}-1,3-oxazol-2-yl)methyl](2,2,2-trifluoroethyl)amino]-2-(trifluoromethyl)benzonitrile

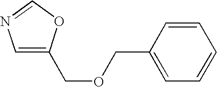

### A. 5-{[(Phenylmethyl)oxy]methyl}-1,3-oxazole

To a slurry of hexanes-washed NaH (0.049 g of a 60% suspension in mineral oil, 1.2 mmol) in anhyd DMF (1 mL) at 0° C. was added a solution of 1,3-oxazol-5-ylmethanol (0.101 g; 1.01 mmol; Example 101C) in anhyd DMF (1 mL), dropwise over 3 min. The mixture was warmed to rt and stirred 45 min. Benzyl bromide (0.14 mL, 1.2 mmol) was added and the mixture was stirred 12 h at rt. An additional portion of NaH (0.040 g, 1.0 mmol) was added, the mixture was stirred 10 min, and an additional portion of benzyl bromide (0.12 mL, 1.0 mmol) was added. The mixture was stirred 30 min, poured into water and extracted with Et₂O (×3). Combined organics were washed (H₂O, brine), dried over Na₂SO₄, filtered and concentrated in vacuo. The residue was purified by flash chromatography (EtOAc/hexanes), affording 0.160 g of the title compound as a pale yellow oil: MS (ES) m/z 190 (M+1).

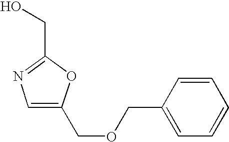

### B. (5-{[(Phenylmethyl)oxy]methyl}-1,3-oxazol-2-yl)methanol

To a solution of 5-{[(phenylmethyl)oxy]methyl}-1,3-oxazole (0.156 g, 0.824 mmol) in anh. THF (4 mL) at rt was added a solution of BH₃-THF (0.82 mL of a 1M solution in THF, 0.82 mmol), dropwise over 3 min. The solution was stirred 1 h, cooled to −78° C. and n-BuLi (0.36 mL of a 2.5M solution in hexanes, 0.91 mmol) was added dropwise over 3 min. The mixture was stirred 50 min and N-formylmorpholine (0.09 mL, 0.9 mmol) was added. The mixture was stirred 1 h at −78° C. and quenched with 4 mL 5% v/v HOAc in EtOH. The mixture was removed from the cooling bath, stirred 15 h at rt and concentrated in vacuo (1×PhMe chase). The residue was slurried in EtOAc, washed (sat'd NaHCO₃, brine), dried over Na₂SO₄, filtered and concentrated in vacuo. The residue was purified by flash chromatography (EtOAc/hexanes), affording 0.0893 g of the title compound as a colorless film: ¹H NMR (400 MHz, CDCl₃) δ 7.28-7.40 (m, 5H), 6.99 (s, 1H), 4.72 (s, 2H), 4.56 (s, 2H), 4.51 (s, 2H).

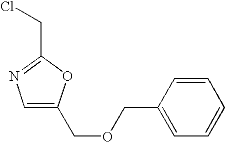

### C. 2-(Chloromethyl)-5-{[(phenylmethyl)oxy]methyl}-1,3-oxazole

To a solution of (5-{[(phenylmethyl)oxy]methyl}-1,3-oxazol-2-yl)methanol (0.087 g, 0.40 mmol) in benzene (2 mL) and CCl₄ (1 mL) at rt was added PPh₃ (0.156 g, 0.60 mmol) in one portion. The mixture was heated at reflux under N₂ 6 h and cooled to rt. Supernatant liquid was decanted away from precipitated solids, solids were washed with benzene (×3) and the washings were combined with the supernatant. Combined organics were adsorbed onto a small amount of silica gel and purified by flash chromatography (EtOAc/hexanes), affording 0.075 g of the title compound as a colorless gum: ¹H NMR (400 MHz, CDCl₃) δ 7.40-7.28 (m, 5H), 7.02 (s, 1H), 4.60 (s, 2H), 4.58 (s, 2H), 4.53 (s, 2H).

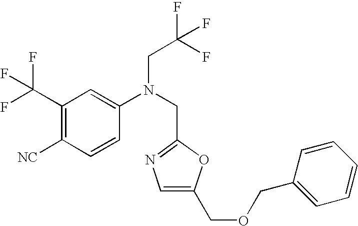

### D. 4-[[(5-{[(Phenylmethyl)oxy]methyl}-1,3-oxazol-2-yl)methyl](2,2,2-trifluoroethyl)amino]-2-(trifluoromethyl)benzonitrile

To a slurry of hexanes-washed NaH (0.023 g of a 60% suspension in mineral oil, 0.57 mmol) in anh. DMF (1 mL) at 0° C. was added a solution of 4-[(2,2,2-trifluoroethyl)amino]-2-(trifluoromethyl)benzonitrile (0.077 g, 0.29 mmol; Example 10A) in anh. DMF (1 mL), dropwise over 3 min. The mixture was stirred 15 min and a solution of 2-(chloromethyl)-5-{[(phenylmethyl)oxy]methyl}-1,3-oxazole (0.075 g, 0.31 mmol) in anh. DMF (1 mL) was added dropwise over 3 min. The mixture was removed from the cooling bath, stirred 60 h at rt, poured into water and extracted with Et₂O (×3). Combined organics were washed (H₂O, brine), dried over Na₂SO₄, filtered and concentrated in vacuo. The residue was purified by flash chromatography (EtOAc/hexanes), affording 0.043 g of the title compound as a pale yellow solid: MS (ES) m/z 470 (M+1).

### Example 103

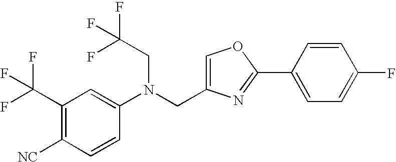

### 4-[{[2-(4-Fluorophenyl)-1,3-oxazol-4-yl]methyl}(2,2,2-trifluoroethyl)amino]-2-(trifluoromethyl)benzonitrile

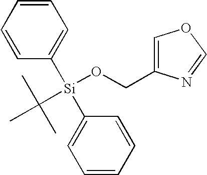

### A. 4-({[(1,1-Dimethylethyl)(diphenyl)silyl]oxy}methyl)-1,3-oxazole

To a solution of 1,3-oxazol-4-ylmethanol (0.520 g, 5.25 mmol; Example 98B) and imidazole (0.790 g, 11.6 mmol) in anh. DMF (10 mL) at rt was added tert-butyldiphenylsilyl chloride (1.5 mL, 5.8 mmol). The mixture was stirred 30 min, poured into water and extracted with Et₂O (×3). Combined organics were washed (H₂O, brine), dried over Na₂SO₄, filtered and concentrated in vacuo. The residue was purified by flash chromatography (EtOAc/hexanes), affording 1.76 g of the title compound as a colorless oil: MS (AP) m/z 360 (M+Na).

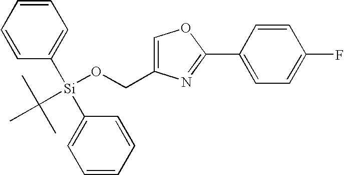

### B. 4-({[(1,1-Dimethylethyl)(diphenyl)silyl]oxy}methyl)-2-(4-fluorophenyl)-1,3-oxazole

To a solution of 4-({[(1,1-dimethylethyl)(diphenyl)silyl]oxy}methyl)-1,3-oxazole (0.341 g, 1.01 mmol) in anhyd THF (5 mL) at −78° C. was added n-BuLi (0.45 mL of a 2.5M solution in hexanes, 1.1 mmol), dropwise over 3 min. The mixture was stirred 45 min, anhyd ZnBr₂ (2.7 mL of a 1.13M solution in THF, 3.0 mmol) was added and the cooling bath was removed. 1-Bromo-4-fluorobenzene (0.13 mL, 1.2 mmol) and Pd(PPh₃)₄ (0.058 g, 0.051 mmol) were added, and the mixture was heated at reflux under N₂. After 3 h the mixture was cooled and concentrated in vacuo. The residue was partitioned between EtOAc/sat'd NH₄Cl, the layers were separated and the aqueous layer was extracted with EtOAc (×2). Combined organics were washed (H₂O, brine), dried over Na₂SO₄, filtered and concentrated in vacuo. The residue was purified by flash chromatography (EtOAc/hexanes), affording 0.304 g of the title compound as a colorless gum: MS (ES) m/z 432 (M+H, 27%), 354 (100%).

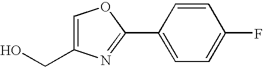

### C. [2-(4-Fluorophenyl)-1,3-oxazol-4-yl]methanol

AcCl (1.00 mL; 14.1 mmol) was added to MeOH (14 mL) at 0° C. and the solution was stirred 5 min. To the above solution was added a solution of 4-({[(1,1-dimethylethyl)(diphenyl)silyl]oxy}methyl)-2-(4-fluorophenyl)-1,3-oxazole (0.304 g; 0.705 mmol) in MeOH (2 mL). The cooling bath was removed, the mixture was stirred at rt for 3 h and concentrated in vacuo. The residue was partitioned between EtOAc/sat'd Na₂CO₃, the layers were separated and the aqueous layer was extracted with EtOAc (×2). Combined organics were washed (H₂O, brine), dried over Na₂SO₄, filtered and concentrated in vacuo. The residue was purified by flash chromatography (EtOAc/hexanes), affording 0.113 g of the title compound as a colorless solid: MS (ES) m/z 194 (M+H).

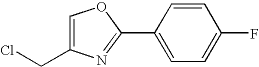

### D. 4-(Chloromethyl)-2-(4-fluorophenyl)-1,3-oxazole

Synthesised as described in Example 102C using [2-(4-fluorophenyl)-1,3-oxazol-4-yl]methanol: MS (ES) m/z 212 (M+H).

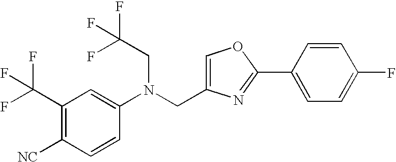

### E. 4-[{[2-(4-Fluorophenyl)-1,3-oxazol-4-yl]methyl}(2,2,2-trifluoroethyl)amino]-2-(trifluoromethyl)benzonitrile

Synthesized as described in Example 102D using 4-(chloromethyl)-2-(4-fluorophenyl)-1,3-oxazole and 4-[(2,2,2-trifluoroethyl)amino]-2-(trifluoromethyl)benzonitrile (Example 10A), with the exceptions that only 1 equiv of 4-(chloromethyl)-2-(4-fluorophenyl)-1,3-oxazole and 1.3 equiv of NaH were used, and after flash chromatography the product was further purified by RP-HPLC (C₁₈ column, MeCN/H₂O) to remove unreacted 4-[(2,2,2-trifluoroethyl)amino]-2-(trifluoromethyl)benzonitrile: MS (AP) m/z 444 (M+H).

### Example 104

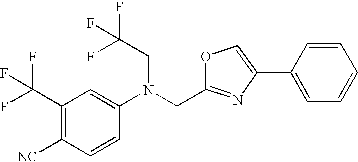

### 4-[[(4-Phenyl-1,3-oxazol-2-yl)methyl](2,2,2-trifluoroethyl)amino]-2-(trifluoromethyl)benzonitrile

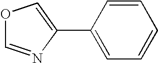

### A. 4-Phenyl-1,3-oxazole

To a solution of benzyl isocyanide (1.22 mL, 10.0 mmol) in anh. THF (10 mL) at −78° C. was added n-BuLi (4.0 mL of a 2.5M solution in hexanes, 10 mmol), dropwise over 4 min. The mixture was stirred an additional 3 min and methyl formate (0.62 mL, 10 mmol) was added dropwise over 3 min. The cooling bath was removed, the mixture was allowed to warm to approx. 0° C. and HOAc (0.57 mL, 10 mmol) was added. The mixture was stirred 20 min at rt and concentrated in vacuo. The residue was purified by flash chromatography (EtOAc/hexanes), affording 0.945 g of the title compound as a pale yellow liquid: MS (ES) m/z 146 (M+H).

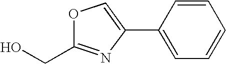

### B. (4-Phenyl-1,3-oxazol-2-yl)methanol

Synthesized as described in Example 102B from 4-phenyl-1,3-oxazole: MS (ES) m/z 176 (M+H, 54%), 130 (100%).

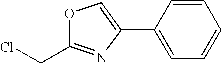

### C. 2-(Chloromethyl)-4-phenyl-1,3-oxazole

Synthesized as describe in Example 102C from 104B, 4-phenyl-1,3-oxazol-2-yl)methanol: MS (ES) m/z 194 (M+H).

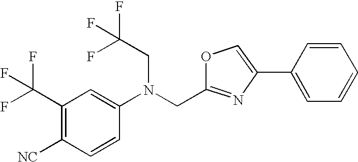

### D. 4-[[(4-Phenyl-1,3-oxazol-2-yl)methyl](2,2,2-trifluoroethyl)amino]-2-(trifluoromethyl)benzonitrile

Synthesized as described in Example 102D using 2-(chloromethyl)-4-phenyl-1,3-oxazole and 4-[(2,2,2-trifluoroethyl)amino]-2-(trifluoromethyl)benzonitrile (Example 10A), with the exceptions that only 1 equiv 2-(chloromethyl)-4-phenyl-1,3-oxazole and 1.2 equiv NaH were used, and after flash chromatography the product was further purified by RP-HPLC (C₁₈ column, MeCN/H₂O) to remove unreacted 4-[(2,2,2-trifluoroethyl)amino]-2-(trifluoromethyl)benzonitriles: MS (AP) m/z 426 (M+H).

### Example 105

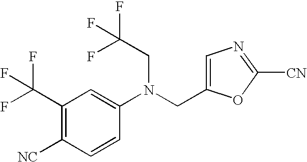

### 5-{[[4-Cyano-3-(trifluoromethyl)phenyl](2,2,2-trifluoroethyl)amino]methyl}-1,3-oxazole-2-carbonitrile

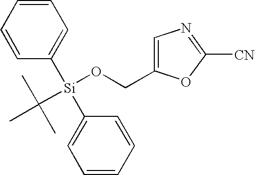

### A. 5-({([(1,1-Dimethylethyl)(diphenyl)silyl]oxy}methyl)-1,3-oxazole-2-carbonitrile

To a solution of [5-({[(1,1-dimethylethyl)(diphenyl)silyl]oxy}methyl)-1,3-oxazol-2-yl]methanol (0.396 g, 1.08 mmol, Example 101E) in CH₂Cl₂ at rt was added Dess-Martin periodinane (0.505 g, 1.19 mmol) in one portion. The mixture was stirred 30 min and washed with 1M NaOH. The aqueous wash was back-extracted with CH₂Cl₂ (×1), combined organics were washed (H₂O, brine), dried over Na₂SO₄, filtered and concentrated in vacuo. The residue was filtered through a pad of silica gel (EtOAc/hexanes eluent) and concentrated in vacuo affording 0.312 g of a colorless oil (aldehyde intermediate). The oil was dissolved in MeOH (5 mL), and to the resulting solution was added NH₂OH.HCl (0.077 g, 1.1 mmol) and K₂CO₃ (0.5 mL of a 10 wt % solution). The mixture was stirred at rt 1.5 h, concentrated in vacuo, partitioned between CH₂Cl₂/water and the layers were separated. The aqueous layer was extracted with CH₂Cl₂ (×1), combined organics were washed (H₂O, brine), dried over Na₂SO₄, filtered and concentrated in vacuo, affording 0.350 g of a colorless gum. The gum was dissolved in CH₂Cl₂ (5 mL), and to the solution was added CDI (0.292 g, 1.8 mmol) in one portion. The mixture was stirred approx. 60 h, adsorbed onto a small amount of silica and purified by flash chromatography (EtOAc/hexanes), affording 0.177 g of the title compound as a colorless oil, followed by 0.074 g unreacted aldehyde intermediate. The recovered aldehyde was re-subjected to oxime formation and dehydrating conditions described above, affording an additional 0.057 g title compound. Thus, total yield was 0.234 g: ¹H NMR (400 MHz, CDCl₃) δ 7.67-7.63 (m, 4H), 7.50-7.45 (m, 2H), 7.44-7.39 (m, 4H), 7.05 (unresolved t, J=0.9 Hz, 1H), 4.72 (d, J=0.9 Hz, 2H), 1.07 (s, 9H).

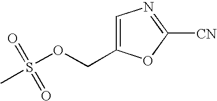

### B. (2-Cyano-1,3-oxazol-5-yl)methyl methanesulfonate

To a solution of 5-({[(1,1-dimethylethyl)(diphenyl)silyl]oxy}methyl)-1,3-oxazole-2-carbonitrile (0.234 g, 0.645 g) in THF (3 mL) at rt was added TBAF (0.65 mL of a 1.0M solution in THF, 0.65 mmol). The solution was stirred 10 min and quenched with sat'd NH₄Cl. The whole was partitioned between EtOAc/sat'd NH₄Cl and the layers were separated. The organic layer was washed with brine, dried over Na₂SO₄, filtered and concentrated in vacuo. The residue was purified by flash chromatography (EtOAc/hexanes), affording 0.064 g of a colorless gum (alcohol intermediate). The gum was dissolved in anh. CH₂Cl₂ (4 mL), cooled to 0° C. under N₂, and to the solution was added triethylamine (0.08 mL, 0.57 mmol) and MsCl (0.044 mL, 0.57 mmol). The mixture was stirred 1.5 h, poured into water, the layers were separated and the aqueous layer was extracted with CH₂Cl₂ (×2). Combined organics were washed (H₂O, brine), dried over Na₂SO₄, filtered and concentrated in vacuo, affording 0.092 g of the title compound as a colorless gum which was used directly without further purification: ¹H NMR (400 MHz, CDCl₃) δ 7.45 (s, 1H), 5.29 (s, 2H), 3.10 (s, 3H).

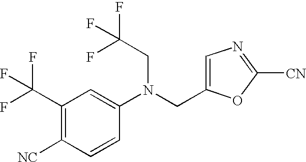

### C. 5-[([[4-Cyano-3-(trifluoromethyl)phenyl](2,2,2-trifluoroethyl)amino]methyl]-1,3-oxazole-2-carbonitrile

Synthesized as described in Example 10B using (2-cyano-1,3-oxazol-5-yl)methyl methanesulfonate and 4-[(2,2,2-trifluoroethyl)amino]-2-(trifluoromethyl)benzonitrile (Example 10A) with the exceptions that only 1 equiv 2-cyano-1,3-oxazol-5-yl)methyl methanesulfonate and 1.5 equiv Cs₂CO₃ were used: ¹H NMR (400 MHz, CDCl₃) δ 7.73 (d, J=8.8 Hz, 1H), 7.22 (s, 1H), 7.15 (d, J=2.8 Hz, 1H), 7.02 (dd, J=8.8, 2.8 Hz, 1H), 4.83 (s, 2H), 4.11 (q, J=8.4 Hz, 2H).

### Example 106

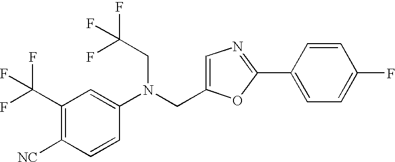

### 4-[{[2-(4-Fluorophenyl)-1,3-oxazol-5-yl]methyl}(2,2,2-trifluoroethyl)amino]-2-(trifluoromethyl)benzonitrile

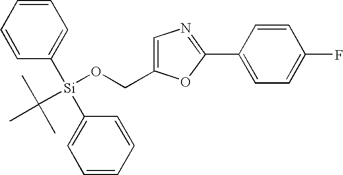

### A. 5-({[(1,1-Dimethylethyl)(diphenyl)silyl]oxy}methyl)-2-(4-fluorophenyl)-1,3-oxazole

Synthesized as described in Example 103B from 5-({[(1,1-dimethylethyl)(diphenyl)silyl]oxy}methyl)-1,3-oxazole (Example 101D) and 1-bromo-4-fluorobenzene: MS (AP) m/z 432 (M+H).

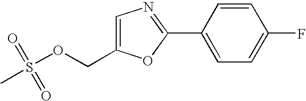

### B. [2-(4-Fluorophenyl)-1,3-oxazol-5-yl]methyl methanesulfonate

Synthesized as described in Example 105B from 5-({[(1,1-dimethylethyl)(diphenyl)silyl]oxy}methyl)-2-(4-fluorophenyl)-1,3-oxazole. ¹H NMR of the material obtained after aqueous workup of the mesylation reaction was complex, indicating a mixture of compounds. Purification was not attempted, and the material was used directly for step C below.

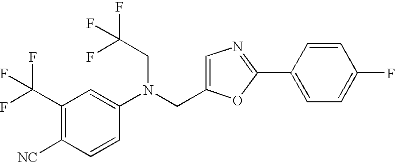

### C. 4-[{[2-(4-Fluorophenyl)-1,3-oxazol-5-yl]methyl}(2,2,2-trifluoroethyl)amino]-2-(trifluoromethyl)benzonitrile

Synthesized as described in Example 10B using [2-(4-fluorophenyl)-1,3-oxazol-5-yl]methyl methanesulfonate and 4-[(2,2,2-trifluoroethyl)amino]-2-(trifluoromethyl)benzonitrile (Example 10A) with the exceptions that only 1 equiv [2-(4-fluorophenyl)-1,3-oxazol-5-yl]methyl methanesulfonate and 1.5 equiv Cs₂CO₃ were used: ¹H NMR (400 MHz, CDCl₃) δ 7.98-7.92 (m, 2H), 7.72 (d, J=8.8 Hz, 1H), 7.73 (d, J=2.7 Hz, 1H), 7.18-7.12 (m, 2H), 7.12 (s, 1H, partially overlapping multiplet at 7.18-7.12), 7.09 (dd, J=8.8, 2.8 Hz, 1H), 4.79 (s, 2H), 4.08 (q, J=8.5 Hz, 2H).

### Example 107

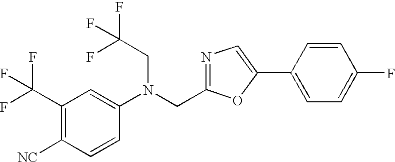

### 4-[{[5-(4-Fluorophenyl)-1,3-oxazol-2-yl]methyl}(2,2,2-trifluoroethyl)amino]-2-(trifluoromethyl)benzonitrile

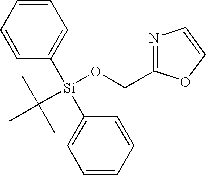

### A. 2-({[(1,1-Dimethylethyl)(diphenyl)silyl]oxy}methyl)-1,3-oxazole

Synthesized as described in Example 103A from 1,3-oxazol-2-ylmethanol (Example 97A): ¹H NMR (400 MHz, CDCl₃) δ 7.73-7.69 (m, 4H), 7.63 (d, J=0.8 Hz, 1H), 7.42-7.37 (m, 6H), 7.07 (d, J=0.8 Hz, 1H), 4.76 (s, 2H), 1.07 (s, 9H).

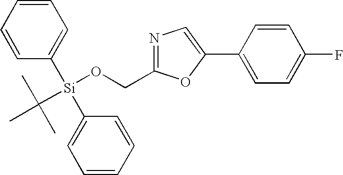

### B. 2-({[(1,1-Dimethylethyl)(diphenyl)silyl]oxy}methyl)-5-(4-fluorophenyl)-1,3-oxazole

Synthesized as described in Example 103B from 2-({[(1,1-dimethylethyl)(diphenyl)silyl]oxy}methyl)-1,3-oxazole with the exception that only 1.1 equiv ZnBr₂ was used for metathesis of the initially formed lithiooxazole intermediate: MS (AP) m/z 432 (M+H, 10%), 454 (M+Na, 63%), 354 (100%).

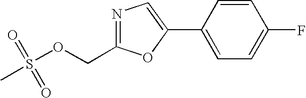

### C. [5-(4-Fluorophenyl)-1,3-oxazol-2-yl]methyl methanesulfonate

Synthesized as described in Example 105B from 2-({[(1,1-dimethylethyl)(diphenyl)silyl]oxy}methyl)-5-(4-fluorophenyl)-1,3-oxazole: ¹H NMR (400 MHz, CDCl₃) δ 7.68-7.62 (m, 2H), 7.32 (s, 1H), 7.18-7.11 (m, 2H), 5.35 (s, 2H), 3.13 (s, 3H).

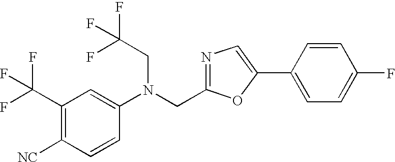

### D. 4-[{[5-(4-Fluorophenyl)-1,3-oxazol-2-yl]methyl}(2,2,2-trifluoroethyl)amino]-2-(trifluoromethyl)benzonitrile

Synthesized as described in Example 10B using [5-(4-fluorophenyl)-1,3-oxazol-2-yl]methyl methanesulfonate and 4-[(2,2,2-trifluoroethyl)amino]-2-(trifluoromethyl)benzonitrile (Example 10A) with the exceptions that only 1 equiv [5-(4-fluorophenyl)-1,3-oxazol-2-yl]methyl methanesulfonate and 1.5 equiv Cs₂CO₃ were used: MS (ES) m/z 444 (M+H, 8%), 466 (M+Na, 7%), 176 (100%).

### Example 108

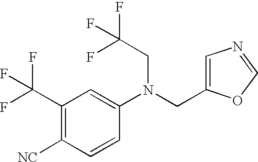

### 4-[(1,3-Oxazol-5-ylmethyl)(2,2,2-trifluoroethyl)amino]-2-(trifluoromethyl)benzonitrile

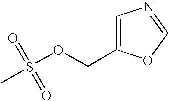

### A. 1,3-Oxazol-5-ylmethyl methanesulfonate

Synthesized as described in Example 97B from 1,3-oxazol-5-ylmethanol (Example 101C): ¹H NMR (400 MHz, CDCl₃) δ 7.97 (s, 1H), 7.29 (s, 1H), 5.29 (s, 2H), 3.03 (s, 3H).

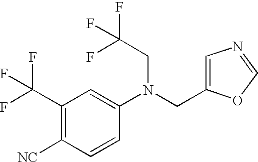

### B. 4-[(1,3-Oxazol-5-ylmethyl)(2,2,2-trifluoroethyl)amino]-2-(trifluoromethyl)benzonitrile

Synthesized as described in Example 102D from 1,3-oxazol-5-ylmethyl methanesulfonate and 4-[(2,2,2-trifluoroethyl)amino]-2-(trifluoromethyl)benzonitrile: MS (ES) m/z 350 (M+H).

### Example 109

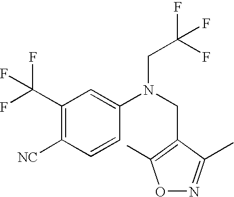

### 4-[[(3,5-Dimethylisoxazol-4-yl)methyl](2,2,2-trifluoroethyl)amino]-2-(trifluoromethyl)benzonitrile

Synthesized as described in Example 10B using 4-(chloromethyl)-3,5-dimethyl-isoxazole: MS (ES) m/z 378 (M+1).

### Example 110

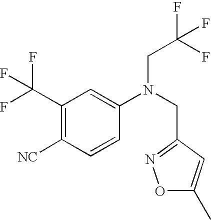

### 4-[[(5-Methylisoxazol-3-yl)methyl](2,2,2-trifluoroethyl)amino]-2-(trifluoromethyl)benzonitrile

Synthesized as described in Example 10B using 3-(chloromethyl)-5-methylisoxazole: MS (ES) m/z 364 (M+1).

### Example 111

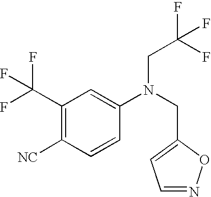

### 4-[(5-Isoxazolylmethyl)(2,2,2-trifluoroethyl)amino]-2-(trifluoromethyl)benzonitrile

To an ice-cold mixture of 4-(2,2,2-trifluoro-ethylamino)-2-trifluoromethyl-benzonitrile (Example 10A) (0.05 g, 0.187 mmol) and 5-(bromomethyl)isoxazole (0.03 g, 0.185 mmol) in DMF (1 mL), under N₂, was added NaH (60% in oil, 0.008 g, 0.206 mmol). After stirring for 45 min, water (1 mL) was added dropwise, and the mixture was partitioned between 0.1N HCl and Et₂O. The organic phase was washed with 0.1N HCl and sat'd brine, dried over Na₂SO₄, filtered and concentrated in vacuo. The residue was purified by silica gel chromatography (5-40% EtOAc-hexanes gradient) and the product was crystallized from CH₂Cl₂-hexanes to give the title compound as a white solid (0.022 g, 34% yield): MS (AP) m/z 350 (M+1).

### Example 112

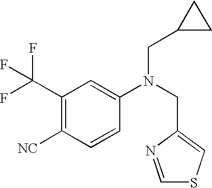

### 4-[(Cyclopropylmethyl)(1,3-thiazol-4-ylmethyl)amino]-2-(trifluoromethyl)benzonitrile

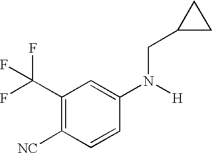

### A. 4-[(Cyclopropylmethyl)amino]-2-(trifluoromethyl)benzonitrile

Synthesized as described in Example 1A using (cyclopropylmethyl)amine: MS (ES) m/z 241 (M+1).

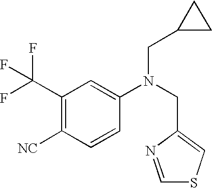

### B. 4-[(Cyclopropylmethyl)(1,3-thiazol-4-ylmethyl)amino]-2-(trifluoromethyl)benzonitrile

Synthesized as described in Example 1B using Example 112A and 4-(chloromethyl)-1,3-thiazole hydrochloride: MS (ES) m/z 338 (M+1).

### Example 113

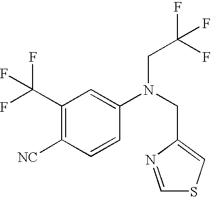

### 4-[(1,3-Thiazol-4-ylmethyl)(2,2,2-trifluoroethyl)amino]-2-(trifluoromethyl)benzonitrile

Synthesized as described in Example 10B using 4-(chloromethyl)-1,3-thiazole hydrochloride: MS (ES) m/z 366 (M+1).

### Example 114

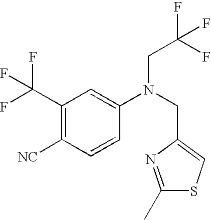

### 4-[[(2-Methyl-1,3-thiazol-4-yl)methyl](2,2,2-trifluoroethyl)amino]-2-(trifluoromethyl)benzonitrile

Synthesized as described in Example 10B using 4-(chloromethyl)-2-methyl-1,3-thiazole hydrochloride: MS (ES) m/z 380 (M+1).

### Example 115

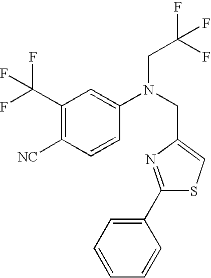

### 4-[[(2-Phenyl-1,3-thiazol-4-yl)methyl](2,2,2-trifluoroethyl)amino]-2-(trifluoromethyl)benzonitrile

Synthesized as described in Example 10B using 4-(chloromethyl)-2-phenyl-1,3-thiazole: MS (ES) m/z 442 (M+1).

### Example 116

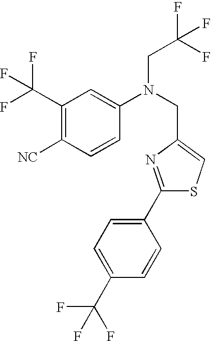

### 4-[(2,2,2-Trifluoroethyl)({2-[4-(trifluoromethyl)phenyl]-1,3-thiazol-4-yl}methyl)amino]-2-(trifluoromethyl)benzonitrile

Synthesized as described in Example 10B using 4-(chloromethyl)-2-[4-(trifluoromethyl)phenyl]-1,3-thiazole: MS (ES) m/z 510 (M+1).

### Example 117

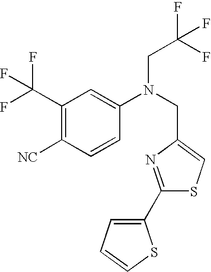

### 4-[[(2-Thien-2-yl-1,3-thiazol-4-yl)methyl](2,2,2-trifluoroethyl)amino]-2-(trifluoromethyl)benzonitrile

Synthesized in a manner similar to Example 1B using Example 10A and 4-(chloromethyl)-2-(2-thienyl)-1,3-thiazole: MS (ES) m/z 448 (M+1).

### Example 118

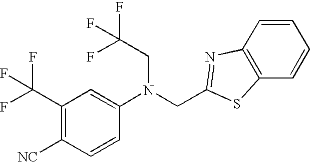

### 4-[(1,3-Benzothiazol-2-ylmethyl)(2,2,2-trifluoroethyl)amino]-2-(trifluoromethyl)benzonitrile

Synthesized as described in Example 10B from 4-[(2,2,2-trifluoroethyl)amino]-2-(trifluoromethyl)benzonitrile (Example 10A) and 2-bromomethylbenzothiazole, with the exception that 1.2 equiv bromomethylbenzothiazole and 2.2 equiv Cs₂CO₃ were used: MS (ES) m/z 416 (M+H, 34%), 119 (100%).

### Example 119

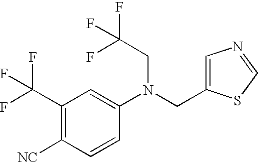

### 4-[(1,3-Thiazol-5-ylmethyl)(2,2,2-trifluoroethyl)amino]-2-(trifluoromethyl)benzonitrile

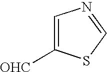

### A. 1,3-Thiazole-5-carbaldehyde

To a solution of 2-(trimethylsilyl)-1,3-thiazole (0.49 mL, 3.0 mmol) in anh. Et₂O (15 mL) at −78° C. was added n-BuLi (1.2 mL of a 2.5M solution in hexanes, 3.0 mmol), dropwise over 3 min. The mixture was stirred 40 min and N-formylmorpholine (0.32 mL, 3.2 mmol) was added dropwise. The mixture was gradually warmed to approx. 0° C. over 2 h, quenched with 10% HCl, poured into sat'd NaHCO₃ and extracted with Et₂O (×2). Combined organics were dried over Na₂SO₄, filtered and concentrated in vacuo. The residue was dissolved in THF (10 mL), 10% HCl (1.5 mL) was added, the mixture was stirred 1.5 h at rt and concentrated in vacuo. The residue was partitioned between EtOAc/sat'd NaHCO₃, the layers were separated and the aqueous layer was extracted with EtOAc (×2). Combined organics were washed (H₂O, brine), dried over Na₂SO₄, filtered and concentrated in vacuo. The residue was purified by flash chromatography (EtOAc/hexanes), affording 0.105 g of the title compound as a pale yellow oil. The aqueous layer was further extracted with EtOAc (×3), affording an additional 0.180 g title compound (after flash chromatography). Thus, total yield was 0.285 g: ¹H NMR (400 MHz, CDCl₃) δ 10.13 (d, J=1.1 Hz, 1H), 9.13 (m, 1H), 8.55 (partially resolved d, J=0.5 Hz, 1H).

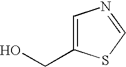

### B. 1,3-Thiazol-5-ylmethanol

To a solution of 1,3-thiazole-5-carbaldehyde (0.077 g, 0.87 mmol) in MeOH (2 mL) at rt was added MP-Borohydride (0.270 g, ca. 3.2 mmol BH₄^{-/g resin, ca.} 0.87 mmol, Argonaut Technologies Inc.) in one portion. The mixture was stirred 1.5 h, the resin was removed by filtration and the filtrate was concentrated in vacuo, affording 0.0836 g of the title compound as a colorless gum: ¹H NMR (400 MHz, CDCl₃) δ8.75 (partially resolved d, J=0.7 Hz, 1H), 7.74 (partially resolved app q, J=0.8 Hz, 1H), 4.90 (partially resolved d, J=0.9 Hz, 2H).

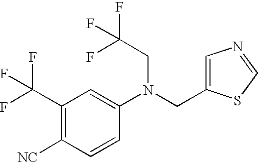

### C. 4-[(1,3-Thiazol-5-ylmethyl)(2,2,2-trifluoroethyl)amino]-2-(trifluoromethyl)benzonitrile

To a solution of 1,3-thiazol-5-ylmethanol (0.0693 g, 0.60 mmol) and TEA (0.092 mL, 0.66 mmol) in anh. CH₂Cl₂ (4 mL) at 0° C. was added MsCl (0.051 mL, 0.66 mmol), dropwise. The mixture was stirred 15 min and washed with sat'd NH₄Cl. The aqueous wash was back-extracted with CH₂Cl₂ (×1). Combined organics were washed (sat'd NaHCO₃, brine), dried over Na₂SO₄, filtered and concentrated in vacuo, affording 0.0913 g of a reddish gum. The gum was dissolved in anh. MeCN (3 mL) and to the solution was added 4-[(2,2,2-trifluoroethyl)amino]-2-(trifluoromethyl)benzonitrile (0.126 g, 0.47 mmol, Example 10A) and anh. Cs₂CO₃ (0.310 g, 0.95 mmol). The mixture was heated at 80° C. for 30 min, cooled and concentrated in vacuo. The residue was partitioned between EtOAc/H₂O, the layers were separated and the aqueous layer was extracted with EtOAc (×2). Combined organics were washed (H₂O, brine), dried over Na₂SO₄, filtered and concentrated in vacuo. The residue was purified by flash chromatography (EtOAc/hexanes), affording 0.0136 g of the title compound as an opaque gum: MS (AP) m/z 366 (M+H).

### Example 120

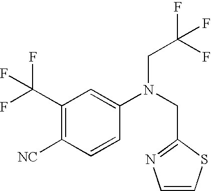

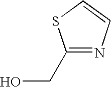

### A. 1,3-Thiazol-2-ylmethanol

To a solution of 1,3-thiazole-2-carbaldehyde (1.95 g, 17.3 mmol) in MeOH at rt was added NaBH₄ (0.783 g, 20.7 mmol), portionwise over 5 min. The mixture was stirred 15 min, then 10% HCl was added dropwise until evolution of gas ceased. The mixture was concentrated in vacuo, the residue was partitioned between EtOAc/brine and the layers were separated. The aqueous layer was extracted with EtOAc (×2). The combined organic portions were dried over Na₂SO₄, filtered, and concentrated in vacuo. The residue was purified by flash chromatography (EtOAc/hexanes), affording 1.42 g of the title compound as a waxy yellow solid: ¹H NMR (400 MHz, CDCl₃) δ 7.73 (d, J=3.3 Hz, 1H), 7.32 (d, J=3.2 Hz, 1H), 4.96 (s, 2H), 3.93 (bs, 1H).

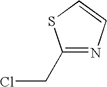

### B. 2-(Chloromethyl)-1,3-thiazole

A slurry of 1,3-thiazol-2-ylmethanol (0.542 g, 4.71 mmol) in CCl₄/toluene (1:1, 16 mL) was heated to 60° C. until complete dissolution was realized (ca. 10 min). The solution was cooled to rt, PPh₃ (1.85 g, 7.07 mmol) was added in one portion, and the mixture was brought to reflux under N₂. After 2 h, the mixture was cooled to rt and filtered through a pad of Celite. The filtrate was concentrated in vacuo and purified by flash chromatography (EtOAc/hexanes), affording 0.315 g of the title compound as a colorless oil: ¹H NMR (400 MHz, CDCl₃) δ 7.77 (d, J=3.2 Hz, 1H), 7.39 (d, J=3.2 Hz, 1H), 4.88 (s, 2H).

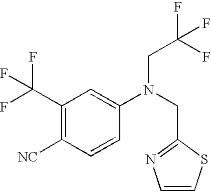

### C. 4-[(1,3-Thiazol-2-ylmethyl)(2,2,2-trifluoroethyl)amino]-2-(trifluoromethyl)benzonitrile

Synthesized as described in Example 10B from 2-(chloromethyl)-1,3-thiazole, Example 120B: ¹H NMR (400 MHz, CDCl₃) δ 7.78 (d, J=3.2 Hz, 1H), 7.66 (d, J=8.8 Hz, 1H), 7.35 (d, J=3.2 Hz, 1H), 7.20 (d, J=2.4 Hz, 1H), 7.04 (dd, J=8.8, 2.5 Hz, 1H), 5.03 (s, 2H), 4.20 (q, J=8.5 Hz, 2H).

### Example 121

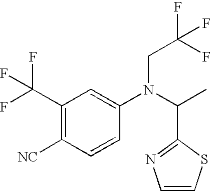

### 4-[[1,3-Thiazol-2-yl)ethyl](2,2,2-trifluoroethyl)amino]-2-(trifluoromethyl)benzonitrile

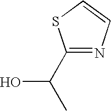

### A. 1-(1,3-Thiazol-2-yl)ethanol

Synthesized as described in step A of Example 120 from 2-acetylthiazole: ¹H NMR (400 MHz, CDCl₃) δ 7.71 (d, J=3.1 Hz, 1H), 7.29 (d, J=3.2 Hz, 1H), 5.22-5.13 (m, 1H), 3.47 (d, J=3.7 Hz, 1H), 1.65 (d, J=6.5 Hz, 3H).

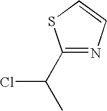

### B. 2-(1-Chloroethyl)-1,3-thiazole

Synthesized as described in step B of Example 120 from 1-(1,3-thiazol-2-yl)ethanol: ¹H NMR (400 MHz, CDCl₃) δ 7.74 (d, J=3.3 Hz, 1H), 7.35 (d, J=3.2 Hz, 1H), 5.37 (q, J=6.9 Hz, 1H), 1.99 (d, J=6.8 Hz, 3H).

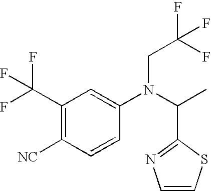

### C. 4-[[1-(1,3-Thiazol-2-yl)ethyl](2,2,2-trifluoroethyl)amino]-2-(trifluoromethyl)benzonitrile

Synthesized as described in Example 10B from 2-(1-chloroethyl)-1,3-thiazole, Example 121B: ¹H NMR (400 MHz, CDCl₃) δ 7.76 (d, J=3.3 Hz, 1H), 7.63 (d, J=8.8 Hz, 1H), 7.35 (d, J=3.3 Hz, 1H), 7.20 (d, J=2.6 Hz, 1H), 7.03 (dd, J=8.7, 2.6 Hz, 1H), 5.35 (q, J=7.0 Hz, 1H), 4.13 (q, J=8.5 Hz, 2H), 1.90 (d, J=7.0 Hz, 3H).

### Example 122

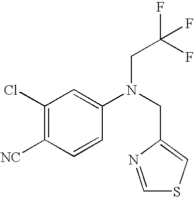

### 2-Chloro-4-[(1,3-thiazol-4-ylmethyl)(2,2,2-trifluoroethyl)amino]benzonitrile

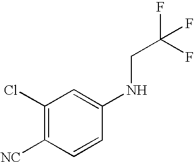

### A. 2-Chloro-4-[(2,2,2-trifluoroethyl)amino]benzonitrile

Synthesized in a manner similar to Example 10A using 4-amino-2-chlorobenzonitrile: MS (ES) m/z 235 (M+1).

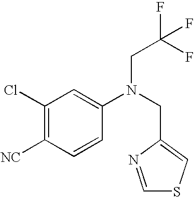

### B. 2-Chloro-4-[(1,3-thiazol-4-ylmethyl)(2,2,2-trifluoroethyl)amino]benzonitrile

To 2-chloro-4-[(2,2,2-trifluoroethyl)amino]benzonitrile (0.1 g, 0.426 mmol) in CH₃CN (2 mL) was added 4-(chloromethyl)-1,3-thiazole hydrochloride (0.13 g, 0.762 mmol) and Cs₂CO₃ (0.497 g, 1.524 mmol). The reaction was heated to 80° C. for 14 h in a sealed tube. Concentration was followed by purification (SiO₂ and purify eluting with EtOAc/hexanes) to afford the title compound (0.083 g): MS (APCI) m/z 332 (M+1).

### Example 123

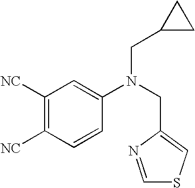

### 4-[(Cyclopropylmethyl)(1,3-thiazol-4-ylmethyl)amino]-1,2-benzenedicarbonitrile

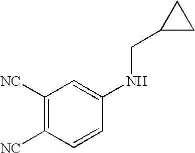

### A. 4-[(Cyclopropylmethyl)amino]-1,2-benzenedicarbonitrile

Synthesized in a manner similar to Example 1A using 4-fluoro-1,2-benzenedicarbonitrile: MS (ES) m/z 198 (M+1).

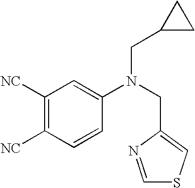

### B. 4-[(Cyclopropylmethyl)(1,3-thiazol-4-ylmethyl)amino]-1,2-benzenedicarbonitrile

To 4-[(cyclopropylmethyl)amino]-1,2-benzenedicarbonitrile (0.1 g, 0.508 mmol) in CH₃CN (2 mL), was added 4-(chloromethyl)-1,3-thiazole hydrochloride (0.13 g, 0.762 mmol) and Cs₂CO₃ (0.497 g, 1.524 mmol). The reaction was heated to 80° C. for 14 h in a sealed tube. Concentration was followed by purification (SiO₂ and purify eluting with EtOAc/hexanes) to afford the title compound (0.030 g): MS (APCI) m/z 295 (M+1).

### Example 124

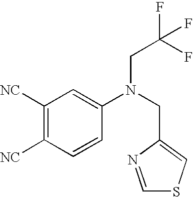

### 4-[(1,3-Thiazol-4-ylmethyl)(2,2,2-trifluoroethyl)amino]-1,2-benzenedicarbonitrile

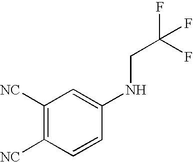

### A. 4-[(2,2,2-Trifluoroethyl)amino]-1,2-benzenedicarbonitrile

Synthesized in a manner similar to Example 10A using 4-amino-1,2-benzenedicarbonitrile: ¹H NMR (400 MHz, CDCl₃) δ 7.59 (d, J=8.6 Hz, 1H), 6.99 (d, J=2.6 Hz, 1H), 6.90 (dd, J=8.6, 2.6 Hz, 1H), 4.73 (broad t, NH), 3.86 (q, J=8.6 Hz, 2H).

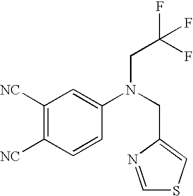

### B. 4-[(1,3-Thiazol-4-ylmethyl)(2,2,2-trifluoroethyl)amino]-1,2-benzenedicarbonitrile

To 4-[(2,2,2-trifluoroethyl)amino]-1,2-benzenedicarbonitrile (0.1 g, 0.444 mmol) in CH₃CN (2 mL) was added 4-(chloromethyl)-1,3-thiazole hydrochloride (0.13 g, 0.762 mmol) and Cs₂CO₃ (0.497 g, 1.524 mmol). The reaction was heated to 80° C. for 14 h in a sealed tube. The mixture was concentrated and purified by flash chromatography eluting with EtOAc/hexanes to afford the title compound (0.062 g): MS (APCI) m/z 323 (M+1).

### Example 125

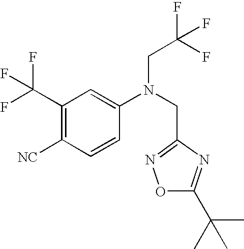

### 4-[[(5-tert-Butyl-1,2,4-oxadiazol-3-yl)methyl](2,2,2-trifluoroethyl)amino]-2-(trifluoromethyl)benzonitrile

Synthesized as described in Example 10B using 5-tert-butyl-3-(chloromethyl)-1,2,4-oxadiazole: MS (ES) m/z 407 (M+1).

### Example 126

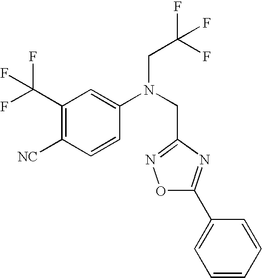

### 4-[[(5-Phenyl-1,2,4-oxadiazol-3-yl)methyl](2,2,2-trifluoroethyl)amino]-2-(trifluoromethyl)benzonitrile

Synthesized as described in Example 10B using 3-(chloromethyl)-5-phenyl-1,2,4-oxadiazole: MS (ES) m/z 427 (M+1).

### Example 127

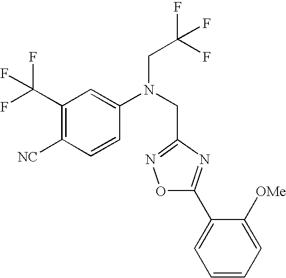

### 4-[{[5-(2-Methoxyphenyl)-1,2,4-oxadiazol-3-yl]methyl}(2,2,2-trifluoroethyl)amino]-2-(trifluoromethyl)benzonitrile

Synthesized as described in Example 10B using 3-(chloromethyl)-5-(2-methoxyphenyl)-1,2,4-oxadiazole: MS (ES) m/z 457 (M+1).

### Example 128

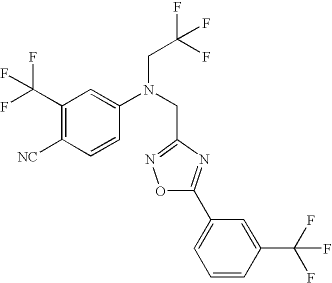

### 4-[(2,2,2-Trifluoroethyl)({5-[3-(trifluoromethyl)phenyl]-1,2,4-oxadiazol-3-yl}methyl)amino]-2-(trifluoromethyl)benzonitrile

Synthesized as described in Example 10B using 3-(chloromethyl)-5-[3-(trifluoromethyl)phenyl]-1,2,4-oxadiazole: MS (ES) m/z 493 (M−1).

### Example 129

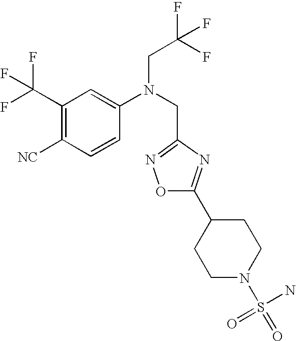

### 4-(3-{[[4-Cyano-3-(trifluoromethyl)phenyl](2,2,2-trifluoroethyl)amino]methyl}-1,2,4-oxadiazol-5-yl)-1-piperidinesulfonamide

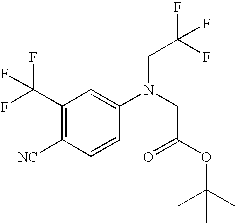

### A. 1,1-Dimethylethyl N-[4-Cyano-3-(trifluoromethyl)phenyl]-N-(2,2,2-trifluoroethyl)glycinate

Synthesized as described for Example 1B using 1,1-dimethylethyl bromoacetate and Example 10A: ¹H NMR (300 MHz, CD₃OD) δ 7.70 (d, J=8.8 Hz, 1H), 7.09-7.01 (m, 2H), 4.26 (q, J=8.9 Hz, 2H), 4.23 (s, 2H, overlapped with 4.26), 1.44 (s, 9H).

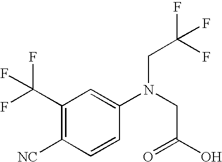

### B. N-[4-Cyano-3-(trifluoromethyl)phenyl]-N-(2,2,2-trifluoroethyl)glycine

A mixture of 1,1-dimethylethyl N-[4-cyano-3-(trifluoromethyl)phenyl]-N-(2,2,2-trifluoroethyl)glycinate, Example 129A, (8.38 g, 22.0 mmol), TFA (200 mL), and triethylsilane (35 mL) in CH₂Cl₂ (200 mL) was stirred at ambient temperature for 3 h. When the TLC indicated no remaining starting material, the solvents were removed under reduced pressure, and the residue was stirred in hexanes (200 mL) for 12 h. The solid precipitate was filtered and dried to obtain 6.78 g (95%) of the title compound as a white solid: ¹H NMR (300 MHz, CD₃OD) δ 7.67 (d, J=8.6 Hz, 1H), 7.07 (d, J=2.8 Hz, 1H), 7.03 (dd, J=8.6, 2.8 Hz, 1H), 4.28 (s, overlapped with 4.24, 2H), 4.24 (q, J=8.8 Hz, 2H).

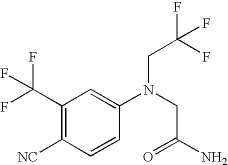

### C. N²-[4-cyano-3-(trifluoromethyl)phenyl]-#-(2,2,2-trifluoroethyl)glycinamide

A mixture of N-[4-cyano-3-(trifluoromethyl)phenyl]-N-(2,2,2-trifluoroethyl)glycine (0.325 g, 1 mmol), di-tert-butyl dicarbonate (0.436 g, 2 mmol), pyridine (0.100 g, 1.25 mmol), and ammonium hydrocarbonate (0.160 g, 2.25 mmol) was stirred for 24 h in acetonitrile (8 mL). The solvent was removed under reduced pressure, then water (10 mL) was added to the residue. The resulting solid was filtered, washed with water (3×5 mL), and then dried. After drying, the solid was suspended in diethyl ether (10 mL) and stirred for 12 h. The mixture was filtered, washed with diethyl ether (5 mL), and dried to obtain 0.132 g (40%) of the title compound as a white solid: ¹H NMR (300 MHz, MeOH-d₄) δ 7.72 (d, J=8.6 Hz, 1H), 7.15 (d, J=2.5 Hz, 1H), 7.11 (dd, J=8.8, 2.5 Hz, 1H), 4.34 (q, J=8.9 Hz, 2H), 4.27 (s, 2H).

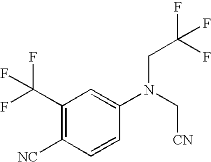

### D. 4-[(Cyanomethyl)(2,2,2-trifluoroethyl)amino]-2-(trifluoromethyl)benzonitrile

A mixture of N²-[4-cyano-3-(trifluoromethyl)phenyl]-N²-(2,2,2-trifluoroethyl)glycinamide (0.060 g, 0.18 mmol), CCl₄ (0.5 mL), DCE (4.5 mL) and polymer-supported triphenyl phosphine (3 mmol/g, 0.123 g, 0.36 mmol) was heated at 87° C. for 3 h. Upon cooling, the resin was filtered off, washed thoroughly with CHCl₃ and the filtrate concentrated in vacuo. The residue was purified by silica gel chromatography (5-40% EtOAc-hexanes gradient) and the product was crystallized from CH₂Cl₂-hexanes to give the title compound as a white solid (0.030 g, 55% yield): ¹HNMR (400 MHz, CDCl₃) δ 7.90 (d, J=8.6 Hz, 1H), 7.20 (d, J=2.6 Hz, 1H), 7.09 (dd, J=8.6, 2.6 Hz, 1H), 4.38 (s, 2H), 4.06 (q, J=8.2 Hz, 2H); MS (ES) m/z 308 (M+1).

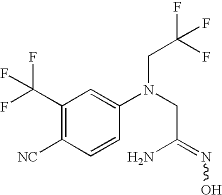

### E. 2-[[4-Cyano-3-(trifluoromethyl)phenyl](2,2,2-trifluoroethyl)amino]-N-hydroxyethanimidamide

To a solution of 4-[(cyanomethyl)(2,2,2-trifluoroethyl)amino]-2-(trifluoromethyl)benzonitrile, Example 129A, (1.0 g, 3.25 mmol) in DMF (12 mL) was added hydroxylamine. HCl (0.45 g, 6.51 mmol) and sodium acetate (0.53 g, 6.51 mmol). After stirring at rt for 15 h, the mixture was partitioned between Et₂O (100 mL) and 1N HCl (100 mL). The aqueous phase was extracted with Et₂O (2×50 mL) and the organic phases discarded. The aqueous phase was basified with 5N NaOH and extracted with Et₂O (×2). The combined organic phases were washed with brine, dried (Na₂SO₄) and concentrated. The residue was crystallized from CH₂Cl₂/hexanes to afford the title compound as a white solid (0.92 g, 84% yield): MS (ES) m/z 341 (M+1).

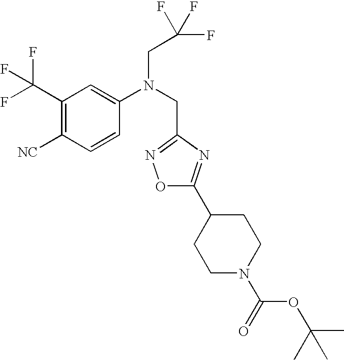

### F. 1,1-Dimethylethyl 4-(3-{[[4-cyano-3-(trifluoromethyl)phenyl](2,2,2-trifluoroethyl)amino]methyl}-1,2,4-oxadiazol-5-yl)-1-piperidinecarboxylate

To a solution of 1-{[(1,1-dimethylethyl)oxy]carbonyl}-4-piperidinecarboxylic acid (0.222 g, 0.97 mmol) in anhydrous THF (3 mL) was added 1,1′-carbonyldiimidazole (0.157 g, 0.97 mmol). After stirring at rt for 45 min, 2-[[4-cyano-3-(trifluoromethyl)phenyl](2,2,2-trifluoroethyl)amino]-N-hydroxyethanimidamide (0.300 g, 0.88 mmol) was added. The mixture was stirred at rt for 15 min and then heated in a microwave at 140° C. for 20 min. Upon cooling, the mixture was concentrated to dryness. The residue was partitioned between CH₂Cl₂ and 0.2N NaOH. The organic phase was washed with 0.2N NaOH and brine, dried (Na₂SO₄) and concentrated. The residue was purified by silica gel chromatography (5-50% EtOAc-hexanes gradient) to afford the title compound as a white solid (0.319 g, 68% yield): MS (ES) m/z 532 (M−1).

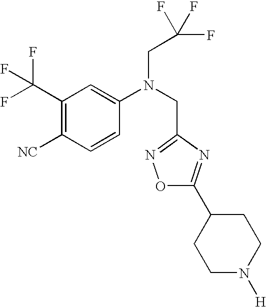

### G. 4-[{[5-(4-Piperidinyl)-1,2,4-oxadiazol-3-yl]methyl}(2,2,2-trifluoroethyl)amino]-2-(trifluoromethyl)benzonitrile

To a solution of 1,1-dimethylethyl 4-(3-{[[4-cyano-3-(trifluoromethyl)phenyl](2,2,2-trifluoroethyl)amino]methyl}-1,2,4-oxadiazol-5-yl)-1-piperidinecarboxylate (0.090 g, 0.17 mmol) in CH₂Cl₂ (8 mL) was added TFA (4 mL). After stirring at rt for 3 h, the mixture was concentrated to dryness. The residue was dissolved in CH₂Cl₂ and washed with 0.2N NaOH and brine, dried (Na₂SO₄) and concentrated to afford the title compound, which was used in the next step without further purification: MS (APCI) m/z 434 (M+1).

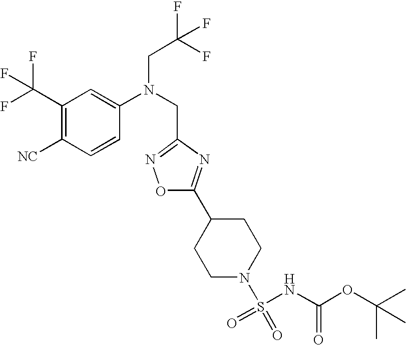

### H. 1,1-Dimethylethyl {[4-(3-{[[4-cyano-3-(trifluoromethyl)phenyl](2,2,2-trifluoroethyl)amino]methyl}-1,2,4-oxadiazol-5-yl)-1-piperidinyl]sulfonyl}carbamate

To an ice-cold solution of t-BuOH (0.016 g, 0.22 mmol) in CH₂Cl₂ (0.3 ml) was added chlorosulfonyl isocyanate (0.029 g, 0.21 mmol). After 5 min, the mixture was added to an ice-cold solution of 4-[{[5-(4-piperidinyl)-1,2,4-oxadiazol-3-yl]methyl}(2,2,2-trifluoroethyl)amino]-2-(trifluoromethyl)benzonitriles, Example 129G, (0.030 g, 0.07 mmol) and TEA (0.014 g, 0.14 mmol) in CH₂Cl₂ (1.5 ml). After stirring at rt for 15 h, the mixture was partitioned between EtOAc and 0.1N HCl. The organic phase was washed with 0.1N HCl, 0.1N NaOH and brine, dried (Na₂SO₄) and concentrated. The residue was purified by silica gel chromatography (10-80% EtOAc-hexane gradient) to afford the title compound (0.010 g, 24% yield): ¹H NMR (400 MHz, CDCl₃) δ 7.67 (d, J=8.8 Hz, 1H), 7.22 (d, J=2.6 Hz, 1H), 7.05 (dd, J=8.8, 2.7 Hz, 1H), 7.00 (s, 1H), 4.76 (s, 2H), 4.17 (q, J=8.4 Hz, 2H), 3.90 (dt, J=13.2, 3.7 Hz, 2H), 3.16-3.02 (m, 3H), 2.19-2.12 (m, 2H), 2.00-1.90 (m, 2H), 1.48 (s, 9H).

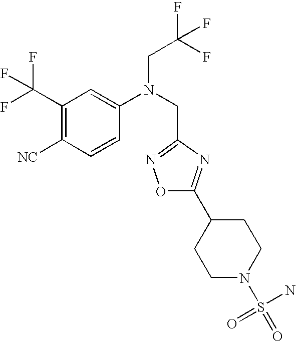

### I. 4-(3-{[[4-Cyano-3-(trifluoromethyl)phenyl](2,2,2-trifluoroethyl)amino]methyl}-1,2,4-oxadiazol-5-yl)-1-piperidinesulfonamide

To a solution of 1,1-dimethylethyl {[4-(3-{[[4-cyano-3-(trifluoromethyl)phenyl](2,2,2-trifluoroethyl)amino]methyl}-1,2,4-oxadiazol-5-yl)-1-piperidinyl]sulfonyl}carbamate (0.010 g, 0.016 mmol) in CH₂Cl₂ (4 mL) was added TFA (2 mL). After stirring for 3 h, the mixture was concentrated to dryness. The residue was partitioned between CH₂Cl₂ and 0.1N NaOH. The organic phase was washed with 0.1N NaOH and brine, dried (Na₂SO₄) and concentrated. The residue was purified by silica gel chromatography (10-100% EtOAc-hexanes gradient) and the product crystallized from CH₂Cl₂-hexanes to afford the title compound as a white solid (0.004 g, 48% yield): MS (ES) m/z 513 (M+1).

### Example 130

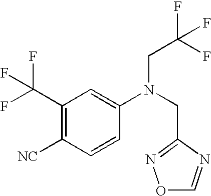

### 4-[(1,2,4-Oxadiazol-3-ylmethyl)(2,2,2-trifluoroethyl)amino]-2-(trifluoromethyl)benzonitrile

To a solution of 2-[[4-cyano-3-(trifluoromethyl)phenyl](2,2,2-trifluoroethyl)amino]-N-hydroxyethanimidamide, Example 129E, (0.020 g, 0.059 mmol) in triethyl orthoformate (3 mL), under N₂, was added boron trifluoride diethyl etherate (4 drops). The mixture was heated in a microwave at 160° C. for 20 min. Upon cooling, the mixture was partitioned between EtOAc and NaHCO₃ solution. The organic phase was washed with sat'd brine, dried over Na₂SO₄, filtered and concentrated in vacuo. The residue was purified by silica gel chromatography (0-30% EtOAc-hexane gradient) and the product was crystallized from CH₂Cl₂-hexanes to give the title compound as a white solid (0.009 g, 42% yield): ¹H NMR (400 MHz, CDCl₃) δ 8.74 (s, 1H), 7.67 (d, J=8.8 Hz, 1H), 7.21 (d, J=2.6 Hz, 1H), 7.06 (dd, J=8.8, 2.6 Hz, 1H), 4.86 (s, 2H), 4.19 (q, J=8.4 Hz, 2H).

### Example 131

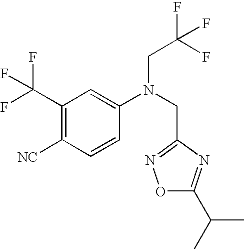

### 4-[{[5-(1-Methylethyl)-1,2,4-oxadiazol-3-yl]methyl}(2,2,2-trifluoroethyl)amino]-2-(trifluoromethyl)benzonitrile

To a solution of 2-[[4-cyano-3-(trifluoromethyl)phenyl](2,2,2-trifluoroethyl)amino]-N-hydroxyethanimidamide, Example 129E, (0.050 g, 0147 mmol) in DMF (2 mL) was added TEA (0.016 g, 0.159 mmol) and isobutyryl chloride (0.017 g, 0.159 mmol). The mixture was stirred at rt for 40 min, and then heated in a microwave at 150° C. for 20 min. Upon cooling, the mixture was partitioned between Et₂O and 0.1N HCl. The organic phase was washed with 0.1N HCl, 0.1N NaOH and sat'd brine, dried over Na₂SO₄, filtered and concentrated. The residue was purified by silica gel chromatography (5-50% EtOAc-hexanes gradient) and the product crystallized from CH₂Cl₂-hexanes to give the title compound as a white solid (0.027 g, 47% yield): MS (AP) m/z 393 (M+1).

### Example 132

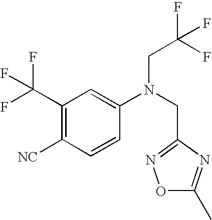

### 4-[[(5-Methyl-1,2,4-oxadiazol-3-yl)methyl](2,2,2-trifluoroethyl)amino]-2-(trifluoromethyl)benzonitrile

Synthesized in a manner similar to Example 131, using acetic anhydride: MS (ES) m/z 365 (M+1).

### Example 133

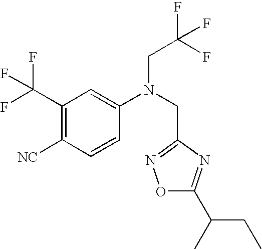

### 4-[{[5-(1-Methylpropyl)-1,2,4-oxadiazol-3-yl]methyl}(2,2,2-trifluoroethyl)amino]-2-(trifluoromethyl)benzonitrile

Synthesized in a manner similar to Example 131, using 2-methylbutyryl chloride: MS (ES) m/z 407 (M+1).

### Example 134

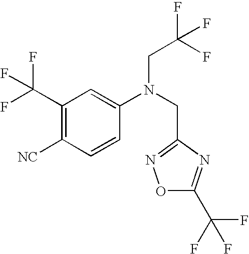

### 4-((2,2,2-Trifluoroethyl){[5-(trifluoromethyl)-1,2,4-oxadiazol-3-yl]methyl}amino)-2-(trifluoromethyl)benzonitrile

Synthesized in a manner similar to Example 131, using trifluoroacetic anhydride: MS (ES) m/z 417 (M−1).

### Example 135

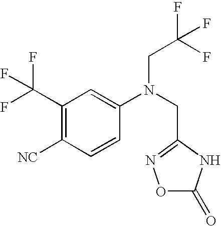

### 4-[[(5-Oxo-2,5-dihydro-1,2,4-oxadiazol-3-yl)methyl](2,2,2-trifluoroethyl)amino]-2-(trifluoromethyl)benzonitrile

A solution of 2-[[4-cyano-3-(trifluoromethyl)phenyl](2,2,2-trifluoroethyl)amino]-N-hydroxyethanimidamide, Example 129E, (0.050 g, 0147 mmol), carbonyl diimidazole (0.029 g, 0.176 mmol) and DBU (0.025 g, 0.162 mmol) in THF (2 mL) was heated in a microwave at 120° C. for 20 min. Upon cooling, the mixture was partitioned between EtOAc and 0.5N HCl. The organic phase was washed with 0.5N HCl and sat'd brine, dried over Na₂SO₄ and concentrated. The residue was crystallized from CH₂Cl₂-MeOH-hexane to give the title compound as a white solid (0.049 g, 90% yield): MS (ES) m/z 365 (M−1).

### Example 136

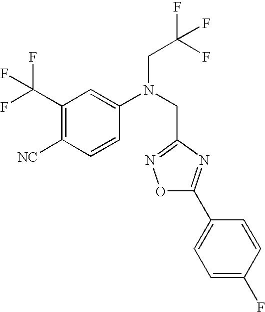

### 4-[{[5-(4-Fluorophenyl)-1,2,4-oxadiazol-3-yl]methyl}(2,2,2-trifluoroethyl)amino]-2-(trifluoromethyl)benzonitrile

Synthesized in a manner similar to Example 131, using 4-fluorobenzoyl chloride: MS (ES) m/z 445 (M+1).

### Example 137

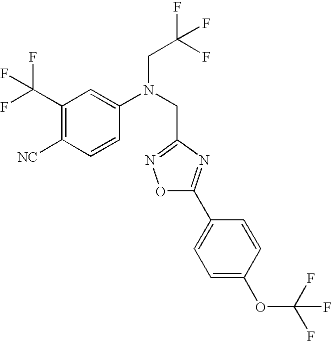

### 4-{(2,2,2-Trifluoroethyl)[(5-{4-[(trifluoromethyl)oxy]phenyl}-1,2,4-oxadiazol-3-yl)methyl]amino}-2-(trifluoromethyl)benzonitrile

Synthesized in a manner similar to Example 131, using 4-(trifluoromethoxy)benzoyl chloride: MS (ES) m/z 511 (M+1).

### Example 138

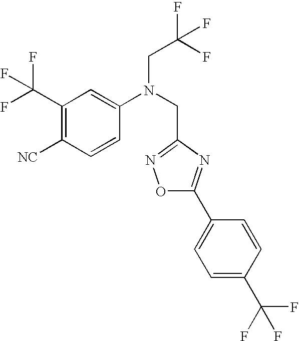

### 4-[(2,2,2-Trifluoroethyl)({5-[4-(trifluoromethyl)phenyl]-1,2,4-oxadiazol-3-yl}methyl)amino]-2-(trifluoromethyl)benzonitrile

Synthesized in a manner similar to Example 10B, using 3-(chloromethyl)-5-[4-(trifluoromethyl)phenyl]-1,2,4-oxadiazole: MS (ES) m/z 493 (M−1).

### Example 139

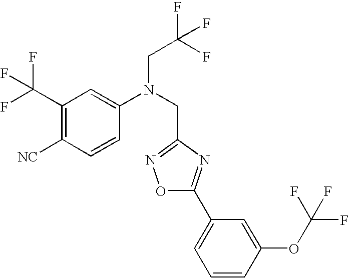

### 4-{(2,2,2-Trifluoroethyl)[(5-{3-[(trifluoromethyl)oxy]phenyl}-1,2,4-oxadiazol-3-yl)methyl]amino}-2-(trifluoromethyl)benzonitrile

Synthesized in a manner similar to Example 131, using 3-(trifluoromethoxy)benzoyl chloride: MS (ES) m/z 511 (M+1).

### Example 140

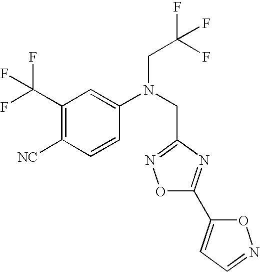

### 4-[{[5-(5-Isoxazolyl)-1,2,4-oxadiazol-3-yl]methyl}(2,2,2-trifluoroethyl)amino]-2-(trifluoromethyl)benzonitrile

Synthesized in a manner similar to Example 131, using 5-isoxazolecarbonyl chloride: MS (ES) m/z 418 (M+1).

### Example 141

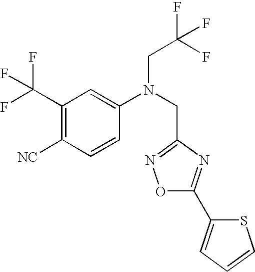

### 4-[{[5-(2-Thienyl)-1,2,4-oxadiazol-3-yl]methyl}(2,2,2-trifluoroethyl)amino]-2-(trifluoromethyl)benzonitrile

Synthesized in a manner similar to Example 10B, using 3-(chloromethyl)-5-(2-thienyl)-1,2,4-oxadiazole: MS (ES) m/z 433 (M+1).

### Example 142

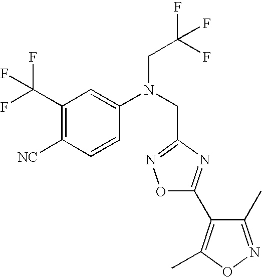

### 4-[{[5-(3,5-Dimethyl-4-isoxazolyl)-1,2,4-oxadiazol-3-yl]methyl}(2,2,2-trifluoroethyl)amino]-2-(trifluoromethyl)benzonitrile

Synthesized in a manner similar to Example 10B, using 3-(chloromethyl)-5-(3,5-dimethyl-4-isoxazolyl)-1,2,4-oxadiazole: MS (ES) m/z 468 (M+Na).

### Example 143

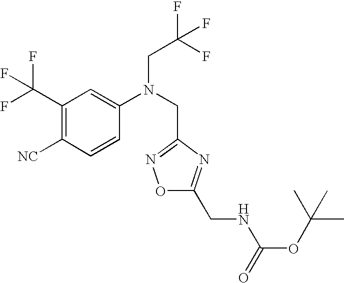

### 1,1-Dimethylethyl[(3-{[[4-cyano-3-(trifluoromethyl)phenyl](2,2,2-trifluoroethyl)amino]methyl}-1,2,4-oxadiazol-5-yl)methyl]carbamate

To a solution of N-{([(1,1-dimethylethyl)oxy]carbonyl}glycine (0.056 g, 0.318 mmol) in DMF (1 mL) was added EDCl (0.061 g, 0.318 mmol). After stirring at rt for 10 min, 2-[[4-cyano-3-(trifluoromethyl)phenyl](2,2,2-trifluoroethyl)amino]-N-hydroxyethanimidamide, Example 129E, (0.10 g, 0.294 mmol) was added. After stirring at rt for 40 min, the reaction mixture was diluted with 2 mL of DMF and heated in a microwave at 150° C. for 20 min. Upon cooling, the mixture was partitioned between Et₂O and 0.1N HCl. The organic phase was washed with 0.1N HCl, 0.1N NaOH and sat'd brine, dried over Na₂SO₄, filtered and concentrated. The residue was purified by silica gel chromatography (5-60% EtOAc-hexane gradient) to give the title compound (0.077 g, 55% yield): MS (ES) m/z 480 (M+1).

### Example 144

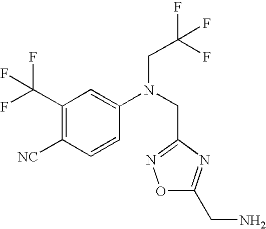

### 4-[{[5-(Aminomethyl)-1,2,4-oxadiazol-3-yl]methyl}(2,2,2-trifluoroethyl)amino]-2-(trifluoromethyl)benzonitrile

To a solution of 1,1-dimethylethyl[(3-{[[4-cyano-3-(trifluoromethyl)phenyl](2,2,2-trifluoroethyl)amino]methyl}-1,2,4-oxadiazol-5-yl)methyl]carbamate, Example 143, (0.072 g, 0.15 mmol) in CH₂Cl₂ (6 mL) was added TFA (3 mL). After stirring at rt for 6 h, the mixture was concentrated. The residue was dissolved in CH₂Cl₂ and washed with 0.5N NaOH (twice) and sat'd brine, dried over Na₂SO₄, filtered and concentrated to give the title compound (0.051 g, 90% yield): MS (ES) m/z 380 (M+1).

### Example 145

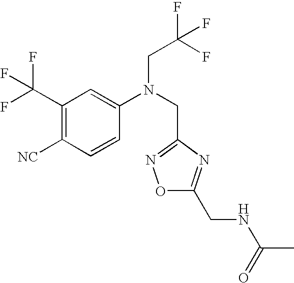

### N-[(3-{[[4-Cyano-3-(trifluoromethyl)phenyl](2,2,2-trifluoroethyl)amino]methyl}-1,2,4-oxadiazol-5-yl)methyl]acetamide

To a solution of 4-[{[5-(aminomethyl)-1,2,4-oxadiazol-3-yl]methyl}(2,2,2-trifluoroethyl)amino]-2-(trifluoromethyl)benzonitrile, Example 144, (0.016 g, 0.042 mmol) and TEA (0.0052 g, 0.051 mmol) in CH₂Cl₂ (2 mL) was added acetyl chloride (0.004 g, 0.051 mmol). After stirring at rt for 1.5 h, the mixture was partitioned between EtOAc and 0.1N HCl. The organic phase was washed with 0.1N HCl and sat'd brine, dried over Na₂SO₄, filtered and concentrated. The residue was purified by silica gel chromatography (1-6% MeOH—CH₂Cl₂ gradient) to give the title compound (0.014 g, 79% yield): MS (ES) m/z 422 (M+1).

### Example 146

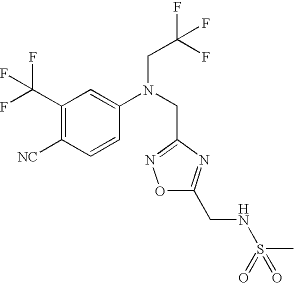

### N-[(3-{[[4-Cyano-3-(trifluoromethyl)phenyl](2,2,2-trifluoroethyl)amino]methyl}-1,2,4-oxadiazol-5-yl)methyl]methanesulfonamide

Synthesized in a manner similar to Example 145, using methanesulfonyl chloride: MS (ES) m/z 458 (M+1).

### Examples 147 and 148

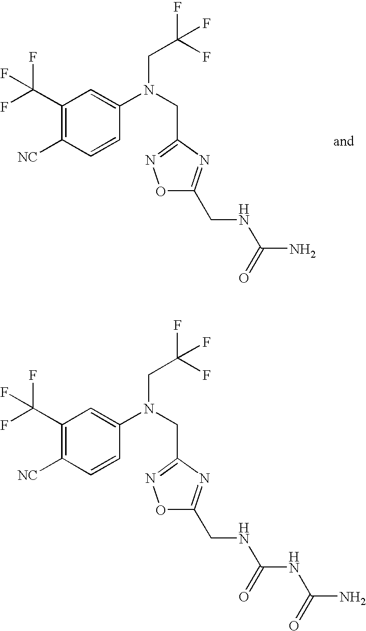

### N-[(3-{[[4-Cyano-3-(trifluoromethyl)phenyl](2,2,2-trifluoroethyl)amino]methyl}-1,2,4-oxadiazol-5-yl)methyl]urea (Example 147) and N-[(3-{[[4-cyano-3-(trifluoromethyl)phenyl](2,2,2-trifluoroethyl)amino]methyl}-1,2,4-oxadiazol-5-yl)methyl]dicarbonimidic diamide (Example 148)

Synthesized in a manner similar to Example 145, using trimethylsilyl isocyanate. The residue obtained after the reaction workup was purified by silica gel chromatography (1-10% MeOH—CH₂Cl₂) gradient) and the separated products were crystallized from CH₂Cl₂-hexanes to give the title compounds as white solids: Example 147 (0.003 g, 17% yield), MS (ES) m/z 423 (M+1) and Example 148 (0.010 g, 53% yield), MS (ES) m/z 466 (M+1).

### Example 149

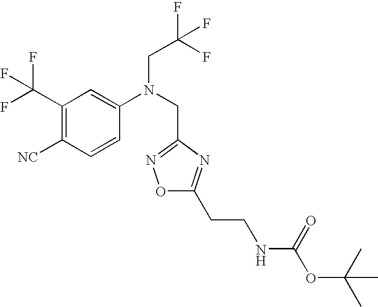

### 1,1-Dimethylethyl[2-(3-{[[4-cyano-3-(trifluoromethyl)phenyl](2,2,2-trifluoroethyl)amino]methyl}-1,2,4-oxadiazol-5-yl)ethyl]carbamate

Synthesized in a manner similar to Example 143, using N-{[(1,1-dimethylethyl)oxy]carbonyl}-β-alanine: MS (ES) m/z 494 (M+1).

### Example 150

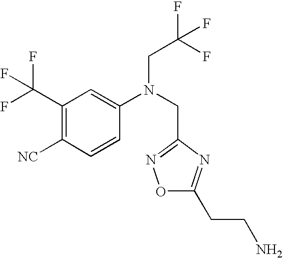

### 4-[{[5-(2-Aminoethyl)-1,2,4-oxadiazol-3-yl]methyl}(2,2,2-trifluoroethyl)amino]-2-(trifluoromethyl)benzonitrile

Synthesized in a manner similar to Example 144 using Example 149: MS (ES) m/z 394 (M+1).

### Example 151

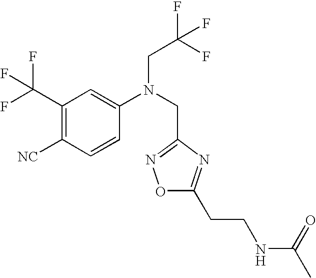

### N-[2-(3-{[[4-Cyano-3-(trifluoromethyl)phenyl](2,2,2-trifluoroethyl)amino]methyl}-1,2,4-oxadiazol-5-yl)ethyl]acetamide

Synthesized in a manner similar to Example 145, using Example 150: MS (ES) m/z 436 (M+1).

### Example 152

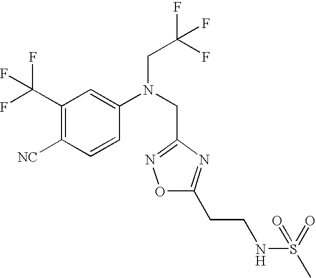

### N-[2-(3-{[[4-Cyano-3-(trifluoromethyl)phenyl](2,2,2-trifluoroethyl)amino]methyl}-1,2,4-oxadiazol-5-yl)ethyl]methanesulfonamide

Synthesized in a manner similar to Example 145, using Example 150 and methanesulfonyl chloride: MS (ES) m/z 472 (M+1).

### Examples 153 and 154

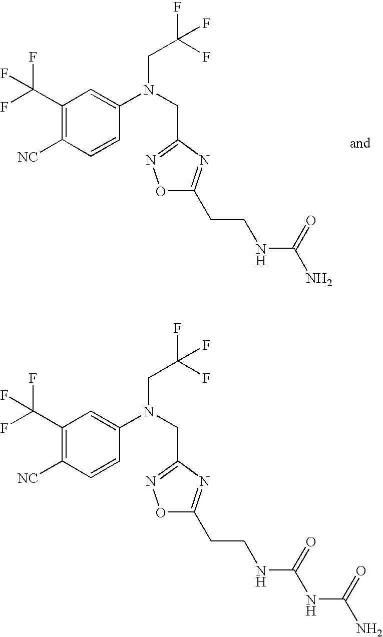

### N-[2-(3-{[[4-Cyano-3-(trifluoromethyl)phenyl](2,2,2-trifluoroethyl)amino]methyl}-1,2,4-oxadiazol-5-yl)ethyl]urea (Example 153) and N-[2-(3-{[[4-cyano-3-(trifluoromethyl)phenyl](2,2,2-trifluoroethyl)amino]methyl}-1,2,4-oxadiazol-5-yl)ethyl]dicarbonimidic diamide (Example 154)

Synthesized in a manner similar to Examples 147 and 148 using Example 150: Example 153, MS (ES) m/z 437 (M+1) and Example 154, MS (ES) m/z 480 (M+1).

### Example 155

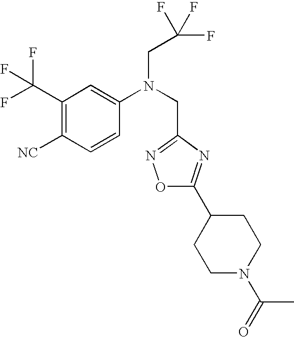

### 4-[{[5-(1-Acetyl-4-piperidinyl)-1,2,4-oxadiazol-3-yl]methyl}(2,2,2-trifluoroethyl)amino]-2-(trifluoromethyl)benzonitrile

Synthesized in a manner similar to Example 129F using 1-acetyl-4-piperidinecarboxylic acid: MS (ES) m/z 476 (M+1).

### Example 156

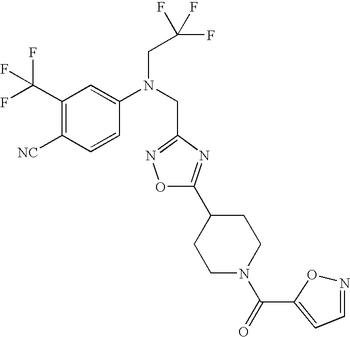

### 4-[({5-[1-(5-Isoxazolylcarbonyl)-4-piperidinyl]-1,2,4-oxadiazol-3-yl}methyl)(2,2,2-trifluoroethyl)amino]-2-(trifluoromethyl)benzonitrile

Synthesized in a manner similar to Example 145 using 4-[{[5-(4-piperidinyl)-1,2,4-oxadiazol-3-yl]methyl}(2,2,2-trifluoroethyl)amino]-2-(trifluoromethyl)benzonitrile, Example 129G, and 5-isoxazolecarbonyl chloride: MS (ES) m/z 529 (M+1).

### Example 157

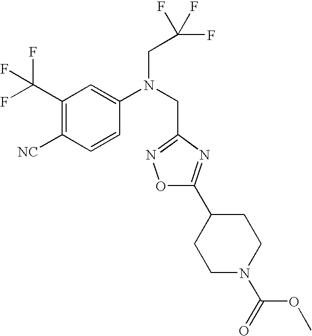

### Methyl 4-(3-{[[4-cyano-3-(trifluoromethyl)phenyl](2,2,2-trifluoroethyl)amino]methyl}-1,2,4-oxadiazol-5-yl)-1-piperidinecarboxylate

Synthesized in a manner similar to Example 145 using 4-[{[5-(4-piperidinyl)-1,2,4-oxadiazol-3-yl]methyl}(2,2,2-trifluoroethyl)amino]-2-(trifluoromethyl)benzonitrile, Example 129G, and methyl chloroformate: MS (ES) m/z 492 (M+1).

### Example 158

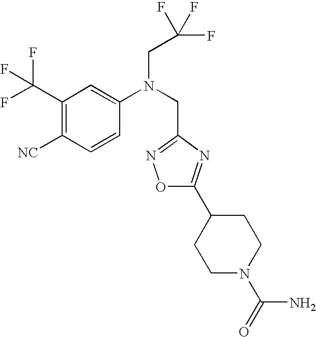

### 4-(3-{[[4-Cyano-3-(trifluoromethyl)phenyl](2,2,2-trifluoroethyl)amino]methyl}-1,2,4-oxadiazol-5-yl)-1-piperidinecarboxamide

To a solution of 4-[{[5-(4-piperidinyl)-1,2,4-oxadiazol-3-yl]methyl}(2,2,2-trifluoroethyl)amino]-2-(trifluoromethyl)benzonitrile, Example 129G (0.043 g, 0.099 mmol) in CH₂Cl₂ (2 mL) was added trimethylsilyl isocyanate (0.02 g, 0.174 mmol), and the mixture was stirred at rt. The reaction was monitored by TLC, and additional isocyanate reagent was added accordingly. The mixture was concentrated and the residue purified by silica gel chromatography to give the title compound (0.044 g, 93% yield): MS (ES) m/z 477 (M+1).

### Example 159

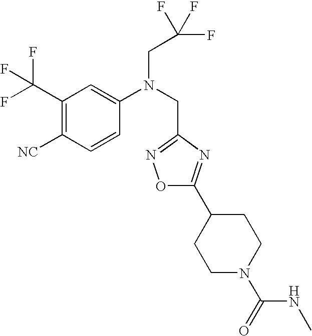

### 4-(3-{[[4-Cyano-3-(trifluoromethyl)phenyl](2,2,2-trifluoroethyl)amino]methyl}-1,2,4-oxadiazol-5-yl)-N-methyl-1-piperidinecarboxamide

Synthesized in a manner similar to Example 158 using methyl isocyanate: MS (ES) m/z 491 (M+1).

### Example 160

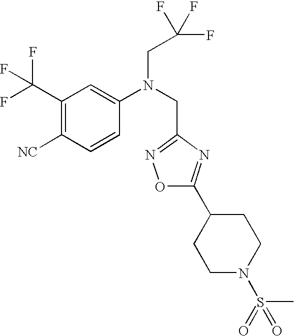

### 4-[({5-[1-(Methylsulfonyl)-4-piperidinyl]-1,2,4-oxadiazol-3-yl}methyl)(2,2,2-trifluoroethyl)amino]-2-(trifluoromethyl)benzonitrile

Synthesized in a manner similar to Example 145 using 4-[{[5-(4-piperidinyl)-1,2,4-oxadiazol-3-yl]methyl}(2,2,2-trifluoroethyl)amino]-2-(trifluoromethyl)benzonitrile, Example 129G and methanesulfonyl chloride: MS (ES) m/z 512 (M+1).

### Example 161

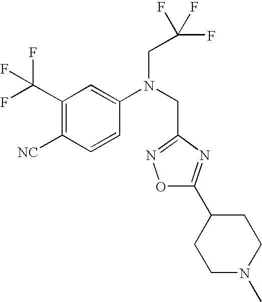

### 4-[{[5-(1-Methyl-4-piperidinyl)-1,2,4-oxadiazol-3-yl]methyl}(2,2,2-trifluoroethyl)amino]-2-(trifluoromethyl)benzonitrile

To a solution of 4-[{[5-(4-piperidinyl)-1,2,4-oxadiazol-3-yl]methyl}(2,2,2-trifluoroethyl)amino]-2-(trifluoromethyl)benzonitrile, Example 129G, (0.060 g, 0.139 mmol) in MeOH (3 mL) was added formaldehyde (0.016 mL of a 38% aqueous solution, 0.208 mmol), acetic acid (0.042 g, 0.695 mmol) and sodium cyanoborohydride (0.013 g, 0.208 mmol). After stirring at rt for 15 h, the mixture was concentrated. The residue was partitioned between CH₂Cl₂ and 0.1N NaOH. The organic phase was washed with sat'd brine, dried over Na₂SO₄, filtered and concentrated. The residue was purified by silica gel chromatography (0-7% MeOH—CH₂Cl₂) to give the title compound (0.048 g, 77% yield): MS (ES) m/z 448 (M+1).

### Example 162

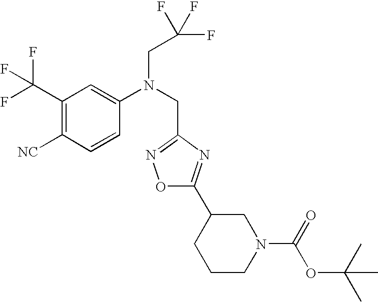

### 1,1-Dimethylethyl 3-(3-{[[4-cyano-3-(trifluoromethyl)phenyl](2,2,2-trifluoroethyl)amino]methyl}-1,2,4-oxadiazol-5-yl)-1-piperidinecarboxylate

Synthesized in a manner similar to Example 129F using 1-{[(1,1-dimethylethyl)oxy]carbonyl}-3-piperidinecarboxylic acid: MS (ES) m/z 534 (M+1).

### Example 163

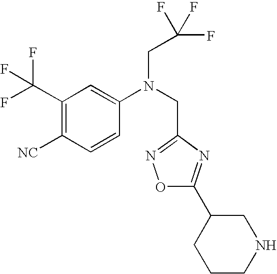

### 4-[{[5-(3-Piperidinyl)-1,2,4-oxadiazol-3-yl]methyl}(2,2,2-trifluoroethyl)amino]-2-(trifluoromethyl)benzonitrile

Synthesized in a manner similar to Example 129D using Example 162: ¹H NMR (400 MHz, CDCl₃) δ 7.64 (d, J=8.8 Hz, 1H), 7.22 (d, J=2.6 Hz, 1H), 7.05 (dd, J=8.8; 2.6 Hz, 1H), 4.74 (s, 2H), 4.17 (q, J=8.6 Hz, 2H), 3.29 (dd, J=12.2, 3.1 Hz, 1H), 3.08-3.02 (m, 1H), 3.01-2.91 (m, 2H), 2.72-2.66 (m, 1H), 2.14-2.11 (m, 1H), 1.83-1.69 (m, 3H), 1.58-1.50 (m, 1H).

### Example 164

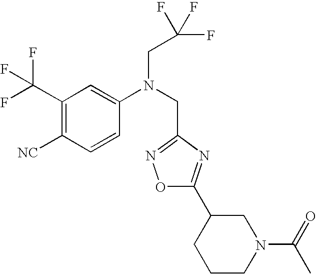

### 4-[{[5-(1-Acetyl-3-piperidinyl)-1,2,4-oxadiazol-3-yl]methyl}(2,2,2-trifluoroethyl)amino]-2-(trifluoromethyl)benzonitrile

Synthesized in a manner similar to Example 145, using 4-[{[5-(3-piperidinyl)-1,2,4-oxadiazol-3-yl]methyl}(2,2,2-trifluoroethyl)amino]-2-(trifluoromethyl)benzonitrile, Example 163: MS (ES) m/z 476 (M+1).

### Example 165

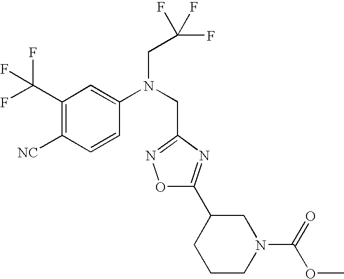

### Methyl 3-(3-{[[4-cyano-3-(trifluoromethyl)phenyl](2,2,2-trifluoroethyl)amino]methyl}-1,2,4-oxadiazol-5-yl)-1-piperidinecarboxylate

Synthesized in a manner similar to Example 145, using 4-[{[5-(3-piperidinyl)-1,2,4-oxadiazol-3-yl]methyl}(2,2,2-trifluoroethyl)amino]-2-(trifluoromethyl)benzonitrile, Example 163, and methyl chloroformate: MS (ES) m/z 492 (M+1).

### Example 166

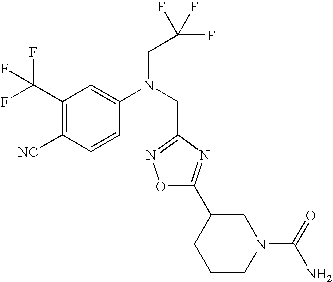

### 3-(3-{[[4-Cyano-3-(trifluoromethyl)phenyl](2,2,2-trifluoroethyl)amino]methyl}-1,2,4-oxadiazol-5-yl)-1-piperidinecarboxamide

Synthesized in a manner similar to Example 158, using 4-[{[5-(3-piperidinyl)-1,2,4-oxadiazol-3-yl]methyl}(2,2,2-trifluoroethyl)amino]-2-(trifluoromethyl)benzonitrile, Example 163: MS (AP) m/z 477 (M+1).

### Example 167

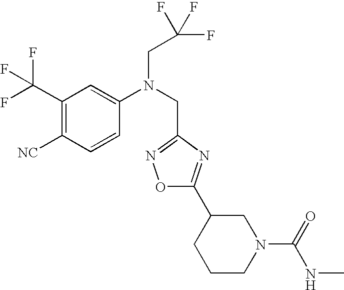

### 3-(3-{[[4-Cyano-3-(trifluoromethyl)phenyl](2,2,2-trifluoroethyl)amino]methyl}-1,2,4-oxadiazol-5-yl)-N-methyl-1-piperidinecarboxamide

Synthesized in a manner similar to Example 158, using 4-[{[5-(3-piperidinyl)-1,2,4-oxadiazol-3-yl]methyl}(2,2,2-trifluoroethyl)amino]-2-(trifluoromethyl)benzonitrile, Example 163, and methyl isocyanate: MS (AP) m/z 491 (M+1).

### Example 168

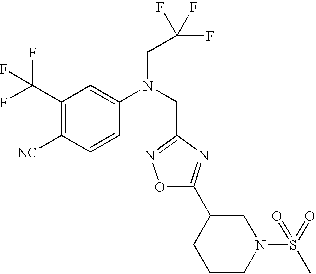

### 4-[({5-[1-(Methylsulfonyl)-3-piperidinyl]-1,2,4-oxadiazol-3-yl}methyl)(2,2,2-trifluoroethyl)amino]-2-(trifluoromethyl)benzonitrile

Synthesized in a manner similar to Example 145, using 4-[{[5-(3-piperidinyl)-1,2,4-oxadiazol-3-yl]methyl}(2,2,2-trifluoroethyl)amino]-2-(trifluoromethyl)benzonitrile, Example 163, and methanesulfonyl chloride: MS (AP) m/z 512 (M+1).

### Example 169

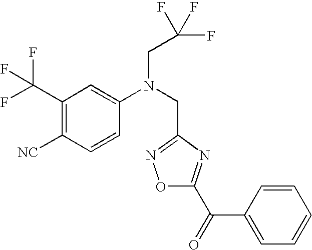

### 4-[{[5-(Phenylcarbonyl)-1,2,4-oxadiazol-3-yl]methyl}(2,2,2-trifluoroethyl)amino]-2-(trifluoromethyl)benzonitrile

Synthesized in a manner similar to Example 129F using benzoylformic acid: MS (ES) m/z 455 (M+1).

### Example 170

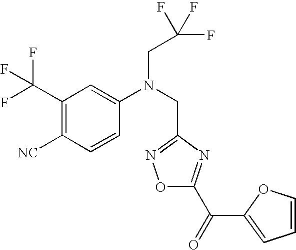

### 4-[{[5-(2-Furanylcarbonyl)-1,2,4-oxadiazol-3-yl]methyl}(2,2,2-trifluoroethyl)amino]-2-(trifluoromethyl)benzonitrile

Synthesized in a manner similar to Example 129F using 2-furanyl(oxo)acetic acid: MS (ES) m/z 445 (M+1).

### Example 171

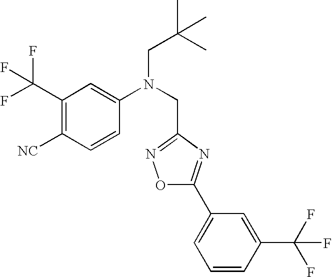

### 4-[(2,2-Dimethylpropyl)({5-[3-(trifluoromethyl)phenyl]-1,2,4-oxadiazol-3-yl}methyl)amino]-2-(trifluoromethyl)benzonitrile

Synthesized as described in Example 10B using 4-[(2,2-dimethylpropyl)amino]-2-(trifluoromethyl)benzonitrile and 3-(chloromethyl)-5-[3-(trifluoromethyl)phenyl]-1,2,4-oxadiazole: MS (ES) m/z 483 (M+1).

### Example 172

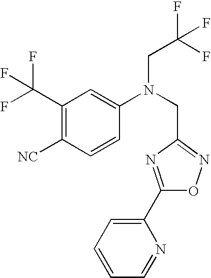

### 4-[{[5-(2-Pyridinyl)-1,2,4-oxadiazol-3-yl]methyl}(2,2,2-trifluoroethyl)amino]-2-(trifluoromethyl)benzonitrile

A CH₃CN (3 mL) solution of 2-pyridinecarboxylic acid (0.04 g, 0.323 mmol) was treated with CDI (0.052 g, 0.323 mmol) followed by 2-[[4-cyano-3-(trifluoromethyl)phenyl](2,2,2-trifluoroethyl)amino]-N-hydroxyethanimidamide, Example 129E (0.1 g, 0.294 mmol) and the resulting mixture was stirred for 30 min at rt. Next, the mixture was heated to 80° C. for 14 h. Upon cooling, the reaction was partitioned between EtOAc/H₂O. The organic portion was washed with brine, dried over Na₂SO₄, filtered and concentrated in vacuo. Purification over SiO₂ eluting with EtOAc/hexanes afforded the title compound (0.008 g): MS (ESI) m/z 428 (M+1).

### Example 173

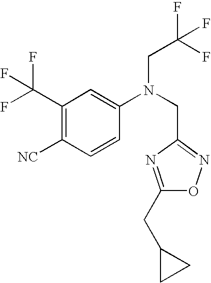

### 4-[{[5-(Cyclopropylmethyl)-1,2,4-oxadiazol-3-yl]methyl}(2,2,2-trifluoroethyl)amino]-2-(trifluoromethyl)benzonitrile

Synthesized as described in Example 172 from cyclopropylacetic acid: MS (ESI) m/z 405 (M+1).

### Example 174

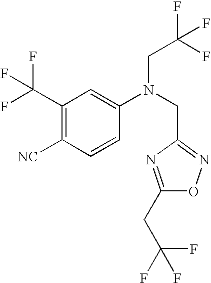

### 4-((2,2,2-Trifluoroethyl){[5-(2,2,2-trifluoroethyl)-1,2,4-oxadiazol-3-yl]methyl}amino)-2-(trifluoromethyl)benzonitrile

To 3,3,3-trifluoropropanoic acid (0.04 g, 0.309 mmol) in DCE (3 mL) was added EDCl (0.062 g, 0.323 mmol) followed by 2-[[4-cyano-3-(trifluoromethyl)phenyl](2,2,2-trifluoroethyl)amino]-N-hydroxyethanimidamide, Example 129E, (0.1 g, 0.294 mmol). The resulting mixture was stirred at rt for 30 min and then at 80° C. for another 14 h. Upon cooling, the reaction was partitioned between EtOAc/H₂O. The organic portion was washed with brine, dried over Na₂SO₄, filtered and concentrated in vacuo. Purification over SiO₂ eluting with EtOAc/hexanes afforded the title compound (0.049 g): MS (ESI) m/z 431 (M−1).

### Example 175

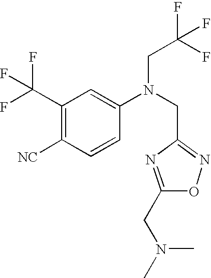

### 4-[({5-[(Dimethylamino)methyl]-1,2,4-oxadiazol-3-yl}methyl)(2,2,2-trifluoroethyl)amino]-2-(trifluoromethyl)benzonitrile

Synthesized as described in Example 174 from N,N-dimethylglycine: MS (ESI) m/z 408 (M+1).

### Example 176

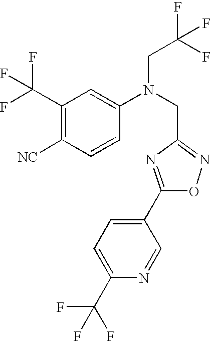

### 4-[(2,2,2-Trifluoroethyl)({5-[6-(trifluoromethyl)-3-pyridinyl]-1,2,4-oxadiazol-3-yl}methyl)amino]-2-(trifluoromethyl)benzonitrile

Synthesized as described in Example 174 from 6-(trifluoromethyl)-3-pyridinecarboxylic acid: MS (ESI) m/z 494 (M−1).

### Example 177

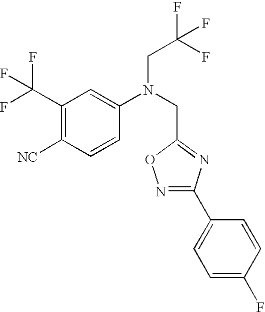

### 4-[{[3-(4-Fluorophenyl)-1,2,4-oxadiazol-5-yl]methyl}(2,2,2-trifluoroethyl)amino]-2-(trifluoromethyl)benzonitrile

To a suspension of N-[4-cyano-3-(trifluoromethyl)phenyl]-N-(2,2,2-trifluoroethyl)glycine, Example 129B (0.050 g, 0.153 mmol) in CH₂Cl₂ (1.5 mL) was added EDCl (0.033 g, 0.169 mmol). After 5 min, 4-fluorobenzamidoxime (0.025 g, 0.161 mmol) was added and stirred at rt for 45 min. The mixture was concentrated and toluene (2 mL) and pyridine (0.5 mL) were added to the residue. The heterogeneous mixture was heated at 100° C. for 2 h. Upon cooling, the mixture was partitioned between EtOAc and 0.5N HCl. The organic phase was washed twice with 0.5N HCl, once with brine, dried (Na₂SO₄) and concentrated in vacuo. The residue was purified by silica gel chromatography (0-40% EtOAc-hexanes gradient), followed by a second chromatography (10-100% CH₂Cl₂-hexanes gradient) to give the title compound (0.053 g, 78% yield): MS (ES) m/z 445 (M+1).

### Example 178

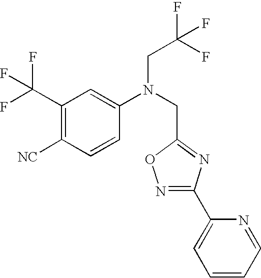

### 4-[{[3-(2-Pyridinyl)-1,2,4-oxadiazol-5-yl]methyl}(2,2,2-trifluoroethyl)amino]-2-(trifluoromethyl)benzonitrile

Synthesized as described in Example 177 using pyridine-2-amidoxime: MS (ES) m/z 428 (M+1).

### Example 179

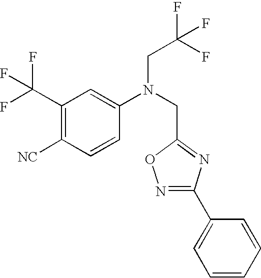

### 4-[[(3-Phenyl-1,2,4-oxadiazol-5-yl)methyl](2,2,2-trifluoroethyl)amino]-2-(trifluoromethyl)benzonitrile

To a suspension of N-[4-cyano-3-(trifluoromethyl)phenyl]-N-(2,2,2-trifluoroethyl)glycine, Example 129B (0.050 g, 0.153 mmol) in DCE (3 mL) was added EDCl (0.033 g, 0.168 mmol). After stirring at rt for 5 min, benzamidoxime (0.022 g, 0.161 mmol) was added. The mixture was stirred at rt for 45 min and then heated in a microwave at 120° C. for 20 min. Upon cooling, the mixture was partitioned between EtOAc and 0.5N HCl. The organic phase was washed with 0.5N HCl and sat'd brine, dried over Na₂SO₄ and concentrated in vacuo. The residue was purified by silica gel chromatography (0-50% EtOAc-hexanes gradient), followed by a second chromatography (60-100% CH₂Cl₂-hexanes gradient) and crystallization from CH₂Cl₂-hexanes to give the title compound as a white solid (0.044 g, 68% yield): MS (ES) m/z 425 (M−1).

### Example 180

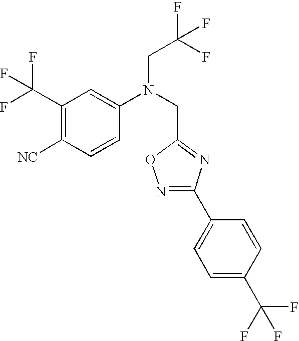

### 4-[(2,2,2-Trifluoroethyl)({3-[4-(trifluoromethyl)phenyl]-1,2,4-oxadiazol-5-yl}methyl)amino]-2-(trifluoromethyl)benzonitrile

Synthesized in a manner similar to Example 179 using N-hydroxy-4-(trifluoromethyl)benzenecarboximidamide: MS (ES) m/z 493 (M−1).

### Example 181

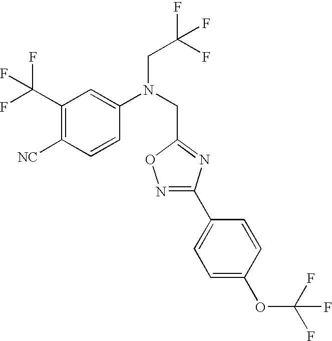

### 4-{(2,2,2-Trifluoroethyl)[(3-{4-[(trifluoromethyl)oxy]phenyl}-1,2,4-oxadiazol-5-yl)methyl]amino}-2-(trifluoromethyl)benzonitrile

Synthesized in a manner similar to Example 179 using N-hydroxy-4-[(trifluoromethyl)oxy]benzenecarboximidamide: MS (ES) m/z 509 (M−1).

### Example 182

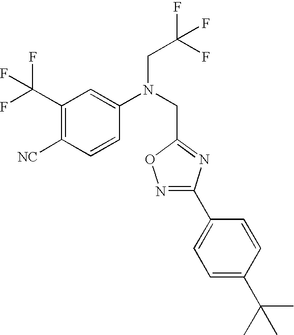

### 4-[({3-[4-(1,1-Dimethylethyl)phenyl]-1,2,4-oxadiazol-5-yl}methyl)(2,2,2-trifluoroethyl)amino]-2-(trifluoromethyl)benzonitrile

Synthesized in a manner similar to Example 10B using 5-(chloromethyl)-3-[4-(1,1-dimethylethyl)phenyl]-1,2,4-oxadiazole: ¹H NMR (400 MHz, CDCl₃) δ 7.94 (d, J=8.4 Hz, 2H), 7.69 (d, J=8.8 Hz, 1H), 7.49 (d, J=8.4 Hz, 2H), 7.25 (s, 1H, overlaps with CHCl₃ signal), 7.05 (dd, J=8.8, 2.8 Hz, 1H), 4.95 (s, 2H), 4.23 (q, J=8.6 Hz, 2H), 1.34 (s, 9H).

### Example 183

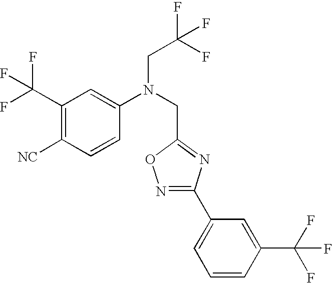

### 4-[(2,2,2-Trifluoroethyl)({3-[3-(trifluoromethyl)phenyl]-1,2,4-oxadiazol-5-yl}methyl)amino]-2-(trifluoromethyl)benzonitrile

Synthesized in a manner similar to Example 179 using N-hydroxy-3-(trifluoromethyl)benzenecarboximidamide: MS (ES) m/z 493 (M−1).

### Example 184

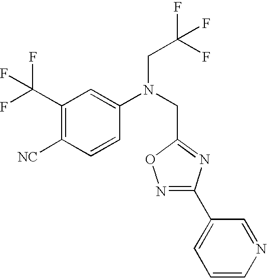

### 4-[{[3-(3-Pyridinyl)-1,2,4-oxadiazol-5-yl]methyl}(2,2,2-trifluoroethyl)amino]-2-(trifluoromethyl)benzonitrile

Synthesized in a manner similar to Example 179 using N-hydroxy-3-pyridinecarboximidamide: MS (ES) m/z 428 (M+1).

### Example 185

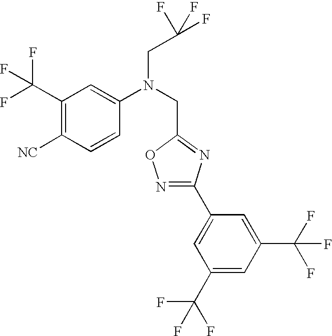

### 4-[({3-[3,5-Bis(trifluoromethyl)phenyl]-1,2,4-oxadiazol-5-yl}methyl)(2,2,2-trifluoroethyl)amino]-2-(trifluoromethyl)benzonitrile

Synthesized as described in Example 177 using 3,5-bis(trifluoromethyl)-benzamidoxime: MS (ES) m/z 561 (M−1).

### Example 186

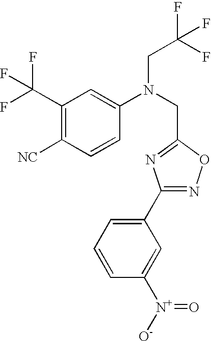

### 4-[{[3-(3-Nitrophenyl)-1,2,4-oxadiazol-5-yl]methyl}(2,2,2-trifluoroethyl)amino]-2-(trifluoromethyl)benzonitrile

To N-[4-cyano-3-(trifluoromethyl)phenyl]-N-(2,2,2-trifluoroethyl)glycine (Example 129B) (0.075 g, 0.231 mmol) in DCE (3 mL) was added EDCl (0.049 g, 0.254 mmol) followed by N-hydroxy-3-nitrobenzenecarboximidamide (0.044 g, 0.243 mmol). The reaction mixture was stirred at rt for 30 min followed by 80° C. for 14 h. The organic portion was washed with brine, dried over Na₂SO₄, filtered and concentrated in vacuo. Purification over SiO₂ eluting with EtOAc/hexanes afforded the title compound (0.047 g): MS (ESI) m/z 470 (M−1).

### Example 187

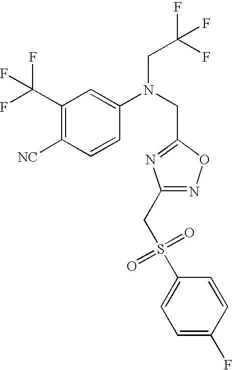

### 4-[[(3-{[(4-Fluorophenyl)sulfonyl]methyl}-1,2,4-oxadiazol-5-yl)methyl](2,2,2-trifluoroethyl)amino]-2-(trifluoromethyl)benzonitrile

Synthesized as described in Example 186 from N-[4-cyano-3-(trifluoromethyl)phenyl]-N-(2,2,2-trifluoroethyl)glycine (Example 129B) and 2-[(4-fluorophenyl)sulfonyl]-N-hydroxyethanimidamide: MS (ESI) m/z 523 (M+1).

### Example 188

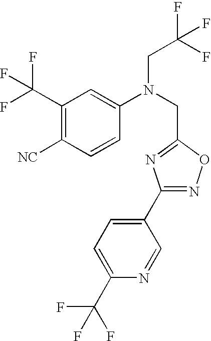

### 4-[(2,2,2-Trifluoroethyl)({3-[6-(trifluoromethyl)-3-pyridinyl]-1,2,4-oxadiazol-5-yl}methyl)amino]-2-(trifluoromethyl)benzonitrile

Synthesized as described in Example 186 from N-[4-cyano-3-(trifluoromethyl)phenyl]-N-(2,2,2-trifluoroethyl)glycine (Example 129B) and N-hydroxy-6-(trifluoromethyl)-3-pyridinecarboximidamide: MS (ESI) m/z 496 (M+1).

### Example 189

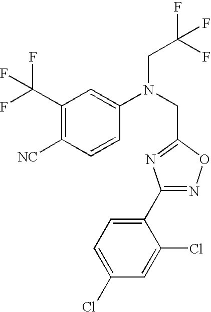

### 4-[{[3-(2,4-Dichlorophenyl)-1,2,4-oxadiazol-5-yl]methyl}(2,2,2-trifluoroethyl)amino]-2-(trifluoromethyl)benzonitrile

Synthesized as described in Example 186 from N-[4-cyano-3-(trifluoromethyl)phenyl]-N-(2,2,2-trifluoroethyl)glycine (Example 129B) and 2,4-dichloro-N-hydroxybenzenecarboximidamide: MS (ESI) m/z 495 (M+1).

### Example 190

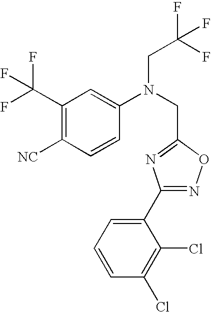

### 4-[{[3-(2,3-Dichlorophenyl)-1,2,4-oxadiazol-5-yl]methyl}(2,2,2-trifluoroethyl)amino]-2-(trifluoromethyl)benzonitrile

Synthesized as described in Example 186 from N-[4-cyano-3-(trifluoromethyl)phenyl]-N-(2,2,2-trifluoroethyl)glycine (Example 129B) and 2,3-dichloro-N-hydroxybenzenecarboximidamide: MS (ESI) m/z 495 (M+1).

### Example 191

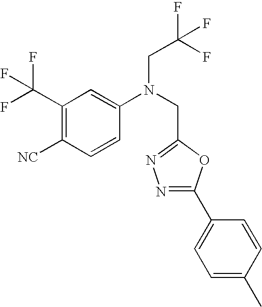

### 4-[{[5-(4-Methylphenyl)-1,3,4-oxadiazol-2-yl]methyl}(2,2,2-trifluoroethyl)amino]-2-(trifluoromethyl)benzonitrile

Synthesized as described in Example 10B using 2-(chloromethyl)-5-(4-methylphenyl)-1,3,4-oxadiazole: MS (ES) m/z 441 (M+1).

### Example 192

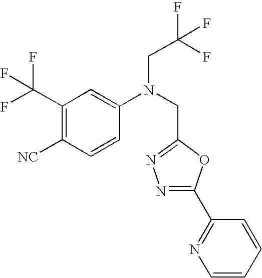

### 4-[{[5-(2-Pyridinyl)-1,3,4-oxadiazol-2-yl]methyl}(2,2,2-trifluoroethyl)amino]-2-(trifluoromethyl)benzonitrile

To a solution of N-[4-cyano-3-(trifluoromethyl)phenyl]-N-(2,2,2-trifluoroethyl)glycine, Example 129B, (0.050 g, 0.308 mmol) in a microwave vial in acetonitrile (3 mL) was added EDCl (0.032 g, 0.339 mmol) followed by 2-pyridinecarbohydrazide (0.023 g, 0.323 mmol). The resulting mixture was stirred for 30 min to form the N′-{2-[[4-cyano-3-(trifluoromethyl)phenyl](2,2,2-trifluoroethyl)amino]acetyl}-2-pyridinecarbohydrazide intermediate. To this solution was added THF (3 mL) followed by tosyl chloride (0.37 mmol) and P-BEMP (1.54 mmol, 2.2 mmol/g resin load). The sealed vial was heated to 100° C. in a microwave for 5 min. The cooled reaction mixture was filtered, washed twice with CH₂Cl₂, and concentrated via rotary evaporator. Purification by silica gel chromatography (eluting with 25% EtOAc/hexanes) provided the title compound (0.019 g, 45%): MS (ES) m/z 428 (M+1).

### Example 193

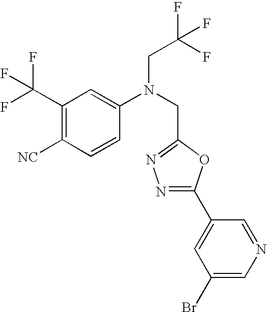

### 4-[{[5-(5-Bromo-3-pyridinyl)-1,3,4-oxadiazol-2-yl]methyl}(2,2,2-trifluoroethyl)amino]-2-(trifluoromethyl)benzonitrile

Synthesized as described for Example 192 using 5-bromo-3-pyridinecarbohydrazide: MS (ES) m/z 506 and 508, (M+1 isotopes).

### Example 194

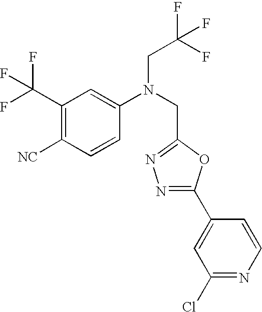

### 4-[{[5-(2-Chloro-4-pyridinyl)-1,3,4-oxadiazol-2-yl]methyl}(2,2,2-trifluoroethyl)amino]-2-(trifluoromethyl)benzonitrile

Synthesized as described for Example 192 using 2-chloro-4-pyridinecarbohydrazide: MS (ES) m/z 462 (M+1).

### Example 195

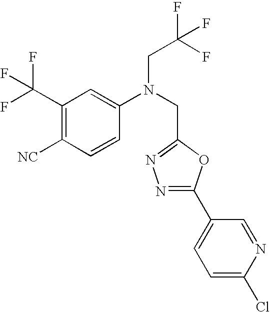

### 4-[{[5-(6-Chloro-3-pyridinyl)-1,3,4-oxadiazol-2-yl]methyl}(2,2,2-trifluoroethyl)amino]-2-(trifluoromethyl)benzonitrile

Synthesized as described for Example 192 using 6-chloro-3-pyridinecarbohydrazide: MS (ES) m/z 462 (M+1).

### Example 196

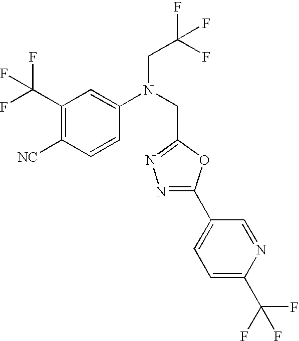

### 4-[(2,2,2-Trifluoroethyl)({5-[6-(trifluoromethyl)-3-pyridinyl]-1,3,4-oxadiazol-2-yl}methyl)amino]-2-(trifluoromethyl)benzonitrile

Synthesized as described for Example 192 using 6-(trifluoromethyl)-3-pyridinecarbohydrazide: MS (ES) m/z 496 (M+1).

### Example 197

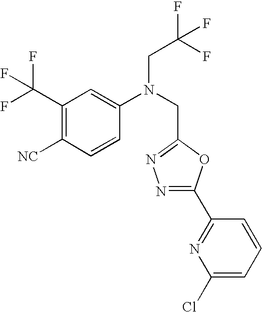

### 4-[{[5-(6-Chloro-2-pyridinyl)-1,3,4-oxadiazol-2-yl]methyl}(2,2,2-trifluoroethyl)amino]-2-(trifluoromethyl)benzonitrile

Synthesized as described for Example 192 using 6-chloro-2-pyridinecarbohydrazide: MS (ES) m/z 462 (M+1).

### Example 198

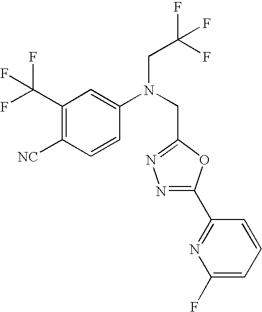

### 4-[{[5-(6-Fluoro-2-pyridinyl)-1,3,4-oxadiazol-2-yl]methyl}(2,2,2-trifluoroethyl)amino]-2-(trifluoromethyl)benzonitrile

Synthesized as described for Example 192 using 6-fluoro-2-pyridinecarbohydrazide: MS (ES) m/z 446 (M+1).

### Example 199

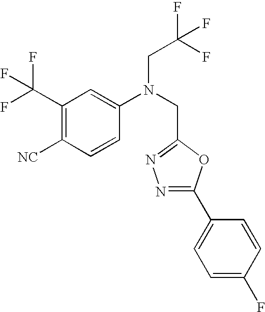

### 4-[{[5-(4-Fluorophenyl)-1,3,4-oxadiazol-2-yl]methyl}(2,2,2-trifluoroethyl)amino]-2-(trifluoromethyl)benzonitrile

Synthesized as described for Example 192 using 4-fluorophenylcarbohydrazide: MS (ES) m/z 445 (M+1).

### Example 200

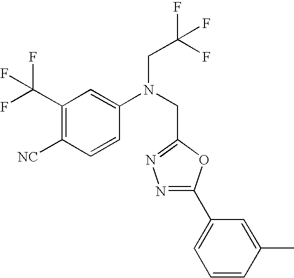

### 4-[{[5-(3-Methylphenyl)-1,3,4-oxadiazol-2-yl]methyl}(2,2,2-trifluoroethyl)amino]-2-(trifluoromethyl)benzonitrile

To N-[4-cyano-3-(trifluoromethyl)phenyl]-N-(2,2,2-trifluoroethyl)glycine, Example 129B, (0.05 g, 0.1 mmol) in a microwave vial, was added CH₃CN (3 mL) followed by EDCl (0.032 g, 0.169 mmol) and the resulting mixture was stirred at rt for 3 min. 3-Methylbenzohydrazide (0.024 g, 0.162 mmol) was added next and the mixture was stirred for 30 min to form the N′-{[[4-cyano-3-(trifluoromethyl)phenyl](2,2,2-trifluoroethyl)amino]acetyl}-3-methylbenzohydrazide intermediate. To this solution, was added THF (3 mL) followed by tosyl chloride (0.035 g, 0.185 mmol) and P-BEMP (0.35 g, 0.77 mmol, 2.2 mmol/g resin load). The reaction vial was sealed and heated to 100° C. in a microwave for 5 min. The resin was filtered off, washed twice with CH₂Cl₂ and concentrated via rotary evaporator. Purification via silica gel eluting with 25% EtOAc/hexanes afforded 0.018 g of pure product: MS (ESI) m/z 441 (M+1).

### Example 201

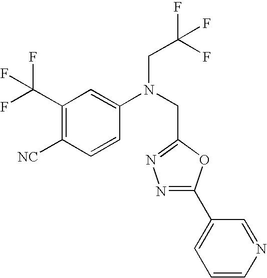

### 4-[{[5-(3-Pyridinyl)-1,3,4-oxadiazol-2-yl]methyl}(2,2,2-trifluoroethyl)amino]-2-(trifluoromethyl)benzonitrile

Synthesized as described in Example 200 from 3-pyridinecarbohydrazide: MS (ESI) m/z 428 (M+1).

### Example 202

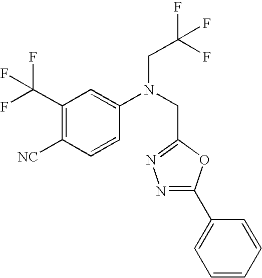

### 4-[[(5-Phenyl-1,3,4-oxadiazol-2-yl)methyl](2,2,2-trifluoroethyl)amino]-2-(trifluoromethyl)benzonitrile

Synthesized in a manner similar to Example 192 using benzohydrazide: MS (ES) m/z 427 (M+1).

### Example 203

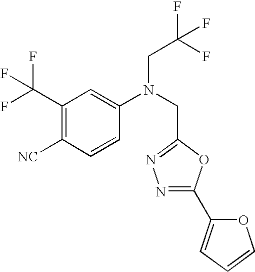

### 4-[{[5-(2-Furanyl)-1,3,4-oxadiazol-2-yl]methyl}(2,2,2-trifluoroethyl)amino]-2-(trifluoromethyl)benzonitrile

Synthesized in a manner similar to Example 192 using 2-furancarbohydrazide: MS (ES) m/z 417 (M+1).

### Example 204

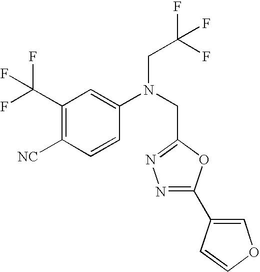

### 4-[{[5-(3-Furanyl)-1,3,4-oxadiazol-2-yl]methyl}(2,2,2-trifluoroethyl)amino]-2-(trifluoromethyl)benzonitrile

Synthesized in a manner similar to Example 192 using 3-furancarbohydrazide: MS (ES) m/z 417 (M+1).

### Example 205

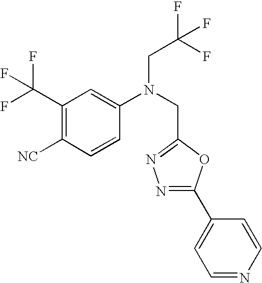

### 4-[{[5-(4-Pyridinyl)-1,3,4-oxadiazol-2-yl]methyl}(2,2,2-trifluoroethyl)amino]-2-(trifluoromethyl)benzonitrile

Synthesized as described in Example 200 from 4-pyridinecarbohydrazide: MS (ESI) m/z 428 (M+1).

### Example 206

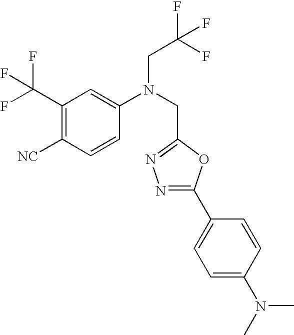

### 4-[({5-[4-(Dimethylamino)phenyl]-1,3,4-oxadiazol-2-yl}methyl)(2,2,2-trifluoroethyl)amino]-2-(trifluoromethyl)benzonitrile

Synthesized as described in Example 200 from 4-(dimethylamino)benzohydrazide: MS (ESI) m/z 470 (M+1).

### Example 207

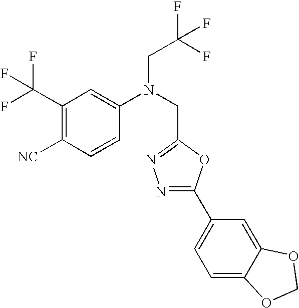

### 4-[{[5-(1,3-Benzodioxol-5-yl)-1,3,4-oxadiazol-2-yl]methyl}(2,2,2-trifluoroethyl)amino]-2-(trifluoromethyl)benzonitrile

Synthesized as described in Example 200 from 3,4-bis(methyloxy)benzohydrazide: MS (ESI) m/z 471 (M+1).

### Example 208

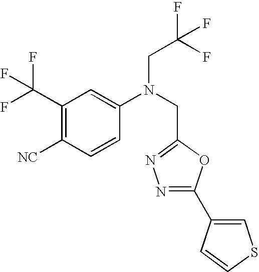

### 4-[{[5-(3-Thienyl)-1,3,4-oxadiazol-2-yl]methyl}(2,2,2-trifluoroethyl)amino]-2-(trifluoromethyl)benzonitrile

Synthesized as described in Example 200 from 3-thiophenecarbohydrazide: MS (ESI) m/z 433 (M+1).

### Example 209

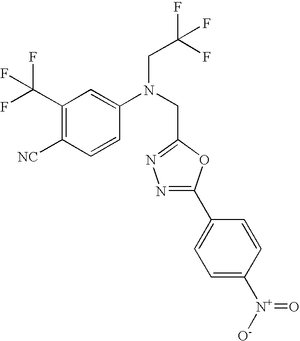

### 4-[{[5-(4-Nitrophenyl)-1,3,4-oxadiazol-2-yl]methyl}(2,2,2-trifluoroethyl)amino]-2-(trifluoromethyl)benzonitrile

Synthesized as described in Example 200 from 4-nitrobenzohydrazide: MS (APCI) m/z 472 (M+1).

### Example 210

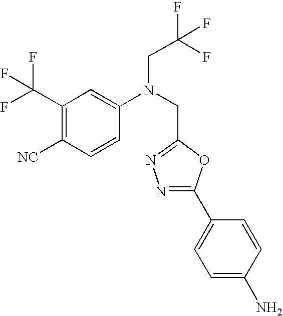

### 4-{2-[5-(4-Aminophenyl)-1,3,4-oxadiazol-2-yl]-1-[(trifluoromethyl)amino]ethyl}-2-(trifluoromethyl)benzonitrile

4-[{[5-(4-Nitrophenyl)-1,3,4-oxadiazol-2-yl]methyl}(2,2,2-trifluoroethyl)amino]-2-(trifluoromethyl)benzonitrile, Example 209, (0.083 g, 0.18 mmol) in EtOAc (5 mL) was degassed with N₂. 20% Pd(OH)₂/C (0.013 g, 0.018 mmol) was added and the reaction flask was fitted with an H₂ filled balloon. The reaction was stirred at ambient temperature for 14 h. The mixture was filtered through celite and the filtrate was concentrated in vacuo to obtain 0.057 g of product: MS (APCI) m/z 442 (M+1).

### Example 211

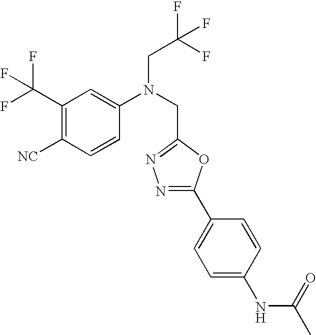

### N-[4-(5-{2-[4-Cyano-3-(trifluoromethyl)phenyl]-2-[(trifluoromethyl)amino]ethyl}-1,3,4-oxadiazol-2-yl)phenyl]acetamide

To 4-{2-[5-(4-aminophenyl)-1,3,4-oxadiazol-2-yl]-1-[(trifluoromethyl)amino]ethyl}-2-(trifluoromethyl)benzonitrile, Example 210, (0.047 g, 0.106 mmol) in THF (3 mL) was added LiHDMS (IM THF, 0.117 ml, 0.117 mmol) and acetyl chloride (0.0083 ml, 0.117 mmol). The reaction was stirred at rt for 1 h. Partitioning between EtOAc/H₂O, washing the organic layer with brine, drying over Na₂SO₄, and filtration was followed by concentration in vacuo to obtain 0.034 g of the title compound: MS (ESI) m/z 484 (M+1).

### Example 212

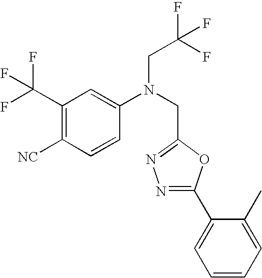

### 4-[{[5-(2-Methylphenyl)-1,3,4-oxadiazol-2-yl]methyl}(2,2,2-trifluoroethyl)amino]-2-(trifluoromethyl)benzonitrile

Synthesized as described in Example 200 from 2-methylbenzohydrazide: MS (ESI) m/z 441 (M+1).

### Example 213

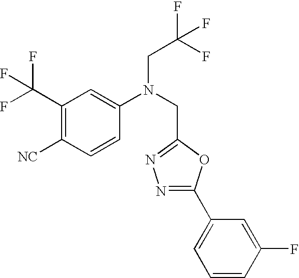

### 4-[{[5-(3-Fluorophenyl)-1,3,4-oxadiazol-2-yl]methyl}(2,2,2-trifluoroethyl)amino]-2-(trifluoromethyl)benzonitrile

Synthesized as described in Example 200 from 3-fluorobenzohydrazide: MS (ESI) m/z 445 (M+1).

### Example 214

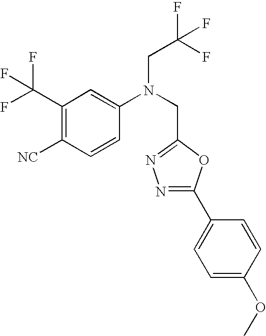

### 4-[({5-[4-(Methyloxy)phenyl]-1,3,4-oxadiazol-2-yl}methyl)(2,2,2-trifluoroethyl)amino]-2-(trifluoromethyl)benzonitrile

Synthesized as described in Example 200 from 4-(methyloxy)benzohydrazide: MS (APCI) m/z 457 (M+1).

### Example 215

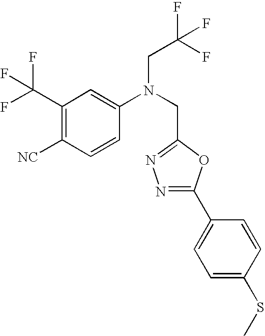

### 4-[({5-[4-(Methylthio)phenyl]-1,3,4-oxadiazol-2-yl}methyl)(2,2,2-trifluoroethyl)amino]-2-(trifluoromethyl)benzonitrile

Synthesized as described in Example 200 from 4-(methylthio)benzohydrazide: MS (APCI) m/z 473 (M+1).

### Example 216

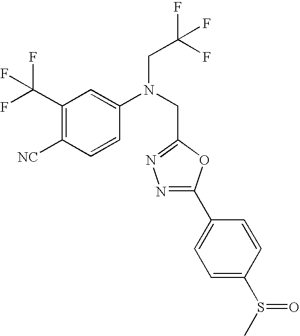

### 4-{2-{5-[4-(Methylsulfinyl)phenyl]-1,3,4-oxadiazol-2-yl}-1-[(trifluoromethyl)amino]ethyl}-2-(trifluoromethyl)benzonitrile

To a cooled solution (ice bath) of 4-[({5-[4-(methylthio)phenyl]-1,3,4-oxadiazol-2-yl}methyl)(2,2,2-trifluoroethyl)amino]-2-(trifluoromethyl)benzonitrile, Example 215, (0.028 g, 0.0593 mmol) in CH₂Cl₂ (3 mL) was add MCPBA (0.015 g, 0.0623 mmol). Stirring was continued for 30 min at 0° C. The mixture was quenched with aqueous Na₂CO₃ and extracted with EtOAc. The organic portion was washed with brine, dried over Na₂SO₄, filtered, and concentrated in vacuo at 25° C. Purification across silica gel eluting with EtOAc/hexanes afforded the title compound (0.0226 g): MS (ESI) m/z 489 (M+1).

### Example 217

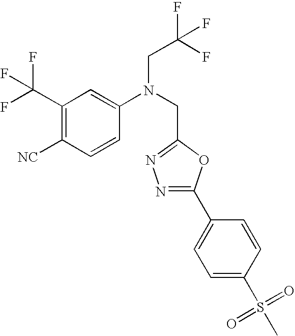

### 4-{2-{5-[4-(Methylsulfonyl)phenyl]-1,3,4-oxadiazol-2-yl}-1-[(trifluoromethyl)amino]ethyl}-2-(trifluoromethyl)benzonitrile

Synthesized as described in Example 216 from 4-[({5-[4-(methylthio)phenyl]-1,3,4-oxadiazol-2-yl}methyl)(2,2,2-trifluoroethyl)amino]-2-(trifluoromethyl)benzonitrile, Example 216, except 2 equiv. of MCPBA were used and the reaction was stirred at rt for 60 min: MS (ESI) m/z 505 (M+1).

### Example 218

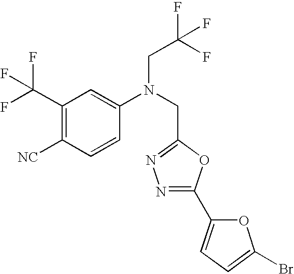

### 4-[{[5-(5-Bromo-2-furanyl)-1,3,4-oxadiazol-2-yl]methyl}(2,2,2-trifluoroethyl)amino]-2-(trifluoromethyl)benzonitrile

Synthesized as described in Example 200 from 5-bromo-2-furancarbohydrazide: MS (APCI) m/z 495 (M+1).

### Example 219

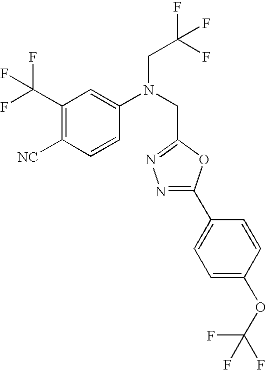

### 4-{(2,2,2-Trifluoroethyl)[(5-{4-[(trifluoromethyl)oxy]phenyl}-1,3,4-oxadiazol-2-yl)methyl]amino}-2-(trifluoromethyl)benzonitrile

Synthesized as described in Example 200 from 4-[(trifluoromethyl)oxy]benzohydrazide: MS (APCI) m/z 511 (M+1).

### Example 220

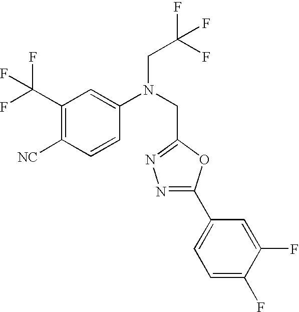

### 4-[{[5-(3,4-Difluorophenyl)-1,3,4-oxadiazol-2-yl]methyl}(2,2,2-trifluoroethyl)amino]-2-(trifluoromethyl)benzonitrile

Synthesized as described in Example 200 from 3,4-difluorobenzohydrazide: MS (ESI) m/z 463 (M+1).

### Example 221

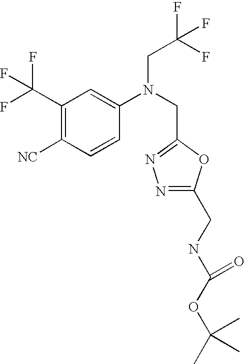

### 1,1-Dimethylethyl[(5-{[[4-cyano-3-(trifluoromethyl)phenyl](2,2,2-trifluoroethyl)amino]methyl}-1,3,4-oxadiazol-2-yl)methyl]carbamate

Synthesized as described in Example 200 from 1,1-dimethylethyl (2-hydrazino-2-oxoethyl)carbamate: MS (ESI) m/z 478 (M−1).

### Example 222

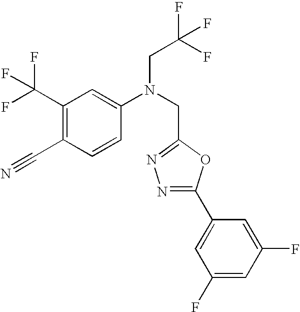

### 4-[{[5-(3,5-Difluorophenyl)-1,3,4-oxadiazol-2-yl]methyl}(2,2,2-trifluoroethyl)amino]-2-(trifluoromethyl)benzonitrile

Synthesized as described in Example 200 from 3,5-difluorobenzohydrazide: MS (ESI) m/z 463 (M+1).

### Example 223

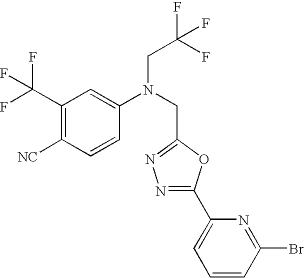

### 4-[{[5-(6-Bromo-2-pyridinyl)-1,3,4-oxadiazol-2-yl]methyl}(2,2,2-trifluoroethyl)amino]-2-(trifluoromethyl)benzonitrile

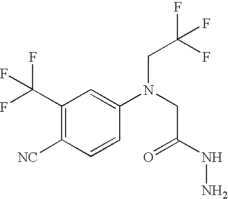

### A. 2-[[4-Cyano-3-(trifluoromethyl)phenyl](2,2,2-trifluoroethyl)amino]acetohydrazide

To a solution of N-[4-cyano-3-(trifluoromethyl)phenyl]-N-(2,2,2-trifluoroethyl)glycine, Example 129B (2 g, 6.154 mmol) in THF (35 mL) was added CDI (1.05 g, 6.462 mmol) at rt in portions. After stirring for 30 min at rt, anhydrous hydrazine (1.93 mL, 61.54 mmol) was added and the reaction was stirred at rt for 60 min. The concentrated mixture was slurried in H₂O, filtered, washed with H₂O, washed with hexanes, and dried under vacuum to afford the title compound (1.82 g): MS (APCI) m/z 341 (M+1).

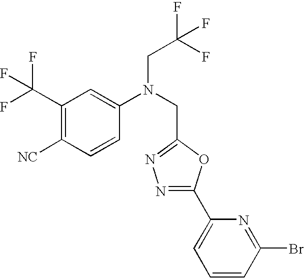

### B. 4-[{[5-(6-Bromo-2-pyridinyl)-1,3,4-oxadiazol-2-yl]methyl}(2,2,2-trifluoroethyl)amino]-2-(trifluoromethyl)benzonitrile

Synthesized as described in Example 200 from 2-[[4-Cyano-3-(trifluoromethyl)phenyl](2,2,2-trifluoroethyl)amino]acetohydrazide (Example 228A) and 6-bromo-2-pyridinecarboxylic acid: MS (APCI) m/z 506 and 508, M+1 isotopes.

### Example 224

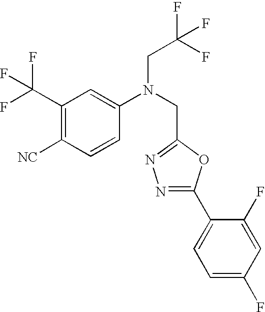

### 4-[{[5-(2,4-Difluorophenyl)-1,3,4-oxadiazol-2-yl]methyl}(2,2,2-trifluoroethyl)amino]-2-(trifluoromethyl)benzonitrile

Synthesized as described in Example 223B from 2,4-difluorobenzoic acid: MS (ESI) m/z 463 (M+1).

### Example 225

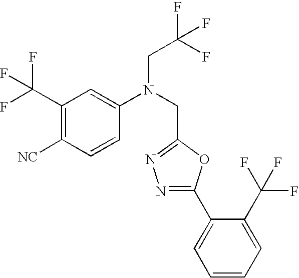

### 4-[(2,2,2-Trifluoroethyl)({5-[2-(trifluoromethyl)phenyl]-1,3,4-oxadiazol-2-yl}methyl)amino]-2-(trifluoromethyl)benzonitrile

Synthesized as described in Example 223B from 2-(trifluoromethyl)benzoic acid: MS (APCI) m/z 495 (M+1).

### Example 226

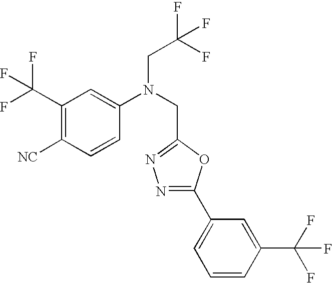

### 4-[(2,2,2-Trifluoroethyl)({5-[3-(trifluoromethyl)phenyl]-1,3,4-oxadiazol-2-yl}methyl)amino]-2-(trifluoromethyl)benzonitrile

Synthesized as described in Example 223B from 2 3-(trifluoromethyl)benzoic acid: MS (APCI) m/z 495 (M+1).

### Example 227

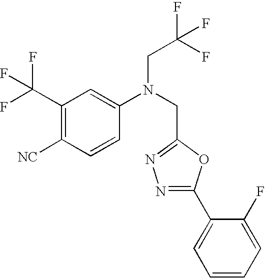

### 4-[{[5-(2-Fluorophenyl)-1,3,4-oxadiazol-2-yl]methyl}(2,2,2-trifluoroethyl)amino]-2-(trifluoromethyl)benzonitrile

Synthesized as described in Example 223B from 2-fluorobenzoic acid: MS (APCI) m/z 445 (M+1).

### Example 228

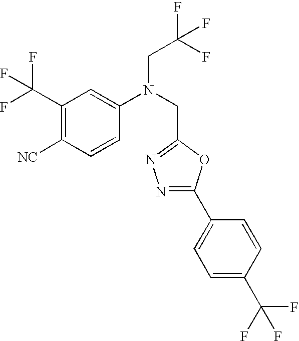

### 4-[(2,2,2-Trifluoroethyl)({5-[4-(trifluoromethyl)phenyl]-1,3,4-oxadiazol-2-yl}methyl)amino]-2-(trifluoromethyl)benzonitrile

Synthesized as described in Example 223B from 4-(trifluoromethyl)benzoic acid: MS (APCI) m/z 495 (M+1).

### Example 229

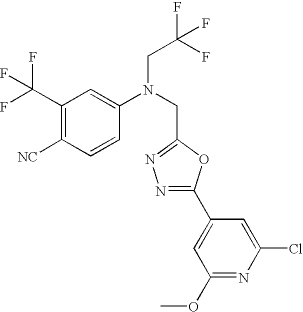

### 4-[({5-[2-Chloro-6-(methyloxy)-4-pyridinyl]-1,3,4-oxadiazol-2-yl}methyl)(2,2,2-trifluoroethyl)amino]-2-(trifluoromethyl)benzonitrile

Synthesized as described in Example 223B from 2-chloro-6-(methyloxy)-4-pyridinecarboxylic acid: MS (APCI) m/z 492 (M+1).

### Example 230

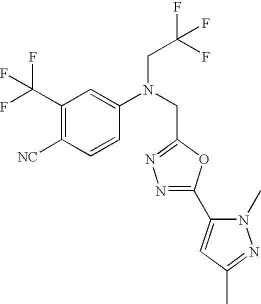

### 4-[{[5-(1,3-Dimethyl-1H-pyrazol-5-yl)-1,3,4-oxadiazol-2-yl]methyl}(2,2,2-trifluoroethyl)amino]-2-(trifluoromethyl)benzonitrile

Synthesized as described in Example 223B from 1,3-dimethyl-1H-pyrazole-5-carboxylic acid: MS (APCI) m/z 445 (M+1).

### Example 231

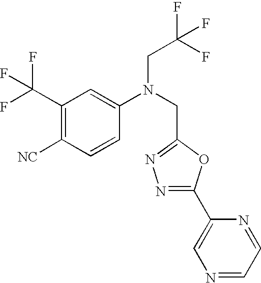

### 4-[{[5-(2-Pyrazinyl)-1,3,4-oxadiazol-2-yl]methyl}(2,2,2-trifluoroethyl)amino]-2-(trifluoromethyl)benzonitrile

Synthesized as described in Example 223B from 2-pyrazinecarboxylic acid: MS (APCI) m/z 429 (M+1).

### Example 232

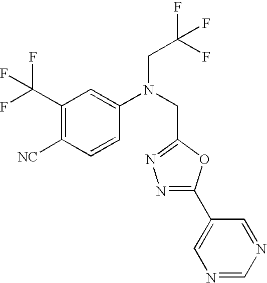

### 4-[{[5-(5-Pyrimidinyl)-1,3,4-oxadiazol-2-yl]methyl}(2,2,2-trifluoroethyl)amino]-2-(trifluoromethyl)benzonitrile

Synthesized as described in Example 223B from 5-pyrimidinecarboxylic acid: MS (ESI) m/z 429 (M+1).

### Example 233

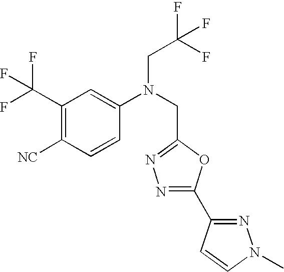

### 4-[{[5-(1-Methyl-1H-pyrazol-3-yl)-1,3,4-oxadiazol-2-yl]methyl}(2,2,2-trifluoroethyl)amino]-2-(trifluoromethyl)benzonitrile

Synthesized as described in Example 223B from 1-methyl-1H-pyrazole-3-carboxylic acid: MS (ESI) m/z 431 (M+1).

### Example 234

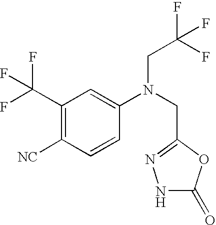

### 4-{2-(5-Oxo-4,5-dihydro-1,3,4-oxadiazol-2-yl)-1-[(trifluoromethyl)amino]ethyl}-2-(trifluoromethyl)benzonitrile

To 2-[[4-cyano-3-(trifluoromethyl)phenyl](2,2,2-trifluoroethyl)amino]acetohydrazide, Example 223A, (0.05 g, 0.147 mmol) in dry THF (2 mL) was added TEA (0.022 ml, 0.162 mmol) followed by CDI (0.031 g, 0.191 mmol) portion-wise. The reaction was stirred at rt for 2 h and then partitioned between EtOAc/1 N HCl. The organic layer was washed with brine, dried over Na₂SO₄, filtered, and concentrated in vacuo. Purification over SiO₂ eluting with EtOAc/hexanes afforded the title compound (0.025 g): MS (ESI) m/z 365 (M−1).

### Example 235

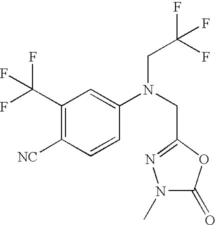

### 4-{2-(4-Methyl-5-oxo-4,5-dihydro-1,3,4-oxadiazol-2-yl)-1-[(trifluoromethyl)amino]ethyl}-2-(trifluoromethyl)benzonitrile

To 4-{2-(5-oxo-4,5-dihydro-1,3,4-oxadiazol-2-yl)-1-[(trifluoromethyl)amino]ethyl}-2-(trifluoromethyl)benzonitrile, Example 234, (0.05 g, 0.137 mmol) in DMF (2 mL) was added MeI (0.01 ml, 0.164 mmol). The reaction was cooled to ca. 0° C. and NaH (60% dispersion in oil, 0.0066 g, 0.164 mmol) was added. After stirring for 30 min at 0° C., the reaction was partitioned between EtOAc/H₂O. The organic portion was washed with brine, dried over Na₂SO₄, filtered, and concentrated in vacuo. Purification over SiO₂ eluting with EtOAc/hexanes afforded the title compound (0.023 g): MS (APCI) m/z 379 (M−1).

### Example 236

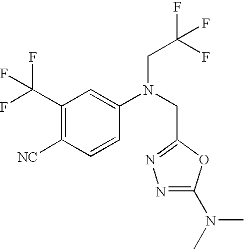

### 4-{2-[5-(Dimethylamino)-1,3,4-oxadiazol-2-yl]-1-[(trifluoromethyl)amino]ethyl}-2-(trifluoromethyl)benzonitrile

To 4-{2-(5-oxo-4,5-dihydro-1,3,4-oxadiazol-2-yl)-1-[(trifluoromethyl)amino]ethyl}-2-(trifluoromethyl)benzonitrile, Example 234, (0.025 g, 0.0683 mmol) was added POCl₃ (1.5 mL) and the reaction was heated to 100° C. for 10 min in microwave. Dimethyl amine (1 N THF, 1.5 mL) was added to the cooled mixture. Heating was continued at 150° C. for another 10 min in a microwave. The excess POCl₃ was removed in vacuo. The residue was partitioned between EtOAc/0.1 N NaOH and the organic portion was washed with brine, dried over Na₂SO₄, filtered and concentrated in vacuo. Purification over SiO₂ eluting with EtOAc/hexanes afforded the title compound (0.017 g): MS (APCI) m/z 394 (M+1).

### Example 237

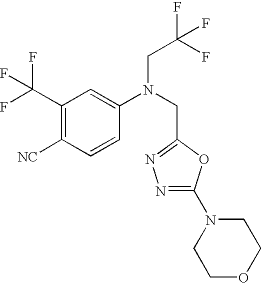

### 4-{2-[5-(4-Morpholinyl)-1,3,4-oxadiazol-2-yl]-1-[(trifluoromethyl)amino]ethyl}-2-(trifluoromethyl)benzonitrile

Synthesized as described in Example 236 from 4-{2-(5-oxo-4,5-dihydro-1,3,4-oxadiazol-2-yl)-1-[(trifluoromethyl)amino]ethyl}-2-(trifluoromethyl)benzonitrile (Example 234) and morpholine: MS (ESI) m/z 436 (M+1).

### Example 238

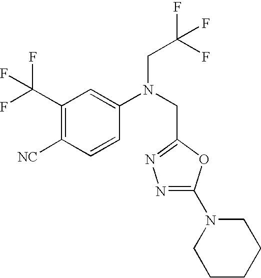

### 4-{2-[5-(1-Piperidinyl)-1,3,4-oxadiazol-2-yl]-1-[(trifluoromethyl)amino]ethyl}-2-(trifluoromethyl)benzonitrile

Synthesized as described in Example 236 from piperidine: MS (ESI) m/z 434 (M+1).

### Example 239

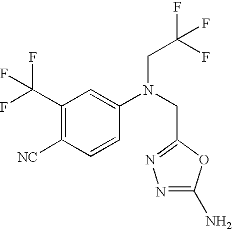

### 4-{2-(5-Amino-1,3,4-oxadiazol-2-yl)-1-[(trifluoromethyl)amino]ethyl}-2-(trifluoromethyl)benzonitrile

To NaHCO₃ (0.075 g, 0.89 mmol) in H₂O (2 mL) was added 2-[[4-cyano-3-(trifluoromethyl)phenyl](2,2,2-trifluoroethyl)amino]acetohydrazide, Example 223A, (0.3 g, 0.88 mmol) in dioxane (3 mL). After 5 min, CNBr (0.115 g, 1.089 mmol) was added and the resulting mixture was stirred at rt for 1 h. The mixture was partitioned between EtOAc/H₂O. The organic portion was washed with brine, dried over Na₂SO₄, filtered and concentrated in vacuo. Purification over SiO₂ eluting with EtOAc/hexanes afforded the title compound (0.028 g): MS (APCI) m/z 366 (M+1).

### Example 240

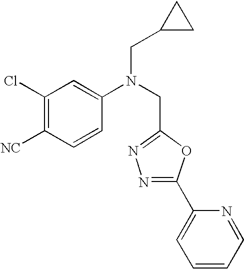

### 2-Chloro-4-((cyclopropylmethyl){[5-(2-pyridinyl)-1,3,4-oxadiazol-2-yl]methyl}amino)benzonitrile

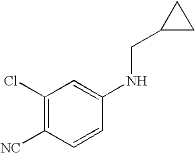

### A. 2-Chloro-4-[(cyclopropylmethyl)amino]benzonitrile

Synthesized in a manner similar to Example 1A using 2-chloro-4-fluorobenzonitrile: MS (ES) m/z 207 (M+1).

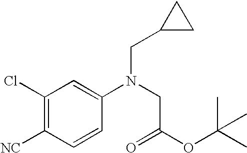

### B. 1,1-Dimethylethyl N-(3-chloro-4-cyanophenyl)-N-(cyclopropylmethyl)glycinate

Synthesized in a manner similar to Example 1B using 2-chloro-4-[(cyclopropylmethyl)amino]benzonitrile and t-butyl bromoacetate: MS (ES) m/z 321 (M+1).

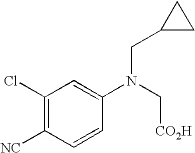

### C. N-(3-Chloro-4-cyanophenyl)-N-(cyclopropylmethyl)glycine

Synthesized in a manner similar to Example 129B using 1,1-dimethylethyl N-(3-chloro-4-cyanophenyl)-N-(cyclopropylmethyl)glycinate: MS (ES) m/z 265 (M+1).

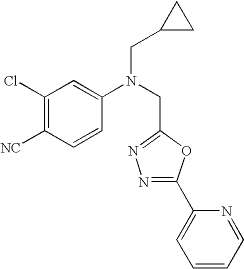

### D. 2-Chloro-4-((cyclopropylmethyl){[5-(2-pyridinyl)-1,3,4-oxadiazol-2-yl]methyl}amino)benzonitrile

To N-(3-chloro-4-cyanophenyl)-N-(cyclopropylmethyl)glycine (0.103 g, 0.392 mmol) in a microwave vial, was added CH₃CN (3 mL) followed by EDCl (0.083 g, 0.431 mmol) and 2-pyridinecarbohydrazide (0.056 g, 0.412 mmol). The reaction mixture was stirred for 30 min. To this solution was added THF (3 mL) followed by tosyl chloride (0.090 g, 0.47 mmol) and P-BEMP (0.89 g, 1.96 mmol, 2.2 mmol/g resin load). The reaction vessel was sealed and heated to 100° C. in microwave for 5 min. The resin was filtered off, washed twice with CH₂Cl₂ and concentrated via rotary evaporator. Purification via silica gel eluting with 25% EtOAc/hexanes afforded the title compound (0.032 g): MS (ESI) m/z 366 (M+1).

### Example 241

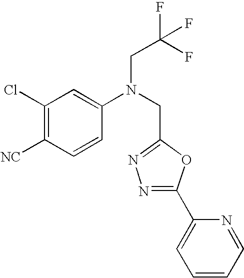

### 2-Chloro-4-[{[5-(2-pyridinyl)-1,3,4-oxadiazol-2-yl]methyl}(2,2,2-trifluoroethyl)amino]benzonitrile

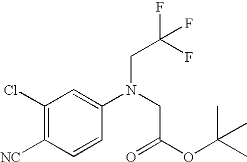

### A. 1,1-Dimethylethyl N-(3-chloro-4-cyanophenyl)-N-(2,2,2-trifluoroethyl)glycinate

Synthesized in a manner similar to Example 1B using 2-chloro-4-[(2,2,2-trifluoroethyl)amino]benzonitrile (Example 122A) and t-butyl bromoacetate: MS (ES) m/z 349 (M+1).

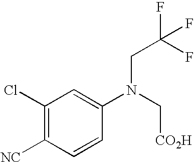

### B. N-(3-Chloro-4-cyanophenyl)-N-(2,2,2-trifluoroethyl)glycine

Synthesized in a manner similar to Example 129B using 1,1-dimethylethyl N-(3-chloro-4-cyanophenyl)-N-(2,2,2-trifluoroethyl)glycinate: MS (ES) m/z 293 (M+1).

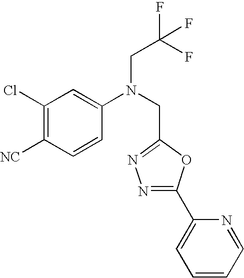

### C. 2-Chloro-4-[{[5-(2-pyridinyl)-1,3,4-oxadiazol-2-yl]methyl}(2,2,2-trifluoroethyl)amino]benzonitrile

To N-(3-chloro-4-cyanophenyl)-N-(2,2,2-trifluoroethyl)glycine (0.114 g, 0.392 mmol) in a microwave vial, was added CH₃CN (3 mL) followed by EDCl (0.083 g, 0.431 mmol) and 2-pyridinecarbohydrazide (0.056 g, 0.412 mmol). The reaction mixture was stirred for 30 min. To this solution, was then added THF (3 mL) followed by tosyl chloride (0.090 g, 0.47 mmol) and P-BEMP (0.89 g, 1.96 mmol, 2.2 mmol/g resin load). The reaction vessel was sealed and heated to 100° C. in microwave for 5 min. The resin was filtered off, washed twice with CH₂Cl₂ and concentrated via rotary evaporator. Purification via silica gel eluting with 25% EtOAc/hexanes afforded the title compound (0.01 g): MS (ESI) m/z 394 (M+1).

### Example 242

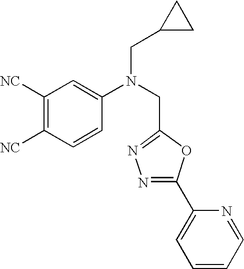

### 4-((Cyclopropylmethyl){[5-(2-pyridinyl)-1,3,4-oxadiazol-2-yl]methyl}amino)-1,2-benzenedicarbonitrile

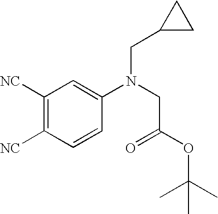

### A. 1,1-Dimethylethyl N-(cyclopropylmethyl)-N-(3,4-dicyanophenyl)glycinate

Synthesized in a manner similar to Example 1B using Example 123A and 1,1-dimethylethyl bromoacetate: MS (ES) m/z 312 (M+1).

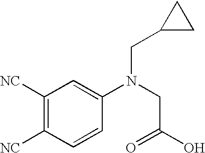

### B. N-(Cyclopropylmethyl)-N-(3,4-dicyanophenyl)glycine

Synthesized in a manner similar to Example 129B using 1,1-dimethylethyl N-(cyclopropylmethyl)-N-(3,4-dicyanophenyl)glycinate: MS (ES) m/z 256 (M+1).

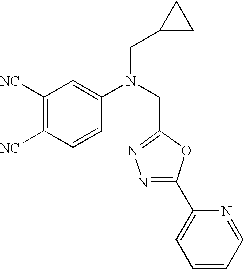

### C. 4-((Cyclopropylmethyl){([5-(2-pyridinyl)-1,3,4-oxadiazol-2-yl]methyl}amino)-1,2-benzenedicarbonitrile

Synthesized as described for Example 192 using Example 242B and 2-pyridinecarbohydrazide: MS (ES) m/z 357 (M+1).

### Example 243

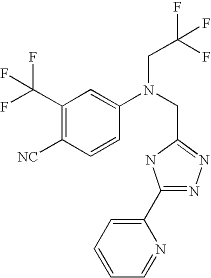

### 4-[{[3-(2-Pyridinyl)-1H-1,2,4-triazol-5-yl]methyl}(2,2,2-trifluoroethyl)amino]-2-(trifluoromethyl)benzonitrile

To 4-[{[5-(2-pyridinyl)-1,3,4-oxadiazol-2-yl]methyl}(2,2,2-trifluoroethyl)amino]-2-(trifluoromethyl)benzonitrile, Example 192, (0.1 g, 0.234 mmol) in a microwave vial, was added EtOH (1.5 mL), 2 M NH₃ in MeOH (0.0012 mL, 2.34 mmol) followed by MgCl₂ (0.002 g, 0.0234 mmol) and vial was sealed. The reaction mixture was heated to 150° C. in microwave for 2 h. Partitioning between EtOAc/H₂O, was followed by washing the organic layer with brine, drying over Na₂SO₄, filtration and concentration in vacuo. Purification via silica gel eluting with 25% EtOAc/hexanes afforded the title compound (0.013 g): MS (APCI) m/z 427 (M+1).

### Example 244

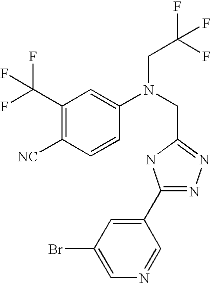

### 4-[{[3-(5-Bromo-3-pyridinyl)-1H-1,2,4-triazol-5-yl]methyl}(2,2,2-trifluoroethyl)amino]-2-(trifluoromethyl)benzonitrile

Synthesized as described in Example 243 from 4-[{[5-(5-bromo-3-pyridinyl)-1,3,4-oxadiazol-2-yl]methyl}(2,2,2-trifluoroethyl)amino]-2-(trifluoromethyl)benzonitrile (Example 193): MS (APCI) m/z 505 (M+1).

### Example 245

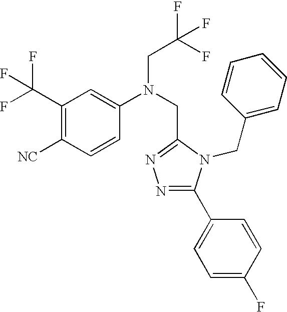

### 4-[{[5-(4-Fluorophenyl)-4-(phenylmethyl)-4H-1,2,4-triazol-3-yl]methyl}(2,2,2-trifluoroethyl)amino]-2-(trifluoromethyl)benzonitrile

To 4-[{[5-(4-fluorophenyl)-1,3,4-oxadiazol-2-yl]methyl}(2,2,2-trifluoroethyl)amino]-2-(trifluoromethyl)benzonitrile, Example 199, (0.07 g, 0.158 mmol) in toluene (0.5 mL), was added benzyl amine (0.2 mL, 0.79 mmol) and MgCl₂ (0.004 g, 0.04 mmol). The reaction was heated to 150° C. in a microwave for 3 h. The mixture was partitioned between EtOAc/H₂O, and the organic layer was washed with brine, dried over Na₂SO₄, filtered and concentrated in vacuo. Purification via silica gel eluting with 25% EtOAc/hexanes afforded the title compound (0.037 g): MS (ESI) m/z 534 (M+1).

### Example 246

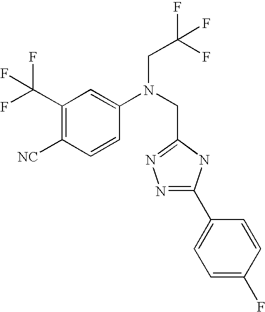

### 4-[{[3-(4-Fluorophenyl)-1H-1,2,4-triazol-5-yl]methyl}(2,2,2-trifluoroethyl)amino]-2-(trifluoromethyl)benzonitrile

A mixture of 4-[{[5-(4-Fluorophenyl)-4-(phenylmethyl)-4H-1,2,4-triazol-3-yl]methyl}(2,2,2-trifluoroethyl)amino]-2-(trifluoromethyl)benzonitrile (Example 245) (0.032 g, 0.06 mmol) and 20% Pd(OH)₂/C (0.005 g, 0.006 mmol) in EtOAc (5 mL) was hydrogenated (balloon) for 2 h. Filtration over a bed of celite was followed by concentration and purification (SiO₂, eluting with EtOAc/hexanes) to afford the title compound (0.016 g): MS (ESI) m/z 444 (M+1).

### Example 247

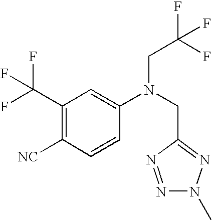

### 4-[[(2-Methyl-2H-tetrazol-5-yl)methyl](2,2,2-trifluoroethyl)amino]-2-(trifluoromethyl)benzonitrile

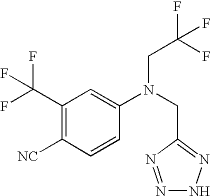

### A. 4-[(1H-Tetrazol-5-ylmethyl)(2,2,2-trifluoroethyl)amino]-2-(trifluoromethyl)benzonitrile

A mixture of 4-[(cyanomethyl)(2,2,2-trifluoroethyl)amino]-2-(trifluoromethyl)benzonitrile, Example 129D (0.050 g, 0.16 mmol), sodium azide (0.013 g, 0.20 mmol), lithium chloride (0.004 g, 0.09 mmol) and ammonium chloride (0.010 g, 0.19 mmol) in DMF (2 ml) was heated under N₂ at 125° C. for 3 h. After cooling, the mixture was partitioned between Et₂O and 0.2 N NaOH. The phases were separated and the organic phase was discarded. The aqueous phase was acidified with 2N HCl and extracted with Et₂O. The organic phase was washed with brine, dried (Na₂SO₄), filtered and concentrated. The crude mixture was crystallized from CH₂Cl₂/hexanes to give the title compound as a white solid (0.049 g, 86% yield): MS (ES) m/z 349 (M−1).

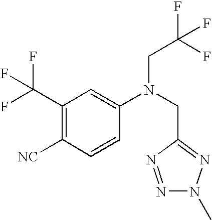

### B. 4-[[(2-Methyl-2H-tetrazol-5-yl)methyl](2,2,2-trifluoroethyl)amino]-2-(trifluoromethyl)benzonitrile

To a solution of 4-[(1H-tetrazol-5-ylmethyl)(2,2,2-trifluoroethyl)amino]-2-(trifluoromethyl)benzonitrile, Example 247A (0.050 g, 0.14 mmol) in MeOH (2 mL) was added trimethylsilyldiazomethane (2M in hexanes, 0.08 mL, 0.16 mmol). The reaction was followed by TLC and additional trimethylsilyldiazomethane was added accordingly. After completion, the mixture was concentrated and partitioned between EtOAc and water. The organic phase was washed with brine, dried (Na₂SO₄) and concentrated. The crude mixture was purified by silica gel chromatography (10-70% EtOAc-hexanes gradient) and the product crystallized from CH₂Cl₂-hexanes to give the title compound as a white solid (0.033 g, 63% yield): MS (ES) m/z 365 (M+1).

### Biological Section

Compounds of the current invention are modulators of the androgen receptor. Additionally, the compounds of the present invention may also prove useful as modulators of the glucocorticoid receptor, the mineralocorticoid receptor, and/or the progesterone receptor. Activity mediated through oxosteroid nuclear receptors was determined using the following in vitro and in vivo assays.

### In Vitro Assays:

The following abbreviations and sources of materials are used
Fluormone PL Red—a commercially available PR fluoroprobe (PanVera Corp, Product No P2965)
Fluormone GS Red—a commercially available GR fluoroprobe (PanVera Corp, Product No P2894)
Fluormone AL Green—a commercially available AR fluoroprobe (PanVera Corp, Product No P3010)
PR-LBD—Purified human progesterone ligand binding domain tagged with Glutathione Transferase (PanVera Corp, Product No P2900)
GR—purified human glucocorticoid receptor (PanVera Corp, Product No P2812)
AR-LBD—Purified rat androgen ligand binding domain tagged with Glutathione Transferase (PanVera Corp, Product No P3009)
PR Screening Buffer—100 mM potassium phosphate (pH 7.4), 100 μG/ml bovine gamma globulin, 15% ethylene glycol, 0.02% NaN₃, 10% glycerol (PanVera Corp Product No P2967) with 0.1% w/v CHAPS
AR Screening Buffer—pH 7.5 containing protein stabilizing agents and glycerol (PanVera Corp Product No P3011)
GR Screening Buffer—100 mM potassium phosphate (pH 7.4), 200 mM Na₂MoO₂, 1 mM EDTA, 20% DMSO (PanVera Corp Product No P2814) with GR stabilizing peptide (100 μM) (PanVera Corp Product No P2815)
DTT—dithiothreitol (PanVera Corp Product No P2325)
Discovery Analyst—is an FP reader
DMSO—dimethylsulphoxide

### Progesterone Receptor Fluorescence Polarization Assay (PR-FPA):

The progesterone receptor fluorescence polarization assay is used to investigate the interaction of the compounds with the progesterone receptor.

Compounds are added to the 384 well black plates to a final volume of 0.5 μL. Sufficient Fluormone PL Red and PR-LBD are defrosted on ice to give a final concentration of 2 nM and 40 nM, respectively. PR screening buffer is chilled to 4° C. prior to addition of DTT to give a final concentration of 1 mM. The Fluormone PL Red and PR-LBD in PR Screening Buffer are added to compound plates to give a final volume of 10 μL. The assay is allowed to incubate at 20-22° C. for 2 hours. The plates are counted in a Discovery Analyst with suitable 535 nM excitation and 590 nM emission interference filters. Compounds that interact with the PR result in a lower fluorescence polarization reading. Test compounds are dissolved and diluted in DMSO. Compounds are assayed in singlicate, a four parameter curve fit of the following form being applied

$$y = \frac{a - d}{1 + {( x / c )}^{b}} + d$$

where a is the minimum, b is the Hill slope, c is the IC₅₀ and d is the maximum. Maximum and minimum values are compared to adhesion in the absence of compound and in the presence of 10⁻⁵M progesterone. Data is presented as the mean pIC₅₀ with the standard error of the mean of n experiments. Compounds with pIC₅₀ greater than 5.0 and a % max greater than 50 are preferred.

### Androgen Receptor Fluorescence Polarization Assay (AR-FPA):

The androgen receptor fluorescence polarization assay is used to investigate the interaction of the compounds with the androgen receptor.

Compounds are added to the 384 well black plates to a final volume of 0.5 μL. Sufficient Fluormone AL Green and AR-LBD are defrosted on ice to give a final concentration of 1 nM and 25 nM, respectively. AR screening buffer is chilled to 4° C. prior to addition of DTT to give a final concentration of 1 mM. The Fluormone AL Green and AR-LBD in AR Screening Buffer are added to compound plates to give a final volume of 10 μL. The assay is allowed to incubate at 20° C. for 5 hours. The plates are counted in a Discovery Analyst with suitable 485 nM excitation and 535 nM emission interference filters. Compounds that interact with the AR result in a lower fluorescence polarization reading. Test compounds are dissolved and diluted in DMSO. Compounds are assayed in singlicate, a four parameter curve fit of the following form being applied

$$y = \frac{a - d}{1 + {( x / c )}^{b}} + d$$

where a is the minimum, b is the Hill slope, c is the IC₅₀ and d is the maximum. Maximum and minimum values are compared to adhesion in the absence of compound and in the presence of 10⁻⁵M dihydrotestosterone. Data is presented as the mean pIC₅₀ with the standard error of the mean of n experiments. Compounds with pIC₅₀ greater than 5.0 and a % max greater than 50 are preferred.

| | pIC50 in AR-FPA (as |
|---|---|
| Example Number | described above) |
| 1-115 | ≧5.0 |

### Glucocorticoid Receptor Fluorescence Polarization Assay (GR-FPA):

The glucocorticoid receptor fluorescence polarization assay is used to investigate the interaction of the compounds with the glucocorticoid receptor.

Compounds are added to the 384 well black plates to a final volume of 0.5 μL. Sufficient Fluormone GS Red and GR are defrosted on ice to give a final concentration of 1 nM and 4 nM, respectively. GR screening buffer is chilled to 4° C. prior to addition of DTT to give a final concentration of 1 mM. The Fluormone GS Red, and GR in GR Screening Buffer are added to compound plates to give a final volume of 10 μL. The assay is allowed to incubate at 4° C. for 12 hours. The plates are counted in a Discovery Analyst with suitable 535 nM excitation and 590 nM emission interference filters. Compounds that interact with the GR result in a lower fluorescence polarization reading. Test compounds are dissolved and diluted in DMSO. Compounds are assayed in singlicate, a four parameter curve fit of the following form being applied

$$y = \frac{a - d}{1 + {( x / c )}^{b}} + d$$

where a is the minimum, b is the Hill slope, c is the EC₅₀ and d is the maximum. Maximum and minimum values are compared to adhesion in the absence of compound and in the presence of 10⁻⁵M dexamethasone. Data is presented as the mean pIC₅₀ with the standard error of the mean of n experiments. Compounds with pIC₅₀ greater than 5.0 and a % max greater than 50 are preferred.

### Transient Transfection Assay:

Cotransfection assays using full-length human AR were performed in CV-1 cells (monkey kidney fibroblasts). The cells were seeded in charcoal-stripped medium in 96-well plates (24,000 cells/well) and incubated overnight. Transient transfections were carried out using the following plasmids: pSG5-AR, MMTV LUC reporter, β-actin SPAP, and pBluescript (filler DNA). The cell plates were then incubated for 6-20 hours. The transfection mixture was washed away and then the cells were drugged with doses ranging from 10⁻¹⁰ to 10⁻⁵. Two replicates were used for each sample. Incubation with drug was continued for 14 hours. A spectrophotometer was used for SPAP measurements, while a topcounter was used to read the results from the luciferase assay. The ratio of luciferase activity to SPAP activity was calculated to normalize the variance in cell number and transfection efficiency.

### Data Analysis:

Data were reduced using RoboFit99. The results were expressed as percent of maximum as calculated by the following formulas:

$$fold activation = \frac{\begin{matrix}( ( ( Luc ) / ( S P A P - S P A P substrate blank avg . ) ) - \\ basal activation )\end{matrix}}{basal {activation}^{*}}$$

*basal activation per plate=(Luc vehicle)/(SPAP vehicle−substrate blank average)% max.=(^{fold activation of unknown}/_{positive control fold activation avg.})×100

Curves were fit from these data using RoboFit to determine EC₅₀'s for agonists and IC₅₀'s for antagonists using the following equation:

Y=((Vmax*x)/(K+x))+Y2

These values were converted to pEC₅₀'s and pIC₅₀'s for posting by using the following equations:

pEC₅₀=−log(EC₅₀)

pIC₅₀=−log(IC₅₀)

For antagonist assays, the percent maximum response antagonist was calculated by the following formula in which Yₘᵢₙ and Yₘₐₓ are curve asymptotes at the maximum or minimum concentration tested:

% max. resp. ant.=100*(1−Yₘᵢₙ/Yₘₐₓ)

For antagonist assays, pKb's were calculated using the following formula:

pKb=IC₅₀ of unknown/((1+*conc.*)/DHT EC₅₀ average)

where *conc.*=concentration of DHT used as the agonist in the medium for the antagonist experiment, expressed in nM. This concentration was set at twice pEC₅₀. This would be 0.2 for AR.

Compounds with a pXC₅₀ greater than 5.0 are preferred.

### Castrated Male Rat Model (ORX Rat)

The activity of the compounds of the present invention as modulators of the androgen receptor was investigated using a castrated male rat model (ORX) as described in C. D. Kockakian, Pharmac. Therap. B 1(2), 149-177 (1975); C. Tobin and Y. Joubert, Developmental Biology 146, 131-138 (1991); J. Antonio, J. D. Wilson and F. W. George, J Appl. Physiol. 87(6) 2016-2019 (1999)) the disclosures of which herein are included by reference.

Androgens have been identified as playing important roles in the maintenance and growth of many tissues in both animals and humans. Muscles, like the levator ani and bulbocavernosus, and sexual accessory organs, such as the prostate glands and seminal vesicles have high expression levels of the androgen receptor and are known to respond quickly to exogenous androgen addition or androgen deprivation through testicular ablation. Castration produces dramatic atrophy of muscle and sexual accessory organs; whereas the administration of exogenous androgens to the castrated animal results in effective hypertrophy of these muscles and sexual accessory organs. Although the levator ani muscle, also known as the dorsal bulbocavernosus, is not ‘true skeletal muscle’ and definitely sex-linked, it is reasonable to use this muscle to screen muscle anabolic activities of test compounds because of its androgen responsiveness and simplicity of removal.

Male Sprague-Dawley rats weighing 160-180 grams were used in the assay. The rats were singly caged upon receiving and throughout the study. Bilateral orchidectomies were performed in sterilized surgical conditions under isoflurane anesthesia. An anteroposterior incision was made in the scrotum. The testicles were exteriorized and the spermatic artery and vas deferens were ligated with 4.0 silk 0.5 cm proximal to the ligation site. The testicles then were removed by a surgical scissors distal to the ligation sites. The tissue stumps were returned to the scrotum, the scrotum and overlying skin were closed by a surgical stapler. The Sham-ORX rats underwent all procedures except ligation and scissors cutting. The rats were assigned randomly into study groups 7-10 days post surgery based on the body weight.

Dihydrotestosterone (DHT) was used as a positive control (1-10 mg/kg s.c.). Compounds of the current invention were administered subcutaneously or orally for 4-28 days. The rats were weighed daily and doses were adjusted accordingly. The general well being of the animal was monitored throughout the course of the study.

At the end of the study, the rats were euthanized in a CO₂ chamber. The ventral prostate glands (VP), seminal vesicles (SV), levator ani muscle (LA) and bulbocavernosus (BC) were carefully dissected. The tissues were blotted dry, the weights were recorded, and then saved for histological and molecular analysis. The VP and SV weights serve as androgenic indicators and LA and BC as anabolic indicators. The ratio of anabolic to androgenic activities was used to evaluate the test compounds. Serum luteinizing hormone (LH), follicle stimulating hormone (FSH) and other potential serum markers of anabolic activities were also analyzed.

In general, preferred compounds show levator ani hypertrophy and very little prostate stimulation.

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included within the scope of the appended claims.

1. A compound of formula (I): 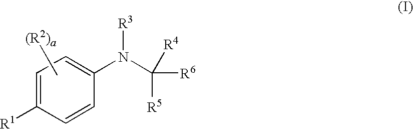
or a salt or solvate thereof, wherein
R¹ is CN;
a is 1;
R² is CF₃;
R³ is —(R^{x})_{f}R⁷;
f is 1;
R^{x} is a C₁-C₂ alkylene chain that may be further optionally substituted with one or more alkyl;
R⁷ is C₁-C₆ alkyl, C₁-C₆ haloalkyl, or C₁-C₆;
each R⁴ and R⁶ independently is H, alkyl, cycloalkyl, or haloalkyl;
R⁶ is aryl, or heterocyclyl, wherein
(i) when R⁶ is aryl, said aryl is optionally substituted with one or more alkyl, halogen, haloalkyl, alkoxy, haloalkoxy, haloalkylthio, heterocyclyl, cyano, alkylsulfonyl, alkoxycarbonyl, nitro, amide, aryl, aryloxy, substituted aryl or substituted aryloxy, where said substituted aryl or substituted aryloxy is substituted with one or more halogen, and
(ii) when R⁶ is heterocyclyl, said heterocyclyl may be optionally substituted with one or more alkyl, cycloalkylalkyl, halogen, haloalkyl, alkoxy, haloalkoxyalkyl, halogen substituted arylsulfonylalkyl, alkoxycarbonyl, oxo, cyano, cyanoalkyl, hydroxyalkyl, amide, amino, aminoalkyl, alkylsulfonyl, aralkoxyalkyl, acyl, heterocyclylcarbonyl, alkoxyalkyl, alkylthioalkyl, aralkyl, heterocyclylalkyl, heterocyclyl, aryl, R⁸, substituted aryl, or substituted heterocyclyl, where said halogen, haloalkyl, cyano, alkoxy, amino, nitro, alkylthio, alkylsulfonyl, alkylsulfoxide, haloalkoxy, alkoxycarbonyl, amide, acyl, heterocyclylcarbonyl, aminosulfonyl, or —NHC(O)CH₃, or
(iii) R⁴ and R⁶ combine with the carbon from which they are substituted, to form a 6- to 12-membered bicyclic ring, which ring may optionally contain one or more heteroatoms selected from O, S, and N;
R⁸ is —(CH₂)ₙ—(R⁹)—(R¹⁰);
n is 0, 1 or 2;
R⁹ is —C(O)—, —S(O)₂, —NHC(O)—, —NHC(O)NHC(O)—, or —NHS(O)₂—; and
R¹⁰ is NH₂, CH₃, alkoxy, or aryl, wherein said aryl may optionally be substituted with alkyl or halogen.

2. A compound of formula (I-A): 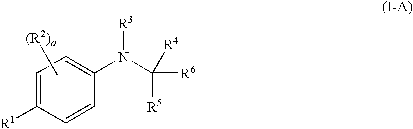
or a salt or solvate thereof, wherein
R¹ is CN;
a is 1;
R² is CF₃;
R³ is —(R^{x})_{f}R⁷;
f is 1;
R^{x} is a C₁-C₄ alkylene chain that may be further optionally substituted with one or more alkyl;
R⁷ is C₁-C₆ alkyl, C₁-C₆ haloalkyl, or C₁-C₆;
each R⁴ and R⁵ independently is H, alkyl, cycloalkyl, or haloalkyl;
R⁶ is aryl, or heterocyclyl, wherein
(i) when R⁶ is aryl, said aryl is optionally substituted with one or more alkyl, halogen, haloalkyl, alkoxy, haloalkoxy, haloalkylthio, heterocyclyl, cyano, alkylsulfonyl, alkoxycarbonyl, nitro, amide, aryl, aryloxy, substituted aryl or substituted aryloxy, where said substituted aryl or substituted aryloxy is substituted with one or more halogen, and
(ii) when R⁶ is heterocyclyl, said heterocyclyl may be optionally substituted with one or more alkyl, cycloalkylalkyl, halogen, haloalkyl, alkoxy, haloalkoxyalkyl, halogen substituted arylsulfonylalkyl, alkoxycarbonyl, oxo, cyano, cyanoalkyl, hydroxyalkyl, amide, amino, aminoalkyl, alkylsulfonyl, aralkoxyalkyl, acyl, heterocyclylcarbonyl, alkoxyalkyl, alkylthioalkyl, aralkyl, heterocyclylalkyl, heterocyclyl, aryl, R⁸, substituted aryl, or substituted heterocyclyl, where said substituted aryl or substituted heterocyclyl is substituted with one or more alkyl, halogen, haloalkyl, cyano, alkoxy, amino, nitro, alkylthio, alkylsulfonyl, alkylsulfoxide, haloalkoxy, alkoxycarbonyl, amide, acyl, heterocyclylcarbonyl, aminosulfonyl, or —NHC(O)CH₃;
R⁸ is —(CH₂)ₙ—(R⁹)—(R¹⁰);
n is 0, 1 or 2;
R⁹ is —C(O)—, —S(O)₂, —NHC(O)—, —NHC(O)NHC(O)—, or —NHS(O)₂—; and
R¹⁰ is NH₂, CH₃, alkoxy, or aryl, wherein said aryl may optionally be substituted with alkyl or halogen.

3-6. (canceled)

7. The compound of claim 1 wherein R⁷ is —CF₃.

8. The compound of claim 1 wherein R⁷ is cyclopropyl.

9. The compound of claim 1 wherein each of R⁴ and R⁵ are H.

10. The compound of claim 1 wherein R⁶ is aryl, or heterocyclyl.

11. The compound of claim 1 wherein R⁶ is aryl.

12. The compound of claim 11 wherein R⁶ is optionally substituted with one or more halogen, C₁-C₆ haloalkyl, or cyano.

13. The compound of claim 1 wherein R⁶ is heterocyclyl.

14. The compound of claim 13 wherein R⁶ is furyl, thiazolyl, oxazolyl, oxadiazolyl, tetrazolyl, triazolyl, thiophenyl, benzofuranyl, isoxazolyl, benzothiazolyl, imidazolyl, benzothiophenyl, or benzimidazolyl.

15. The compound of claim 14 wherein R⁶ is optionally substituted with one or more cyano, alkyl, haloalkyl, aryl, halogen, or heterocyclyl.

16. The compound of claim 13 wherein R⁶ is furyl, thiazolyl, oxazolyl, oxadiazolyl, tetrazolyl.

17. The compound of claim 16 wherein R⁶ is optionally substituted with one or more cyano, alkyl, haloalkyl, aryl, halogen, or heterocyclyl.

18. The compound of claim 17 wherein said aryl is phenyl optionally substituted with one or more cyano, halogen or haloalkyl.

19. The compound of claim 17 wherein said heterocyclyl is pyridyl, isoxazolyl, furyl, thiophenyl, pyrazolyl, or piradazyl, each independently optionally substituted with one or more cyano, halogen or haloalkyl.

20. The compound of claim 17 wherein said heterocyclyl is piperidyl optionally substituted with aminosulfonyl.

21. A compound selected from:
4-{(Cyclopropylmethyl)[(1R)-1-(2-naphthyl)ethyl]amino}-2-(trifluoromethyl)benzonitrile;
4-{[(1R)-1-Phenylpropyl]amino}-2-(trifluoromethyl)benzonitrile;
4-{(Cyclopropylmethyl)[(1S)-2,3-dihydro-1H-inden-1-yl]amino}-2-(trifluoromethyl)benzonitrile;
N-[(1R)-1-(4-Bromophenyl)ethyl]-4-nitro-3-(trifluoromethyl)aniline;
4-{(Cyclopropylmethyl)[(1R)-2,3-dihydro-1H-inden-1-yl]amino}-2-(trifluoromethyl)benzonitrile;
4-[Benzyl(1-cyclopropylethyl)amino]-2-(trifluoromethyl)benzonitrile;
4-{Allyl[(1R)-1-(4-bromophenyl)ethyl]amino}-2-(trifluoromethyl)benzonitrile;
N-Allyl-N-[(1R)-1-(4-bromophenyl)ethyl]-4-nitro-3-(trifluoromethyl)aniline;
4-{Allyl[(1S)-2,3-dihydro-1H-inden-1-yl]amino}-2-(trifluoromethyl)benzonitrile;
4-[Benzyl(2,2,2-trifluoroethyl)amino]-2-(trifluoromethyl)benzonitrile;
4-[(2-Methylbenzyl)(2,2,2-trifluoroethyl)amino]-2-(trifluoromethyl)benzonitrile;
4-{(2,2,2-Trifluoroethyl)[2-(trifluoromethyl)benzyl]amino}-2-(trifluoromethyl)benzonitrile;
4-[(2-Fluorobenzyl)(2,2,2-trifluoroethyl)amino]-2-(trifluoromethyl)benzonitrile;
4-[(3-Methylbenzyl)(2,2,2-trifluoroethyl)amino]-2-(trifluoromethyl)benzonitrile;
4-{(2,2,2-Trifluoroethyl)[3-(trifluoromethyl)benzyl]amino}-2-(trifluoromethyl)benzonitrile;
4-[(3-Fluorobenzyl)(2,2,2-trifluoroethyl)amino]-2-(trifluoromethyl)benzonitrile;
4-[(3-Chlorobenzyl)(2,2,2-trifluoroethyl)amino]-2-(trifluoromethyl)benzonitrile;
4-[(3-Bromobenzyl)(2,2,2-trifluoroethyl)amino]-2-(trifluoromethyl)benzonitrile;
4-[1,1′-Biphenyl-3-ylmethyl)(2,2,2-trifluoroethyl)amino]-2-(trifluoromethyl)benzonitrile;
4-[(3-Methoxybenzyl)(2,2,2-trifluoroethyl)amino]-2-(trifluoromethyl)benzonitrile;
4-{(2,2,2-Trifluoroethyl)[3-(trifluoromethoxy)benzyl]amino}-2-(trifluoromethyl)benzonitrile;
4-[[3-(4-Fluorophenoxy)benzyl](2,2,2-trifluoroethyl)amino]-2-(trifluoromethyl)benzonitrile;
4-[[3-(2-Fluorophenoxy)benzyl](2,2,2-trifluoroethyl)amino]-2-(trifluoromethyl)benzonitrile;
4-((2,2,2-Trifluoroethyl){3-[(trifluoromethyl)thio]benzyl}amino)-2-(trifluoromethyl)benzonitrile;
4-[(3-Cyanobenzyl)(2,2,2-trifluoroethyl)amino]-2-(trifluoromethyl)benzonitrile;
Methyl 3-{[[4-cyano-3-(trifluoromethyl)phenyl](2,2,2-(trifluoroethyl)amino]methyl}benzoate;
3-{[[4-Cyano-3-(trifluoromethyl)phenyl](2,2,2-(trifluoroethyl)amino]methyl}benzamide;
4-[(3-Nitrobenzyl)(2,2,2-trifluoroethyl)amino]-2-(trifluoromethyl)benzonitrile;
4-[(4-Methylbenzyl)(2,2,2-trifluoroethyl)amino]-2-(trifluoromethyl)benzonitrile;
4-[(4-tert-Butylbenzyl)(2,2,2-trifluoroethyl)amino]-2-(trifluoromethyl)benzonitrile;
4-{(2,2,2-Trifluoroethyl)[4-(trifluoromethyl)benzyl]amino}-2-(trifluoromethyl)benzonitrile;
4-[(4-Fluorobenzyl)(2,2,2-trifluoroethyl)amino]-2-(trifluoromethyl)benzonitrile;
4-[(4-Methoxybenzyl)(2,2,2-trifluoroethyl)amino]-2-(trifluoromethyl)benzonitrile;
4-{(2,2,2-Trifluoroethyl)[4-(trifluoromethoxy)benzyl]amino}-2-(trifluoromethyl)benzonitrile;
4-[[4-(Methylsulfonyl)benzyl](2,2,2-trifluoroethyl)amino]-2-(trifluoromethyl)benzonitrile;
4-[(4-Cyanobenzyl)(2,2,2-trifluoroethyl)amino]-2-(trifluoromethyl)benzonitrile;
Methyl 4-{[[4-cyano-3-(trifluoromethyl)phenyl](2,2,2-trifluoroethyl)amino]methyl}benzoate;
4-[(4-Nitrobenzyl)(2,2,2-trifluoroethyl)amino]-2-(trifluoromethyl)benzonitrile;
4-[(3,5-Dimethylbenzyl)(2,2,2-trifluoroethyl)amino]-2-(trifluoromethyl)benzonitrile;
4-[[3,5-bis(Trifluoromethyl)benzyl](2,2,2-trifluoroethyl)amino]-2-(trifluoromethyl)benzonitrile;
4-[(3,5-Dimethoxybenzyl)(2,2,2-trifluoroethyl)amino]-2-(trifluoromethyl)benzonitrile;
4-[3,5-Dichlorobenzyl)(2,2,2-trifluoroethyl)amino]-2-(trifluoromethyl)benzonitrile;
4-[(3,5-Difluorobenzyl)(2,2,2-trifluoroethyl)amino]-2-(trifluoromethyl)benzonitrile;
4-[(2,6-Difluorobenzyl)(2,2,2-trifluoroethyl)amino]-2-(trifluoromethyl)benzonitrile;
4-[(2,4-Difluorobenzyl)(2,2,2-trifluoroethyl)amino]-2-(trifluoromethyl)benzonitrile;
4-[(3,4-Difluorobenzyl)(2,2,2-trifluoroethyl)amino]-2-(trifluoromethyl)benzonitrile;
4-[(2,3-Difluorobenzyl)(2,2,2-trifluoroethyl)amino]-2-(trifluoromethyl)benzonitrile;
4-[[3-Fluoro-4-(trifluoromethyl)benzyl](2,2,2-trifluoroethyl)amino]-2-(trifluoromethyl)benzonitrile;
4-[(2-Naphthylmethyl)(2,2,2-trifluoroethyl)amino]-2-(trifluoromethyl)benzonitrile;
4-[(1-Phenylethyl)(2,2,2-trifluoroethyl)amino]-2-(trifluoromethyl)benzonitrile;
4-[(3-Furylmethyl)(2,2,2-trifluoroethyl)amino]-2-(trifluoromethyl)benzonitrile;
4-[(2-Furylmethyl)(2,2,2-trifluoroethyl)amino]-2-(trifluoromethyl)benzonitrile;
Ethyl 5-{[[4-cyano-3-(trifluoromethyl)phenyl](2,2,2-trifluoroethyl)amino]methyl}-2-furoate;
5-{[[4-Cyano-3-(trifluoromethyl)phenyl](2,2,2-trifluoroethyl)amino]methyl}-2-furamide;
5-{[[4-Cyano-3-(trifluoromethyl)phenyl](2,2,2-trifluoroethyl)amino]methyl}-2-furonitrile;
4-[[(5-Methyl-2-furyl)methyl](2,2,2-trifluoroethyl)amino]-2-(trifluoromethyl)benzonitrile;
4-((2,2,2-Trifluoroethyl){[5-(trifluoromethyl)-2-furyl]methyl}amino)-2-(trifluoromethyl)benzonitrile;
4-[{[5-(Cyanomethyl)-2-furyl]methyl}(2,2,2-trifluoroethyl)amino]-2-(trifluoromethyl)benzonitrile;
4-[{[5-(Methoxymethyl)-2-furyl]methyl}(2,2,2-trifluoroethyl)amino]-2-(trifluoromethyl)benzonitrile;
4-[({5-[(2,2,2-Trifluoroethoxy)methyl]-2-furyl}methyl)(2,2,2-trifluoroethyl)amino]-2-(trifluoromethyl)benzonitrile;
4-[({5-[(Methylthio)methyl]-2-furyl}methyl)(2,2,2-trifluoroethyl)amino]-2-(trifluoromethyl)benzonitrile;
4-[({5-[(Dimethylamino)methyl]-2-furyl}methyl)(2,2,2-trifluoroethyl)amino]-2-(trifluoromethyl)benzonitrile;
4-[{[5-(1H-Imidazol-1-ylmethyl)-2-furyl]methyl}(2,2,2-trifluoroethyl)amino]-2-(trifluoromethyl)benzonitrile;
4-[{[5-(1H-1,2,4-Triazol-1-ylmethyl)-2-furyl]methyl}(2,2,2-trifluoroethyl)amino]-2-(trifluoromethyl)benzonitrile;
4-[{[5-(2-Cyanoethyl)-2-furyl]methyl}(2,2,2-trifluoroethyl)amino]-2-(trifluoromethyl)benzonitrile;
4-[{[5-(3,5-Difluorophenyl)-2-furanyl]methyl}(2,2,2-trifluoroethyl)amino]-2-(trifluoromethyl)benzonitrile;
4-[[(5-Phenyl-2-furanyl)methyl](2,2,2-trifluoroethyl)amino]-2-(trifluoromethyl)benzonitrile;
4-[{[5-(4-Fluorophenyl)-2-furanyl]methyl}(2,2,2-trifluoroethyl)amino]-2-(trifluoromethyl)benzonitrile;
4-[{[5-(3,4-Difluorophenyl)-2-furanyl]methyl}(2,2,2-trifluoroethyl)amino]-2-(trifluoromethyl)benzonitrile;
4-[{[5-(2,4-Difluorophenyl)-2-furanyl]methyl}(2,2,2-trifluoroethyl)amino]-2-trifluoromethyl)benzonitrile;
4-{(2,2,2-Trifluoroethyl)[5-{4-[(trifluoromethyl)oxy]phenyl}-2-furanyl)methyl]amino}-2-(trifluoromethyl)benzonitrile;
N-[4-(5-{[[4-Cyano-3-(trifluoromethyl)phenyl](2,2,2-trifluoroethyl)amino]methyl}-2-furanyl)phenyl]acetamide;
4-[{[5-(4-Cyanophenyl)-2-furanyl]methyl}(2,2,2-trifluoroethyl)amino]-2-(trifluoromethyl)benzonitrile;
4-[({5-[4-(Methylsulfonyl)phenyl]-2-furanyl}methyl)(2,2,2-trifluoroethyl)amino]-2-(trifluoromethyl)benzonitrile;
4-[{[5-(3-Cyanophenyl)-2-furanyl]methyl}(2,2,2-trifluoroethyl)amino]-2-(trifluoromethyl)benzonitrile;
5-(5-{[[4-Cyano-3-(trifluoromethyl)phenyl](2,2,2-trifluoroethyl)amino]methyl}-2-furanyl)-2-thiophenecarbonitrile;
4-[{[5-(5-Pyrimidinyl)-2-furanyl]methyl}(2,2,2-trifluoroethyl)amino]-2-(trifluoromethyl)benzonitrile;
4-[{[5-(3,5-Dimethyl-4-isoxazolyl)-2-furanyl]methyl}(2,2,2-trifluoroethyl)amino]-2-(trifluoromethyl)benzonitrile;
4-[{[4-(4-Cyanophenyl)-2-furanyl]methyl}(2,2,2-trifluoroethyl)amino]-2-(trifluoromethyl)benzonitrile;
Methyl 2-{[[4-cyano-3-(trifluoromethyl)phenyl](2,2,2-trifluoroethyl)amino]methyl}-3-furoate;
2-{[[4-Cyano-3-(trifluoromethyl)phenyl](2,2,2-trifluoroethyl)amino]methyl}-3-furamide;
2-{[[4-Cyano-3-(trifluoromethyl)phenyl](2,2,2-trifluoroethyl)amino]methyl}-3-furonitrile;
4-[{[3-(Hydroxymethyl)-2-furyl]methyl}(2,2,2-trifluoroethyl)amino]-2-(trifluoromethyl)benzonitrile;
4-[[(3-Methyl-2-furyl)methyl](2,2,2-trifluoroethyl)amino]-2-(trifluoromethyl)benzonitrile;
4-[(1-Benzofuran-2-ylmethyl)(2,2,2-trifluoroethyl)amino]-2-(trifluoromethyl)benzonitrile;
4-[[(5-Chlorothien-2-yl)methyl](2,2,2-trifluoroethyl)amino]-2-(trifluoromethyl)benzonitrile;
4-[(Thien-2-ylmethyl)(2,2,2-trifluoroethyl)amino]-2-(trifluoromethyl)benzonitrile;
4-[[(5-Bromothien-2-yl)methyl](2,2,2-trifluoroethyl)amino]-2-(trifluoromethyl)benzonitrile;
5-{[[4-Cyano-3-(trifluoromethyl)phenyl](2,2,2-trifluoroethyl)amino]methyl}thiophene-2-carbonitrile;
4-[{[5-(4-Fluorophenyl)thien-2-yl]methyl}(2,2,2-trifluoroethyl)amino]-2-(trifluoromethyl)benzonitrile;
4-[{[5-(4-Cyanophenyl)thien-2-yl]methyl}(2,2,2-trifluoroethyl)amino]-2-(trifluoromethyl)benzonitrile;
4-[(Thien-3-ylmethyl)(2,2,2-trifluoroethyl)amino]-2-(trifluoromethyl)benzonitrile;
4-[[(5-Chloro-1-benzothien-3-yl)methyl](2,2,2-trifluoroethyl)amino]-2-(trifluoromethyl)benzonitrile;
4-[(1H-Imidazol-2-ylmethyl)(2,2,2-trifluoroethyl)amino]-2-(trifluoromethyl)benzonitrile;
1,1-Dimethylethyl 4-{[[4-cyano-3-(trifluoromethyl)phenyl](2,2,2-trifluoroethyl)amino]methyl}-1H-imidazole-1-carboxylate;
4-[(1H-Imidazol-4-ylmethyl)(2,2,2-trifluoroethyl)amino]-2-(trifluoromethyl)benzonitrile;
4-[(1,3-Oxazol-2-ylmethyl)(2,2,2-trifluoroethyl)amino]-2-(trifluoromethyl)benzonitrile;
4-[(1,3-Oxazol-4-ylmethyl)(2,2,2-trifluoroethyl)amino]-2-(trifluoromethyl)benzonitrile;
4-[{[4-(Hydroxymethyl)-1,3-oxazol-2-yl]methyl}(2,2,2-trifluoroethyl)amino]-2-(trifluoromethyl)benzonitrile;
2-{[[4-Cyano-3-(trifluoromethyl)phenyl](2,2,2-trifluoroethyl)amino]methyl}-1,3-oxazole-4-carbonitrile;
2-{[[4-Cyano-3-(trifluoromethyl)phenyl](2,2,2-trifluoroethyl)amino]methyl}-1,3-oxazole-5-carbonitrile;
4-{[[(5-([(Phenylmethyl)oxy]methyl}-1,3-oxazol-2-yl)methyl](2,2,2-trifluoroethyl)amino]-2-(trifluoromethyl)benzonitrile;
4-[{[2-(4-Fluorophenyl)-1,3-oxazol-4-yl]methyl}(2,2,2-trifluoroethyl)amino]-2-(trifluoromethyl)benzonitrile;
4-[[(4-Phenyl-1,3-oxazol-2-yl)methyl](2,2,2-trifluoroethyl)amino]-2-(trifluoromethyl)benzonitrile;
5-{[[4-Cyano-3-(trifluoromethyl)phenyl](2,2,2-trifluoroethyl)amino]methyl}-1,3-oxazole-2-carbonitrile;
4-[{[2-(4-Fluorophenyl)-1,3-oxazol-5-yl]methyl}(2,2,2-trifluoroethyl)amino]-2-(trifluoromethyl)benzonitrile;
4-[{[5-(4-Fluorophenyl)-1,3-oxazol-2-yl]methyl}(2,2,2-trifluoroethyl)amino]-2-(trifluoromethyl)benzonitrile;
4-[(1,3-Oxazol-5-ylmethyl)(2,2,2-trifluoroethyl)amino]-2-(trifluoromethyl)benzonitrile;
4-[[(3,5-Dimethylisoxazol-4-yl)methyl](2,2,2-trifluoroethyl)amino]-2-(trifluoromethyl)benzonitrile;
4-[[(5-Methylisoxazol-3-yl)methyl](2,2,2-trifluoroethyl)amino]-2-(trifluoromethyl)benzonitrile;
4-[(5-Isoxazolylmethyl)(2,2,2-trifluoroethyl)amino]-2-(trifluoromethyl)benzonitrile;
4-[(Cyclopropylmethyl)(1,3-thiazol-4-ylmethyl)amino]-2-(trifluoromethyl)benzonitrile;
4-[(1,3-Thiazol-4-ylmethyl)(2,2,2-trifluoroethyl)amino]-2-(trifluoromethyl)benzonitrile;
4-[[(Methyl-1,3-thiazol-4-yl)methyl](2,2,2-trifluoroethyl)amino]-2-(trifluoromethyl)benzonitrile;
4-[[(2-Phenyl-1,3-thiazol-4-yl)methyl](2,2,2-trifluoroethyl)amino]-2-(trifluoromethyl)benzonitrile;
4-[(2,2,2-Trifluoroethyl)({2-[4-(trifluoromethyl)phenyl]-1,3-thiazol-4-yl}methyl)amino]-2-(trifluoromethyl)benzonitrile;
4-[[(2-Thien-2-yl-1,3-thiazol-4-yl)methyl](2,2,2-trifluoroethyl)amino]-2-(trifluoromethyl)benzonitrile;
4-[(1,3-Benzothiazol-2-ylmethyl)(2,2,2-trifluoroethyl)amino]-2-(trifluoromethyl)benzonitrile;
4-[(1,3-Thiazol-5-ylmethyl)(2,2,2-trifluoroethyl)amino]-2-(trifluoromethyl)benzonitrile;
4-[(1,3-Thiazol-2-ylmethyl)(2,2,2-trifluoroethyl)amino]-2-(trifluoromethyl)benzonitrile;
4-[[1-(1,3-Thiazol-2-yl)ethyl](2,2,2-trifluoroethyl)amino]-2-(trifluoromethyl)benzonitrile;
2-Chloro-4-[(1,3-thiazol-4-ylmethyl)(2,2,2-trifluoroethyl)amino]benzonitrile;
4-[(Cyclopropylmethyl)(1,3-thiazol-4-ylmethyl)amino]-1,2-benzenedicarbonitrile;
4-[(1,3-Thiazol-4-ylmethyl)(2,2,2-trifluoroethyl)amino]1,2-benzenedicarbonitrile;
4-[[(5-tert-Butyl-1,2,4-oxadiazol-3-yl)methyl](2,2,2-trifluoroethyl)amino]-2-(trifluoromethyl)benzonitrile;
4-[[(5-Phenyl-1,2,4-oxadiazol-3-yl)methyl](2,2,2-trifluoroethyl)amino]-2-(trifluoromethyl)benzonitrile;
4-[{[5-(2-Methoxyphenyl)-1,2,4-oxadiazol-3-yl]methyl}(2,2,2-trifluoroethyl)amino]-2-(trifluoromethyl)benzonitrile;
4-[(2,2,2-Trifluoroethyl)({5-[3-(trifluoromethyl)phenyl]-1,2,4-oxadiazol-3-yl}methyl)amino]-2-(trifluoromethyl)benzonitrile;
4-(3-{[[4-Cyano-3-(trifluoromethyl)phenyl](2,2,2-trifluoroethyl)amino]methyl}-1,2,4-oxadiazol-5-yl)-1-piperidinesulfonamide;
4-[(1,2,4-Oxadiazol-3-ylmethyl)(2,2,2-trifluoroethyl)amino]-2-(trifluoromethyl)benzonitrile;
4-[{[5-(1-Methylethyl)-1,2,4-oxadiazol-3-yl]methyl}(2,2,2-trifluoroethyl)amino]-2-(trifluoromethyl)benzonitrile;
4-[[(5-Methyl-1,2,4-oxadiazol-3-yl)methyl](2,2,2-trifluoroethyl)amino]-2-(trifluoromethyl)benzonitrile;
4-[{[5-(1-Methylpropyl)-1,2,4-oxadiazol-3-yl]methyl}(2,2,2-trifluoroethyl)amino]-2-(trifluoromethyl)benzonitrile;
4-((2,2,2-Trifluoroethyl){[5-(trifluoromethyl)-1,2,4-oxadiazol-3-yl]methyl}amino)-2-(trifluoromethyl)benzonitrile;
4-[[(5-Oxo-2,5-dihydro-1,2,4-oxadiazol-3-yl)methyl](2,2,2-trifluoroethyl)amino]-2-(trifluoromethyl)benzonitrile;
4-[{[5-(4-Fluorophenyl)-1,2,4-oxadiazol-3-yl]methyl}(2,2,2-trifluoroethyl)amino]-2-(trifluoromethyl)benzonitrile;
4-{(2,2,2-Trifluoroethyl)[(5-{4-[(trifluoromethyl)oxy]phenyl}-1,2,4-oxadiazol-3-(yl)methyl]amino}-2-(trifluoromethyl)benzonitrile;
4-[(2,2,2-Trifluoroethyl)({5-[4-(trifluoromethyl)phenyl]-1,2,4-oxadiazol-3-yl}methyl)amino]-2-(trifluoromethyl)benzonitrile;
4-{(2,2,2-Trifluoroethyl)[(5-{3-[(trifluoromethyl)oxy]phenyl}-1,2,4-oxadiazol-3-yl)methyl]amino}-2-(trifluoromethyl)benzonitrile;
4-[{[5-(5-Isoxazolyl)-1,2,4-oxadiazol-3-yl]methyl}(2,2,2-trifluoroethyl)amino]-2-(trifluoromethyl)benzonitrile;
4-[{[5-(2-Thienyl)-1,2,4-oxadiazol-3-yl]methyl}(2,2,2-trifluoroethyl)amino]-2-(trifluoromethyl)benzonitrile;
4-[{[5-(3,5-Dimethyl-4-isoxazolyl)-1,2,4-oxadiazol-3-yl]methyl}(2,2,2-trifluoroethyl)amino]-2-(trifluoromethyl)benzonitrile;
1,1-Dimethylethyl[(3-{[[4-cyano-3-(trifluoromethyl)phenyl](2,2,2-trifluoroethyl)amino]methyl}-1,2,4-oxadiazol-5-yl)methyl]carbamate;
4-[{[5-(Aminomethyl)-1,2,4-oxadiazol-3-yl]methyl}(2,2,2-trifluoroethyl)amino]-2-(trifluoromethyl)benzonitrile;
N-[(3-{[[4-Cyano-3-(trifluoromethyl)phenyl](2,2,2-trifluoroethyl)amino]methyl}-1,2,4-oxadiazol-5-yl)methyl]acetamide;
N-[(3-{[[4-Cyano-3-(trifluoromethyl)phenyl](2,2,2-trifluoroethyl)amino]methyl}-1,2,4-oxadiazol-5-yl)methyl]methanesulfonamide;
N-[(3-{[[4-Cyano-3-(trifluoromethyl)phenyl](2,2,2-trifluoroethyl)amino]methyl}-1,2,4-oxadiazol-5-yl)methyl]urea;
N-[(3-{[[4-cyano-3-(trifluoromethyl)phenyl](2,2,2-trifluoroethyl)amino]methyl}-1,2,4-oxadiazol-5-yl)methyl]dicarabonimidic diamide;
1,1-Dimethylethyl[2-(3-{[[4-cyano-3-(trifluoromethyl)phenyl](2,2,2-trifluoroethyl)amino]methyl}-1,2,4-oxadiazol-5-yl)ethyl]carbamate;
4-[{[5-(2-Aminoethyl)-1,2,4-oxadiazol-3-yl]methyl}(2,2,2-trifluoroethyl)amino]-2-(trifluoromethyl)benzonitrile;
N-[2-(3-{[[4-Cyano-3-(trifluoromethyl)phenyl](2,2,2-trifluoroethyl)amino]methyl}-1,2,4-oxadiazol-5-yl)ethyl]acetamide;
N-[2-(3-{[[4-Cyano-3-(trifluoromethyl)phenyl](2,2,2-trifluoroethyl)amino]methyl}-1,2,4-oxadiazol-5-yl)ethyl]methanesulfonamide;
N-[2-(3-{[[4-Cyano-3-(trifluoromethyl)phenyl](2,2,2-trifluoroethyl)amino]methyl}-1,2,4-oxadiazol-5-yl)ethyl]urea;
N-[2-(3-{[[4-cyano-3-(trifluoromethyl)phenyl](2,2,2-trifluoroethyl)amino]methyl}-1,2,4-oxadiazol-5-yl)ethyl]dicarbonimidic diamide;
4-[{[5-(1-Acetyl-4-piperidinyl)-1,2,4-oxadiazol-3-yl]methyl}(2,2,2-trifluoroethyl)amino]-2-(trifluoromethyl)benzonitrile;
4-[({5-[1-(5-Isoxazolylcarbonyl)-4-piperidinyl]-1,2,4-oxadiazol-3-yl}methyl)(2,2,2-trifluoroethyl)amino]-2-(trifluoromethyl)benzonitrile;
Methyl 4-(3-{[[4-cyano-3-(trifluoromethyl)phenyl](2,2,2-trifluoroethyl)amino]methyl}-1,2,4-oxadiazol-5-yl)-1-piperidinecarboxylate;
4-(3-{[[4-Cyano-3-(trifluoromethyl)phenyl](2,2,2-trifluoroethyl)amino]methyl}-1,2,4-oxadiazol-5-yl)-1-piperidinecarboxamide;
4-(3-{[[4-Cyano-3-(trifluoromethyl)phenyl](2,2,2-trifluoroethyl)amino]methyl}-1,2,4-oxadiazol-5-yl)-N-methyl-1-piperidinecarboxamide;
4-[({5-[1-(Methylsulfonyl)-4-piperidinyl]-1,2,4-oxadiazol-3-yl}methyl)(2,2,2-trifluoroethyl)amino]-2-(trifluoromethyl)benzonitrile;
4-[{[5-(1-Methyl-4-piperidinyl)-1,2,4-oxadiazol-3-yl]methyl}(2,2,2-trifluoroethyl)amino]-2-(trifluoromethyl)benzonitrile;
1,1-Dimethylethyl 3-(3-{[[4-cyano-3-(trifluoromethyl)phenyl](2,2,2-trifluoroethyl)amino]methyl}-1,2,4-oxadiazol-5-yl)-1-piperidinecarboxylate;
4-[{[5-(3-Piperidinyl)-1,2,4-oxadiazol-3-yl]methyl}(2,2,2-trifluoroethyl)amino]-2-(trifluoromethyl)benzonitrile;
4-[{[5-(1-Acetyl-3-piperidinyl)-1,2,4-oxadiazol-3-yl]methyl}(2,2,2-trifluoroethyl)amino]-2-(trifluoromethyl)benzonitrile;
Methyl 3-(3-{[[4-cyano-3-(trifluoromethyl)phenyl](2,2,2-trifluoroethyl)amino]methyl}-1,2,4-oxadiazol-5-yl)-1-piperidinecarboxylate;
3-(3-{[[4-Cyano-3-(trifluoromethyl)phenyl](2,2,2-trifluoroethyl)amino]methyl}-1,2,4-oxadiazol-5-yl)-1-piperidinecarboxamide;
3-(3-{[[4-Cyano-3-(trifluoromethyl)phenyl](2,2,2-trifluoroethyl)amino]methyl}-1,2,4-oxadiazol-5-yl)-N-methyl-1-piperidinecarboxamide;
4-[({5-[1-(Methylsulfonyl)-3-piperidinyl]-1,2,4-oxadiazol-3-yl}methyl)(2,2,2-trifluoroethyl)amino]-2-(trifluoromethyl)benzonitrile;
4-[{[5-(Phenylcarbonyl)-1,2,4-oxadiazol-3-yl]methyl}(2,2,2-trifluoroethyl)amino]-2-(trifluoromethyl)benzonitrile;
4-[{[5-(2-Furanylcarbonyl)-1,2,4-oxadiazol-3-yl]methyl}(2,2,2-trifluoroethyl)amino]-2-(trifluoromethyl)benzonitrile;
4-[(2,2-Dimethylpropyl)({5-[3-(trifluoromethyl)phenyl]-1,2,4-oxadiazol-3-yl}methyl)amino]-2-(trifluoromethyl)benzonitrile;
4-[{[5-(2-Pyridinyl)-1,2,4-oxadiazol-3-yl]methyl}(2,2,2-trifluoroethyl)amino]-2-(trifluromethyl)benzonitrile;
4-[{[5-(Cyclopropylmethyl)-1,2,4-oxadiazol-3-yl]methyl}(2,2,2-trifluoroethyl)amino]-2-(trifluoromethyl)benzonitrile;
4-((2,2,2-Trifluoroethyl){[5-(2,2,2-trifluoroethyl)-1,2,4-oxadiazol-3-yl]methyl}amino)-2-(trifluoromethyl)benzonitrile;
4-[({5-[(Dimethylamino)methyl]-1,2,4-oxadiazol-3-yl}methyl)(2,2,2-trifluoroethyl)amino]-2-(trifluoromethyl)benzonitrile;
4-[(2,2,2-Trifluoroethyl)({5-[6-(trifluoromethyl)-3-pyridinyl]-1,2,4-oxadiazol-3-yl}methyl)amino]-2-(trifluoromethyl)benzonitrile;
4-[{[3-(4-Fluorophenyl)-1,2,4-oxadiazol-5-yl]methyl}(2,2,2-trifluoroethyl)amino]-2-(trifluoromethyl)benzonitrile;
4-[{[3-(2-Pyridinyl)-1,2,4-oxadiazol-5-yl]methyl}(2,2,2-trifluoroethyl)amino]-2-(trifluoromethyl)benzonitrile;
4-[[(3-Phenyl-1,2,4-oxadiazol-5-yl)methyl](2,2,2-trifluoroethyl)amino]-2-(trifluoromethyl)benzonitrile;
4-[(2,2,2-Trifluoroethyl)({3-[4-(trifluormethyl)phenyl]-1,2,4-oxadiazol-5-yl}methyl)amino]-2-(trifluoromethyl)benzonitrile;
4-{(2,2,2-Trifluoroethyl)[(3-{4-[(trifluoromethyl)oxy]phenyl}-1,2,4-oxadiazol-5-yl)methyl]amino}-2-(trifluoromethyl)benzonitrile;
4-[({3-[4-(1,1-Dimethylethyl)phenyl]-1,2,4-oxadiazol-5-yl}methyl)(2,2,2-trifluoroethyl)amino]-2-(trifluoromethyl)benzonitrile;
4-[(2,2,2-Trifluoroethyl)({3-[3-(trifluoromethyl)phenyl]-1,2,4-oxadiazol-5-yl}methyl)amino]-2-(trifluoromethyl)benzonitrile;
4-[{[3-(3-Pyridinyl)-1,2,4-oxadiazol-5-yl]methyl}(2,2,2-trifluoroethyl)amino]-2-(trifluoromethyl)benzonitrile;
4-[({3-[3,5-Bis(trifluoromethyl)phenyl]-1,2,4-oxadiazol-5-yl}methyl)(2,2,2-trifluoroethyl)amino]-2-(trifluoromethyl)benzonitrile;
4-[{[3-(3-Nitrophenyl)-1,2,4-oxadiazol-5-yl]methyl}(2,2,2-trifluoroethyl)amino]-2-(trifluoromethyl)benzonitrile;
4-[[(3-{[(4-Fluorophenyl)sulfonyl]methyl}-1,2,4-oxadiazol-5-yl)methyl](2,2,2-trifluoroethyl)amino]-2-(trifluoromethyl)benzonitrile;
4-[(2,2,2-Trifluoroethyl)({3-[6-(trifluoromethyl)-3-pyridinyl]-1,2,4-oxadiazol-5-yl}methyl)amino]-2-(trifluoromethyl)benzonitrile;
4-[{[3-(2,4-Dichlorophenyl)-1,2,4-oxadiazol-5-yl]methyl}(2,2,2-trifluoroethyl)amino]-2-(trifluoromethyl)benzonitrile;
4-[{[3-(2,3-Dichlorophenyl)-1,2,4-oxadiazol-5-yl]methyl}(2,2,2-trifluoroethyl)amino]-2-(trifluoromethyl)benzonitrile;
4-[{[5-(4-Methylphenyl)-1,3,4-oxadiazol-2-yl]methyl}(2,2,2-trifluoroethyl)amino]-2-(trifluoromethyl)benzonitrile;
4-[{[5-(2-Pyridinyl)-1,3,4-oxadiazol-2-yl]methyl}(2,2,2-trifluoroethyl)amino]-2-(trifluoromethyl)benzonitrile;
4-[{[5-(5-Bromo-3-pyridinyl)-1,3,4-oxadiazol-2-yl]methyl}(2,2,2-trifluoroethyl)amino]-2-(trifluoromethyl)benzonitrile;
4-[{[(2-Chloro-4-pyridinyl)-1,3,4-oxadiazol-2-yl]methyl}(2,2,2-trifluoroethyl)amino]-2-(trifluoromethyl)benzonitrile;
4-[{[5-(6-Chloro-3-pyridinyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[6-(trifluoromethyl)-3-pyridinyl]-1,3,4-oxadiazol-2-yl}methyl)amino]-2-(trifluoromethyl)benzonitrile;
4-[{[5-(6-Chloro-2-pyridinyl)-1,3,4-oxadiazol-2-yl]methyl}(2,2,2-trifluoroethyl)amino]-2-(trifluoromethyl)benzonitrile;
4-[{[5-(6-Fluoro-2-pyridinyl)-1,3,4-oxadiazol-2-yl]methyl}(2,2,2-trifluoroethyl)amino]-2-(trifluoromethyl)benzonitrile;
4-[{[5-(4-Fluorophenyl)-1,3,4-oxadiazol-2-yl]methyl}(2,2,2-trifluoroethyl)amino]-2-(trifluoromethyl)benzonitrile;
4-[{[5-(3-Methylphenyl)-1,3,4-oxadiazol-2-yl]methyl}(2,2,2-trifluoroethyl)amino]-2-(trifluoromethyl)benzonitrile;
4-[{[5-(3-Pyridinyl)-1,3,4-oxadiazol-2-yl]methyl}(2,2,2-trifluoroethyl)amino]-2-(trifluoromethyl)benzonitrile;
4-[[(5-Phenyl-1,3,4-oxadiazol-2-yl)methyl](2,2,2-trifluoroethyl)amino]-2-(trifluoromethyl)benzonitrile;
4-[{[5-(2-Furanyl)-1,3,4-oxadiazol-2-yl]methyl}(2,2,2-trifluoroethyl)amino]-2-(trifluoromethyl)benzonitrile;
4-[{[5-(3-Furanyl)-1,3,4-oxadiazol-2-yl]methyl}(2,2,2-trifluoroethyl)amino]-2-(trifluoromethyl)benzonitrile;
4-[{[5-(4-Pyridinyl)-1,3,4-oxadiazol-2-yl]methyl}(2,2,2-trifluoroethyl)amino]-2-(trifluoromethyl)benzonitrile;
4-[({5-[4-(Dimethylamino)phenyl]-1,3,4-oxadiazol-2-yl}methyl)(2,2,2-trifluoroethyl)amino]-2-(trifluoromethyl)benzonitrile;
4-[{[5-(1,3-Benzodioxol-5-yl)-1,3,4-oxadiazol-2-yl]methyl}(2,2,2-trifluoroethyl)amino]-2-(trifluoromethyl)benzonitrile;
4-[{[5-(3-Thienyl)-1,3,4-oxadiazol-2-yl]methyl}(2,2,2-trifluoroethyl)amino]-2-(trifluoromethyl)benzonitrile;
4-[{[5-(4-Nitrophenyl)-1,3,4-oxadiazol-2-yl]methyl}(2,2,2-trifluoroethyl)amino]-2-(trifluoromethyl)benzonitrile;
4-{2-[5-(4-Aminophenyl)-1,3,4-oxadiazol-2-yl]-1-[(trifluoromethyl)amino]ethyl}-2-(trifluoromethyl)benzonitrile;
N-[4-(5-{2-[4-Cyano-3-(trifluoromethyl)phenyl]-2-[(trifluoromethyl)amino]ethyl}-1,3,4-oxadiazol-2-yl)phenyl]acetamide;
4-[{[5-(2-Methylphenyl)-1,3,4-oxadiazol-2-yl]methyl}(2,2,2-trifluoroethyl)amino]-2-(trifluoromethyl)benzonitrile;
4-[{[5-(3-Fluorophenyl)-1,3,4-oxadiazol-2-yl]methyl}(2,2,2-trifluoroethyl)amino]-2-(trifluoromethyl)benzonitrile;
4-[({5-[4-(Methyloxy)phenyl]-1,3,4-oxadiazol-2-yl}methyl)(2,2,2-trifluoroethyl)amino]-2-(trifluoromethyl)benzonitrile;
4-[({5-[4-(Methylthio)phenyl]-1,3,4-oxadiazol-2-yl}methyl)(2,2,2-trifluoroethyl)amino]-2-(trifluoromethyl)benzonitrile;
4-{2-{5-[4-(Methylsulfinyl)phenyl]-1,3,4-oxadiazol-2-yl}-1-[(trifluoromethyl)amino]ethyl}-2-(trifluoromethyl)benzonitrile;
4-{2-{5-[4-(Methylsulfonyl)phenyl]-1,3,4-oxadiazol-2-yl}-1-[(trifluoromethyl)amino]ethyl}-2-(trifluoromethyl)benzonitrile;
4-[{[5-(5-Bromo-2-furanyl)-1,3,4-oxadiazol-2-yl]methyl}(2,2,2-trifluoroethyl)amino]-2-(trifluoromethyl)benzonitrile;
4-{(2,2,2-Trifluoroethyl)[(5-{4-[trifluoromethyl)oxy]phenyl}-1,3,4-oxadiazol-2-yl)methyl]amino}-2-(trifluoromethyl)benzonitrile;
4-[{[5-(3,4-Difluorophenyl)-1,3,4-oxadiazol-2-yl]methyl}(2,2,2-trifluoroethyl)amino]-2-(trifluoromethyl)benzonitrile;
1,1-Dimethylethyl[(5-{[[4-cyano-3-(trifluoromethyl)phenyl](2,2,2-trifluoroethyl)amino]methyl}-1,3,4-oxadiazol-2-yl)methyl]carbamate;
4-[{[5-(3,5-Difluorophenyl)-1,3,4-oxadiazol-2-yl]methyl}(2,2,2-trifluoroethyl)amino]-2-(trifluoromethyl)benzonitrile;
4-[{[5-(6-Bromo-2-pyridinyl)-1,3,4-oxadiazol-2-yl]methyl}(2,2,2-trifluoroethyl)amino]-2-(trifluoromethyl)benzonitrile;
4-[{[5-(2,4-Difluorophenyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[2-(trifluoromethyl)phenyl]-1,3,4-oxadiazol-2-yl}methyl)amino]-2-(trifluoromethyl)benzonitrile;
4-[(2,2,2-Trifluoroethyl)({5-[3-(trifluoromethyl)phenyl]-1,3,4-oxadiazol-2-yl}methyl)amino]-2-(trifluoromethyl)benzonitrile;
4-[{[5-(2-Fluorophenyl)-1,3,4-oxadiazol-2-yl]methyl}(2,2,2-trifluoroethyl)amino]-2-(trifluoromethyl)benzonitrile;
4-[(2,2,2-Trifluoroethyl)({5-[4-(trifluoromethyl)phenyl]-1,3,4-oxadiazol-2-yl}methyl)amino]-2-(trifluoromethyl)benzonitrile;
4-[({5-[2-Chloro-6-(methyloxy)-4-pyridinyl]-1,3,4-oxadiazol-2-yl}methyl)(2,2,2-trifluoroethyl)amino]-2-(trifluoromethyl)benzonitrile;
4-[{[5-(1,3-Dimethyl-1H-pyrazol-5-yl)-1,3,4-oxadiazol-2-yl]methyl}(2,2,2-trifluoroethyl)amino]-2-(trifluoromethyl)benzonitrile;
4-[{[5-(2-Pyrazinyl)-1,3,4-oxadiazol-2-yl]methyl}(2,2,2-trifluoroethyl)amino]-2-(trifluoromethyl)benzonitrile;
4-[{[5-(5-Pyrimidinyl)-1,3,4-oxadiazol-2-yl]methyl}(2,2,2-trifluoroethyl)amino]-2-(trifluoromethyl)benzonitrile;
4-[{[5-(1-Methyl-1H-pyrazol-3-yl)-1,3,4-oxadiazol-2-yl]methyl}(2,2,2-trifluoroethyl)amino]-2-(trifluoromethyl)benzonitrile;
4-{2-(5-Oxo-4,5-dihydro-1,3,4-oxadiazol-2-yl)-1-[(trifluoromethyl)amino]ethyl}-2-(trifluoromethyl)benzonitrile;
4-{2-(4-Methyl-5-oxo-4,5-dihydro-1,3,4-oxadiazol-2-yl)-1-[(trifluoromethyl)amino]ethyl}-2-(trifluoromethyl)benzonitrile;
4-{2-[5-(Dimethylamino)-1,3,4-oxadiazol-2-yl]-2-[(trifluoromethyl)amino]ethyl}-2-(trifluoromethyl)benzonitrile;
4-{2-[5-(4-Morpholinyl)-1,3,4-oxadiazol-2-yl]-1-[(trifluoromethyl)amino]ethyl}-2-(trifluoromethyl)benzonitrile;
4-{2-[5-(1-Piperidinyl)-1,3,4-oxadiazol-2-yl]-1-[trifluoromethyl)amino]ethyl}-2-(trifluoromethyl)benzonitrile;
4-{2-(5-Amino-1,3,4-oxadiazol-2-yl)-1-[(trifluoromethyl)amino]ethyl}-2-(trifluoromethyl)benzonitrile;
{[5-(2-pyridinyl)-1,3,4-oxadiazol-2-yl]methyl}amino)benzonitrile;
2-Chloro-4-[{[5-(2-pyridinyl)-1,3,4-oxadiazol-2-yl]methyl}(2,2,2-trifluoroethyl)amino]benzonitrile;
4-((Cyclopropylmethyl){[5-(2-pyridinyl)-1,3,4-oxadiazol-2-yl]methyl}amino)-1,2-benzenedicarbonitrile;
4-[{[3-(2-Pyridinyl)-1H-1,2,4-triazol-5-yl]methyl}(2,2,2-trifluoroethyl)amino]-2-(trifluoromethyl)benzonitrile;
4-[{[3-(5-Bromo-3-pyridinyl)-1H-1,3,4-triazol-5-yl]methyl}(2,2,2-trifluoroethyl)amino]-2-(trifluoromethyl)benzonitrile;
4-[{[5-(4-Fluorophenyl)-4-phenylmethyl)-4H-1,2,4-triazol-3-yl]methyl}(2,2,2-trifluoroethyl)amino]-2-(trifluoromethyl)benzonitrile;
4-[{[3-(4-Fluorophenyl)-1H-1,2,4-triazol-5-yl]methyl}(2,2,2-trifluoroethyl)amino]-2-(trifluoromethyl)benzonitrile;
and 4-[[(2-Methyl-2H-tetrazol-5-yl)methyl](2,2,2-trifluoroethyl)amino]-2-(trifluoromethyl)benzonitrile; or a salt or solvate thereof.

22. A compound of formula I-B: 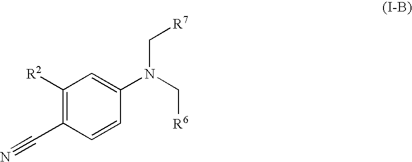
or a salt or solvate thereof, wherein
R² is —CF₃, cyano or chloro;
R⁷ is —CF₃ or cyclopropyl;
R⁶ is aryl or heterocyclyl, wherein
when R⁶ is aryl, said aryl may optionally be substituted with cyano, and
when R⁶ is heterocyclyl, said heterocyclyl, substituted aryl, or substituted heterocyclyl, wherein said substituted aryl or substituted heterocyclyl is substituted with one or more
halogen, haloalkyl, or aminosulfonyl.

23. The compound of claim 22 wherein R⁷ is —CF₃.

24. The compound of claim 22 wherein R⁷ is cyclopropyl.

25. The compound of claim 22 wherein R⁶ is heterocyclyl.

26. The compound of claim 25 wherein R⁶ is furyl, thiazolyl, oxazolyl, oxadiazolyl, or tetrazolyl.

27. The compound of claim 26 wherein R⁶ is optionally substituted with one or more cyano, alkyl, haloalkyl, aryl, or heterocyclyl.

28. The compound of claim 27 wherein said aryl is phenyl optionally substituted with one or more halogen or haloalkyl.

29. The compound of claim 27 wherein said heterocyclyl is pyridyl optionally substituted with one or more halogen or haloalkyl.

30. The compound of claim 27 wherein said heterocyclyl is piperidyl optionally substituted with aminosulfonyl.

31. A compound selected from: 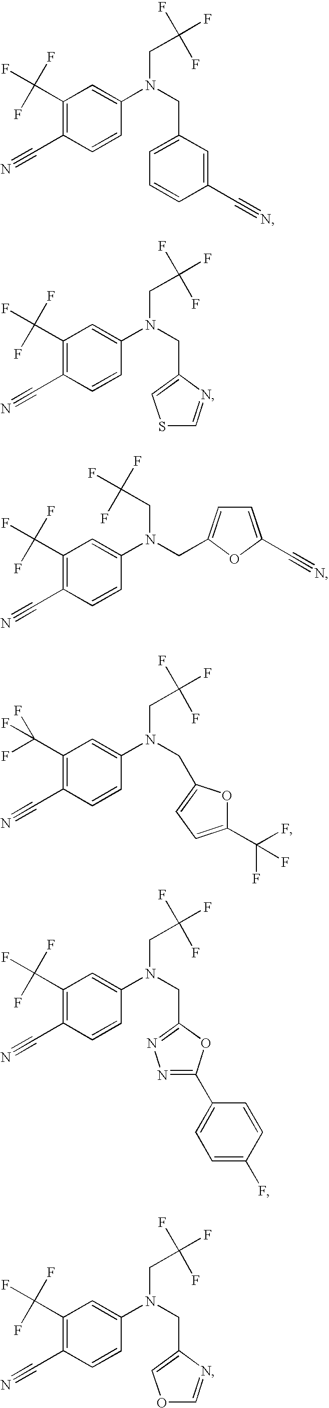 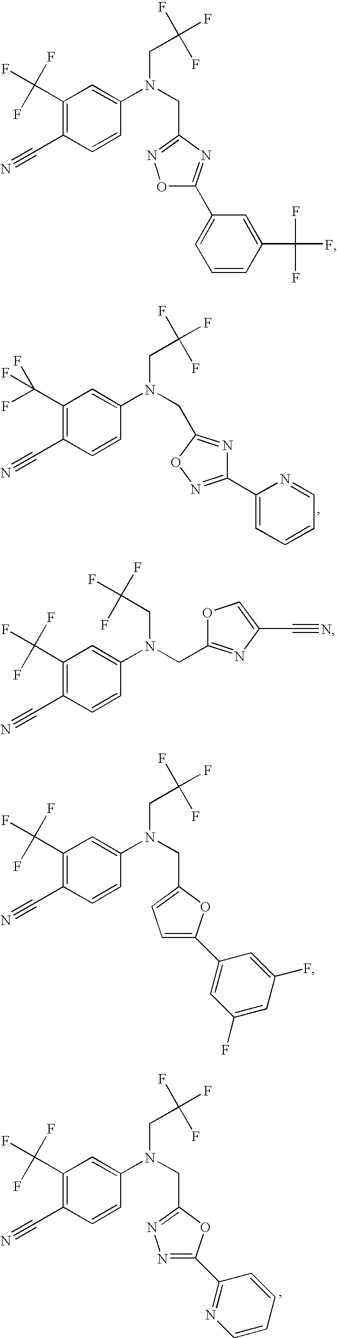 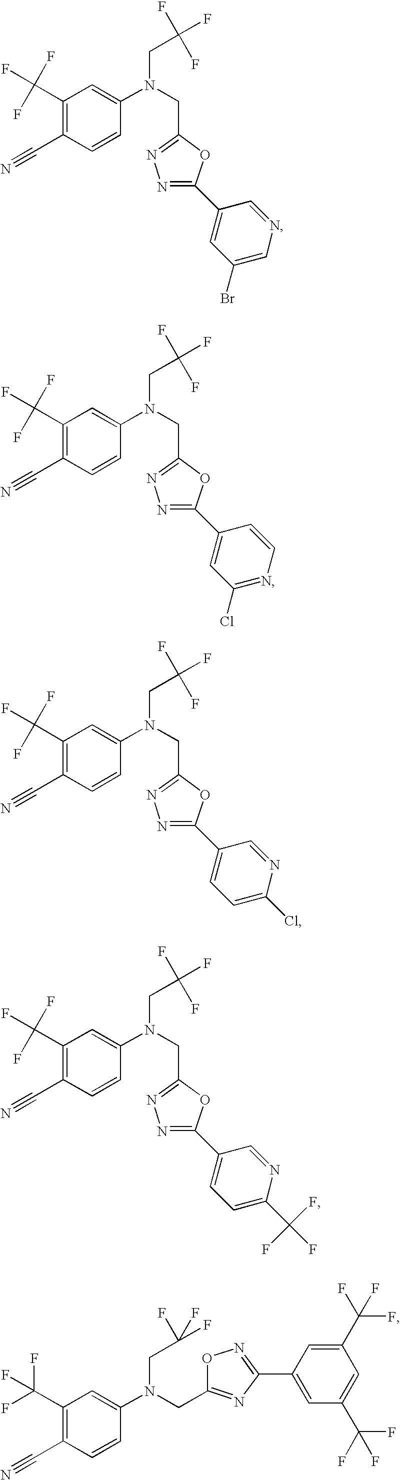 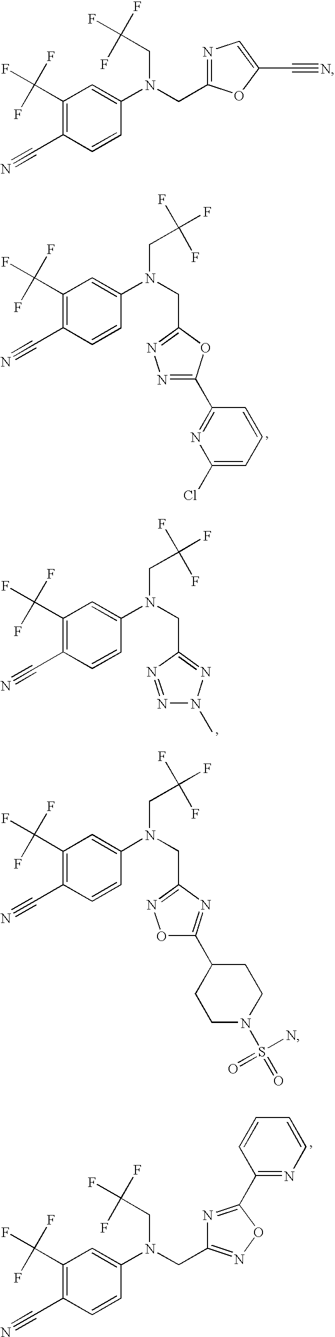 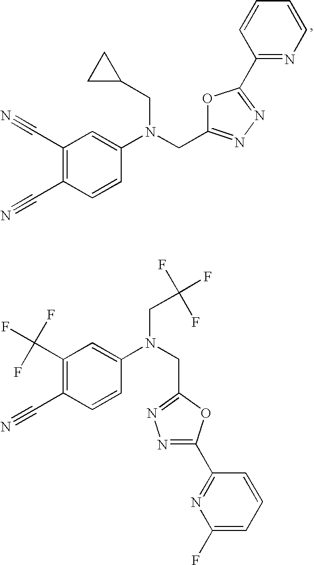
or a salt or solvate thereof.

32. (canceled)

33. A pharmaceutical composition comprising a compound according to claim 1.

34-40. (canceled)

41. A method for the treatment of osteoporosis, muscle wasting, frailty, prostatic hyperplasia, prostate cancer, dyslipidemia, libido enhancement, depression, or ADAM comprising the administration of a compound according to claim 1.
